(19) United States
(12) Patent Application Publication
(10) Pub. No.: US 2026/0325652 A1
(43) Pub. Date: Oct. 22, 2026

(54) NMDA LIGAND CONJUGATED COMPOUNDS AND USES THEREOF

(71) Applicant: ADARX PHARMACEUTICALS, INC., San Diego, CA (US)

(72) Inventors: Mehdi Michel Djamel Numa, San Diego, CA (US); Kin Fong, San Diego, CA (US); Huey-Jing Huang, San Diego, CA (US); Chandramouli Chiruta, San Diego, CA (US); Rui Zhu, San Diego, CA (US)

(73) Assignee: ADARX PHARMACEUTICALS, INC., San Diego, CA (US)

(21) Appl. No.: 19/483,386

(22) PCT Filed: May 10, 2024
(86) PCT No.: PCT/US2024/028958

§ 371 Date: Nov. 11, 2025

Related U.S. Application Data
(60) Provisional application No. 63/502,035, filed on May 12, 2023.

(51) Int. Cl. A61K 47/54; A61K 9/00; A61K 31/713; A61P 25/28

(52) U.S. Cl. CPC A61K 47/548; A61K 9/0085; A61K 31/713; A61P 25/28

(57) ABSTRACT

Provided herein are N-methyl-D-aspartate (NMDA) receptor ligand-containing compounds, methods of delivering said compounds, and methods of treating diseases, disorders, and symptoms (e.g., central nervous system diseases, disorders, and symptoms) in a subject using said compounds.

### RELATED APPLICATIONS

This application claims priority under 35 U.S.C. § 119(e) to U.S. Provisional Application, U.S. Ser. No. 63/502,035, filed May 12, 2023, the contents of which is incorporated herein by reference.

### BACKGROUND

In the use of compounds in therapeutic, prophylactic, or diagnostic applications, it is often desirable that the compounds be delivered to a specific location (for example, to desired cell(s)) to enhance the therapeutic or prophylactic effect or to be advantageous for diagnostic purposes. This is frequently the case when attempting to deliver a therapeutic compound in vivo. Further, being able to efficiently deliver a compound to a specific location can limit or potentially eliminate unintended consequences (such as off-target effects) that may be caused by administration of the compound. One strategy to facilitate delivery of a compound, such as a therapeutic, prophylactic, or diagnostic compound, to a desired location in vivo, is by linking or attaching the compound to a targeting ligand.

One class of compounds that can be targeted using targeting ligands are oligomeric compounds such as, for example, proteins, peptides, antibodies, and oligonucleotides. Oligomeric compounds that include nucleotide sequences (e.g., oligonucleotides) at least partially complementary to a target nucleic acid have been shown to alter the function and activity of the target both in vitro and in vivo. When delivered to a cell containing a target nucleic acid (such as mRNA or pre-mRNA), oligonucleotides have been shown to modulate the expression or activity of the target nucleic acid. In certain instances, the oligonucleotide can reduce the expression of the gene by inhibiting translation of the nucleic acid target and/or triggering the degradation of the target nucleic acid.

If the target nucleic acid is mRNA, one mechanism by which an oligonucleotide can modulate the expression of the mRNA target is through RNA interference. RNA interference is a biological process by which RNA or RNA-like molecules (such as chemically modified RNA molecules) are able to silence gene expression, at least in part, through the RNA-induced silencing Complex (RISC) pathway. Additionally, oligonucleotides can modulate the expression of a target nucleic acid, such as a target mRNA, through an RNase recruitment mechanism, microRNA mechanisms, occupancy-based mechanisms, and editing mechanisms. Oligonucleotides may be single-stranded or double-stranded. Oligonucleotides may comprise DNA, RNA, and RNA-like molecules, which can also include modified nucleosides including one or more modified sugars, modified nucleobases, and modified internucleoside linkages.

Another class of compounds that can be targeted using targeting ligands are small molecule compounds. The small molecule compounds (e.g., an organic compound having a molecular weight of ca. 1000 daltons or less) are typically shown to alter the function and/or activity of the target such that disease and/or disease symptoms are modulated or ameliorated or are typically useful as a diagnostic marker when localized to the target. More efficient delivery of a compound to a specific location can limit or potentially eliminate unintended consequences (such as off-target effects) that may be caused by administration of the compound and provide improved localization of a diagnostic compound.

### SUMMARY

Embodiments provided herein are directed to compounds (e.g., any of those delineated herein) and methods for targeting cells expressing N-methyl-D-aspartate (NMDA) receptor. Certain embodiments provided herein are directed to compounds and methods for delivering an agent to cells expressing NMDA receptor. In certain embodiments, the cell is in the brain. In certain embodiments, the cell is in the frontal cortex. In certain embodiments, the cell is in the striatum. In certain embodiments, the cell is in the cerebellum. In certain embodiments, the cell is in the brain stem. In certain embodiments, the cell is in the hippocampus. In certain embodiments, the cell is in the spinal cord. In certain embodiments, the agent is a therapeutic compound. In certain embodiments, delivery of the agent is for the treatment of diseases, disorders, and symptoms in a subject. In certain embodiments, the agent is a diagnostic compound. In certain embodiments, a compound comprises an NMDA receptor ligand and one or more linker moieties for attachment to a therapeutic, prophylactic, or diagnostic agent. In certain embodiments, a compound comprises an NMDA receptor ligand, one or more linker moieties, and a therapeutic agent. In certain embodiments, the therapeutic agent is selected from a small molecule or an oligomeric compound. In certain embodiments, the oligomeric compound is a protein, a peptide, an antibody, an oligonucleotide, or a combination thereof. In certain embodiments, the NMDA receptor ligand is an NMDA receptor agonist. In certain embodiments, the NMDA receptor ligand is an NMDA receptor antagonist. In certain embodiments, the NMDA receptor ligand is a small molecule, an aptamer, a peptide, or an antibody. In certain embodiments, the NMDA receptor ligand is any of those delineated herein, or a derivative or prodrug thereof.

In certain embodiments, contacting a cell expressing NMDA receptor, such as a brain cell, with a compound provided herein, delivers the agent to the cell. In certain embodiments, contacting a cell expressing NMDA receptor, such as a brain cell, with a compound provided herein, treats a disease, disorder, or symptom in a subject. In certain embodiments, a compound comprising an NMDA receptor ligand selectively or preferentially targets a cell expressing NMDA receptor compared to a cell not expressing NMDA receptor. In certain embodiments, a compound comprising an NMDA receptor ligand selectively or preferentially targets a cell expressing NMDA receptor compared to a compound not comprising an NMDA receptor ligand.

Certain embodiments provided herein are directed to compounds and methods for modulating expression of a nucleic acid target in cells expressing an NMDA receptor. In certain embodiments, the cell is in the brain. In certain embodiments, the cell is in the frontal cortex. In certain embodiments, the cell is in the striatum. In certain embodiments, the cell is in the cerebellum. In certain embodiments, the cell is in the brain stem. In certain embodiments, the cell is in the hippocampus. In certain embodiments, the cell is in the spinal cord. In certain embodiments, contacting a cell expressing an NMDA receptor, such as a brain cell, with a compound provided herein, modulates the expression or activity of a nucleic acid target in the cell. In certain embodiments, a compound comprises an NMDA receptor ligand, one or more linker moieties, and an oligonucleotide.

It is understood that the embodiments provided herein with respect to preferred variable selections can be taken alone or in combination with one or more embodiments, or other preferred variable selections provided herein, as if each combination were explicitly listed herein.

In one aspect, the present disclosure provides compounds of Formula (I) and salts thereof:

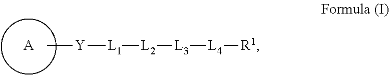

wherein

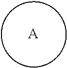

is an N-methyl-D-aspartate (NMDA) receptor ligand;
each of L₁, L₂, L₃, and L₄ is independently a linker (e.g., an optionally substituted alkyl linker, an optionally substituted polyethylene glycol (PEG) linker, an optionally substituted heteroalkyl linker, or an optionally substituted heteroaryl linker), a bond (e.g., a carbon-carbon bond, a phosphodiester bond, or a phosphorothioate bond), or absent;
Y is a bond or —C(═O)—; and
R¹ is one or more oligonucleotides, protecting groups, small molecules, proteins, antibodies, and/or peptides.

In some embodiments, the NMDA receptor ligand is an NMDA receptor agonist. In some embodiments, the NMDA receptor ligand is an NMDA receptor antagonist. In some embodiments, the NMDA receptor ligand is selected from the group consisting of:

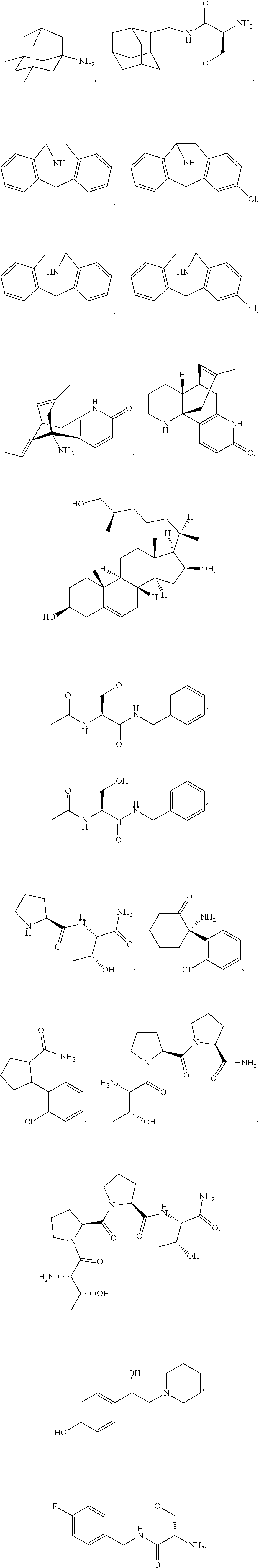

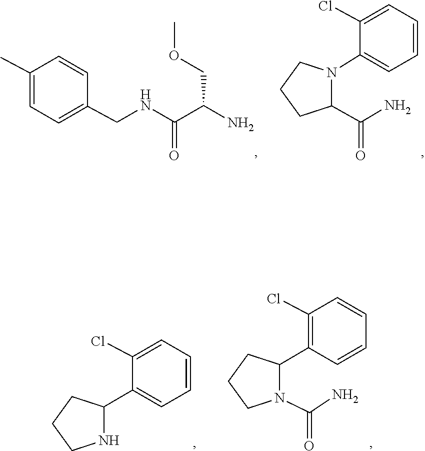

an anti-NMDA receptor antibody, and derivatives thereof.

In some aspects, the present disclosure provides compounds comprising the structure of Formula (II), and salts thereof, wherein L₁, L₂, L₃, L₄, and R¹ are as defined in Formula (I):

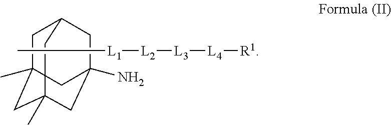

In some embodiments, the compound comprises the structure of Formula (II-a), or a salt thereof, wherein L₁, L₂, L₃, L₄, and R¹ are as defined in Formula (I):

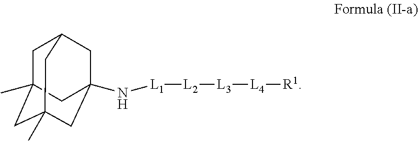

In some aspects, the present disclosure provides compounds comprising the structure of Formula (III), and salts thereof, wherein Y, L₁, L₂, L₃, L₄, and R¹ are as defined in Formula (I) and R² is hydrogen, halogen, —OH, or —OMe:

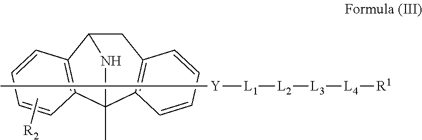

In some aspects, the present disclosure provides compounds comprising the structure of Formula (III-a), and salts thereof, wherein Y, L₁, L₂, L₃, L₄, and R¹ are as defined in Formula (I) and R² is hydrogen, halogen, —OH, or —OMe:

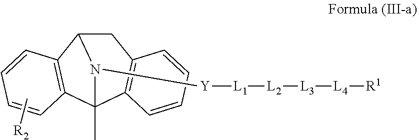

In some embodiments, the compound comprises the structure of Formula (III-b), or a salt thereof, wherein Y, L₁, L₂, L₃, L₄, and R¹ are as defined in Formula (I):

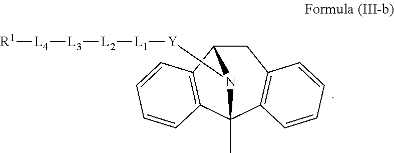

In some embodiments, the compound comprises the structure of Formula (III-c), or a salt thereof, wherein Y, L₁, L₂, L₃, L₄, and R¹ are as defined in Formula (I):

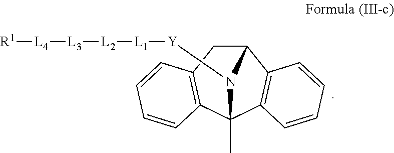

In some embodiments, the compound comprises the structure of Formula (III-d), or a salt thereof, wherein Y, L₁, L₂, L₃, L₄, and R¹ are as defined in Formula (I):

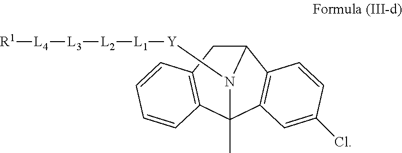

In some embodiments, the compound comprises the structure of Formula (III-e), or a salt thereof, wherein L₁, L₂, L₃, L₄, and R¹ are as defined in Formula (I):

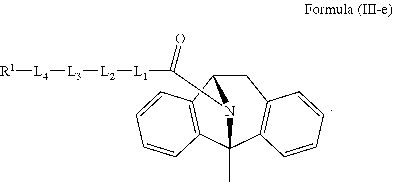

In some embodiments, the compound comprises the structure of Formula (III-f), or a salt thereof, wherein L₁, L₂, L₃, L₄, and R¹ are as defined in Formula (I):

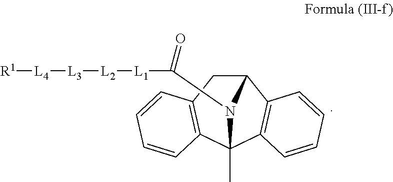

In some embodiments, the compound comprises the structure of Formula (III-g), or a salt thereof, wherein L₁, L₂, L₃, L₄, and R¹ are as defined in Formula (I):

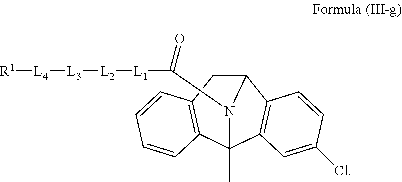

In some embodiments, the compound comprises the structure of Formula (III-h), or a salt thereof, wherein L₁, L₂, L₃, L₄, and R¹ are as defined in Formula (I):

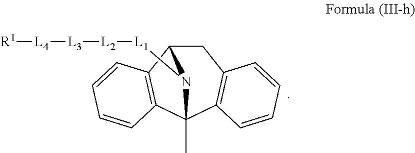

In some embodiments, the compound comprises the structure of Formula (III-i), or a salt thereof, wherein L₁, L₂, L₃, L₄, and R¹ are as defined in Formula (I):

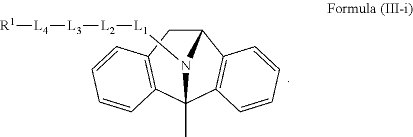

In some embodiments, the compound comprises the structure of Formula (III-j), or a salt thereof, wherein L₁, L₂, L₃, L₄, and R¹ are as defined in Formula (I):

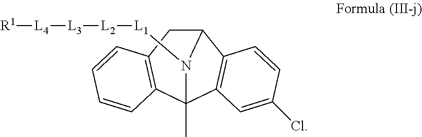

In some embodiments, the compound comprises the structure of Formula (III-k), or a salt thereof, wherein Y, L₁, L₂, L₃, L₄, and R¹ are as defined in Formula (I):

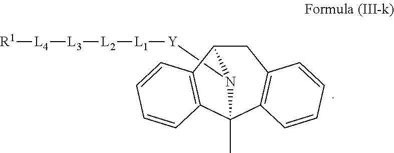

In some embodiments, the compound comprises the structure of Formula (III-1), or a salt thereof, wherein Y, L₁, L₂, L₃, L₄, and R¹ are as defined in Formula (I):

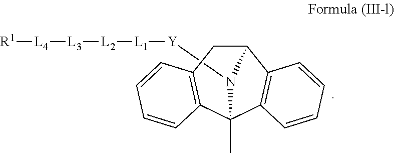

In some embodiments, the compound comprises the structure of Formula (III-m), or a salt thereof, wherein Y, L₁, L₂, L₃, L₄, and R¹ are as defined in Formula (I):

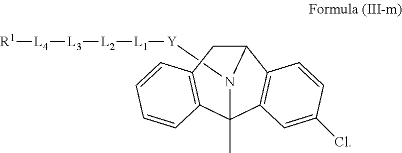

In some embodiments, the compound comprises the structure of Formula (III-n), or a salt thereof, wherein Y, L₁, L₂, L₃, L₄, and R¹ are as defined in Formula (I):

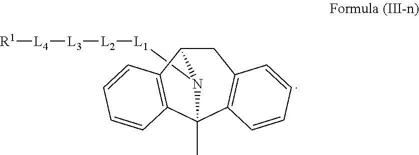

In some embodiments, the compound comprises the structure of Formula (III-o), or a salt thereof, wherein Y, L₁, L₂, L₃, L₄, and R¹ are as defined in Formula (I):

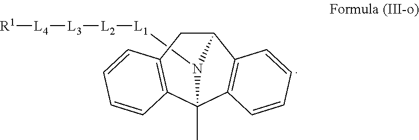

In some embodiments, the compound comprises the structure of Formula (III-p), or a salt thereof, wherein Y, L₁, L₂, L₃, L₄, and R¹ are as defined in Formula (I):

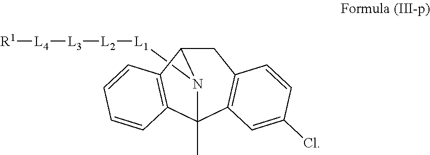

In some aspects, the present disclosure provides compounds comprising the structure of Formula (IV), and salts thereof, wherein L₁, L₂, L₃, L₄, and R¹ are as defined in Formula (I):

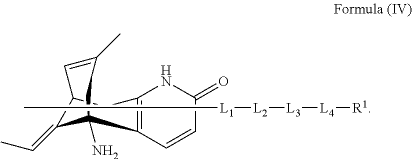

In some embodiments, the compound comprises the structure of Formula (IV-a), or a salt thereof, wherein L₁, L₂, L₃, L₄, and R¹ are as defined in Formula (I):

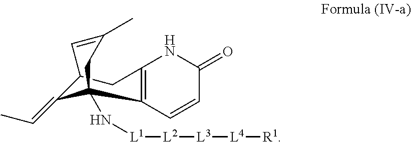

In some embodiments, the compound comprises the structure of Formula (XIX), or a salt thereof, wherein L₁, L₂, L₃, L₄, and R¹ are as defined in Formula (I):

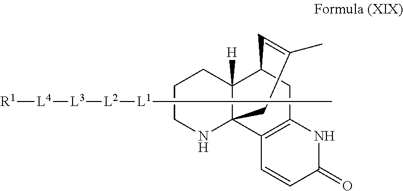

In some embodiments, the compound comprises the structure of Formula (XIX-a), or a salt thereof, wherein L₁, L₂, L₃, L₄, and R¹ are as defined in Formula (I):

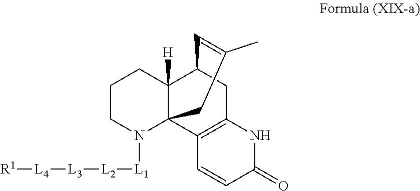

In some aspects, the present disclosure provides compounds comprising the structure of Formula (V), and salts thereof, wherein L₁, L₂, L₃, L₄, and R¹ are as defined in Formula (I):

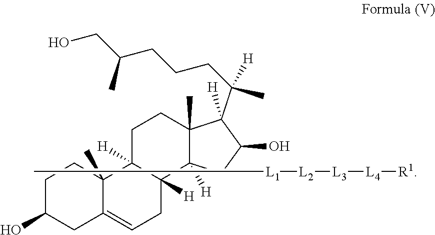

In some embodiments, the compound comprises the structure of Formula (V-a), or a salt thereof, wherein L₁, L₂, L₃, L₄, and R¹ are as defined in Formula (I):

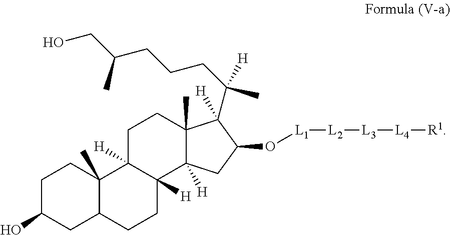

In some aspects, the present disclosure provides compounds comprising the structure of Formula (VI), and salts thereof, wherein L₁, L₂, L₃, L₄, and R¹ are as defined in Formula (I):

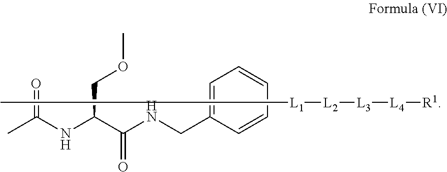

In some embodiments, the compound comprises the structure of Formula (VI-a), or a salt thereof, wherein L₁, L₂, L₃, L₄, and R¹ are as defined in Formula (I):

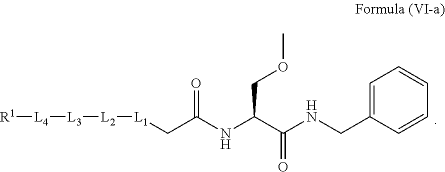

In some embodiments, the compound comprises the structure of Formula (VI-b), or a salt thereof, wherein L₁, L₂, L₃, L₄, and R¹ are as defined in Formula (I):

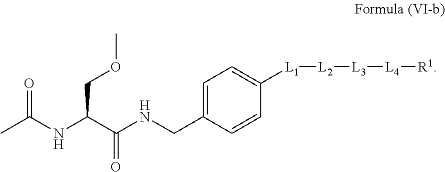

In some aspects, the present disclosure provides compounds comprising the structure of Formula (VII), and salts thereof, wherein L₁, L₂, L₃, L₄, and R¹ are as defined in Formula (I):

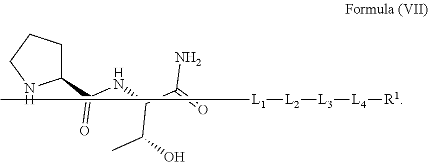

In some embodiments, the compound comprises the structure of Formula (VII-a), or a salt thereof, wherein L₁, L₂, L₃, L₄, and R¹ are as defined in Formula (I):

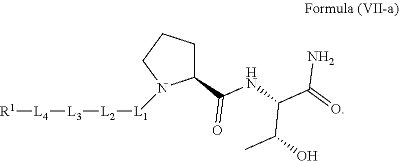

In some aspects, the present disclosure provides compounds comprising the structure of Formula (VIII), and salts thereof, wherein L₁, L₂, L₃, L₄, and R¹ are as defined in Formula (I):

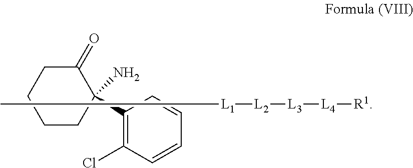

In some embodiments, the compound comprises the structure of Formula (VIII-a), or a salt thereof, wherein L₁, L₂, L₃, L₄, and R¹ are as defined in Formula (I):

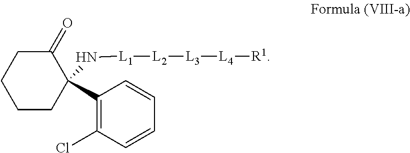

In some embodiments, the compound comprises the structure of Formula (IX), or a salt thereof, wherein L₁, L₂, L₃, L₄, and R¹ are as defined in Formula (I):

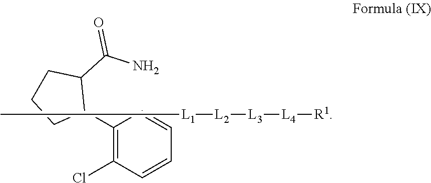

In some embodiments, the compound comprises the structure of Formula (IX-a), or a salt thereof, wherein L₁, L₂, L₃, L₄, and R¹ are as defined in Formula (I):

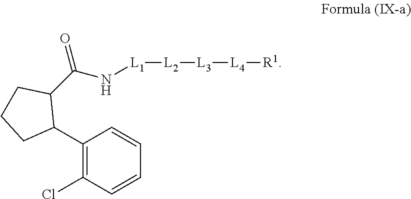

In some embodiments, the compound comprises the structure of Formula (X), or a salt thereof, wherein L₁, L₂, L₃, L₄, and R¹ are as defined in Formula (I):

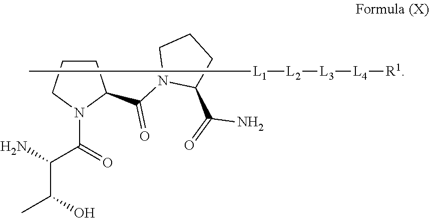

In some embodiments, the compound comprises the structure of Formula (X-a), or a salt thereof, wherein L₁, L₂, L₃, L₄, and R¹ are as defined in Formula (I):

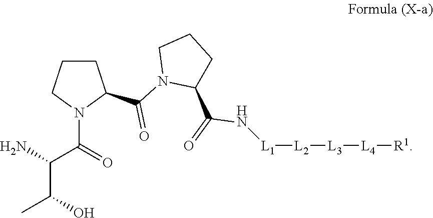

In some embodiments, the compound comprises the structure of Formula (X-b), or a salt thereof, wherein L₁, L₂, L₃, L₄, and R¹ are as defined in Formula (I):

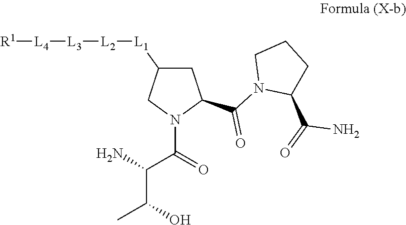

In some embodiments, the compound comprises the structure of Formula (XI), or a salt thereof, wherein L₁, L₂, L₃, L₄, and R¹ are as defined in Formula (I):

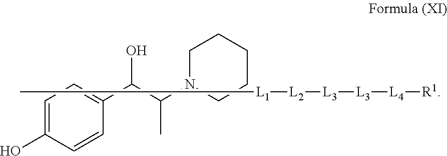

In some embodiments, the compound comprises the structure of Formula (XI-a), or a salt thereof, wherein L₁, L₂, L₃, L₄, and R¹ are as defined in Formula (I):

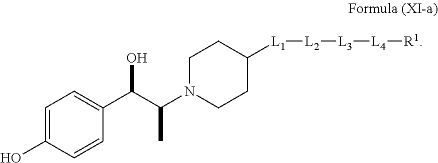

In some embodiments, the compound comprises the structure of Formula (XI-b), or a salt thereof, wherein L₁, L₂, L₃, L₄, and R¹ are as defined in Formula (I):

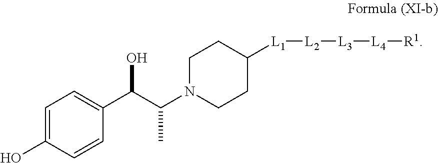

In some embodiments, the compound comprises the structure of Formula (XI-c), or a salt thereof, wherein L₁, L₂, L₃, L₄, and R¹ are as defined in Formula (I):

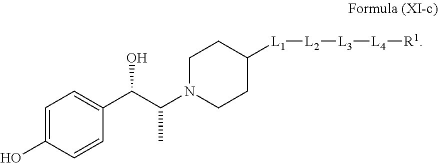

In some embodiments, the compound comprises the structure of Formula (XII), or a salt thereof, wherein L₁, L₂, L₃, L₄, and R¹ are as defined in Formula (I):

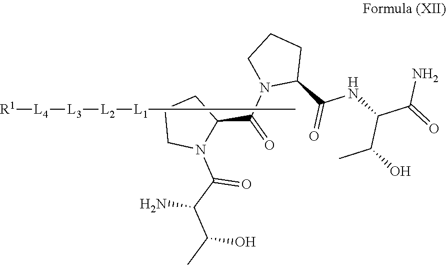

In some embodiments, the compound comprises the structure of Formula (XII-a), or a salt thereof, wherein L₁, L₂, L₃, L₄, and R¹ are as defined in Formula (I):

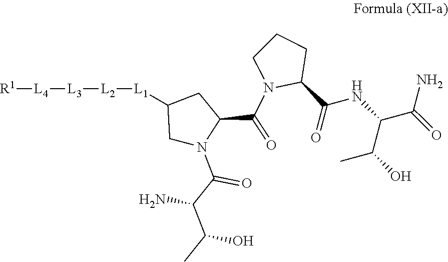

In some embodiments, the compound comprises the structure of Formula (XII-b), or a salt thereof, wherein L₁, L₂, L₃, L₄, and R¹ are as defined in Formula (I):

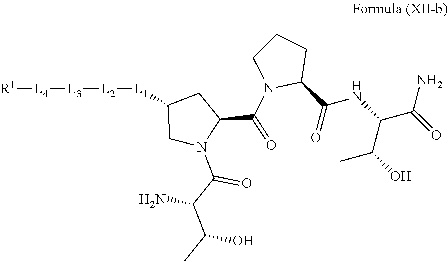

In some embodiments, the compound comprises the structure of Formula (XIII), or a salt thereof, wherein L₁, L₂, L₃, L₄, and R¹ are as defined in Formula (I):

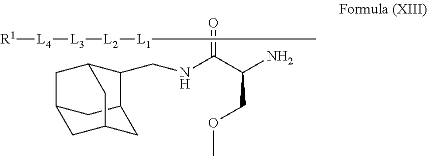

In some embodiments, the compound comprises the structure of Formula (XIII-a), or a salt thereof, wherein L₁, L₂, L₃, L₄, and R¹ are as defined in Formula (I):

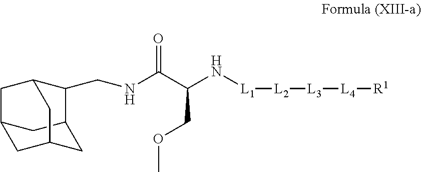

In some embodiments, the compound comprises the structure of Formula (XIV), or a salt thereof, wherein L₁, L₂, L₃, L₄, and R¹ are as defined in Formula (I):

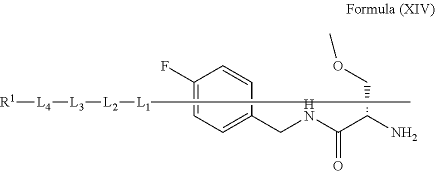

In some embodiments, the compound comprises the structure of Formula (XIV-a), or a salt thereof, wherein L₁, L₂, L₃, L₄, and R¹ are as defined in Formula (I):

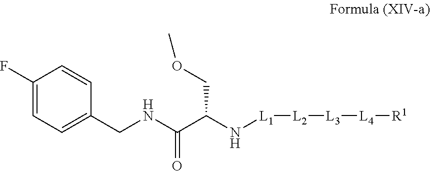

In some embodiments, the compound comprises the structure of Formula (XV), or a salt thereof, wherein L₁, L₂, L₃, L₄, and R¹ are as defined in Formula (I):

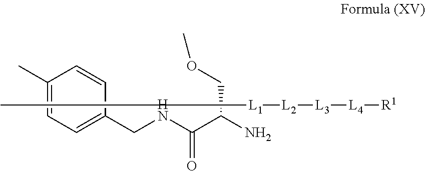

In some embodiments, the compound comprises the structure of Formula (XV-a), or a salt thereof, wherein L₁, L₂, L₃, L₄, and R¹ are as defined in Formula (I):

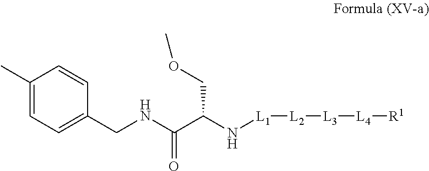

In some embodiments, the compound comprises the structure of Formula (XVI), or a salt thereof, wherein L₁, L₂, L₃, L₄, and R¹ are as defined in Formula (I):

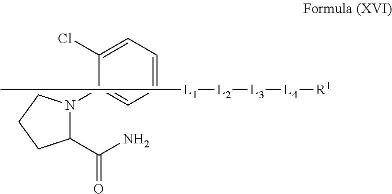

In some embodiments, the compound comprises the structure of Formula (XVI-a), or a salt thereof, wherein L₁, L₂, L₃, L₄, and R¹ are as defined in Formula (I):

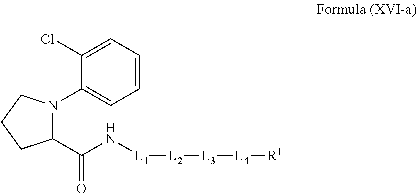

In some embodiments, the compound comprises the structure of Formula (XVII), or a salt thereof, wherein L₁, L₂, L₃, L₄, and R¹ are as defined in Formula (I):

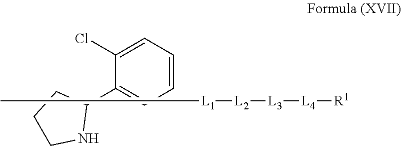

In some embodiments, the compound comprises the structure of Formula (XVII-a), or a salt thereof, wherein L₁, L₂, L₃, L₄, and R¹ are as defined in Formula (I):

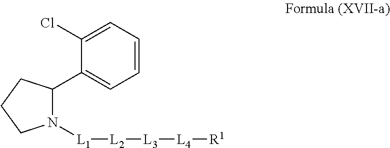

In some embodiments, the compound comprises the structure of Formula (XVIII), or a salt thereof, wherein L₁, L₂, L₃, L₄, and R¹ are as defined in Formula (I):

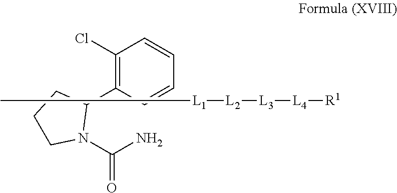

In some embodiments, the compound comprises the structure of Formula (XVIII-a), or a salt thereof, wherein L₁, L₂, L₃, L₄, and R¹ are as defined in Formula (I):

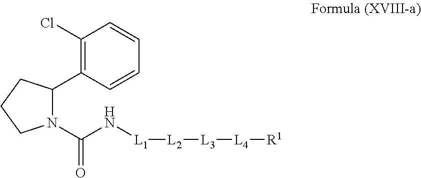

In some embodiments, any of L₁, L₂, L₃, and L₄ may independently be a linker (e.g., an optionally substituted alkyl linker, an optionally substituted polyethylene glycol (PEG) linker, an optionally substituted heteroalkyl linker, or an optionally substituted heteroaryl linker). In some embodiments, any of L₁, L₂, L₃, and L₄ may independently be a bond (e.g., a carbon-carbon bond, a phosphodiester bond, or a phosphorothioate bond). In some embodiments, any of L₁, L₂, L₃, and L₄ may independently be absent.

In some embodiments, L₁ is a bond. In some embodiments, L₁ is an optionally substituted alkyl linker. In some embodiments, L₁ is an optionally substituted C₁-C₆ alkyl linker. In some embodiments, L₁ is a C₁-C₆ alkyl linker substituted with ═O. In certain embodiments, L₁ comprises the structure

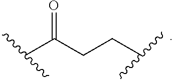 [SMILES: *CCC(*)=O]

In some embodiments, L₂ is an optionally substituted alkyl linker. In some embodiments, L₂ is an optionally substituted C₁-C₁₅ alkyl linker. In some embodiments, L₂ is structure

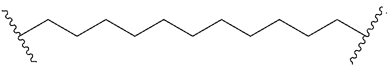 [SMILES: *CCCCCCCCCCC*]

In certain embodiments, L₂ comprises the structure

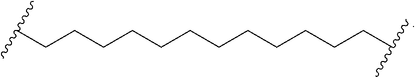 [SMILES: *CCCCCCCCCCCC*]

In some embodiments, L₂ is an optionally substituted PEG linker. In some embodiments, L₂ is an optionally substituted PEG linker comprising one, two, three, four, five, six, seven, or eight PEG units in length, wherein a PEG unit comprises the structure

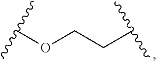 [SMILES: *CCO*]

In certain embodiments, L₂ is an optionally substituted PEG linker comprising three PEG units in length. In certain embodiments, L₂ is an optionally substituted PEG linker comprising four PEG units in length. In certain embodiments, L₂ comprises the structure

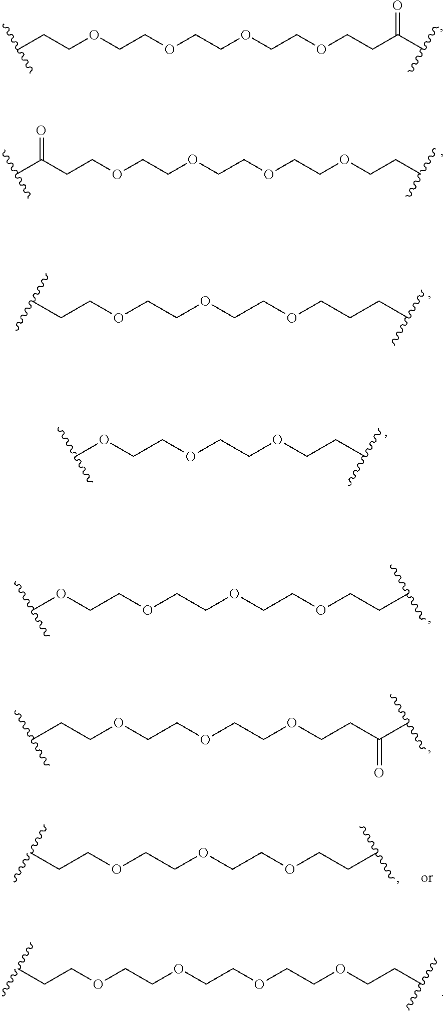

In some embodiments, L₂ is an optionally substituted heteroalkyl linker. In certain embodiments, L₂ comprises the structure

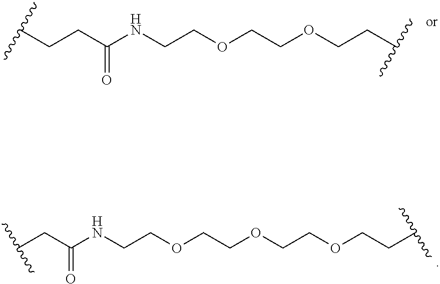 [SMILES: *CCOCCOCCNC(=O)CC* ; *CCOCCOCCOCCNC(=O)C*]

In some embodiments, L₃ is an optionally substituted heteroaryl linker. In some embodiments, L₃ is an optionally substituted partially unsaturated heterocycloalkyl linker or a heteroaryl linker. In certain embodiments, L₃ comprises the structure

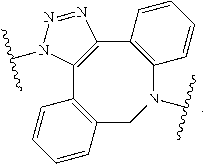 [SMILES: *N1Cc2ccccc2-c2c(nnn2*)-c2ccccc21]

In some embodiments, L₄ is an optionally substituted heteroalkyl linker. In some embodiments, the heteroalkyl linker is substituted with one or more ═O substituents. In certain embodiments, L₄ comprises the structure

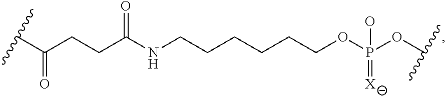

wherein X is O or S. In certain embodiments, L₄ comprises the structure

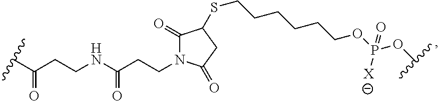

wherein X is O or S.

In some embodiments, L₁, L₂, L₃, and L₄ together comprise the structure

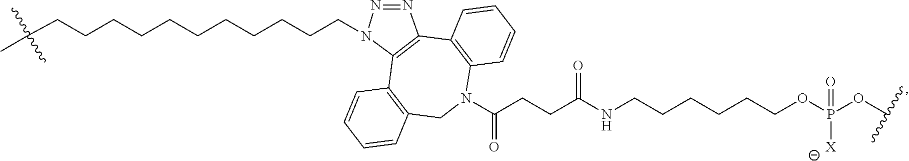

wherein X is O or S. In some embodiments, L₁, L₂, L₃, and L₄ together comprise the structure

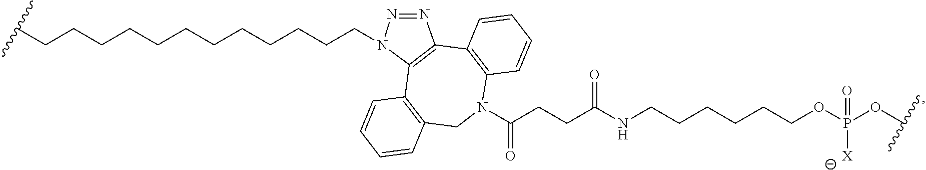

wherein X is O or S. In some embodiments, L₁, L₂, L₃, and L₄ together comprise the structure

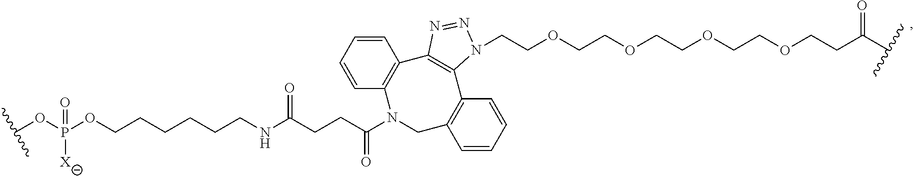

wherein X is O or S. In some embodiments, L₁, L₂, L₃, and L₄ together comprise the structure

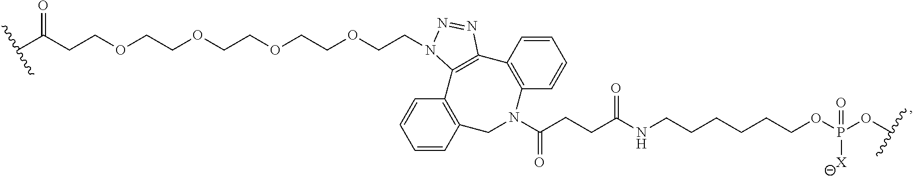

wherein X is O or S. In some embodiments, L₁, L₂, L₃, and L₄ together comprise the structure

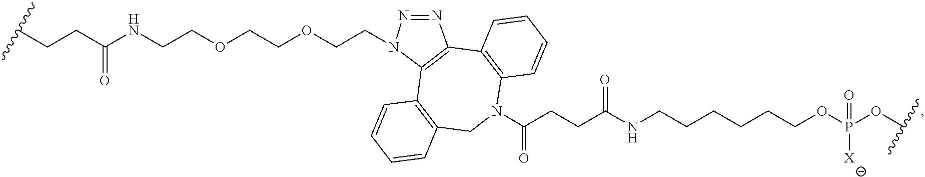

wherein X is O or S. In some embodiments, L₁, L₂, L₃, and L₄ together comprise the structure

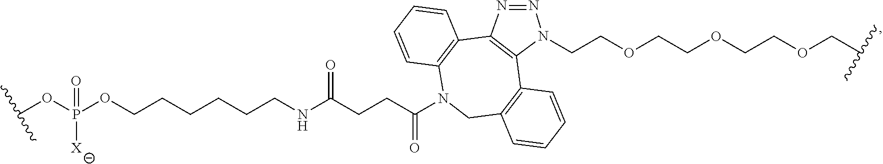

wherein X is O or S. In some embodiments, L₁, L₂, L₃, and L₄ together comprise the structure

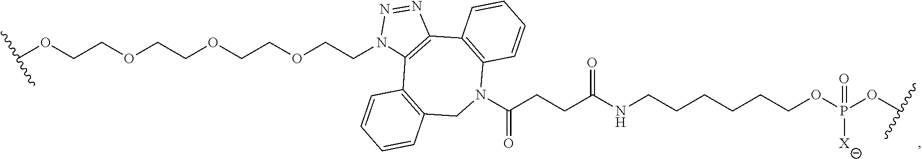

wherein X is O or S. In some embodiments, L₁, L₂, L₃, and L₄ together comprise the structure

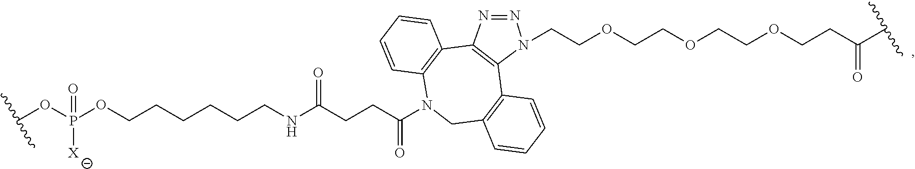

wherein X is O or S. In some embodiments, L₁, L₂, L₃, and L₄ together comprise the structure

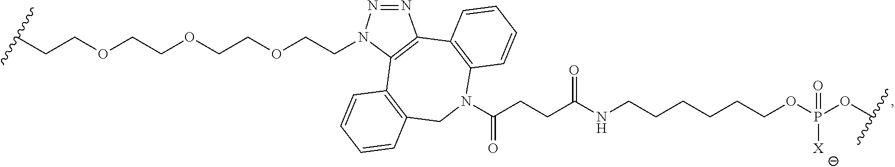

wherein X is O or S. In some embodiments, L₁, L₂, L₃, and L₄ together comprise the structure

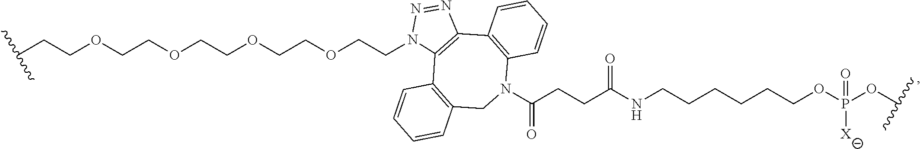

wherein X is O or S. In some embodiments, L₁, L₂, L₃, and L₄ together comprise the structure

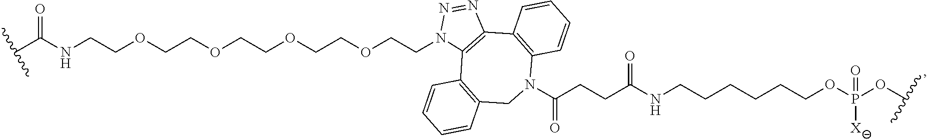

wherein X is O or S. In some embodiments, L₁, L₂, L₃, and L₄ together comprise the structure

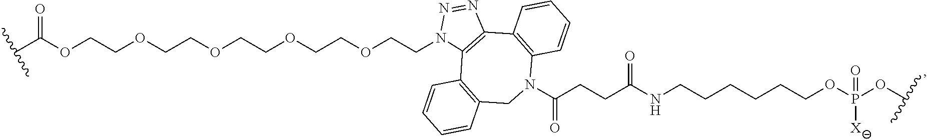

wherein X is O or S. In some embodiments, L₁, L₂, L₃, and L₄ together comprise the structure

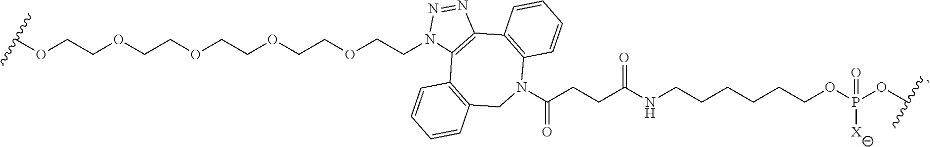

wherein X is O or S. In some embodiments, L₁, L₂, L₃, and L₄ together comprise the structure

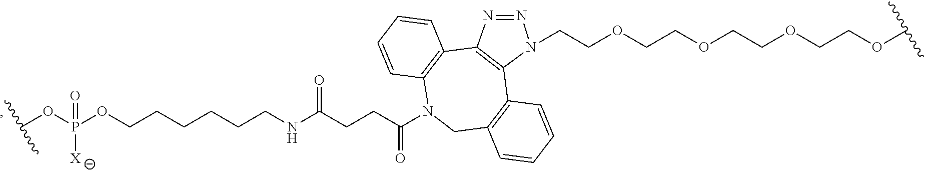

wherein X is O or S. In some embodiments, L₁, L₂, L₃, and L₄ together comprise the structure

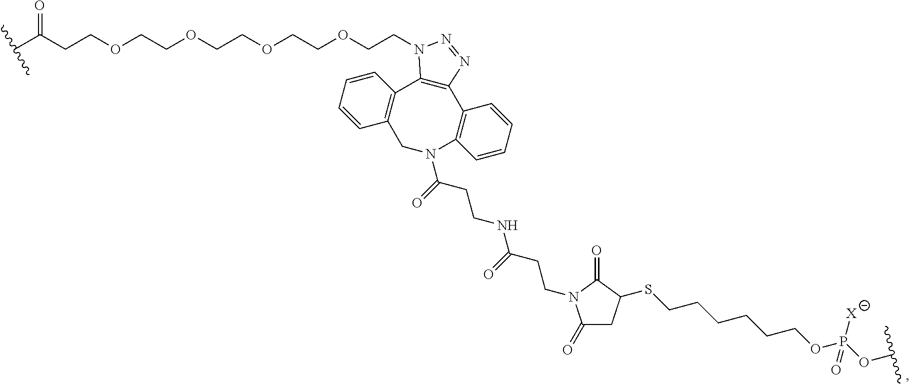

wherein X is O or S.

In certain embodiments, the present disclosure provides compounds of the structure:

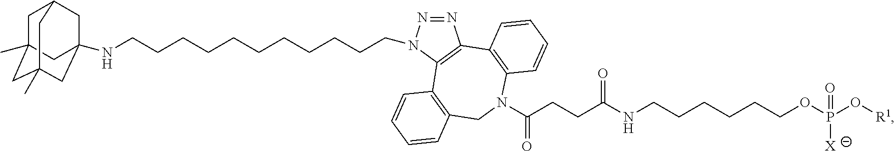

and salts thereof, wherein X is O or S. In certain embodiments the present disclosure provides compounds of the structure:

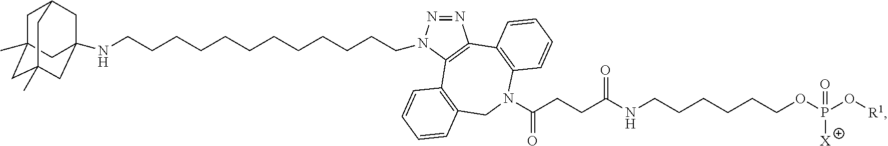

and salts thereof, wherein X is O or S. In certain embodiments, the present disclosure provides compounds of the structure:

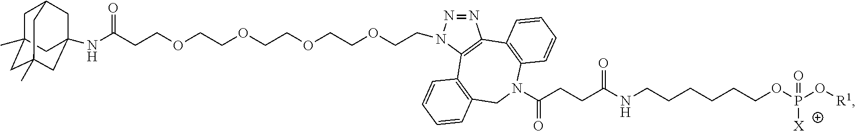

and salts thereof, wherein X is O or S. In certain embodiments, the present disclosure provides compounds of the structure:

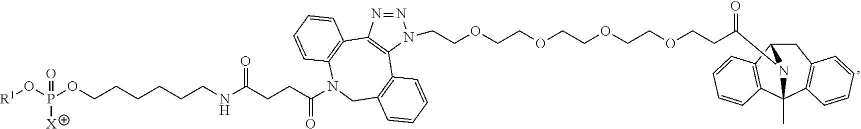

and salts thereof, wherein X is O or S. In certain embodiments, the present disclosure provides compounds of the structure:

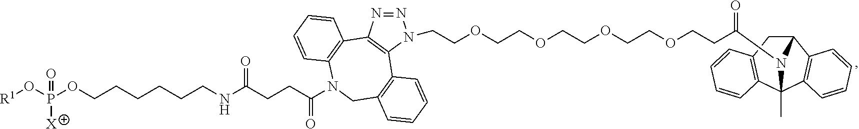

and salts thereof, wherein X is O or S. In certain embodiments, the present disclosure provides compounds of the structure:

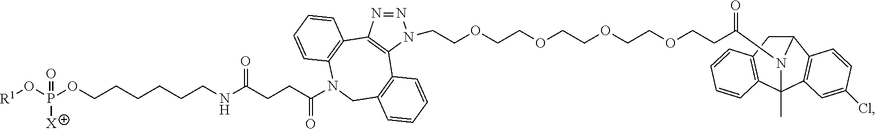

and salts thereof, wherein X is O or S. In certain embodiments, the present disclosure provides compounds of the structure:

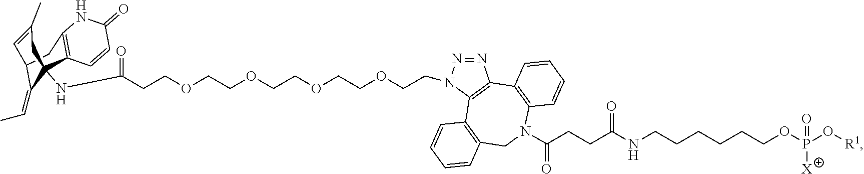

and salts thereof, wherein X is O or S. In certain embodiments, the present disclosure provides compounds of the structure:

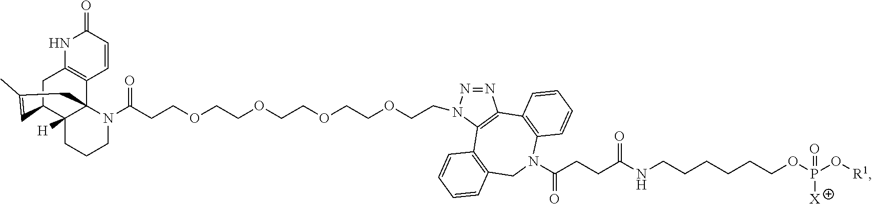

and salts thereof, wherein X is O or S. In certain embodiments, the present disclosure provides compounds of the structure:

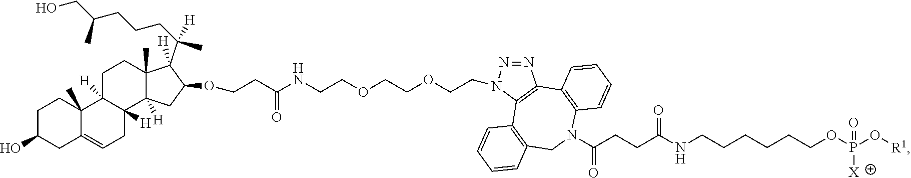

and salts thereof, wherein X is O or S. In certain embodiments, the present disclosure provides compounds of the structure:

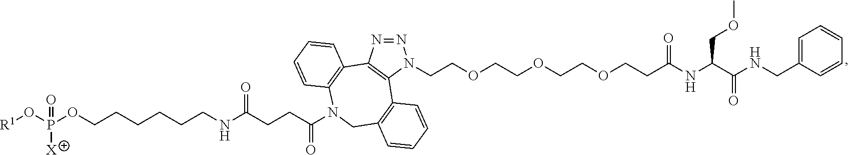

and salts thereof, wherein X is O or S. In certain embodiments, the present disclosure provides compounds of the structure:

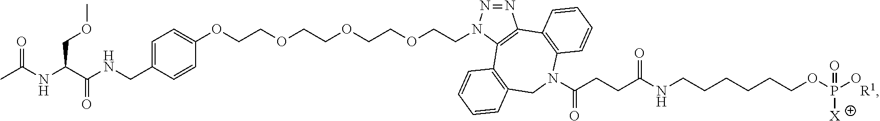

and salts thereof, wherein X is O or S. In certain embodiments, the present disclosure provides compounds of the structure:

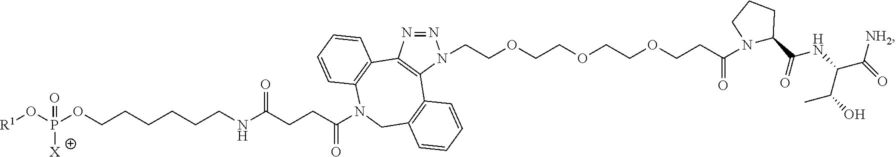

and salts thereof, wherein X is O or S. In certain embodiments, the present disclosure provides compounds of the structure:

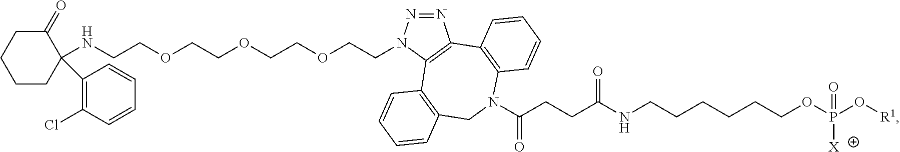

and salts thereof, wherein X is O or S. In certain embodiments, the present disclosure provides compounds of the structure:

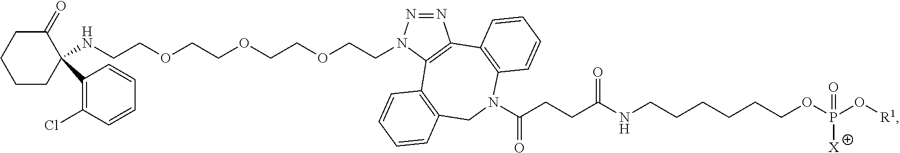

and salts thereof, wherein X is O or S. In certain embodiments, the present disclosure provides compounds of the structure:

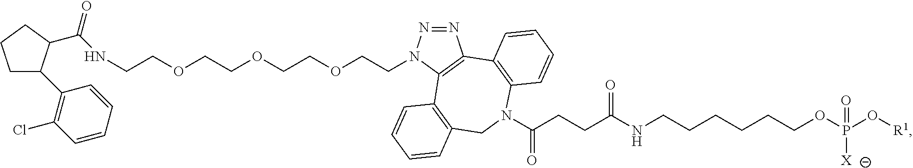

and salts thereof, wherein X is O or S. In certain embodiments, the present disclosure provides compounds of the structure:

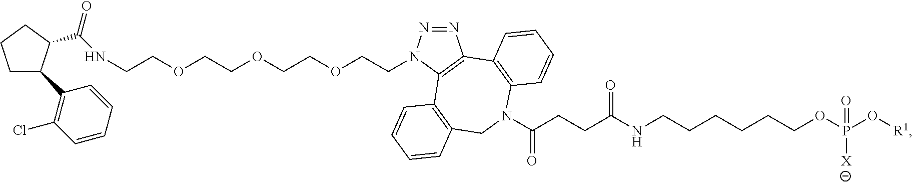

and salts thereof, wherein X is O or S. In certain embodiments, the present disclosure provides compounds of the structure:

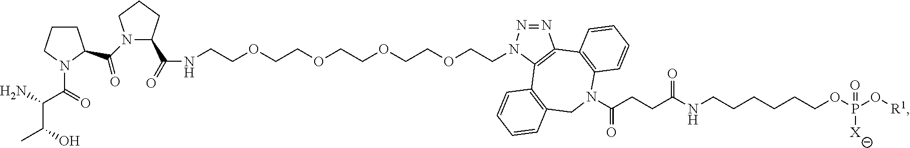

and salts thereof, wherein X is O or S. In certain embodiments, the present disclosure provides compounds of the structure:

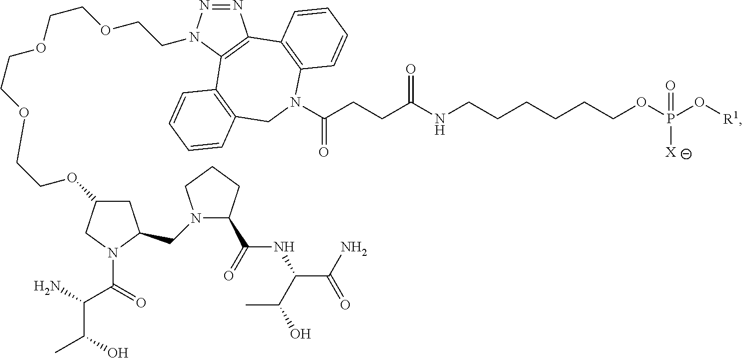

and salts thereof, wherein X is O or S. In certain embodiments, the present disclosure provides compounds of the structure:

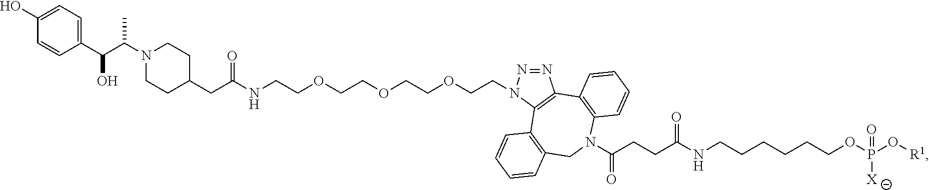

and salts thereof, wherein X is O or S. In certain embodiments, the present disclosure provides compounds of the structure:

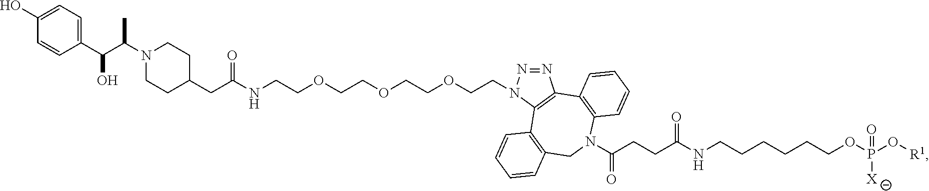

and salts thereof, wherein X is O or S. In certain embodiments, the present disclosure provides compounds of the structure:

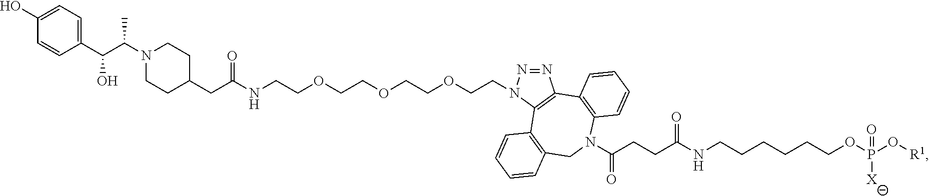

and salts thereof, wherein X is O or S. In certain embodiments, the present disclosure provides compounds of the structure:

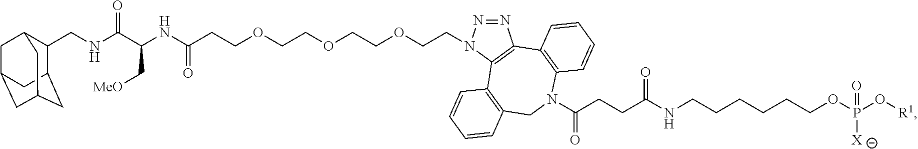

and salts thereof, wherein X is O or S. In certain embodiments, the present disclosure provides compounds of the structure:

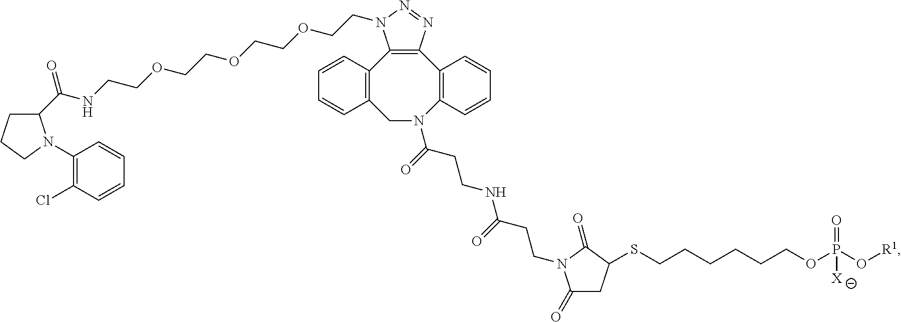

and salts thereof, wherein X is O or S. In certain embodiments, the present disclosure provides compounds of the structure:

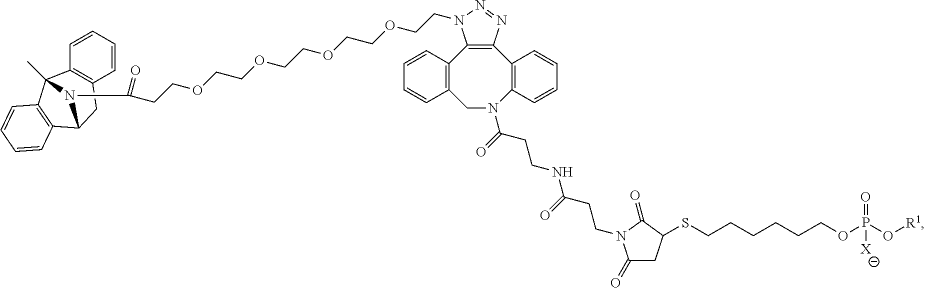

and salts thereof, wherein X is O or S. In certain embodiments, the present disclosure provides compounds of the structure:

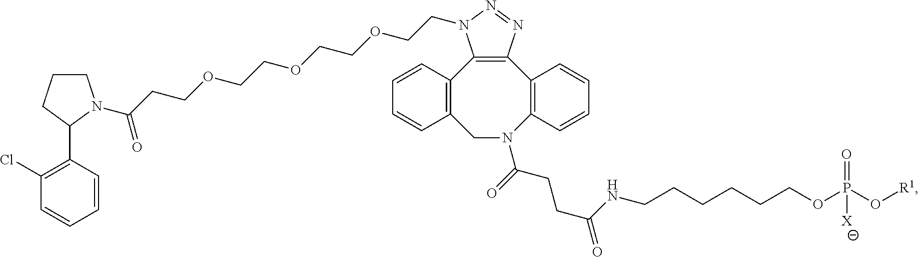

and salts thereof, wherein X is O or S. In certain embodiments, the present disclosure provides compounds of the structure:

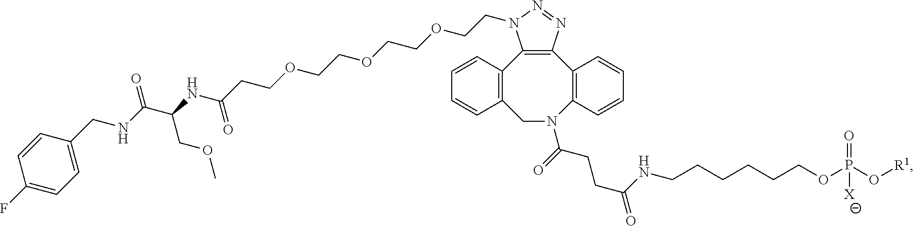

and salts thereof, wherein X is O or S. In certain embodiments, the present disclosure provides compounds of the structure:

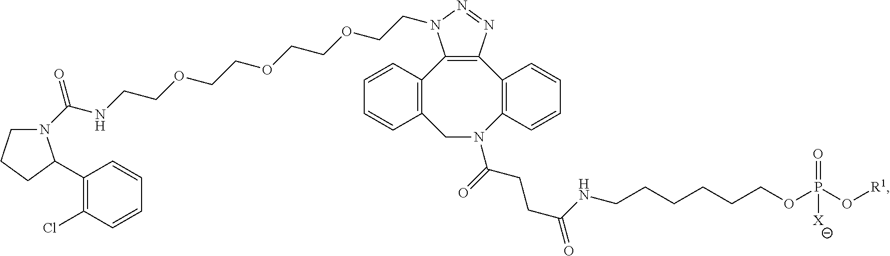

and salts thereof, wherein X is O or S. In certain embodiments, the present disclosure provides compounds of the structure:

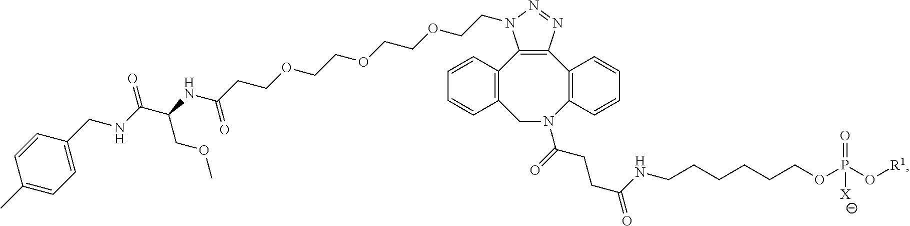

and salts thereof, wherein X is O or S.

In some embodiments, the present disclosure provides compounds comprising the structure of Formula (XX):

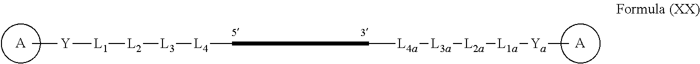

wherein
   each

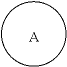

is an N-methyl-D-aspartate (NMDA) receptor ligand;
each of L₁, L₂, L₃, L₄, L₁ₐ, L₂ₐ, L₃ₐ, and L₄ₐ is independently a linker (e.g., an optionally substituted alkyl linker, an optionally substituted polyethylene glycol (PEG) linker, an optionally substituted heteroalkyl linker, or an optionally substituted heteroaryl linker), a bond (e.g., a carbon-carbon bond, a phosphodiester bond, or a phosphorothioate bond), or absent;
each of Y and Yₐ is independently a bond or —C(═O)—; and

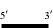

is an oligonucleotide.

In some embodiments, the present disclosure provides compounds comprising the structure of Formula (XXI):

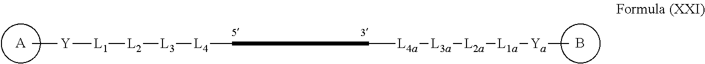

wherein
   each of

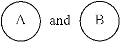

is independently an N-methyl-D-aspartate (NMDA) receptor ligand;
each of L₁, L₂, L₃, L₄, L₁ₐ, L₂ₐ, L₃ₐ, and L₄ₐ is independently a linker (e.g., an optionally substituted alkyl linker, an optionally substituted polyethylene glycol (PEG) linker, an optionally substituted heteroalkyl linker, or an optionally substituted heteroaryl linker), a bond (e.g., a carbon-carbon bond, a phosphodiester bond, or a phosphorothioate bond), or absent;
each of Y and Yₐ is independently a bond or —C(═O)—; and

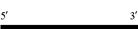

is an oligonucleotide.

In certain embodiments, the present disclosure provides compounds comprising the structure:

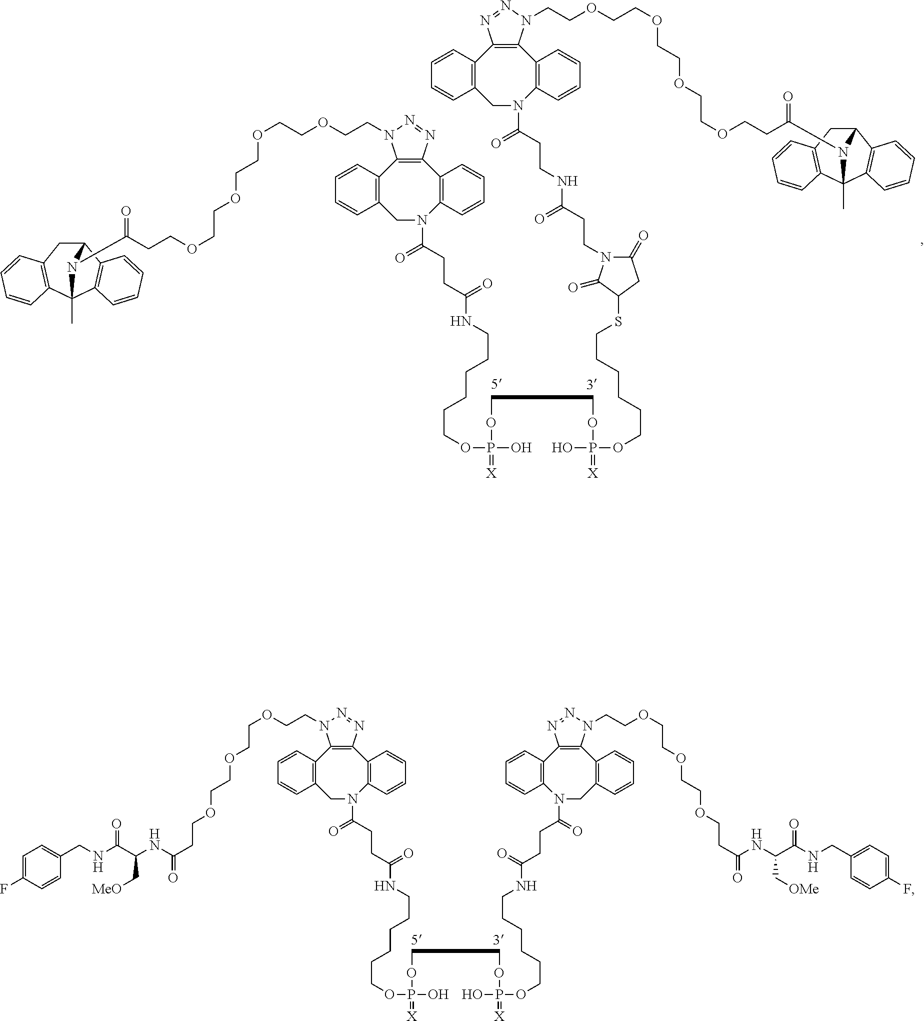

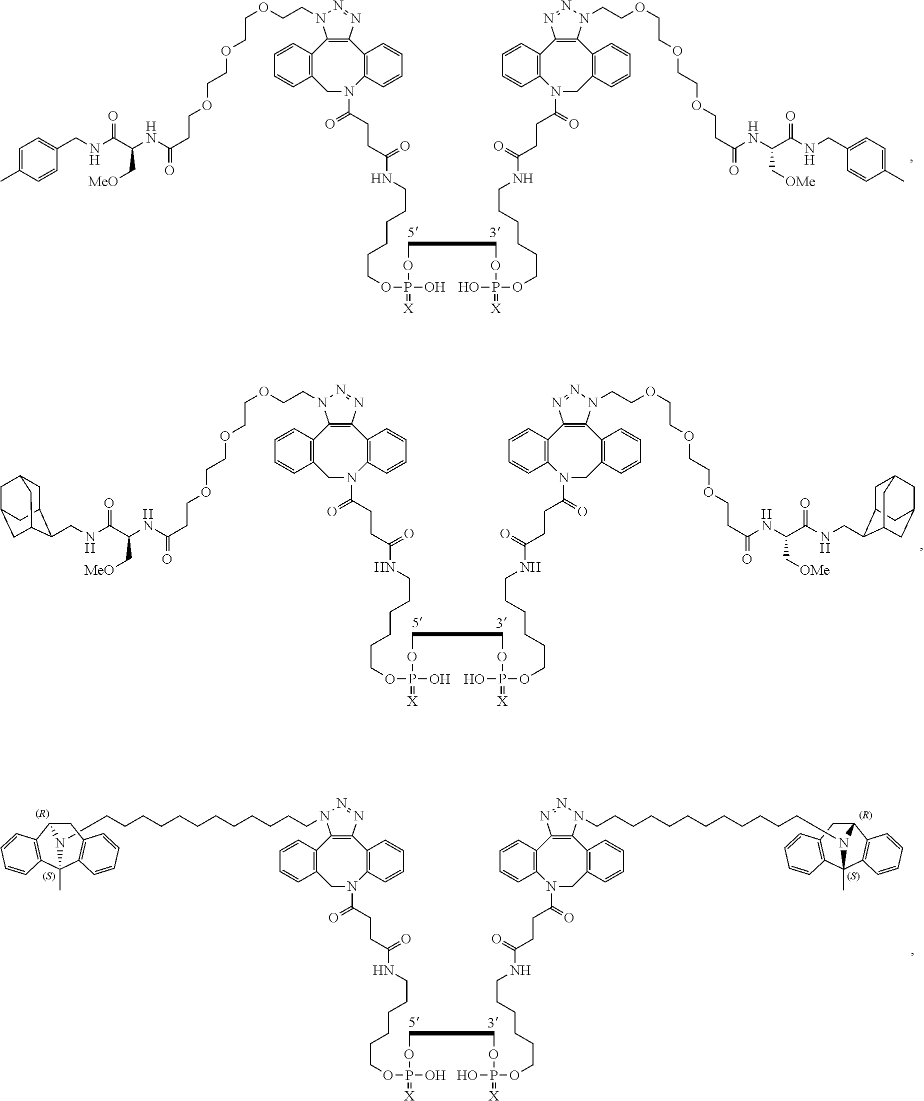

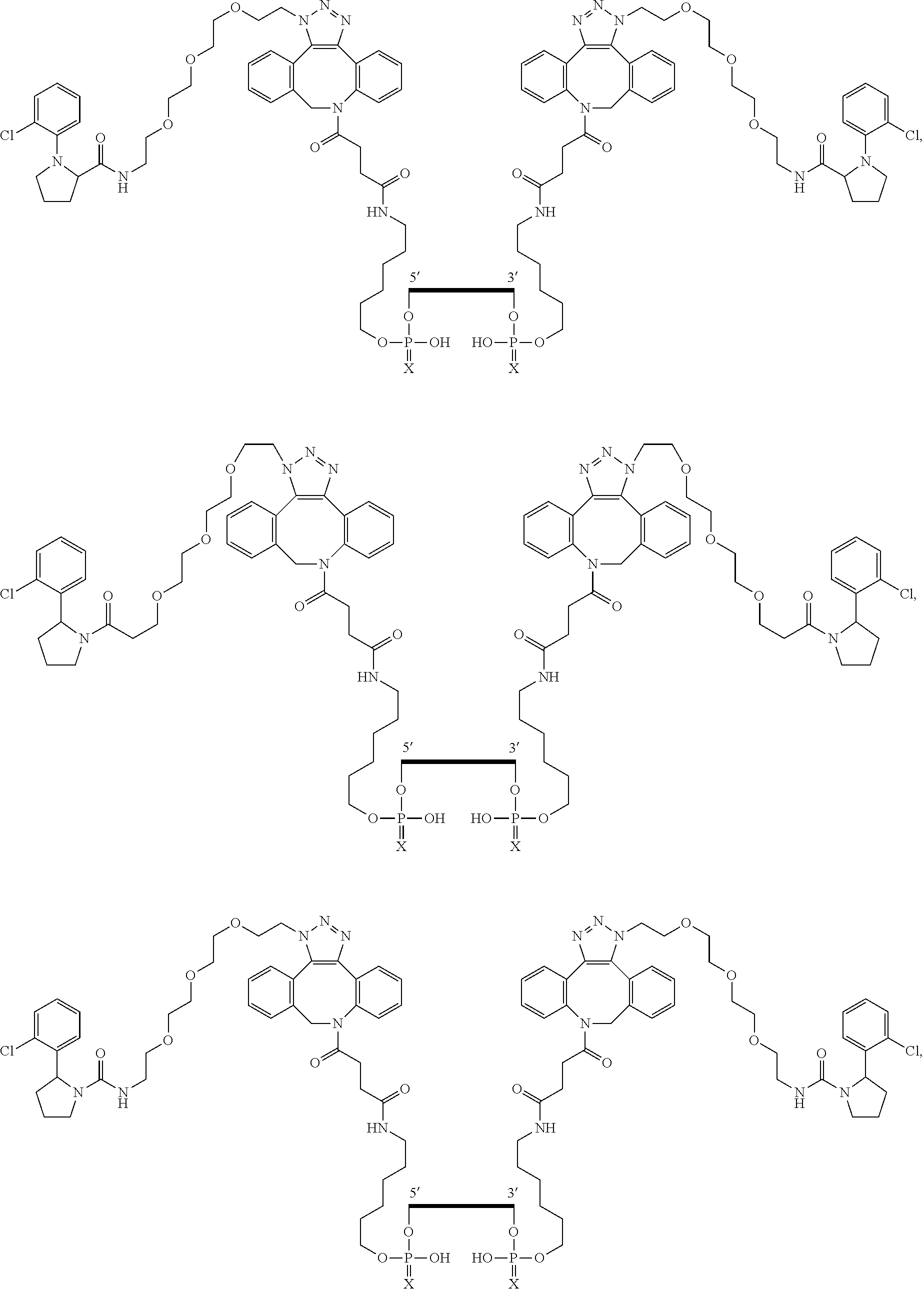

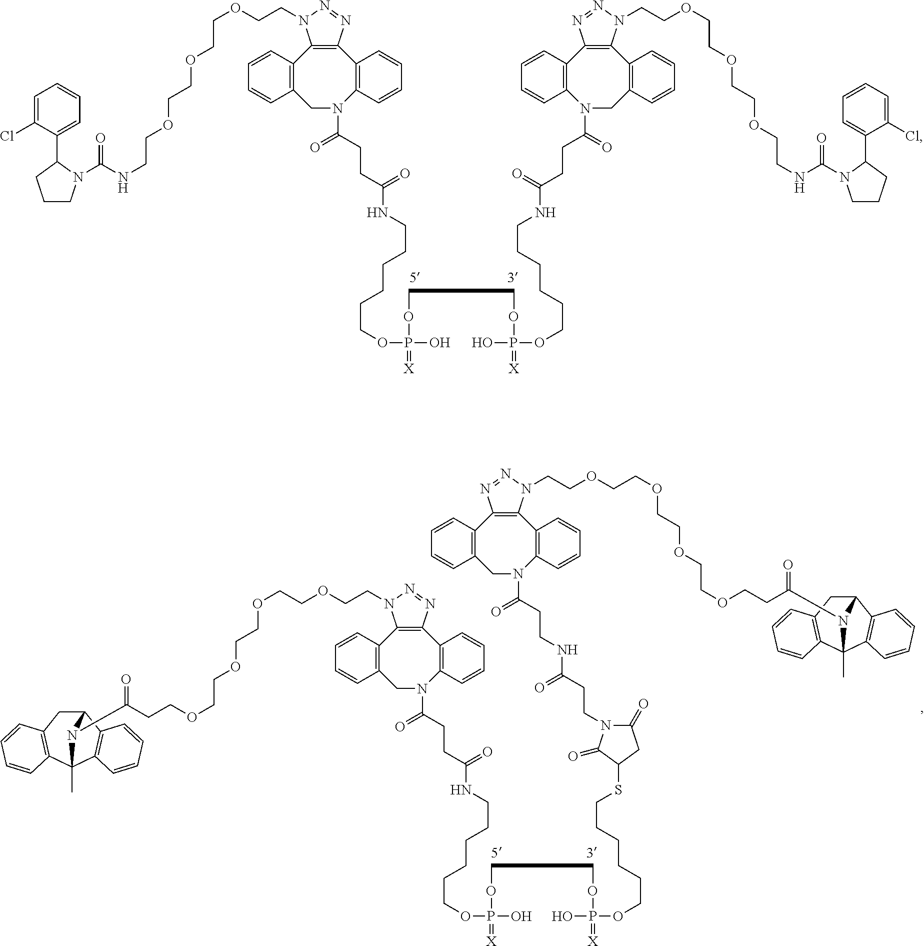

wherein each instance of X is independently O or S, and

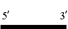

is an oligonucleotide.

In some embodiments, X is O. In some embodiments, X is S.

In some embodiments, R¹ comprises an oligonucleotide. In some embodiments, the oligonucleotide is attached at its 5′ end. In some embodiments, the oligonucleotide is attached at its 3′ end. In some embodiments, the oligonucleotide is attached at an internal position on the oligonucleotide. In some embodiments, the internal position is at an internucleoside linkage. In some embodiments, R¹ comprises an oligonucleotide conjugated to one or more additional NMDA receptor ligands. In some embodiments, the oligonucleotide is conjugated to two, three, four, five, or more than five additional NMDA receptor ligands. In certain embodiments, the additional NMDA receptor ligands are conjugated to the oligonucleotide at the 5′ end of the oligonucleotide, the 3′ end of the oligonucleotide, one or more internal positions on the oligonucleotide, or any combination thereof. In certain embodiments, the oligonucleotide is a modified oligonucleotide.

In another aspect, the present disclosure provides compositions comprising any of the compounds provided herein, and a pharmaceutically acceptable excipient.

In another aspect, the present disclosure provides methods for delivering a therapeutic oligonucleotide to the brain of a subject, comprising administration of any of the compounds or compositions provided herein to the subject. In some embodiments, the therapeutic oligonucleotide is delivered to one or more brain regions selected from the group consisting of the striatum, the cerebellum, the brain stem, the hippocampus, the frontal cortex, and the spinal cord. In another aspect, the present disclosure provides methods for treating or ameliorating a disease, disorder, or symptom thereof in a subject, comprising administration of any of the compounds or compositions provided herein to the subject. In some embodiments, the disease, disorder, or symptom thereof is a central nervous system (CNS) disease, disorder, or symptom thereof. In certain embodiments, the disease, disorder, or symptom thereof is Alzheimer's disease, or a symptom thereof. In some embodiments, the compound is administered to the subject intrathecally.

In another aspect, the present disclosure provides methods for making any of the compounds provided herein, comprising one or more compounds and chemical transformations described herein, including Examples 1-77.

### DETAILED DESCRIPTION

### Definitions

It is to be understood that both the foregoing general description and the following detailed description are exemplary and explanatory only and are not restrictive of the embodiments, as claimed. Herein, the use of the singular includes the plural unless specifically stated otherwise. As used herein, the use of “or” means “and/or” unless stated otherwise. Furthermore, the use of the term “including” as well as other forms, such as “includes” and “included,” is not limiting. The section headings used herein are for organizational purposes only and are not to be construed as limiting the subject matter described.

Unless otherwise indicated, the following terms have the following meanings:

As used herein, the term “treating” a disorder encompasses ameliorating, mitigating and/or managing the disorder and/or conditions that may cause the disorder. The terms “treating” and “treatment” refer to a method of alleviating or abating a disease and/or its attendant symptoms. In accordance with the present disclosure, “treating” includes blocking, inhibiting, attenuating, protecting against, modulating, reversing the effects of, and reducing the occurrence of, e.g., the harmful effects of a disorder. As used herein, “inhibiting” encompasses preventing, reducing, and halting progression.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high-performance liquid chromatography (HPLC). Particularly, in certain embodiments, the compound is at least 85% pure, more preferably at least 90% pure, more preferably at least 95% pure, and most preferably at least 99% pure.

The term “administration” or “administering” includes routes of introducing the compound(s) to a subject to perform their intended function. Examples of routes of administration which can be used include injection (subcutaneous, intravenous, parenteral, intraperitoneal, intrathecal), topical, oral, inhalation, rectal, and transdermal.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response. An effective amount is also one in which any non-tolerable or detrimental effects (e.g., side effects) of the compound are outweighed by the therapeutically beneficial effects.

The phrases “systemic administration,” “administered systemically,” “peripheral administration,” and “administered peripherally” as used herein mean the administration of a compound(s), oligonucleotide(s), drug, or other material, such that it enters the patient's circulatory system and, thus, is subject to metabolism and other like processes.

The term “therapeutically effective amount” refers to the amount of the compound being administered sufficient to prevent development of or alleviate to some extent one or more of the symptoms of the condition or disorder being treated.

A therapeutically effective amount of compound (i.e., an effective dosage) may range from about 0.005 g/kg to about 200 mg/kg, preferably about 0.01 mg/kg to about 200 mg/kg, and more preferably about 0.015 mg/kg to about 30 mg/kg of body weight. In other embodiments, the therapeutically effect amount may range from about 1.0 μM to about 10 μM. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound can include a single treatment or, preferably, can include a series of treatments. In one example, a subject is treated with a compound in the range of between about 0.005 g/kg to about 200 mg/kg of body weight, daily, weekly, monthly, quarterly, or yearly. In another example, a subject may be treated daily, weekly, monthly, quarterly, or yearly for several years in the setting of a chronic condition or illness. It will also be appreciated that the effective dosage of a compound used for treatment may increase or decrease over the course of a particular treatment.

The term “chiral” refers to molecules that have the property of non-superimposability of the mirror image partner, while the term “achiral” refers to molecules that are superimposable on their mirror image partner.

Certain compounds of the present disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)-for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents or resolved using conventional techniques. When the compounds described herein contain olefinic bonds or other centers of geometric asymmetry, and unless specified otherwise, it is intended that the compounds include both E and Z geometric isomers.

The term “tautomer,” as used herein, refers to one of two or more structural isomers which exist in equilibrium, and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure (i.e., the R and S configurations for each asymmetric center). Therefore, single stereochemical isomers as well as enantiomeric and diastereomeric mixtures of the present compounds are within the scope of the disclosure.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon, are within the scope of this disclosure.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the results of a synthetic method that is not designed to control the stereochemical configuration. In certain embodiments, a stereorandom chiral center is a stereorandom phosphorothioate internucleoside linkage.

The term “diastereomers” refers to stereoisomers with two or more centers of dissymmetry and whose molecules are not mirror images of one another.

The term “enantiomers” refers to two stereoisomers of a compound that are non-superimposable mirror images of one another. An equimolar mixture of two enantiomers is called a “racemic mixture” or a “racemate.”

The term “isomers” or “stereoisomers” refers to compounds that have identical chemical constitution but differ with regard to the arrangement of the atoms or groups in space.

The term “prodrug” is meant to indicate a compound that may be converted under physiological conditions or by solvolysis to a biologically active form of the compound (e.g., biologically active form of a nucleic acid) or analogue thereof as described herein. Thus, the term “prodrug” refers to a precursor of a biologically active compound (e.g., nucleic acid) or analogue thereof that is pharmaceutically acceptable. A prodrug may be inactive when administered to a subject, but is converted in vivo to an active compound, for example, by hydrolysis. The prodrug compound often offers advantages of solubility, tissue compatibility or delayed release in a mammalian organism (see, e.g., Bundgaard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. The term “prodrug” is also meant to include any covalently bonded carriers, which release the active compound in vivo when such prodrug is administered to a mammalian subject. Prodrugs of an active compound, as described herein, may be prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of suitable prodrugs include, but are not limited to glutathione, acyloxy, thioacyloxy, 2-carboalkoxyethyl, disulfide, thiaminal, and enol ester derivatives of a phosphorus atom-modified nucleic acid. The term “pro-oligonucleotide” or “pronucleotide” or “nucleic acid prodrug” refers to an oligonucleotide which has been modified to be a prodrug of the oligonucleotide. Phosphonate and phosphate prodrugs can be found, for example, in Wiener et al., “Prodrugs or phosphonates and phosphates: crossing the membrane” Top. Curr. Chem. 2015, 360:115-160, the entirety of which is herein incorporated by reference. Prodrugs that are converted to active forms through other mechanisms in vivo are also included. In aspects, the compounds of the present disclosure are prodrugs of any of the formulae herein.

The term “subject” refers to animals such as mammals, including, but not limited to, primates (e.g., humans), cows, sheep, goats, horses, dogs, cats, rabbits, rats, mice, and the like. In certain embodiments, the subject is a human.

The terms “a,” “an,” and “the” refer to “one or more” when used in this application, including the claims. Thus, for example, reference to “a sample” includes a plurality of samples, unless the context clearly is to the contrary (e.g., a plurality of samples), and so forth.

Throughout this specification and the claims, the words “comprise,” “comprises,” and “comprising” are used in a non-exclusive sense, except where the context requires otherwise.

As used herein, the term “about,” when referring to a value, is meant to encompass variations of, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

As used herein, the term “alkyl,” by itself or as part of another substituent, means, unless otherwise stated, a straight-chained (i.e., unbranched) or branched carbon chain (or carbon), or combination thereof, which may be fully saturated, mono-, (e.g., alkene or alkenyl) or polyunsaturated (e.g., alkyne or alkynyl) and can include mono-, di- and multivalent radicals, having the number of carbon atoms designated. For example, C₁-C₂₄ means 1 to 24 carbon atoms. A specified number of carbon atoms within this range includes, for example, C₁-C₂₀ alkyl (having 1-20 carbon atoms), C₁-C₁₂ alkyl (having 1-12 carbon atoms) and C₁-C₄ alkyl (having 1-4 carbon atoms).

The term “alkenyl” refers to an unsaturated hydrocarbon chain that may be a straight chain or branched chain, containing 2 to 12 carbon atoms and at least one carbon-carbon double bond. Alkenyl groups may be optionally substituted with one or more substituents.

The term “alkynyl” refers to an unsaturated hydrocarbon chain that may be a straight chain or branched chain, containing the 2 to 12 carbon atoms and at least one carbon-carbon triple bond. Alkynyl groups may be optionally substituted with one or more substituents.

The term “lower alkyl” refers to a C₁-C₆ alkyl chain. Examples of alkyl groups include methyl, ethyl, n-propyl, isopropyl, tert-butyl, and n-pentyl. Alkyl groups may be optionally substituted with one or more substituents.

The term “heteroalkyl” by itself or in combination with another term, means, unless otherwise stated, a stable straight or branched chain, or combinations thereof, including at least one carbon atom and at least one heteroatom (e.g., 0, N, P, Si, and/or S), and wherein the nitrogen and sulfur atoms may optionally be oxidized, and the nitrogen heteroatom may optionally be quaternized. The heteroatom(s) (e.g., 0, N, P, Si, and/or S) may be placed at any interior position of the heteroalkyl group or at the position at which the alkyl group is attached to the remainder of the molecule. Heteroalkyl is an uncyclized chain. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, S(O)—CH₃, —CH₂—CH₂—S(O)₂—CH₃, —CH═CHO—CH₃, —Si(CH₃)₃, —CH₂—CH═N—OCH₃, —CH═CH N(CH₃)—CH₃, —O—CH₃, —OCH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, B, or P). A heteroalkyl moiety may include two optionally different heteroatoms (e.g., O, N, S, Si, B, and/or P). A heteroalkyl moiety may include three optionally different heteroatoms (e.g., O, N, S, Si, B, and/or P). A heteroalkyl moiety may include four optionally different heteroatoms (e.g., O, N, S, Si, B, and/or P). A heteroalkyl moiety may include five optionally different heteroatoms (e.g., O, N, S, Si, B, and/or P). A heteroalkyl moiety may include up to 8 or more optionally different heteroatoms (e.g., O, N, S, Si, B, and/or P).

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′—, —C(O)NR′—, —NR′R^{aa}, —OR^{aa}, —SR^{aa}, and/or —SO₂R′. Where “heteroalkyl” is recited, followed by recitations of specific heteroalkyl groups, such as —NR′R″ or the like, it will be understood that the terms heteroalkyl and NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The term “haloalkyl” refers to an alkyl group that is substituted by one or more halo substituents. Examples of haloalkyl groups include fluoromethyl, difluoromethyl, trifluoromethyl, bromomethyl, chloromethyl, and 2,2,2-trifluoroethyl.

The term “arylalkenyl” refers to an unsaturated hydrocarbon chain that may be a straight chain or branched chain, containing 2 to 12 carbon atoms and at least one carbon-carbon double bond wherein one or more of the sp² hybridized carbons of the alkenyl unit attaches to an aryl moiety. Alkenyl groups may be optionally substituted with one or more substituents.

The term “arylalkynyl” refers to an unsaturated hydrocarbon chain that may be a straight chain or branched chain, containing 2 to 12 carbon atoms and at least one carbon-carbon triple bond wherein one or more of the sp hybridized carbons of the alkynyl unit attaches to an aryl moiety. Alkynyl groups may be optionally substituted with one or more substituents.

The sp²- or sp-hybridized carbons of an alkenyl group and an alkynyl group, respectively, may optionally be the point of attachment of the alkenyl or alkynyl groups.

The term “alkoxy” refers to an —O-alkyl substituent.

As used herein, the term “halogen,” “hal,” or “halo” means —F, —Cl, —Br or —I.

The term “alkylthio” refers to an —S-alkyl substituent.

The term “alkoxyalkyl” refers to an -alkyl-O-alkyl substituent.

The term “haloalkoxy” refers to an —O-alkyl that is substituted by one or more halo substituents. Examples of haloalkoxy groups include trifluoromethoxy, and 2,2,2-trifluoroethoxy.

The term “haloalkoxyalkyl” refers to an -alkyl-O-alkyl′ where the alkyl′ is substituted by one or more halo substituents.

The term “haloalkylaminocarbonyl” refers to a —C(O)-amino-alkyl where the alkyl is substituted by one or more halo substituents.

The term “haloalkylthio” refers to an —S-alkyl that is substituted by one or more halo substituents. Examples of haloalkylthio groups include trifluoromethylthio, and 2,2,2-trifluoroethylthio.

The term “haloalkylcarbonyl” refers to an —C(O)-alkyl that is substituted by one or more halo substituents. An example of a haloalkylcarbonyl group includes trifluoroacetyl.

The term “cycloalkyl” refers to a hydrocarbon 3-8 membered monocyclic or 7-14 membered bicyclic ring system having at least one saturated ring or having at least one non-aromatic ring, wherein the non-aromatic ring may have some degree of unsaturation. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclopentenyl, cyclopentadienyl, cyclohexenyl, cyclohexadienyl, and the like.

The term “cycloalkoxy” refers to an —O-cycloalkyl substituent.

The term “cycloalkoxyalkyl” refers to an -alkyl-O-cycloalkyl substituent.

The term “cycloalkylalkoxy” refers to an —O-alkyl-cycloalkyl substituent.

The term “cycloalkylaminocarbonyl” refers to an —C(O)—NH-cycloalkyl substituent.

The term “aryl” refers to a hydrocarbon monocyclic, bicyclic, or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

The term “aryloxy” refers to an —O-aryl substituent.

The term “arylalkoxy” refers to an —O-alkyl-aryl substituent.

The term “arylalkylthio” refers to an —S-alkyl-aryl substituent.

The term “arylthioalkyl” refers to an -alkyl-S-aryl substituent.

The term “arylalkylaminocarbonyl” refers to a —C(O)-amino-alkyl-aryl substituent.

The term “arylalkylsulfonyl” refers to an —S(O)₂-alkyl-aryl substituent.

The term “arylalkylsulfinyl” refers to an —S(O)-alkyl-aryl substituent.

The term “aryloxyalkyl” refers to an -alkyl-O-aryl substituent.

The term “alkylaryl” refers to an -aryl-alkyl substituent.

The term “arylalkyl” refers to an -alkyl-aryl substituent.

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, said heteroatoms selected from O, N, or S, and the remainder ring atoms being carbon (with appropriate hydrogen atoms unless otherwise indicated). Heteroaryl groups may be optionally substituted with one or more substituents. In one embodiment, 0, 1, 2, 3, or 4 atoms of each ring of a heteroaryl group may be substituted by a substituent. Heteroaryl groups may be fully unsaturated, or they may be partially unsaturated and partially saturated. Examples of heteroaryl groups include pyridyl, furanyl, thienyl, pyrrolyl, oxazolyl, oxadiazolyl, imidazolyl thiazolyl, isoxazolyl, quinolinyl, pyrazolyl, isothiazolyl, pyridazinyl, pyrimidinyl, pyrazinyl, triazinyl, isoquinolinyl, indazolyl, and the like.

The term “heteroarylalkyl” refers to an -alkyl-heteroaryl substituent.

The term “heteroaryloxy” refers to an —O-heteroaryl substituent.

The term “heteroarylalkoxy” refers to an —O-alkyl-heteroaryl substituent.

The term “heteroaryloxyalkyl” refers to an -alkyl-O-heteroaryl substituent.

The term “nitrogen-containing heteroaryl” refers to a heteroaryl group having 1-4 ring nitrogen heteroatoms if monocyclic, 1-6 ring nitrogen heteroatoms if bicyclic, or 1-9 ring nitrogen heteroatoms if tricyclic.

The term “heterocycloalkyl” refers to a nonaromatic 3-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si, wherein the nonaromatic ring system is completely saturated. Heterocycloalkyl groups may be optionally substituted with one or more substituents. In one embodiment, 0, 1, 2, 3, or 4 atoms of each ring of a heterocycloalkyl group may be substituted by a substituent. Representative heterocycloalkyl groups include piperidinyl, piperazinyl, tetrahydropyranyl, morpholinyl, thiomorpholinyl, 1,3-dioxolane, tetrahydrofuranyl, tetrahydrothienyl, thiirenyl, and the like.

The term “heterocycloalkylalkyl” refers to an -alkyl-heterocycloalkyl substituent.

The term “alkylamino” refers to an amino substituent which is further substituted with one or two alkyl groups. The term “aminoalkyl” refers to an alkyl substituent which is further substituted with one or more amino groups. The term “hydroxyalkyl” or “hydroxylalkyl” refers to an alkyl substituent which is further substituted with one or more hydroxyl groups. The alkyl or aryl portion of alkylamino, aminoalkyl, mercaptoalkyl, hydroxyalkyl, mercaptoalkoxy, sulfonylalkyl, sulfonylaryl, alkylcarbonyl, and alkylcarbonylalkyl may be optionally substituted with one or more substituents.

The symbol “ [symbol] ” denotes the point of attachment of a chemical moiety to the remainder of a molecule or chemical formula.

The term “nucleobase” refers to nitrogen-containing biological compounds that form nucleosides. They include purine bases and pyrimidine bases. Five nucleobases-adenine (A), cytosine (C), guanine (G), thymine (T), and uracil (U)—are referred to as primary or canonical nucleobases. When a nucleobase is listed in a formula definition, it refers to that moiety covalently bonded to the recited formula.

The term “modified nucleobase” refers to derivatives of a nucleobase. Examples of modified nucleobases include, but are not limited to, xanthine, hypoxanthine, 7-methylguanine, 5,6-dihydrouracil, 5-methylcytosine, 5-hydroxymethylcytosine, purine, 2,6-diaminopurine, and 6,8-diaminopurine. When a modified nucleobase is listed in a formula definition, it refers to that moiety covalently bonded to the recited formula.

The term “substituent” and “substituent group” means an atom or group that replaces the atom or group of a named parent compound. For example, a substituent of a modified nucleoside is an atom or group that differs from the atom or group found in a naturally occurring nucleoside (e.g., a modified 2′-substituent is any atom or group at the 2′-position of a nucleoside other than H or OH). Substituent groups can be protected or unprotected. Substituents may also be further substituted with other substituent groups and may be attached directly or via a linking group such as an alkyl or hydrocarbyl group to the parent compound. Similarly, as used herein, “substituent” in reference to a chemical functional group means an atom or group of atoms that differs from the atom or group of atoms normally present in the named functional group. In certain embodiments, substituents on any group (such as, for example, alkyl, alkenyl, alkynyl, aryl, aralkyl, heteroaryl, heteroaralkyl, cycloalkyl, heterocycloalkyl) can be at any atom of that group, wherein any group that can be substituted (such as, for example, alkyl, alkenyl, alkynyl, aryl, aralkyl, heteroaryl, heteroaralkyl, cycloalkyl, heterocycloalkyl) can be optionally substituted with one or more substituents (which may be the same or different), each replacing a hydrogen atom. Examples of suitable substituents include, but are not limited to alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, aryloxycarbonyl, heteroaryloxy, heteroaryloxycarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, amido, alkylaminosulfonyl, arylaminosulfonyl, dialkylaminosulfonyl, alkylsulfonylamino, arylsulfonylamino, imino, carboxamido, carbamido, carbamyl, thioureido, thiocyanato, sulfoamido, sulfonylalkyl, sulfonylaryl, mercaptoalkoxy, N-hydroxyamidinyl, or N′-aryl, N″-hydroxyamidinyl. In certain embodiments, substituents on any group include alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, thiocarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, or amido. In certain embodiments, substituents on any group include alkyl, halogen, haloalkyl, cyano, nitro, alkoxy, hydroxyl, hydroxylalkyl, carboxyl, formyl, alkylcarbonyl, alkoxycarbonyl, alkylcarbonyloxy, thio, mercapto, mercaptoalkyl, amino, aminoalkyl, dialkylamino, alkylcarbonylamino, alkylaminocarbonyl, or alkylamino.

The term “protecting group” or “protecting moiety” refers to a substituent that is commonly employed to block or protect a particular functionality while reacting other functional groups on the compound, a derivative thereof, or a conjugate thereof, and includes a nitrogen protecting group when attached to a nitrogen atom, or an oxygen protecting group when attached to an oxygen atom. Nitrogen and oxygen protecting groups are well known in the art and include those described in detail in Protecting Groups in Organic Synthesis, T. W. Greene and P. G. M. Wuts, 3rd edition, John Wiley & Sons, 1999, incorporated herein by reference.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein each R^{aa}, R^{bb}, and R^{cc} is independently alkyl, cycloalkyl, aryl, or heteroaryl, each of which may be optionally substituted with 1-3 independent R^{dd}, and each R^{dd} is independently alkyl, alkenyl, alkynyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, aryloxycarbonyl, heteroaryloxy, heteroaryloxycarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, amido, alkylaminosulfonyl, arylaminosulfonyl, dialkylaminosulfonyl, alkylsulfonylamino, arylsulfonylamino, imino, carbamido, carbamyl, thioureido, thiocyanato, sulfoamido, sulfonylalkyl, sulfonylaryl, or mercaptoalkoxy. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Amide nitrogen protecting groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine, o-nitrobenzamide, and o-(benzoyloxymethyl)benzamide.

Carbamate nitrogen protecting groups (e.g., —C(═O)OR^{aa}) include, but are not limited to, methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Sulfonamide nitrogen protecting groups (e.g., —S(═O)₂R′) include, but are not limited to, p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to as a hydroxyl protecting group). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR—, —C(═NR^{bb})N(R^{bb})₂, —S(═O)R^{aa}, —SO₂R^{aa}, —Si(R^{aa})₃, —P(R^{cc})₂, —P(R^{cc})₃, —P(═O)₂R^{aa}, —P(═O)(R^{aa})₂, —P(═O)(OR″)₂, —P(═O)₂N(R^{bb})₂, and —P(═O)(NR^{bb})₂, wherein R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, a-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), t-butyl carbonate (BOC), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, a-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a thiol protecting group). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{cc})₃, —P(R^{cc})₂, —P(R^{cc})₃, —P(═O)₂R^{aa}, —P(═O)(R^{cc})₂, —P(═O)(OR^{cc})₂, —P(═O)₂N(R^{bb})₂, and —P(═O)(NR^{bb})₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

The term “antisense oligonucleotide” or “antisense strand” means an oligonucleotide which includes a region that is complementary to a target nucleic acid.

The term “composition” or “pharmaceutical composition” means a mixture of substances suitable for administering to a subject. For example, a composition may comprise one or more compounds or salt thereof and a sterile aqueous solution.

The term “nucleic acid” refers to molecules composed of linked monomeric nucleotides or nucleosides. A nucleic acid includes, but is not limited to, ribonucleic acids (RNA), deoxyribonucleic acids (DNA), single-stranded nucleic acids, and double-stranded nucleic acids.

The term “nucleobase sequence” means the order of contiguous nucleobases in a nucleic acid or oligonucleotide independent of any sugar or internucleoside linkage.

The term “nucleoside” means a compound comprising a nucleobase and a sugar moiety. The nucleobase and sugar moiety are each, independently, unmodified or modified. “Modified nucleoside” means a nucleoside comprising a modified nucleobase and/or a modified sugar moiety. Modified nucleosides include abasic nucleosides, which lack a nucleobase.

The term “oligomeric compound” means a polymer of linked subunits. With reference to a protein, peptide, polypeptide, or antibody, “subunit” refers to an amino acid or peptide bond. With reference to an oligonucleotide, “subunit” refers to a nucleotide, nucleoside, nucleobase, or sugar, or a modified nucleotide, nucleoside, nucleobase, or sugar as provided herein.

The term “oligonucleotide” means a polymer of linked nucleosides (e.g., polynucleotide, nucleic acid, polymer of nucleotides), each of which can be modified or unmodified, independent from one another. Without limitation, an oligonucleotide may be comprised of ribonucleic acids (e.g., comprised of ribonucleosides), deoxyribonucleic acids (e.g., comprised of deoxyribonucleosides), modified nucleic acids (e.g., comprised of modified nucleobases, sugars, and/or phosphate groups), or a combination thereof. Examples of oligonucleotide compounds include single-stranded and double-stranded compounds, such as oligonucleotides, antisense oligonucleotides, interfering RNA compounds (RNAi compounds), microRNA (miRNA) targeting oligonucleotides, miRNA mimics, occupancy-based compounds (e.g., mRNA processing or translation blocking compounds and splicing compounds) and editing compounds (e.g., ADAR recruiting molecules, ADAR targeting molecules, single-stranded guide nucleic acids, or a combination thereof). RNAi compounds include double-stranded compounds (e.g., short-interfering RNA (siRNA) and double-stranded RNA (dsRNA)) and single-stranded compounds (e.g., single-stranded siRNA (ssRNA), single-stranded RNAi (ssRNAi), short hairpin RNA (shRNA), and microRNA mimics) which work at least in part through the RNA-induced silencing complex (RISC) pathway resulting in sequence specific degradation and/or sequestration of a target nucleic acid through a process known as RNA interference (RNAi). The term “RNAi compound” is meant to be equivalent to other terms used to describe nucleic acid compounds that are capable of mediating sequence-specific RNA interference, for example, interfering RNA (iRNA), iRNA agent, RNAi agent, small interfering RNA, short interfering RNA, short interfering oligonucleotide, short interfering nucleic acid, short interfering modified oligonucleotide, chemically modified siRNA, and others. Additionally, the term “RNAi” is meant to be equivalent to other terms used to describe sequence-specific RNA interference.

The term “target nucleic acid,” “target RNA,” and “nucleic acid target” all mean a nucleic acid capable of being targeted by compounds described herein.

The term “therapeutic compound” includes any pharmaceutical agent or compound that provides a therapeutic benefit to a subject. Therapeutic compounds include nucleic acids, oligomeric compounds, oligonucleotides, proteins, peptides, antibodies, small molecules, and other such agents.

“Target region” means a portion of a target nucleic acid to which one or more compounds is targeted.

“Targeting moiety” means a conjugate group that provides an enhanced affinity for a selected target, e.g., molecule, cell or cell type, compartment, e.g., a cellular or organ compartment, tissue, organ, or region of the body, as, e.g., compared to a compound absent such a moiety.

“Terminal group” means a chemical group or group of atoms that is covalently linked to a terminus of an oligonucleotide.

The term “ligand” refers to a substance that binds to or otherwise interacts with a protein, nucleic acid, or other biological molecule. In some embodiments, a ligand is a small molecule. In some embodiments, a ligand binds to a protein (e.g., a receptor). In certain embodiments, a ligand binds to an NMDA receptor.

The term “conjugated” refers to two molecules (e.g., an oligonucleotide and a ligand as described herein) that are joined together by a covalent bond. The covalent bond to, for example, a ligand or an oligonucleotide, can be attached at any atom of the ligand or the oligonucleotide (i.e., a radical of the ligand or the oligonucleotide).

The term “N-methyl-D-aspartate receptor” or “NMDA receptor” refers to the glutamate receptor and ion channel that is found in neurons, e.g., in humans. Many ligands of the NMDA receptor are known in the art including, for example, those disclosed in Neuropharmacology 2007, 53(6), 699-723; J. Med. Chem. 1990, 33(2), 789-808; Neuroscience 2001, 105(3), 663-669; J. Med. Chem. 2022, 65(13), 9063-9075; Drugs Fut. 2004, 29(10), 992; Drugs Fut. 2004, 29(10), 993; and British Journal of Pharmacology 2022, 179(6), 1146-1187, each of which is incorporated by reference herein.

The term “sense oligonucleotide” or “sense strand” means the strand of a double-stranded compound that includes a region that is substantially complementary to a region of the antisense strand of the double-stranded compound.

The terms “microRNA” and “miRNA,” as may be used interchangeably herein, refer to short (e.g., about 20 to about 24 nucleotides in length) non-coding ribonucleic acids (RNAs) that are involved in post-transcriptional regulation of gene expression in multicellular organisms by affecting both the stability and translation of mRNAs. miRNAs are transcribed by RNA polymerase II as part of capped and polyadenylated primary transcripts (pri-miRNAs) that can be either protein-coding or non-coding. The primary transcript is cleaved by the Drosha ribonuclease III enzyme to produce a stem-loop precursor miRNA (pre-miRNA) approximately 70 nucleotides in length, which is further processed in the RNAi pathway. As part of this pathway, the pre-miRNA is cleaved by the cytoplasmic Dicer ribonuclease to generate the mature miRNA and antisense miRNA star (miRNA*) products. The mature miRNA is incorporated into an RNA-induced silencing complex (RISC), which recognizes target mRNAs through imperfect base pairing (i.e., partial complementarity) with the miRNA and most commonly results in translational inhibition or destabilization of the target mRNA. This mechanism is most often seen through the binding of the miRNA on the 3′ untranslated region (UTR) of the target mRNA, which can decrease gene expression by either inhibiting translation (for example, by blocking the access of ribosomes for translation) or directly causing degradation of the transcript. The term (i.e., miRNA) may be used herein to refer to any form of the subject miRNA (e.g., precursor, primary, and/or mature miRNA).

The terms “small interfering RNA” “short interfering RNA” and “siRNA,” as may be used interchangeably herein, refer to RNA molecules that present as non-coding double-stranded RNA (dsRNA) molecules of about 20 to about 24 nucleotides in length and are useful in RNA interference (RNAi). siRNA are often found with phosphorylated 5′ ends and hydroxylated 3′ ends, which 3′ ends typically have a 2-nucleotide overhang beyond the 5′ end of the anti-parallel strand (e.g., complementary strand of the dsRNA molecule). siRNA can interfere with the expression of specific genes through binding of target sequences (e.g., target nucleic acid sequences) to which they are complementary and promoting (e.g., facilitating, triggering, initiating) degradation of the mRNA, thereby preventing (e.g., inhibiting, silencing, interfering with) translation. After integration and separation into the RISC complex, siRNAs base-pair (e.g., full complementarity) to their target mRNA and cleave it, thereby preventing it from being used as a translation template. As discussed herein above, also part of the RNAi pathway, a miRNA-loaded RISC complex scans cytoplasmic mRNAs for potential complementarity (e.g., partial complementarity).

The term “ADAR recruiting molecule,” as may be used herein, refers to a nucleic acid that is configured to increase the concentration of Adenosine Deaminase Acting on Ribonucleic Acid (ADAR) enzyme in a locality around the nucleic acid. In some embodiments, an increased concentration is relative to the concentration in a given locality absent the ADAR recruiting molecule. In some embodiments, an ADAR recruiting molecule comprises a double-stranded RNA duplex.

The term “ADAR targeting molecule,” as may be used herein, refers to a nucleic acid that is configured to direct an ADAR molecule to a desirable location (e.g., locality). As used herein, the term “direct” refers to increasing the concentration of ADAR in the desirable location as compared to the concentration absent the ADAR targeting molecule. In some embodiments, the ADAR targeting molecule can be configured to control the desirable location by altering the sequence and/or properties of the nucleic acid (e.g., by modifications to the nucleobase, sugar, internucleoside linkage, or other component). In some embodiments, an ADAR targeting molecule comprises an ADAR recruiting molecule and a single-stranded guide nucleic acid. In some embodiments, an ADAR targeting molecule comprises a double-stranded RNA duplex and a single-stranded guide nucleic acid.

The term “single-stranded guide nucleic acid” or “guide RNA” as may be used herein, refers to a nucleic acid of a single strand, which comprises a specific sequence that is at least partially complementary to a target sequence. In some embodiments, the target sequence is at, adjacent to, or in proximity to, a locality where it is desirable to modulate ADAR concentration. In some embodiments, the level of complementarity is sufficient to facilitate binding (e.g., annealing) of the single-stranded guide nucleic acid to the target sequence.

The compounds of the present disclosure may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present disclosure, whether radioactive or not, are encompassed within the scope of the present disclosure.

The term “isotopic variant” refers to a therapeutic agent (e.g., a compound and/or modified oligonucleotide disclosed herein) that contains an unnatural proportion of an isotope at one or more of the atoms that constitute such a therapeutic agent. In certain embodiments, an “isotopic variant” of a therapeutic agent contains unnatural proportions of one or more isotopes, including, but not limited to, hydrogen (H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine 123 (¹²³I), iodine-125 (¹²⁵I), iodine-127 (¹²⁷I), iodine-129 (¹²⁹I), and iodine-131 (¹³¹I). In certain embodiments, an “isotopic variant” of a therapeutic agent contains unnatural proportions of one or more isotopes, including, but not limited to, hydrogen (H), deuterium (²H), tritium (³H), carbon-11 (¹¹C), carbon-12 (¹²C), carbon-13 (¹³C), carbon-14 (¹⁴C), nitrogen-13 (¹³N), nitrogen-14 (¹⁴N), nitrogen-15 (¹⁵N), oxygen-14 (¹⁴O), oxygen-15 (¹⁵O), oxygen-16 (¹⁶O), oxygen-17 (¹⁷O), oxygen-18 (¹⁸O), fluorine-17 (¹⁷F), fluorine-18 (¹⁸F), phosphorus-31 (³¹P), phosphorus-32 (³²P), phosphorus-33 (³³P), sulfur-32 (³²S), sulfur-33 (³³S), sulfur-34 (³⁴S), sulfur-35 (³⁵S), sulfur-36 (³⁶S), chlorine-35 (³⁵Cl), chlorine-36 (³⁶Cl), chlorine-37 (³⁷Cl), bromine-79 (⁷⁹Br), bromine-81 (⁸¹Br), iodine 123 (¹²³I), iodine-125 (¹²⁵I), iodine-127 (¹²⁷I), iodine-129 (¹²⁹I), and iodine-131 (¹³¹I).

It will be understood that, in a therapeutic agent (e.g., a compound and/or modified oligonucleotide disclosed herein), any hydrogen can be ²H, for example, or any carbon can be ¹³C, for example, or any nitrogen can be ¹⁵N, for example, or any oxygen can be ¹⁸O, for example, where feasible according to the judgment of one of skill. In certain embodiments, an “isotopic variant” of a therapeutic agent contains unnatural proportions of deuterium (D).

“Modified oligonucleotide” means an oligonucleotide, wherein at least one sugar, nucleobase, or internucleoside linkage is modified.

“Nucleobase sequence” means the order of contiguous nucleobases in a nucleic acid or oligonucleotide independent of any sugar or internucleoside linkage.

The term “oligomeric duplex” means a duplex formed by two oligomeric compounds having complementary nucleobase sequences. Each oligomeric compound of an oligomeric duplex may be referred to as a “duplexed oligomeric compound.” The oligonucleotides of each oligomeric compound of an oligomeric duplex may include non-complementary overhanging nucleosides. In some embodiments, the terms “duplexed oligomeric compound” and “modified oligonucleotide” are used interchangeably. In other embodiments, the terms “oligomeric duplex” and “compound” are used interchangeably.

“Phosphorothioate linkage” means a modified phosphate linkage in which one of the non-bridging oxygen atoms is replaced with a sulfur atom.

The terms “RNA interference compound,” “RNAi compound,” and/or “iRNA agent” mean a compound that acts, at least in part, through an RNA-induced silencing complex (RISC) pathway or Ago2, but not through RNase H, to modulate a target nucleic acid and/or protein encoded by a target nucleic acid. RNAi compounds include, but are not limited to, double-stranded siRNA, single-stranded siRNA, and microRNA, including microRNA mimics.

### Certain Embodiments

In certain embodiment, a compound comprises an NMDA receptor ligand and one or more linker moieties. In certain embodiments, the compound is selected from any of formulae I, II, II-a, III, III-a, IV, IV-a, V, V-a, VI, VI-a, VI-b, VII, VII-a, VIII, VIII-a, IX, IX-a, X, X-a, or salts thereof, as described herein. In certain embodiments, the one or more linker moieties (L₁, L₂, L₃, L₄, etc.) links the NMDA receptor ligand to a therapeutic, prophylactic, or diagnostic agent. In certain embodiments, the compound further comprises one or more therapeutic, prophylactic, or diagnostic agents. In certain embodiments, a therapeutic, prophylactic, or diagnostic agent is a small molecule, or an oligomeric compound. In certain embodiments, the oligomeric compound comprises a protein, a peptide, an antibody, an oligonucleotide, or a combination thereof.

In certain embodiments, an oligomeric compound is any of those described herein. In certain embodiments, the oligomeric compound is about 10-50 subunits in length. In certain embodiments the oligomeric compound is an oligonucleotide. In certain embodiments, an oligonucleotide is any of those described herein. In certain embodiments, the oligonucleotide is 8 to 80 linked nucleosides in length, 12-50 linked nucleosides in length, 12-30 linked nucleosides in length, or 15-30 linked nucleosides in length.

In certain embodiments, the oligonucleotide is a modified oligonucleotide comprising at least one modified internucleoside linkage, at least one modified sugar, or at least one modified nucleobase.

In certain embodiments, the oligonucleotide is single-stranded. In certain embodiments, the oligonucleotide is double-stranded. In certain embodiments, the oligonucleotide is double-stranded over a portion of its length. In certain embodiments, the oligonucleotide comprises ribonucleic acids (e.g., comprised of ribonucleosides), deoxyribonucleic acids (e.g., comprised of deoxyribonucleosides), or a combination thereof.

In certain embodiments, the oligonucleotide is a small interfering RNA (siRNA), a microRNA (miRNA) antagonist, a miRNA mimic, an ADAR recruiting molecule, an ADAR targeting molecule, a guide RNA, an antisense oligonucleotide, a short hairpin RNA (shRNA), or combinations thereof.

In some embodiments, a linker is an optionally substituted alkyl linker. In some embodiments, a linker is an optionally substituted C₁-C₆ alkyl linker. In some embodiments, a linker is a C₁-C₆ alkyl linker substituted with ═O. In certain embodiments, a linker comprises the structure

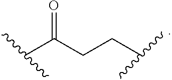 [SMILES: *CCC(*)=O]

In some embodiments, a linker is an optionally substituted C₁-C₁₅ alkyl linker. In some embodiments, a linker is an optionally substituted C₅-C₁₂ alkyl linker. In certain embodiments, a linker comprises the structure

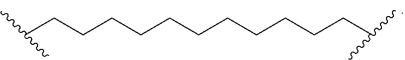 [SMILES: *CCCCCCCCCCC*]

In certain embodiments, a linker comprises the structure

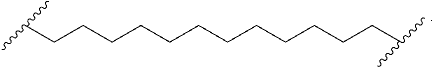 [SMILES: *CCCCCCCCCCCC*]

In some embodiments, a linker is a bond. In some embodiments, a linker is an optionally substituted PEG linker. In some embodiments, a linker is an optionally substituted PEG linker comprising one, two, three, four, five, six, seven, or eight PEG units in length, wherein a PEG unit comprises the structure

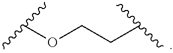 [SMILES: *CCO*]

In certain embodiments, a linker is an optionally substituted PEG linker comprising three PEG units in length. In certain embodiments, a linker is an optionally substituted PEG linker comprising four PEG units in length. In certain embodiments, a linker comprises the structure

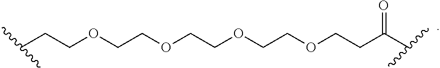 [SMILES: *CCOCCOCCOCCOCCC(*)=O]

In certain embodiments, a linker comprises the structure

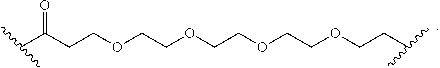 [SMILES: *CCOCCOCCOCCOCCC(*)=O]

In certain embodiments, a linker comprises the structure

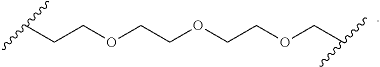 [SMILES: *CCOCCOCCOC*]

In certain embodiments, a linker comprises the structure

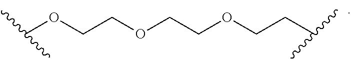 [SMILES: *CCOCCOCCO*]

In certain embodiments, a linker comprises the structure

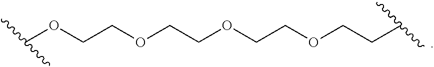 [SMILES: *CCOCCOCCOCCO*]

In certain embodiments, a linker comprises the structure

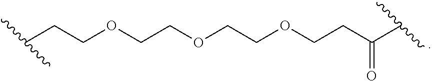 [SMILES: *CCOCCOCCOCCC(*)=O]

In certain embodiments, a linker comprises the structure

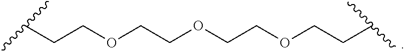 [SMILES: *CCOCCOCCOCC*]

In certain embodiments, a linker comprises the structure

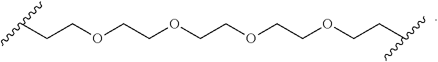 [SMILES: *CCOCCOCCOCCOCC*]

In some embodiments, a linker is an optionally substituted heteroalkyl linker. In certain embodiments, a linker comprises the structure

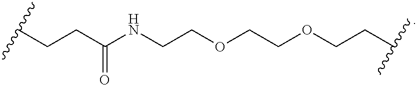 [SMILES: *CCOCCOCCNC(=O)CC*]

In certain embodiments, a linker comprises the structure

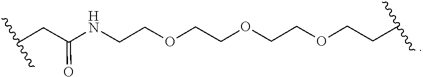 [SMILES: *CCOCCOCCOCCNC(=O)C*]

In some embodiments, a linker is an optionally substituted heteroaryl linker. In some embodiments, a linker is an optionally substituted partially unsaturated heterocycloalkyl linker or a heteroaryl linker. In some embodiments, a linker comprises the structure

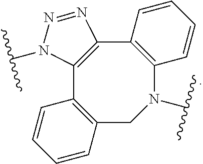 [SMILES: *N1Cc2ccccc2-c2c(nnn2*)-c2ccccc21]

In some embodiments, a linker is an optionally substituted heteroalkyl linker. In some embodiments, a linker is substituted with one or more ═O substituents. In certain embodiments, a linker comprises the structure

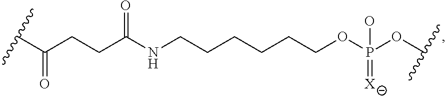

wherein X is O or S. In certain embodiments, a linker comprises the structure

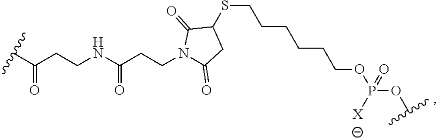

wherein X is O or S.

In some embodiments, a linker comprises the structure

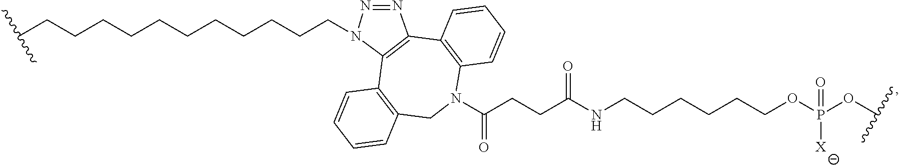

wherein X is O or S. In some embodiments, a linker comprises the structure

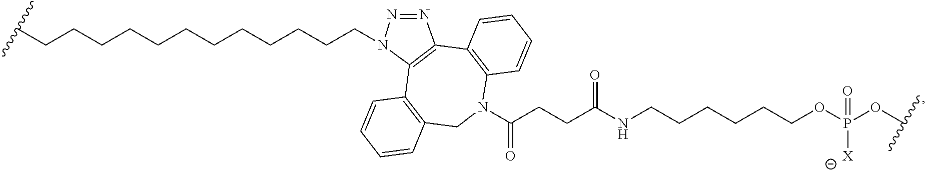

wherein X is O or S. In some embodiments, a linker comprises the structure

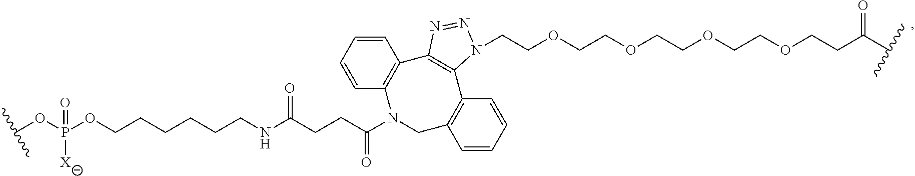

wherein X is O or S. In some embodiments, a linker comprises the structure

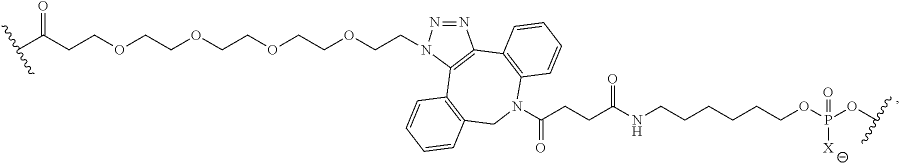

wherein X is O or S. In some embodiments, a linker comprises the structure

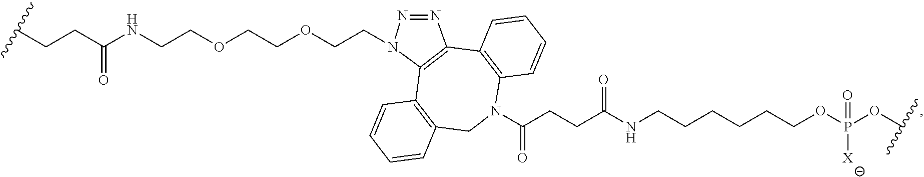

wherein X is O or S. In some embodiments, a linker comprises the structure

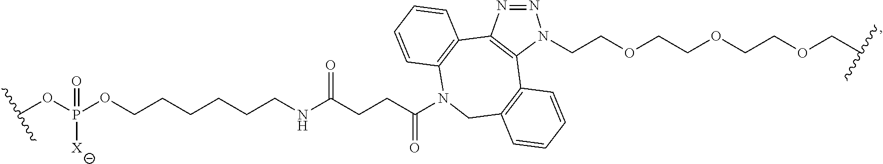

wherein X is O or S. In some embodiments, a linker comprises the structure

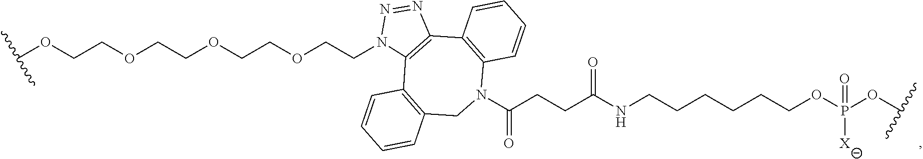

wherein X is O or S. In some embodiments, a linker comprises the structure

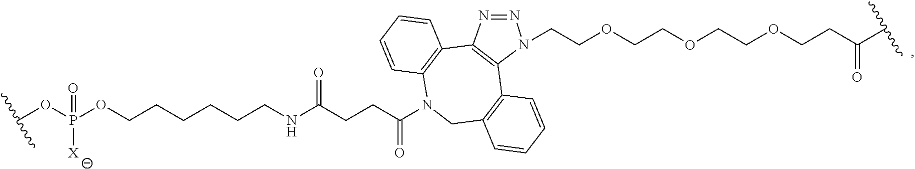

wherein X is O or S. In some embodiments, a linker comprises the structure

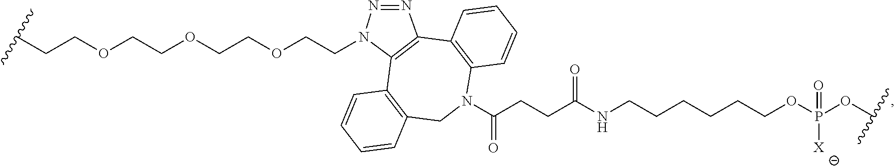

wherein X is O or S. In some embodiments, a linker comprises the structure

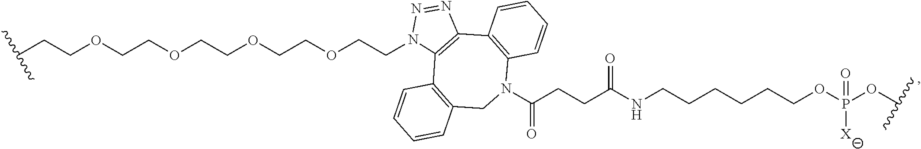

wherein X is O or S. In some embodiments, a linker comprises the structure

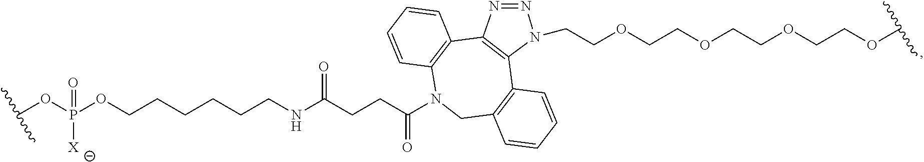

wherein X is O or S. In some embodiments, a linker comprises the structure

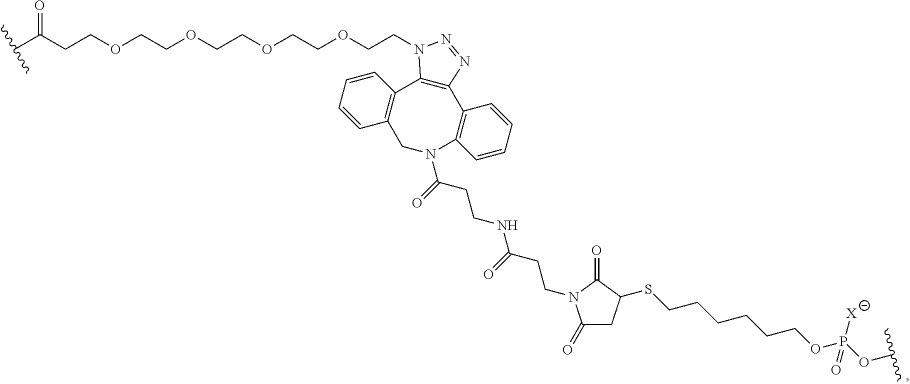

wherein X is O or S.

In certain embodiments, a compound comprises or consists of one of the structures:

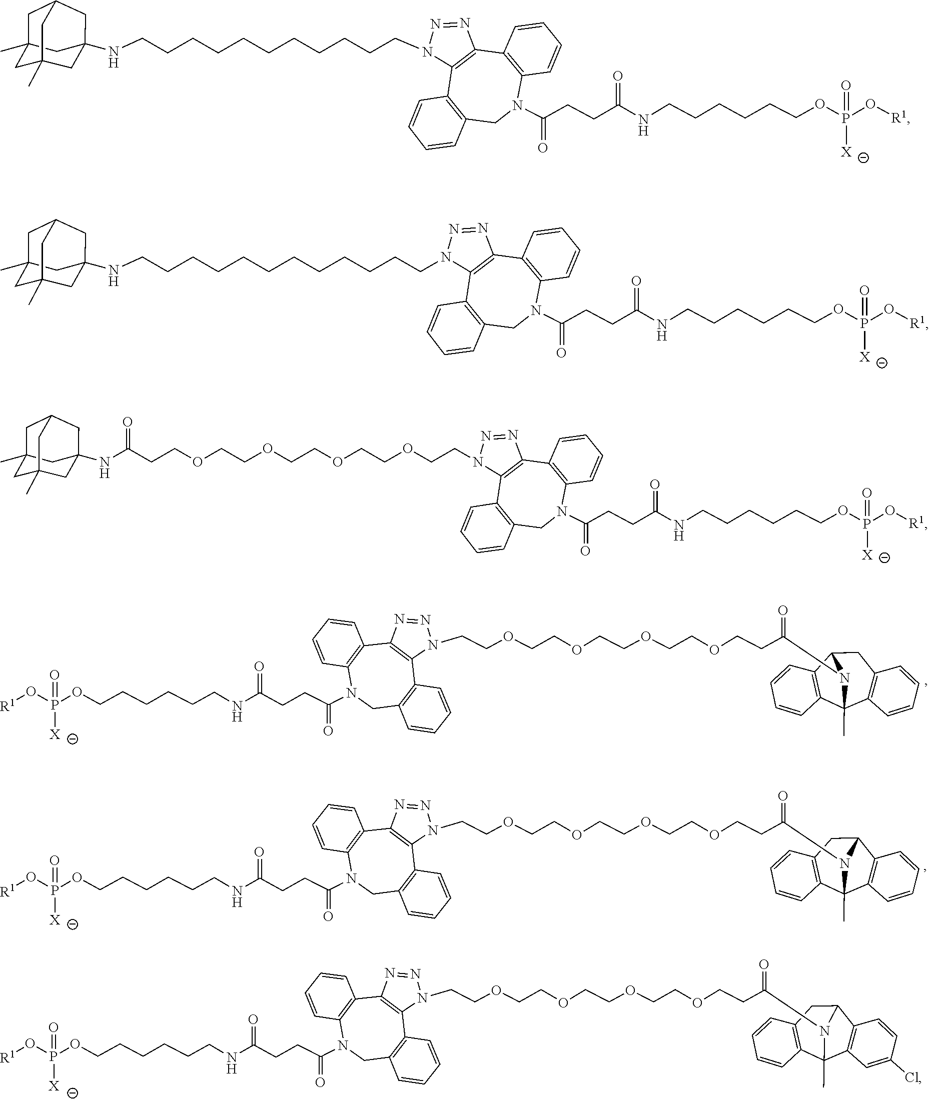

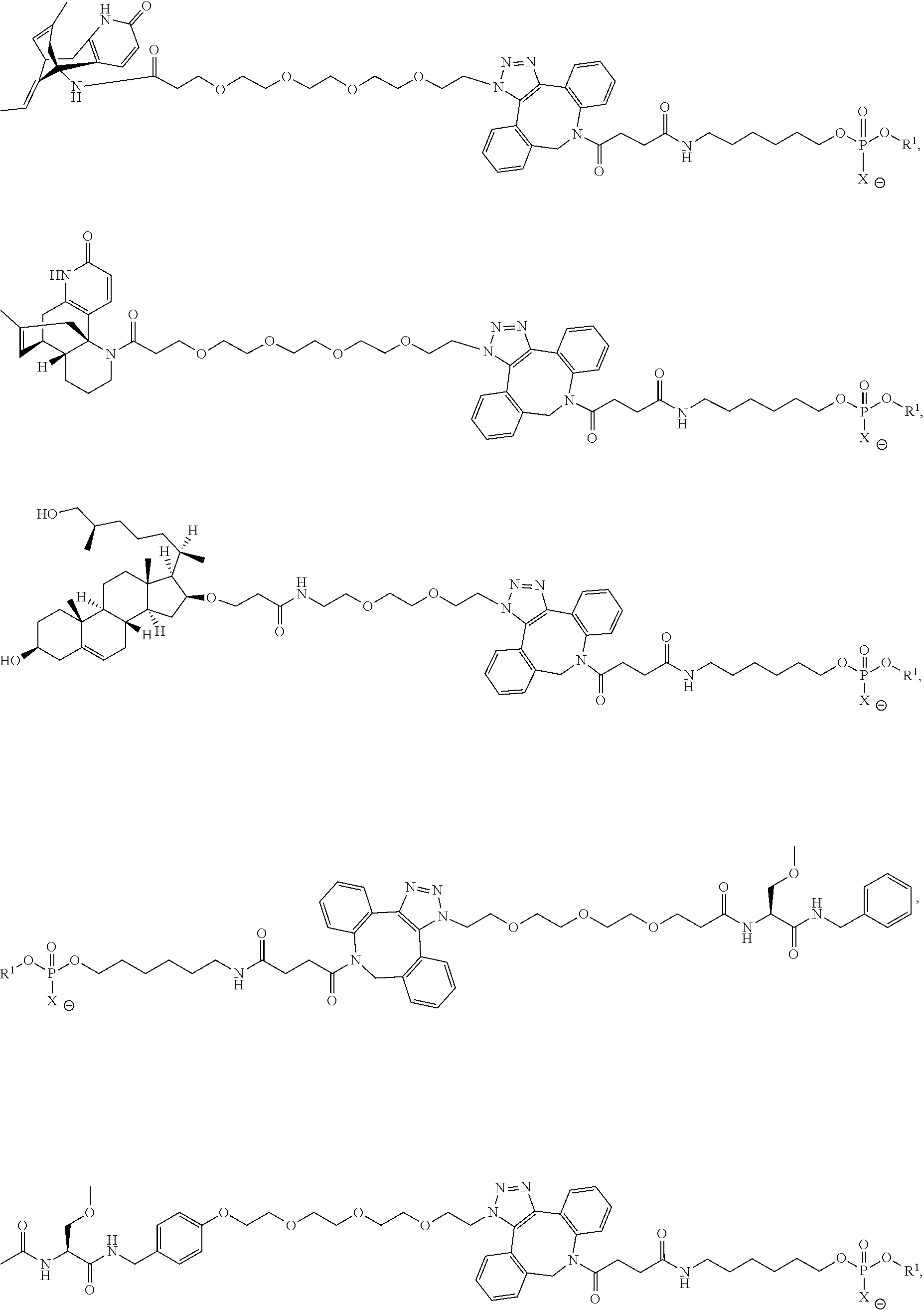

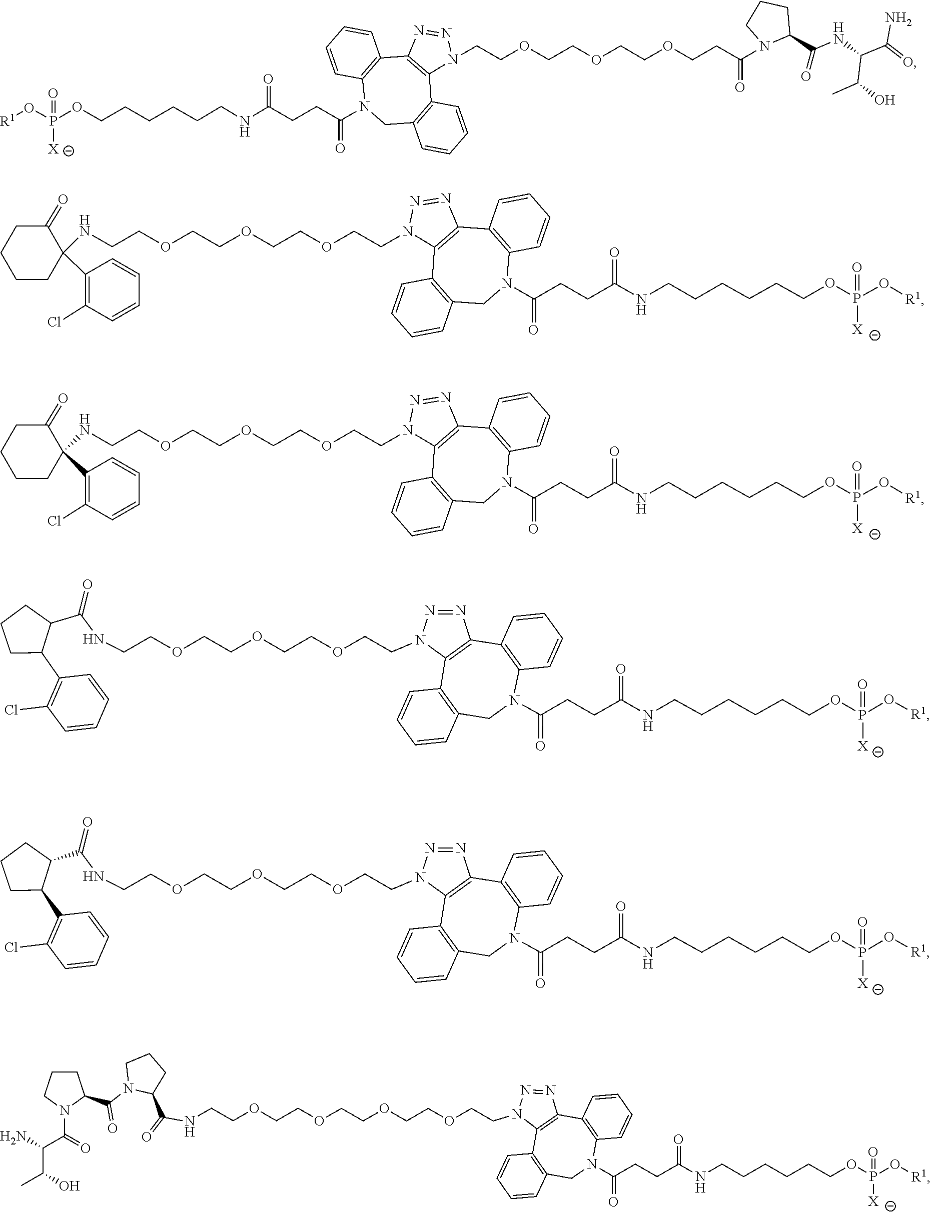

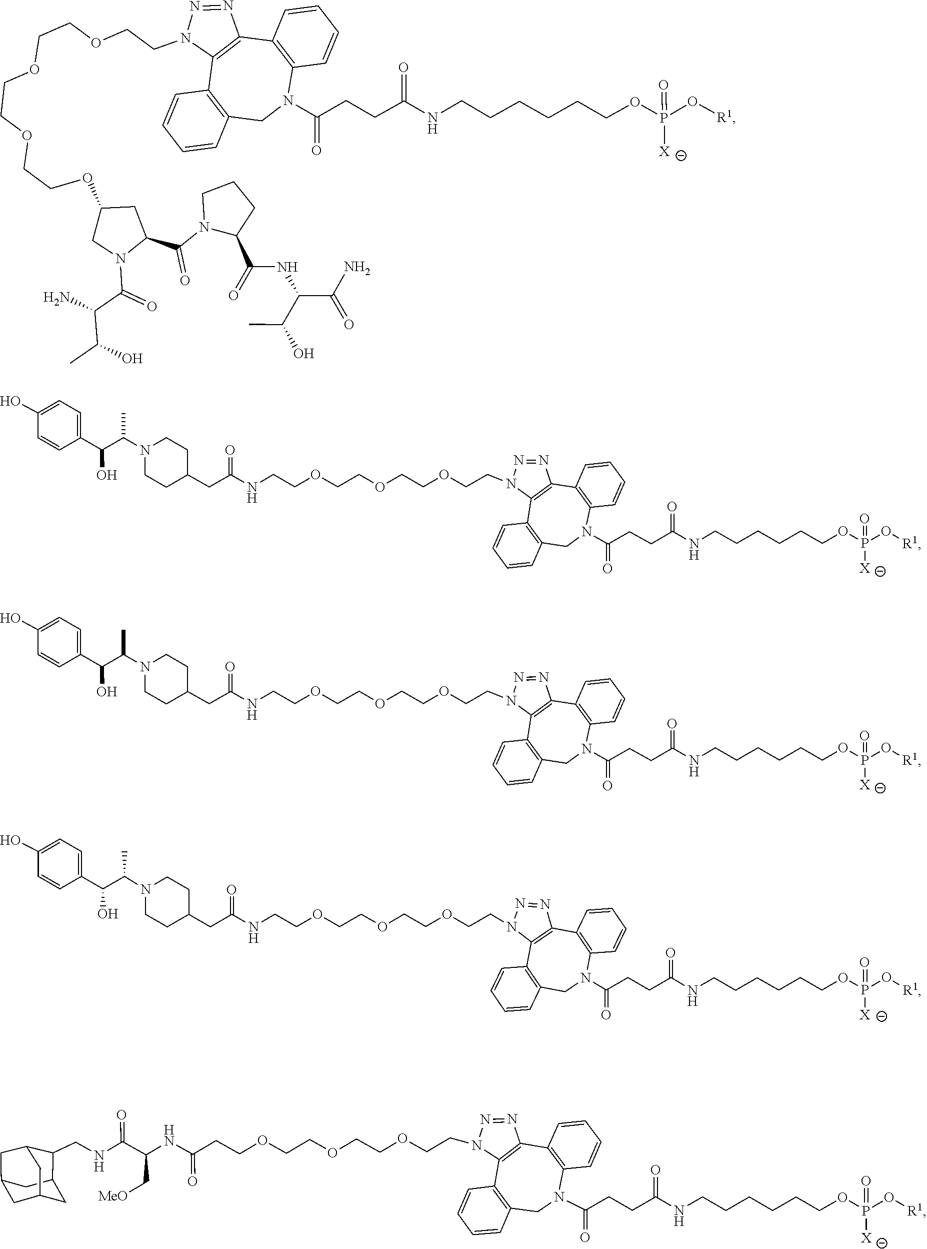

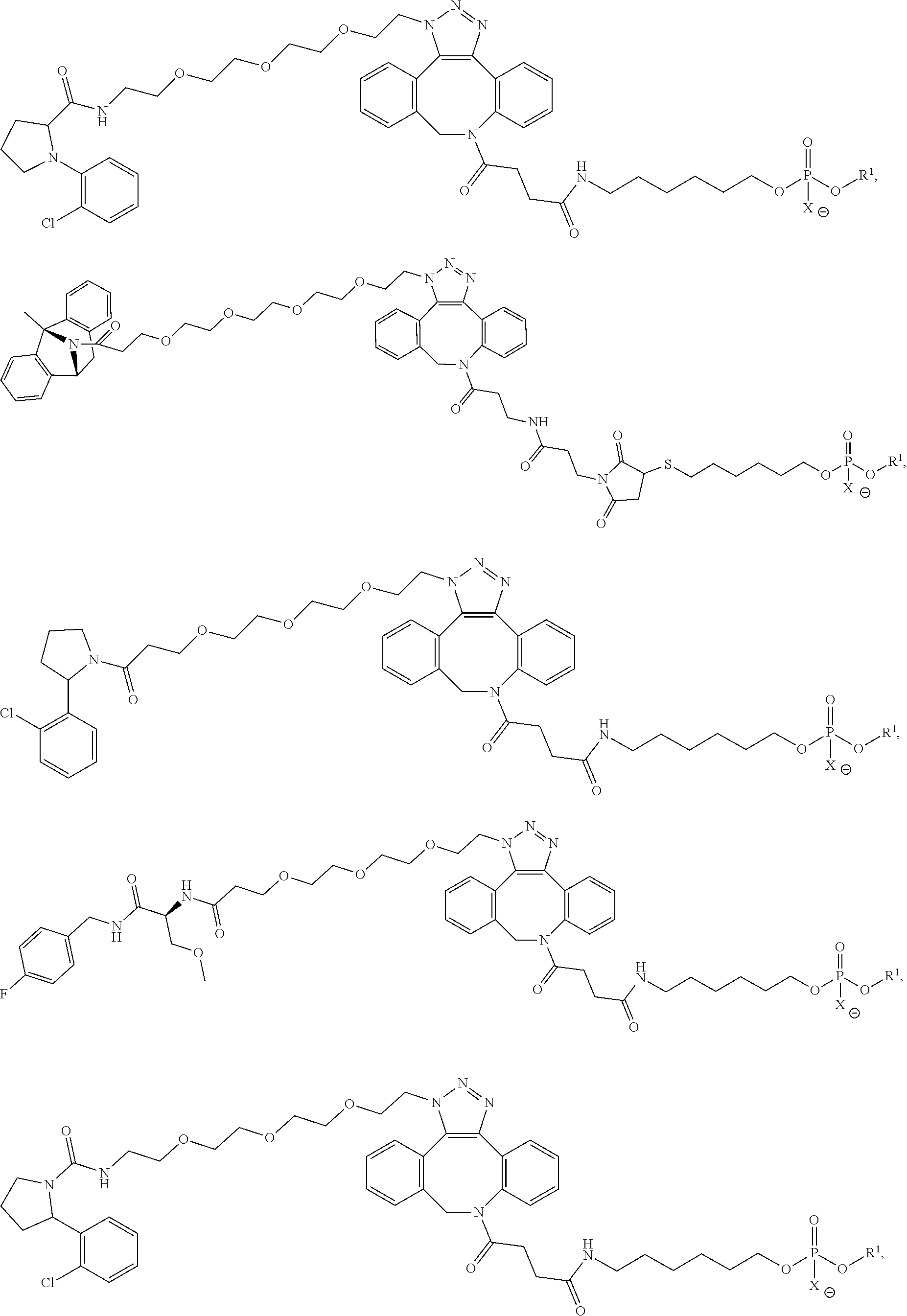

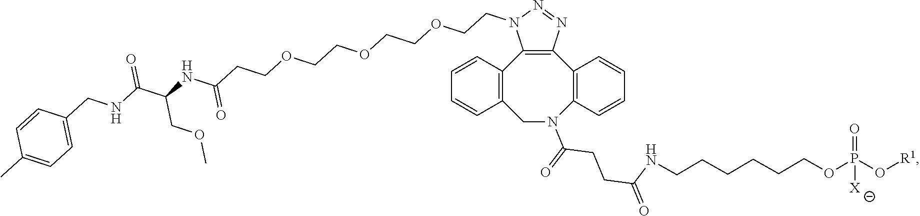

or a salt thereof, wherein X is O or S.

In some embodiments, X is O. In some embodiments, X is S.

In some embodiments, R¹ comprises an oligonucleotide. In some embodiments, the oligonucleotide is attached at its 5′ end. In some embodiments, the oligonucleotide is attached at its 3′ end. In some embodiments, the oligonucleotide is attached at an internal position on the oligonucleotide. In some embodiments the internal position is at an internucleoside linkage. In some embodiments, R¹ comprises an oligonucleotide conjugated to one or more additional NMDA receptor ligands. In some embodiments, the oligonucleotide is conjugated to two, three, four, five, or more than five additional NMDA receptor ligands. In certain embodiments, the additional NMDA receptor ligands are conjugated to the oligonucleotide at the 5′ end of the oligonucleotide, the 3′ end of the oligonucleotide, one or more internal positions on the oligonucleotide, or any combination thereof. In certain embodiments, the oligonucleotide is a modified oligonucleotide.

Certain embodiments provide a composition comprising a compound of any embodiment herein, and a pharmaceutically acceptable carrier or excipient.

Certain embodiments provide a composition comprising a compound of any embodiment herein, for use in therapy.

In certain embodiments, a method for delivering an agent to cell comprises contacting the cell with the compound of any of the embodiments herein, thereby delivering the agent to the cell. In certain embodiments, the cell expresses NMDA receptor on the surface of the cell. In certain embodiments, the cell is a brain cell. In certain embodiments the cell is a cell of the frontal cortex. In certain embodiments, the cell is a cell of the striatum. In certain embodiments, the cell is a cell of the cerebellum. In certain embodiments, the cell is a cell of the brain stem. In certain embodiments, the cell is a cell of the hippocampus. In certain embodiments, the cell is a cell of the spinal cord. In certain embodiments, the agent is a therapeutic agent or diagnostic agent. In certain embodiments, the cell is in an animal.

In certain embodiments, a method of modulating the expression of a nucleic acid target in a cell comprises contacting the cell with the compound of any of the embodiments herein, thereby modulating expression of the nucleic acid target in the cell. In certain embodiments, the cell expresses NMDA receptor on the surface of the cell. In certain embodiments, the cell is a brain cell. In certain embodiments the cell is a cell of the frontal cortex. In certain embodiments, the cell is a cell of the striatum. In certain embodiments, the cell is a cell of the cerebellum. In certain embodiments, the cell is a cell of the brain stem. In certain embodiments, the cell is a cell of the hippocampus. In certain embodiments, the cell is a cell of the spinal cord. In certain embodiments, the agent is a therapeutic agent or a diagnostic agent. In certain embodiments, contacting the cell with the compound of any of the embodiments herein inhibits expression of the nucleic acid target. In certain embodiments, the nucleic acid target is pre-mRNA, mRNA, non-coding RNA, or miRNA. In certain embodiments, the cell is in an animal.

In certain embodiments, a method of modulating the expression of a nucleic acid target in a subject comprises administering to the subject any of the compounds or compositions provided herein, thereby modulating expression of the nucleic acid target in the subject. In certain embodiments, the expression of the nucleic acid is modulated in a cell of the subject that expresses NMDA receptor on the surface of the cell. In certain embodiments, the expression of the nucleic acid is modulated in a brain cell. In certain embodiments, the cell expressing NMDA receptor on its surface is a brain cell. In certain embodiments, the brain cell is a cell of the frontal cortex. In certain embodiments, the brain cell is a cell of the striatum. In certain embodiments, the brain cell is a cell of the cerebellum. In certain embodiments, the brain cell is a cell of the brain stem. In certain embodiments, the brain cell is a cell of the hippocampus. In certain embodiments, the brain cell is a cell of the spinal cord. In certain embodiments, the nucleic acid target is pre-mRNA, mRNA, non-coding RNA, or miRNA. In certain embodiments, the compound is administered to the subject intrathecally.

In certain embodiments, a method of treating or ameliorating a disease, disorder, or symptom thereof in a subject, comprises administering to the subject any of the compounds or compositions provided herein, thereby treating, preventing, or ameliorating a disease, disorder, or symptom in the subject. In certain embodiments, the disease, disorder, or symptom thereof is a central nervous system (CNS) disease, disorder, or symptom thereof. In certain embodiments, the disease, disorder, or symptom thereof is Alzheimer's disease, or a symptom thereof. In certain embodiments, the compound is administered to the subject intrathecally. In certain embodiments, the compound or composition is administered to the subject in a therapeutically effective amount.

In certain embodiments, a compound comprising an NMDA receptor ligand selectively or preferentially targets a cell expressing NMDA receptor compared to a cell not expressing NMDA receptor. In certain embodiments, a compound comprising an NMDA receptor ligand selectively or preferentially targets a cell expressing NMDA receptor compared to a compound not comprising an NMDA receptor ligand.

Also provided herewith is the use of a compound as described herein for the manufacture of a medicament in the treatment of a disease or disorder.

In another aspect, the present disclosure provides methods for making any of the compounds provided herein, comprising one or more compounds and chemical transformations described herein, including Examples 1-77.

### Certain Compounds Comprising an Oligonucleotide

In certain embodiment, compounds described herein comprise oligonucleotides. In certain embodiments, an oligonucleotide has a nucleobase sequence that is at least partially complementary to a target nucleic acid sequence (e.g., an expressed target nucleic acid within a cell). In some embodiments, the oligonucleotide, upon delivery to a cell expressing a target nucleic acid, is able to modify the expression of the underlying gene. In some embodiments, the oligonucleotide, upon delivery to a cell expressing a target nucleic acid, is able to inhibit the expression of the underlying gene. The gene expression can be modified or inhibited in vitro or in vivo. In certain embodiments, an oligonucleotide comprises one or more ribonucleic acids (e.g., one or more ribonucleosides), deoxyribonucleic acids (e.g., one or more deoxyribonucleosides), modified nucleic acids (e.g., one or more modified nucleobases, sugars, and/or internucleoside linkages), or a combination thereof. In some embodiments, an oligonucleotide comprises a ribonucleic acid (RNA). In some embodiments, an oligonucleotide comprises a deoxyribonucleic acid (DNA). In some embodiments, an oligonucleotide comprises a modification (e.g., modified nucleobase, modified sugar, or modified internucleoside linkage).

In certain embodiments, an oligonucleotide is single-stranded. In some embodiments, a single-stranded oligonucleotide is single-stranded RNA (ssRNA), ssDNA, or a ssRNA/DNA hybrid (e.g., a single-stranded oligonucleotide comprised of both ribonucleosides (modified or unmodified) and deoxyribonucleosides (modified or unmodified)). In some embodiments, an oligonucleotide is double-stranded (e.g., comprised of two single-stranded nucleic acids). Such double-stranded oligonucleotides comprise a first oligonucleotide having a region complementary to a target nucleic acid and a second oligonucleotide having a region complementary to the first oligonucleotide. The first and second oligonucleotides can be independently modified. In certain embodiments, the first oligonucleotide is linked to one or more NMDA receptor ligands. In certain embodiments, the second oligonucleotide is linked to one or more NMDA receptor ligands.

In some embodiments, an oligonucleotide is at least 2 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109,110,111,112,113,114,115,116,117,118,119,120,121,122,123,124,125,126,127, 128, 129, 130, 131, 132, 133, 134, 135, 136, 137, 138, 139, 140, 141, 142, 143, 144, 145, 146, 147, 148, 149, 150, or more) nucleotides in length. In some embodiments, an oligonucleotide is at least 5 nucleotides in length. In some embodiments, an oligonucleotide is at least 10 nucleotides in length. In some embodiments, an oligonucleotide is at least 15 nucleotides in length. In some embodiments, an oligonucleotide is at least 16 nucleotides in length. In some embodiments, an oligonucleotide is at least 17 nucleotides in length. In some embodiments, an oligonucleotide is at least 18 nucleotides in length. In some embodiments, an oligonucleotide is at least 19 nucleotides in length. In some embodiments, an oligonucleotide is at least 20 nucleotides in length. In some embodiments, an oligonucleotide is at least 21 nucleotides in length. In some embodiments, an oligonucleotide is at least 22 nucleotides in length. In some embodiments, an oligonucleotide is at least 23 nucleotides in length. In some embodiments, an oligonucleotide is at least 24 nucleotides in length. In some embodiments, an oligonucleotide is at least 25 nucleotides in length. In some embodiments, an oligonucleotide is at least 26 nucleotides in length. In some embodiments, an oligonucleotide is at least 27 nucleotides in length. In some embodiments, an oligonucleotide is at least 28 nucleotides in length. In some embodiments, an oligonucleotide is at least 29 nucleotides in length. In some embodiments, an oligonucleotide is at least 30 nucleotides in length. In some embodiments, an oligonucleotide is at least 40 nucleotides in length. In some embodiments, an oligonucleotide is at least 50 nucleotides in length. In some embodiments, an oligonucleotide is at least 60 nucleotides in length. In some embodiments, an oligonucleotide is at least 70 nucleotides in length. In some embodiments, an oligonucleotide is at least 80 nucleotides in length. In some embodiments, an oligonucleotide is at least 90 nucleotides in length. In some embodiments, an oligonucleotide is at least 100 nucleotides in length. In some embodiments, an oligonucleotide is at least 150 nucleotides in length.

In some embodiments, an oligonucleotide is less than or equal to 150 (e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106,107,108,109,110,111,112,113,114,115,116,117,118,119,120,121,122,123,124, 125, 126, 127, 128, 129, 130, 131, 132, 133, 134, 135, 136, 137, 138, 139, 140, 141, 142, 143, 144, 145, 146, 147, 148, 149, 150) nucleotides in length. In some embodiments, an oligonucleotide is less than or equal to 150 nucleotides in length. In some embodiments, an oligonucleotide is less than or equal to 100 nucleotides in length. In some embodiments, an oligonucleotide is less than or equal to 90 nucleotides in length. In some embodiments, an oligonucleotide is less than or equal to 80 nucleotides in length. In some embodiments, an oligonucleotide is less than or equal to 70 nucleotides in length. In some embodiments, an oligonucleotide is less than or equal to 60 nucleotides in length. In some embodiments, an oligonucleotide is less than or equal to 50 nucleotides in length. In some embodiments, an oligonucleotide is less than or equal to 40 nucleotides in length. In some embodiments, an oligonucleotide is less than or equal to 30 nucleotides in length. In some embodiments, an oligonucleotide is less than or equal to 29 nucleotides in length. In some embodiments, an oligonucleotide is less than or equal to 28 nucleotides in length. In some embodiments, an oligonucleotide is less than or equal to 27 nucleotides in length. In some embodiments, an oligonucleotide is less than or equal to 26 nucleotides in length. In some embodiments, an oligonucleotide is less than or equal to 25 nucleotides in length. In some embodiments, an oligonucleotide is less than or equal to 24 nucleotides in length. In some embodiments, an oligonucleotide is less than or equal to 23 nucleotides in length. In some embodiments, an oligonucleotide is less than or equal to 22 nucleotides in length. In some embodiments, an oligonucleotide is less than or equal to 21 nucleotides in length. In some embodiments, an oligonucleotide is less than or equal to 20 nucleotides in length. In some embodiments, an oligonucleotide is less than or equal to 19 nucleotides in length. In some embodiments, an oligonucleotide is less than or equal to 18 nucleotides in length. In some embodiments, an oligonucleotide is less than or equal to 17 nucleotides in length. In some embodiments, an oligonucleotide is less than or equal to 16 nucleotides in length. In some embodiments, an oligonucleotide is less than or equal to 15 nucleotides in length. In some embodiments, an oligonucleotide is less than or equal to 10 nucleotides in length. In some embodiments, an oligonucleotide is less than or equal to 5 nucleotides in length.

In some embodiments, an oligonucleotide is about 5 nucleotides in length to about 150 nucleotides in length. In some embodiments, an oligonucleotide is about 10 nucleotides in length to about 100 nucleotides in length. In some embodiments, an oligonucleotide is about 20 nucleotides in length to about 90 nucleotides in length. In some embodiments, an oligonucleotide is about 30 nucleotides in length to about 80 nucleotides in length. In some embodiments, an oligonucleotide is about 40 nucleotides in length to about 70 nucleotides in length. In some embodiments, an oligonucleotide is about 50 nucleotides in length to about 60 nucleotides in length.

In some embodiments, an oligonucleotide is a therapeutic oligonucleotide. A therapeutic oligonucleotide may comprise, for example, without limitation, a small interfering RNA (siRNA), a microRNA (miRNA) antagonist, a miRNA mimic, an ADAR recruiting molecule, an ADAR targeting molecule, a guide RNA, an antisense oligonucleotide, a short hairpin RNA (shRNA), or combinations thereof.

In certain embodiments, a miRNA is a precursor, primary, and/or mature miRNA.

In certain embodiments, an oligonucleotide comprises or consists of an antisense oligonucleotide. In certain embodiments, an antisense oligonucleotide is complementary to an mRNA. In certain embodiments, an antisense oligonucleotide is complementary to a pre-mRNA. In certain embodiments, an antisense oligonucleotide blocks translation and promotes degradation of the mRNA transcript. In certain embodiments, an antisense oligonucleotide recruits RNase H and promotes degradation of the mRNA transcript. In certain embodiments, an antisense oligonucleotide targets miRNA, inhibiting the miRNA from modulating mRNA expression and promoting degradation of the miRNA.

### Certain Modifications

In certain aspects, the disclosure relates to compounds that comprise oligonucleotides. In certain embodiments, oligonucleotides may be unmodified RNA or DNA, or the oligonucleotides may be modified. In certain embodiments, the oligonucleotides are modified oligonucleotides. In certain embodiments, the modified oligonucleotides comprise at least one modified sugar, modified nucleobase, or modified internucleoside linkage relative to an unmodified RNA or DNA. In certain embodiments, an oligonucleotide has a modified nucleoside. A modified nucleoside may comprise a modified sugar, a modified nucleobase, or both a modified sugar and a modified nucleobase. Modified oligonucleotides may also include end modifications, e.g., 5′-end modifications and 3′-end modifications.

### Sugar Modifications and Motifs

In certain embodiments, a modified sugar is a substituted furanosyl sugar or non-bicyclic modified sugar. In certain embodiments, a modified sugar is a bicyclic or tricyclic modified sugar. In certain embodiments, a modified sugar is a sugar surrogate. A sugar surrogate may comprise one or more substitutions described herein.

In certain embodiments, a modified sugar is a substituted furanosyl or non-bicyclic modified sugar. In certain embodiments, the furanosyl sugar is a ribosyl sugar. In certain embodiments, the furanosyl sugar comprises one or more substituent groups, including, but not limited to, substituent groups at the 2′, 3′, 4′, and 5′ positions.

In certain embodiments, substituents at the 2′ position include, but are not limited to, F and OCH₃ (“OMe”, “O-methyl” or “methoxy”). In certain embodiments, substituent groups at the 2′ position suitable for non-bicyclic modified sugars include, but are not limited to, halo, allyl, amino, azido, SH, CN, OCN, CF₃, OCF₃, F, Cl, Br, SCH₃, SOCH₃, SO₂CH₃, ONO₂, NO₂, N₃, and NH₂. In certain embodiments, substituent groups at the 2′ position include, but are not limited to, O—(C₁-C₁₀) alkoxy, alkoxyalkyl, O-alkyl, S-alkyl, N-alkyl, O-alkenyl, S-alkenyl, N-alkenyl, O-alkynyl, S-alkynyl, N-alkynyl, O-alkyl-O-alkyl, alkynyl, wherein the alkyl, alkenyl and alkynyl can be substituted or unsubstituted C₁ to C₁₀ alkyl or C₂ to C₁₀ alkenyl and alkynyl. In certain embodiments, substituent groups at the 2′ position include, but are not limited to, alkaryl, aralkyl, O-alkaryl, and O-aralkyl. In certain embodiments, these 2′ substituent groups can be further substituted with one or more substituent groups independently selected from hydroxyl, alkoxy, carboxy, benzyl, phenyl, nitro (NO₂), thiol, thioalkoxy, thioalkyl, halogen, alkyl, aryl, alkenyl, and alkynyl. In certain embodiments, substituent groups at the 2′ position include, but are not limited to, O[(CH₂)ₙO]ₘCH₃, O(CH₂)ₙOCH₃, O(CH₂)ₙCH₃, O(CH₂)ₙONH₂, O(CH₂)ₙNH₂, O(CH₂)ₙSCH₃, and O(CH₂)ₙON[(CH₂)·CH₃)]₂, where n and m are independently from 1 to about 10. In certain embodiments, substituent groups at the 2′ position include, but are not limited to, OCH₂CH₂OCH₃ (“MOE”), O(CH₂)₂ON(CH₃)₂ (“DMAOE”), O(CH₂)₂O(CH₂)₂N(CH₃)₂ (“DMAEOE”), and OCH₂C(═O)—N(H)CH₃ (“NMA”).

In certain embodiments, substituent groups at the 4′ position suitable for non-bicyclic modified sugars include, but are not limited to, alkoxy (e.g., methoxy), alkyl, and those described in Manoharan et al., WO 2015/106128. In certain embodiments, substituent groups at the 5′ position suitable for non-bicyclic modified sugars include, but are not limited to, methyl (“Me”) (R or S), vinyl, and methoxy. In certain embodiments, one or more sugars comprise a 5′-vinylphosphonate modification. In certain embodiments, substituents described herein for the 2′, 4′, and 5′ position can be added to other specific positions on the sugar. In certain embodiments, such substituents may be added to the 3′ position of the sugar on the 3′ terminal nucleoside or the 5′ position of the 5′ terminal nucleoside. In certain embodiments, a non-bicyclic modified sugar may comprise more than one non-bridging sugar substituent. In certain such embodiments, non-bicyclic modified sugar substituents include, but are not limited to, 5′-Me-2′-F, 5′-Me-2′-OMe (including both R and S isomers). In certain embodiments, modified sugar substituents include those described in Migawa et al., WO 2008/101157 and Rajeev et al., US2013/0203836.

In certain embodiments, a modified sugar is a bicyclic sugar. A bicyclic sugar is a modified sugar comprising two rings, wherein the second ring is formed via a bridge connecting two of the atoms in the first ring, thereby forming a bicyclic structure. In certain embodiments, a bicyclic sugar comprises a bridging substituent that bridges two atoms of the furanosyl ring to form a second ring. In certain embodiments, a bicyclic sugar does not comprise a furanosyl moiety. A “bicyclic nucleoside” (“BNA”) is a nucleoside having a bicyclic sugar. In certain embodiments, the bicyclic sugar comprises a bridge between the 4′ and 2′ furanose ring atoms. In certain embodiments, the bicyclic sugar comprises a bridge between the 5′ and 3′ furanose ring atoms. In certain such embodiments, the furanose ring is a ribose ring. In certain embodiments, 4′ to 2′ bridging substituents include, but are not limited to, 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (“constrained ethyl” or “cEt” when in the S configuration), 4′-CH₂O—CH₂-2′, 4′-CH₂—N(R)-2′, 4′-CH(CH₂OCH₃)—O-2′ (“constrained MOE” or “cMOE”) and analogs thereof (e.g., U.S. Pat. No. 7,399,845), 4′-C(CH₃)(CH₃)—O-2′ and analogs thereof (e.g., U.S. Pat. No. 8,278,283), 4′-CH₂—N(OCH₃)-2′ and analogs thereof (e.g., U.S. Pat. No. 8,278,425), 4′-CH₂—O—N(CH₃)-2′ (e.g., U.S. Patent Publication No. 2004/0171570), 4′-CH₂—N(R)—O-2′, wherein R is H, C₁-C₁₂ alkyl, or a protecting group (e.g., U.S. Pat. No. 7,427,672), 4′-CH₂—C(H)(CH₃)-2′ (e.g., Chattopadhyaya et al., J. Org. Chem., 2009, 74, 118-134), and 4′-CH₂—C(═CH₂)-2′ and analogs thereof (e.g., U.S. Pat. No. 8,278,426). The entire contents of each of the foregoing are hereby incorporated herein by reference. Additional representative U.S. Patents and U.S. Patent Publications that teach the preparation of bicyclic nucleic acid nucleotides include, but are not limited to, the following: U.S. Pat. Nos. 6,268,490; 6,525,191; 6,670,461; 6,770,748; 6,794,499; 6,998,484; 7,053,207; 7,034,133; 7,084,125; 7,399,845; 7,427,672; 7,569,686; 7,741,457; 8,022,193; 8,030,467; 8,278,425; 8,278,426; 8,278,283; US 2008/0039618; and US 2009/0012281, US 2013/0190383; and WO 2013/036868, the entire contents of each of which are hereby incorporated herein by reference. Any of the foregoing bicyclic nucleosides can be prepared having one or more stereochemical sugar configurations including, for example, a-L-ribofuranose and β-D-ribofuranose (see, e.g., WO 99/14226). Specified bicyclic nucleosides herein are in the β-D configuration, unless otherwise specified.

In certain embodiments, a modified sugar is a sugar surrogate. In certain embodiments, a sugar surrogate has the oxygen atom replaced, e.g., with a sulfur, carbon or nitrogen atom. In certain such embodiments, the sugar surrogate may also comprise bridging and/or non-bridging substituents as described herein. In certain embodiments, sugar surrogates comprise rings having other than 5 atoms. In certain such embodiments, the sugar surrogate comprises a cyclobutyl moiety in place of the pentofuranosyl sugar. In certain embodiments, the sugar surrogate comprises a six membered ring in place of the pentofuranosyl sugar. In certain embodiments, the sugar surrogate comprises a tetrahydropyran (“THP”) in place of the pentofuranosyl sugar. In certain embodiments, the sugar surrogate comprises a morpholino in place of the pentofuranosyl sugar. Representative U.S. patents that teach the preparation of such modified sugar structures include, but are not limited to, U.S. Pat. Nos. 4,981,957; 5,118,800; 5,166,315; 5,185,444; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,700,920; 7,875,733; 7,939,677, 8,088,904; 8,440,803; and 9,005,906, the entire contents of each of the foregoing are hereby incorporated herein by reference.

In some embodiments, sugar surrogates comprise acyclic moieties. In certain embodiments, the sugar surrogate is an unlocked nucleic acid (“UNA”). A UNA is unlocked acyclic nucleic acid, wherein any of the bonds of the sugar has been removed, forming an unlocked “sugar” residue. In one example, UNA also encompasses a monomer where the bonds between C1′-C4′ have been removed (i.e., the covalent carbon-oxygen-carbon bond between the C1′ and C4′ carbons). In another example, the C2′-C3′ bond (i.e., the covalent carbon-carbon bond between the C2′ and C3′ carbons) of the sugar has been removed. Representative U.S. publications that teach the preparation of UNA include, but are not limited to, U.S. Pat. No. 8,314,227; and U.S. Patent Publication Nos. 2013/0096289; 2013/0011922; and 2011/0313020, the entire contents of each of which are hereby incorporated herein by reference. In certain embodiments, sugar surrogates comprise peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., US2013/130378, the entire contents of which is incorporated herein by reference. Many other bicyclic and tricyclic sugar and sugar surrogate ring systems are known in the art that can be used in modified nucleosides.

In certain aspects, the disclosure relates to compounds comprising at least one oligonucleotide, wherein the nucleosides of such oligonucleotides comprise one or more types of modified sugars and/or unmodified sugars arranged along the oligonucleotide or region thereof in a defined pattern or “sugar motif”. In certain instances, such sugar motifs include, but are not limited to, any of the patterns of sugar modifications described herein.

In certain embodiments, an oligonucleotide comprises a gapmer sugar motif. A gapmer oligonucleotide comprises or consists of a region having two external “wing” regions and a central or internal “gap” region. The gap and wing regions form a contiguous sequence of nucleosides, wherein the majority of nucleoside sugars of each of the wings differ from the majority of nucleoside sugars of the gap. In certain embodiments, the wing regions comprise a majority of modified sugars, and the gap comprises a majority of unmodified sugars. In certain embodiments, the nucleosides of the gap are deoxynucleosides. Compounds with a gapmer sugar motif are described in, for example, U.S. Pat. No. 8,790,919, the contents of which is incorporated herein by reference.

In certain embodiments, one or both oligonucleotides of a double-stranded compound comprise a triplet sugar motif. An oligonucleotide with a triplet sugar motif comprises three identical sugar modifications on three consecutive nucleosides. In certain embodiments, the triplet is at or near the cleavage site of the oligonucleotide. In certain embodiments, an oligonucleotide of a double-stranded compound may contain more than one triplet sugar motif. In certain embodiments, the identical sugar modification of the triplet sugar motif is a 2′-F modification. Compounds with a triplet sugar motif are disclosed, for example, in U.S. Pat. No. 10,668,170, the contents of which is incorporated herein by reference.

In certain embodiments, one or both oligonucleotides of a double-stranded compound comprise a quadruplet sugar motif. An oligonucleotide with a quadruplet sugar motif comprises four identical sugar modifications on four consecutive nucleosides. In certain embodiments, the quadruplet is at or near the cleavage site. In certain embodiments, an oligonucleotide of a double-stranded compound may contain more than one quadruplet sugar motif. In certain embodiments, the identical sugar modification of the quadruplet sugar motif is a 2′-F modification. For a double-stranded compound having a duplex region of 19-23 nucleotides in length, the cleavage site of the antisense oligonucleotide is typically around the 10, 11, and 12 positions from the 5′-end. In certain embodiments, the quadruplet sugar motif is at the 8, 9, 10, 11 positions; the 9, 10, 11, 12 positions; the 10, 11, 12, 13 positions; the 11, 12, 13, 14 positions; or the 12, 13, 14, 15 positions of the sense oligonucleotide, counting from the first nucleoside of the 5′-end of the sense oligonucleotide, or, the count starting from the first paired nucleotide within the duplex region from the 5′-end of the sense oligonucleotide. In certain embodiments, the quadruplet sugar motif is at the 8, 9, 10, 11 positions; the 9, 10, 11, 12 positions; the 10, 11, 12, 13 positions; the 11, 12, 13, 14 positions; or the 12, 13, 14, 15 positions of the antisense oligonucleotide, counting from the first nucleoside of the 5′-end of the antisense oligonucleotide, or, the count starting from the first paired nucleotide within the duplex region from the 5′-end of the antisense oligonucleotide. The cleavage site may change according to the length of the duplex region of the double-stranded compound and may change the position of the quadruplet accordingly.

In certain embodiments, an oligonucleotide comprises an alternating sugar motif. In certain embodiments, one or both oligonucleotides of a double-stranded compound comprise an alternating sugar motif. An oligonucleotide with an alternating sugar motif comprises at least two different sugar modifications, wherein one or more consecutive nucleosides comprising a first sugar modification alternates with one or more consecutive nucleosides comprising a second sugar modification, and one or more consecutive nucleosides comprising a third sugar modification, etc. For example, if A, B, and C each represent one type of modification to the nucleoside, the alternating motif can be “ABABABABABAB . . . ,” “AABBAABBAABB . . . ,” “AABAABAABAAB “AAABAAABAAAB . . . ,” “AAABBBAAABBB . . . ,” or “ABCABCABCABC . . . ” etc. In certain embodiments, the alternating sugar motif is repeated for at least 2, 3, 4, 5, 6, 7, 8, 9, 10, 11, 12, 13, 14, 15, 16, 17, 18, 19, 20, 21, 22, or 23 contiguous nucleobases along an oligonucleotide. In certain embodiments, the alternating sugar motif is comprised of two different sugar modifications. In certain embodiments, the alternating sugar motif comprises 2′-OMe and 2′-F sugar modifications.

In certain embodiments, each nucleoside of an oligonucleotide is independently modified with one or more sugar modifications provided herein. In certain embodiments, each oligonucleotide of a double-stranded compound independently has one or more sugar motifs provided herein. In certain embodiments, an oligonucleotide containing a sugar motif is fully modified in that each nucleoside other than the nucleosides comprising the sugar motif comprises a sugar modification.

### Nucleobase Modifications and Motifs

In certain embodiments, modified oligonucleotides comprise one or more nucleosides comprising a modified nucleobase. In certain embodiments, modified oligonucleotides comprise one or more nucleosides that do not comprise a nucleobase, referred to as an abasic nucleoside.

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, 5-methylcytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (C≡C≡CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly, 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size expanded bases, and fluorinated bases. Further modified nucleobases include tricyclic pyrimidines, such as 1,3-diazaphenoxazine-2-one, 1,3-diazaphenothiazine-2-one, and 9-(2-aminoethoxy)-1,3-diazaphenoxazine-2-one (G-clamp). Modified nucleobases may also include those in which the purine or pyrimidine base is replaced with other heterocycles, for example, 7-deaza-adenine, 7-deazaguanosine, 2-aminopyridine and 2-pyridone.

Further nucleobases include those disclosed in U.S. Pat. No. 3,687,808; Modified Nucleosides in Biochemistry, Biotechnology and Medicine, Herdewijn, P. ed. Wiley-VCH, 2008; The Concise Encyclopedia Of Polymer Science And Engineering, pages 858-859; Kroschwitz, J. L., Ed., John Wiley & Sons, 1990, 858-859; Englisch et al., Angewandte Chemie, International Edition, 1991, 30, 613; Sanghvi, Y. S., Chapter 15, dsRNA Research and Applications, pages 289-302; Antisense Research and Applications, Crooke, S. T. and Lebleu, B., Eds., CRC Press, 1993, 273-288; Antisense Drug Technology, Crooke S. T., Ed., CRC Press, 2008, 163-166 and 442-443 (Chapters 6 and 15), each of which are incorporated herein by reference.

Publications that teach the preparation of certain of the above noted modified nucleobases, as well as other modified nucleobases, include without limitation, U.S. Patent Application Publication Nos. 2003/0158403 and 2003/0175906; U.S. Pat. Nos. 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121; 5,596,091; 5,614,617; 5,645,985; 5,681,941; 5,811,534; 5,750,692; 5,948,903; 5,587,470; 5,457,191; 5,763,588; 5,830,653; 5,808,027; 6,005,096, 6,015,886; 6,147,200; 6,166,197; 6,166,199; 6,222,025; 6,235,887; 6,380,368; 6,528,640; 6,639,062; 6,617,438; 7,045,610; 7,427,672; and 7,495,088, the contents of each of which are incorporated herein by reference.

In certain embodiments, oligonucleotides comprise modified and/or unmodified nucleobases arranged along the oligonucleotide or region thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines.

In certain embodiments, modified oligonucleotides comprise a block of modified nucleobases. In certain such embodiments, the block is at the 3′-end of the oligonucleotide. In certain embodiments, the block is within 3 nucleosides of the 3′-end of the oligonucleotide. In certain embodiments, the block is at the 5′-end of the oligonucleotide. In certain embodiments, the block is within 3 nucleosides of the 5′-end of the oligonucleotide.

### Internucleoside Linkage Modifications and Motifs

A 3′ to 5′ phosphodiester linkage is the naturally occurring internucleoside linkage of RNA and DNA. In certain embodiments, an oligonucleotide has one or more modified, i.e., non-naturally occurring, internucleoside linkages. Certain non-naturally occurring internucleoside linkages may impart desirable properties such as, for example, enhanced cellular uptake, enhanced affinity for target nucleic acids, and increased stability in the presence of nucleases. Representative phosphorus-containing modified internucleoside linkages include, but are not limited to, phosphotriesters, alkylphosphonates (e.g., methylphosphonates), phosphoramidates, and phosphorothioates (“P═S”), and phosphorodithioates (“HS—P═S”). Representative non-phosphorus containing internucleoside linking groups include, but are not limited to, methylenemethylimino (—CH₂—N(CH₃)—O—CH₂), thiodiester, thionocarbamate (—O—C(═O)(NH)—S—); siloxane (—O—SiH₂—O—); and N,N′-dimethylhydrazine (—CH₂—N((CH₃)—N((CH₃)—). Methods of preparation of phosphorous-containing and non-phosphorous-containing internucleoside linkages are well known to those skilled in the art. Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl, and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (See, for example: Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and —CH₂ component parts.

In certain embodiments, an oligonucleotide comprises at least one modified internucleoside linkage. A modified internucleoside linkage may be placed at any position of an oligonucleotide. For double-stranded compounds, a modified internucleoside linkage may be placed within the sense oligonucleotide, antisense oligonucleotide, or both oligonucleotides of the double-stranded compound.

In certain embodiments, the internucleoside linkage modification may occur on every nucleoside of an oligonucleotide. In certain embodiments, internucleoside linkage modifications may occur in an alternating pattern along an oligonucleotide. In certain embodiments, essentially each internucleoside linking group is a phosphate internucleoside linkage (P═O). In certain embodiments, each internucleoside linking group of a modified oligonucleotide is a phosphorothioate (P═S). In certain embodiments, each internucleoside linking group of a modified oligonucleotide is independently selected from a phosphorothioate and phosphate internucleoside linkage. In certain embodiments, the pattern of the internucleoside linkage modification on each oligonucleotide of a double-stranded compound is the same. In certain embodiments, the pattern of the internucleoside linkage modification on each oligonucleotide of a double-stranded compound is different. In certain embodiments, a double-stranded compound comprises 6-8 modified internucleoside linkages. In certain embodiments, the 6-8 modified internucleoside linkages are phosphorothioate internucleoside linkages or alkylphosphonate internucleoside linkages. In certain embodiments, the sense oligonucleotide comprises at least two modified internucleoside linkages at either or both the 5′-end and the 3′-end. In certain such embodiments, the modified internucleoside linkages are phosphorothioate internucleoside linkages or alkylphosphonate internucleoside linkages. In certain embodiments, the antisense oligonucleotide comprises at least two modified internucleoside linkages at either or both the 5′-end and the 3′-end. In certain such embodiments, the modified internucleoside linkages are phosphorothioate internucleoside linkages or alkylphosphonate internucleoside linkages.

In certain embodiments, a double-stranded compound comprises an overhang region. In certain embodiments, a double-stranded compound comprises a phosphorothioate or alkylphosphonate internucleoside linkage modification in the overhang region. In certain embodiments, a double-stranded compound comprises a phosphorothioate or alkylphosphonate internucleotide linkage linking the overhang nucleotide with a paired nucleotide that is next to the overhang nucleotide. For instance, there may be at least two phosphorothioate internucleoside linkages between the terminal three nucleosides, in which two of the three nucleosides are overhang nucleosides, and the third is a paired nucleoside next to the overhang nucleoside. These terminal three nucleosides may be at the 3′-end of the antisense oligonucleotide, the 3′-end of the sense oligonucleotide, the 5′-end of the antisense oligonucleotide, or the 5′-end of the antisense oligonucleotide.

In certain embodiments, modified oligonucleotides comprise one or more internucleoside linkages having chiral centers. Representative chiral internucleoside linkages include, but are not limited to, alkylphosphonates and phosphorothioates. Modified oligonucleotides comprising internucleoside linkages having chiral centers can be prepared as populations of modified oligonucleotides comprising stereorandom internucleoside linkages, or as populations of modified oligonucleotides comprising phosphorothioate linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate linkage. As is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkages in a particular, independently selected stereochemical configuration. In certain embodiments, the particular configuration of the particular phosphorothioate linkage is present in at least 65% of the molecules in the population. In certain embodiments, the particular configuration of the particular phosphorothioate linkage is present in at least 70% of the molecules in the population. In certain embodiments, the particular configuration of the particular phosphorothioate linkage is present in at least 80% of the molecules in the population. In certain embodiments, the particular configuration of the particular phosphorothioate linkage is present in at least 90% of the molecules in the population. In certain embodiments, the particular configuration of the particular phosphorothioate linkage is present in at least 99% of the molecules in the population. Such enriched populations of modified oligonucleotides can be generated using synthetic methods known in the art, e.g., methods described in Oka et al., JACS 125, 8307 (2003), Wan et al. Nuc. Acid. Res. 42, 13456 (2014), and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration.

### NMDA Receptor Ligands

In some embodiments, the compounds provided herein comprise an NMDA receptor ligand. In some embodiments, an NMDA receptor ligand is useful for directing a therapeutic, prophylactic, or diagnostic agent. In certain embodiments, a therapeutic agent is an oligonucleotide (e.g., a therapeutic oligonucleotide). In some embodiments, an NMDA receptor ligand directs an oligonucleotide to a locality. In some embodiments, an NMDA receptor ligand targets tissues. In some embodiments, the tissue is brain tissue. In some embodiments, an NMDA receptor ligand targets a cell receptor. In some embodiments, a cell receptor is an NMDA receptor. In some embodiments, an NMDA receptor is in the brain. In some embodiments, an NMDA receptor is in the frontal cortex. In some embodiments, an NMDA receptor is in the striatum. In some embodiments, an NMDA receptor is in the cerebellum. In some embodiments, an NMDA receptor is in the brain stem. In some embodiments, an NMDA receptor is in the hippocampus. In some embodiments, an NMDA receptor is in the spinal cord.

The use of any NMDA receptor ligand in the compounds provided herein is contemplated by the present disclosure. NMDA receptor ligands are known in the art, and a person of ordinary skill in the art would be capable of identifying additional NMDA receptor ligands for use in the compounds described herein beyond those explicitly provided by the present disclosure. The present disclosure also contemplates the use of derivatives and prodrugs of any NMDA receptor ligand provided herein or known in the art in the presently described compounds, and a person of ordinary skill in the art would know how to make such derivatives and prodrugs.

In some embodiments, an NMDA receptor ligand is an NMDA receptor agonist. In some embodiments, an NMDA receptor ligand is an NMDA receptor antagonist. In some embodiments, an NMDA receptor ligand is memantine, MK-801, huperzine (e.g., huperzine A, huperzine B), cholesterol, lacosamide, rapastinel, ketamine, or a structural analog or derivative thereof. In some embodiments, an NMDA receptor ligand is any of those disclosed in Neuropharmacology 2007, 53(6), 699-723; J. Med. Chem. 1990, 33(2), 789-808; Neuroscience 2001, 105(3), 663-669; J. Med. Chem. 2022, 65(13), 9063-9075; Drugs Fut. 2004, 29(10), 992; Drugs Fut. 2004, 29(10), 993; and British Journal of Pharmacology 2022, 179(6), 1146-1187, each of which is incorporated by reference herein. Exemplary NMDA receptor ligands for use in the present disclosure include, but are not limited to, any of the following NMDA receptor ligands, and derivatives thereof:

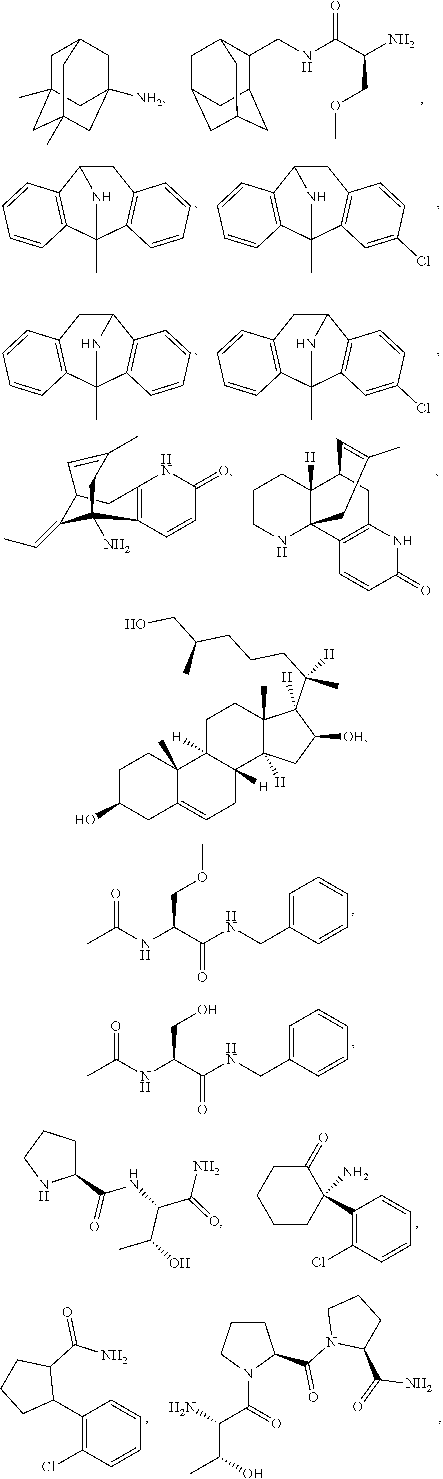

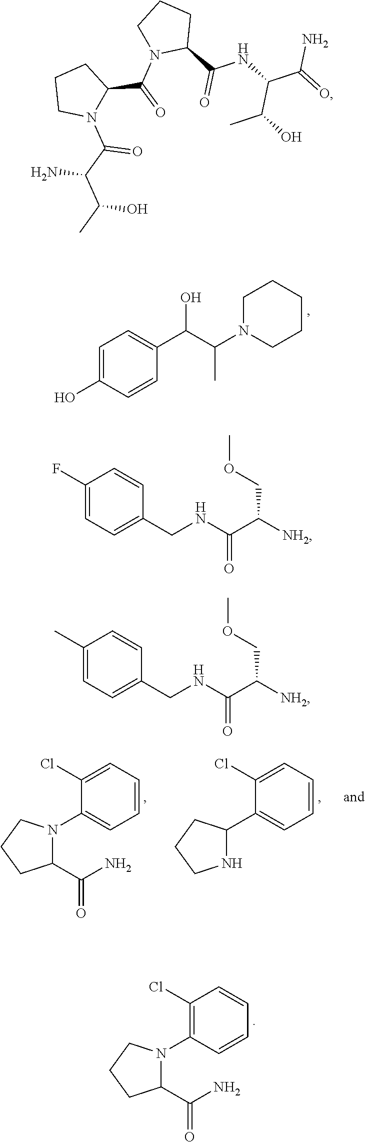

In some embodiments, an NMDA receptor ligand is an anti-NMDA receptor antibody. In certain embodiments, an NMDA receptor ligand is an anti-NMDA receptor antibody fragment, or an anti-NMDA receptor antibody variant. An “anti-NMDA receptor antibody” refers to an immune system protein that recognizes, binds to, or otherwise interacts with an NMDA receptor.

In certain embodiments, an NMDA receptor ligand is conjugated (e.g., linked, connected, attached, associated with) to and one or more agent moieties. In certain embodiments, the agent moiety is a therapeutic, prophylactic, diagnostic, or imaging agent. In certain embodiments, the agent is a small molecule or oligomeric compound. In certain embodiments, the agent moiety is a protein, a peptide, an antibody, an oligonucleotide, a small molecule, a large molecule, or a combination thereof.

In some embodiments, more than one NMDA receptor ligand is conjugated to an agent moiety. In some embodiments, at least two NMDA receptor ligands (e.g., 2, 3, 4, 5, 6, 7, 8, 9, 10, or more NMDA receptor ligands) are conjugated to an agent moiety. In some embodiments, two NMDA receptor ligands are conjugated to an agent moiety. In some embodiments, three NMDA receptor ligands are conjugated to an agent moiety. In some embodiments, four NMDA receptor ligands are conjugated to an agent moiety. In some embodiments, five NMDA receptor ligands are conjugated to an agent moiety. In some embodiments, more than five NMDA receptor ligands are conjugated to an agent moiety. In some embodiments, at least one to about five NMDA receptor ligands are conjugated to an agent moiety. In some embodiments, at least one to about four NMDA receptor ligands are conjugated to an agent moiety. In some embodiments, at least one to about three NMDA receptor ligands are conjugated to an agent moiety. In some embodiments, at least one to about two NMDA receptor ligands are conjugated to an agent moiety.

When an agent moiety is conjugated to multiple NMDA receptor ligands, all of the NMDA receptor ligands may be conjugated at or near the same position on the agent moiety, or the NMDA receptor ligands may be conjugated to multiple different positions on the agent moiety.

In some embodiments, an oligonucleotide is conjugated (e.g., connected, attached, associated with) to an NMDA receptor ligand through either a 5′ end and/or a 3′ end of the oligonucleotide, or at an internal position in an oligonucleotide (i.e., at a nucleotide on the oligonucleotide other than the 5′ or 3′ nucleotide). In some embodiments, an oligonucleotide is conjugated to an NMDA receptor ligand through the 5′ end of the oligonucleotide. In some embodiments, an oligonucleotide is conjugated to an NMDA receptor ligand through the 3′ end of the oligonucleotide. In some embodiments, an oligonucleotide is conjugated to NMDA receptor ligands through both the 5′ end and the 3′ end of the oligonucleotide. In some embodiments, an oligonucleotide is conjugated to an NMDA receptor ligand at an internal position within the oligonucleotide (e.g., in an “internally-modified oligonucleotide”).

In some embodiments, an oligonucleotide is conjugated to more than one NMDA receptor ligand. In some embodiments, an oligonucleotide is conjugated to at least two NMDA receptor ligands (e.g., 2, 3, 4, 5, 6, 7, 8, 9, 10, or more NMDA receptor ligands). In some embodiments, an oligonucleotide is conjugated to two NMDA receptor ligands. In some embodiments, an oligonucleotide is conjugated to three NMDA receptor ligands. In some embodiments, an oligonucleotide is conjugated to four NMDA receptor ligands. In some embodiments, an oligonucleotide is conjugated to five NMDA receptor ligands. In some embodiments, an oligonucleotide is conjugated to more than five NMDA receptor ligands. In some embodiments, an oligonucleotide is conjugated to at least one to about five NMDA receptor ligands. In some embodiments, an oligonucleotide is conjugated to at least one to about four NMDA receptor ligands. In some embodiments, an oligonucleotide is conjugated to at least one to about three NMDA receptor ligands. In some embodiments, an oligonucleotide is conjugated to at least one to about two NMDA receptor ligands.

When an oligonucleotide is conjugated to multiple NMDA receptor ligands, all of the NMDA receptor ligands may be conjugated at or near the same position on the oligonucleotide, or the NMDA receptor ligands may be conjugated to multiple different positions on the oligonucleotide. In some embodiments, multiple NMDA receptor ligands (i.e., two, three, four, five, or more NMDA receptor ligands) are conjugated at the 5′ end of the oligonucleotide. In some embodiments, multiple NMDA receptor ligands (i.e., two, three, four, five, or more NMDA receptor ligands) are conjugated at the 3′ end of the oligonucleotide. In some embodiments, multiple NMDA receptor ligands (i.e., two, three, four, five, or more NMDA receptor ligands) are conjugated at one or more internal positions of the oligonucleotide. In some embodiments, an oligonucleotide is conjugated to one or more NMDA receptor ligands at the 5′ end of the oligonucleotide and/or one or more NMDA receptor ligands at the 3′ end of the oligonucleotide and/or one or more NMDA receptor ligands at an internal position, or multiple internal positions, of the oligonucleotide.

### Linkers

In certain embodiments, conjugates of the compound formulae described herein are provided. In certain embodiments, the conjugates comprise an NMDA receptor ligand covalently coupled to an agent moiety. In certain embodiments, the conjugates provided herein comprise one or more linker moieties. In certain embodiments, the one or more linker moieties link an NMDA receptor ligand to an agent moiety. In certain embodiments, the agent moiety is a protein, peptide, antibody, nucleic acid, small molecule, large molecule, therapeutic, prophylactic, diagnostic, or imaging agent. In some embodiments, a compound is conjugated to an oligonucleotide. In certain embodiments, an NMDA receptor ligand is conjugated to an oligonucleotide. In certain embodiments, a compound comprises one or more NMDA receptor ligands, one or more linker moieties, and one or more agent moieties, wherein the NMDA receptor ligands are conjugated (e.g., linked, connected, attached, associated with) to the one of more agent moieties through one or more linker moieties.

Conjugates as disclosed herein can be manufactured using any available method. When associating compounds provided herein with agent moieties (e.g., an NMDA receptor ligand with an oligonucleotide), the moieties may be linked directly or indirectly (e.g., through a linker moiety; that is, the linker is covalently bonded to each of the oligonucleotide and the NMDA receptor ligand; in some formulae herein “-Lₙ-” wherein n is a number (e.g., L₁, L₂, L₃, L₄)). For example, the oligonucleotide and NMDA receptor ligand may be directly associated with one another, e.g., by one or more covalent bonds, or may be associated by means of one or more linkers. A “linker” refers to any chemical moiety (e.g., a combination of atoms having appropriate valency according to known chemistry principles) used to conjugate two components of the compounds provided herein (e.g., an NMDA receptor ligand and an oligonucleotide) to one another. Each of the two components may be connected to any portion of any of the linkers provided herein. In some embodiments, one component of the compounds provided herein (e.g., an NMDA receptor ligand or an oligonucleotide) is connected by a bond to one end of a linker, and the other component is connected by a bond to the other end of the linker. In some embodiments, one or both components of the compounds provided herein may be connected by a bond to an internal position within any of the linkers described herein. For example, in the context of an “alkyl linker,” an NMDA receptor ligand may be joined by a bond to a carbon at one end of the alkyl linker, and an oligonucleotide may be joined by a bond to a carbon at the other end of the alkyl linker. In some embodiments, a linker is a bond (including, e.g., phosphodiester and phosphorothioate bonds). In some embodiments, a linker is an optionally substituted alkyl linker (i.e., an alkyl chain is used to join two moieties, which may each be conjugated to opposite ends of the alkyl linker, or one or both moieties may be conjugated to an internal carbon on the alkyl linker). In some embodiments, a linker is an optionally substituted polyethylene glycol (PEG) linker (i.e., a PEG chain is used to join two moieties, which may each be conjugated to opposite ends of the PEG linker, or one or both moieties may be conjugated to an internal position on the PEG linker). In some embodiments, a linker is an optionally substituted heteroalkyl linker (i.e., a heteroalkyl chain is used to join two moieties, which may each be conjugated to opposite ends of the heteroalkyl linker, or one or both moieties may be conjugated to an internal position on the heteroalkyl linker). In some embodiments, a linker is an optionally substituted heteroaryl linker (i.e., a heteroaryl group is used to join two moieties, which may each be conjugated to any position on the heteroaryl group).

In certain embodiments, the compounds provided herein comprise one or more linking groups. In certain embodiments, each of L₁, L₂, L₃, and L₄ comprises a linking group. In certain embodiments, a linking group is covalently bound to an NMDA receptor ligand. In certain embodiments, a linking group is covalently bound to an oligonucleotide. In certain embodiments, a linking group is covalently bound to a cleavable moiety. In certain embodiments, a linking group comprises a cleavable bond. In certain embodiments, a linking group does not comprise a cleavable moiety. In certain embodiments, a linking group comprises a covalent attachment to a solid support. In certain embodiments, a linking group includes multiple positions for attachment of NMDA receptor ligands.

In certain embodiments, a linking group comprises a chain structure, such as a hydrocarbyl chain, or an oligomer of repeating units or combination of such repeating units. In certain embodiments, a linking group comprises 1 to 50 repeating units, 1 to 40 repeating units, 1 to 25 repeating units, 1 to 20 repeating units, 1 to 15 repeating units, 1 to 10 repeating units, or 1 to 5 repeating units. In certain embodiments, a linking group is 1 to 50 atoms long, 1 to 40 atoms long, 1 to 25 atoms long, 1 to 20 atoms long, 1 to 15 atoms long, 1 to 10 atoms long, or 1 to 5 atoms long.

In certain embodiments, a linking group contains carbon atoms. In certain embodiments, a linking group contains heteroatoms (e.g., nitrogen, oxygen, sulfur, etc.). In certain embodiments, a linking group forms amide linkages, ester linkages, or disulfide linkages. In certain embodiments, a linking group forms hydrazone linkages, oxime linkages, imine linkages, guanidine linkages, urea linkages, carbamate linkages, unsaturated alkyl linkages, sulfonamide linkages or 4-8 membered hetero cyclic linkages. In certain embodiments, a linking group comprises one or more groups selected from alkyl, amino, oxo, amide, disulfide, polyethylene glycol, ether, thioether, and hydroxylamino. In certain embodiments, a linking group comprises at least one phosphorus group. In certain embodiments, a linking group comprises at least one phosphate group. In certain embodiments, a linking group includes at least one neutral linking group. In certain embodiments, a linking group is substituted with various substituents including, but not limited to, hydrogen atoms, alkyl, alkenyl, alkynyl, amino, alkylamino, dialkylamino, trialkylamino, hydroxyl, alkoxy, halogen, aryl, heterocyclic, aromatic heterocyclic, cyano, amide, carbamoyl, carboxylic acid, ester, thioether, alkylthioether, thiol, and ureido groups. As would be appreciated by one of skill in this art, each of these groups may in turn be substituted.

In certain embodiments, a linking group includes, but is not limited to, substituted or unsubstituted C₁-C₁₀ alkyl, substituted or unsubstituted C₂-C₁₀ alkenyl, or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl, and alkynyl. In certain embodiments, a linking group is an aliphatic or heteroaliphatic. For example, the linking group can be a polyalkyl linking group. The linking group can be a polyether linking group. The linking group can be a polyethylene linking group, such as PEG.

In certain embodiments, the linking group is a short peptide chain. In certain embodiments, a linking group comprises 1 to 40 amino acids, 1 to 25 amino acids, 1 to 20 amino acids, 1 to 15 amino acids, 1 to 10 amino acids, or 1 to 5 amino acids.

In certain embodiments, a linking group comprises linker-nucleosides. In certain embodiments, a linking group comprises 1 to 40 linker-nucleosides, 1 to 25 linker-nucleosides, 1 to 20 linker-nucleosides, 1 to 15 linker-nucleosides, 1 to 10 linker-nucleosides, or 1 to 5 linker-nucleosides. In certain embodiments, such linker-nucleosides may be modified or unmodified nucleosides. It is typically desirable for linker-nucleosides to be cleaved from the compound after it reaches a target tissue. Accordingly, linker-nucleosides herein can be linked to one another and to the remainder of the compound through cleavable bonds. Herein, linker-nucleosides are not considered to be part of an oligonucleotide payload. Accordingly, in embodiments in which a compound comprises an oligonucleotide consisting of a specified number or range of linked nucleosides and/or a specified percent complementarity to a reference nucleic acid, and the compound also comprises an NMDA receptor ligand comprising a linking group comprising linker-nucleosides, those linker-nucleosides are not counted toward the length of the oligonucleotide and are not used in determining the percent complementarity of the oligonucleotide for the reference nucleic acid.

In certain embodiments, the linking group includes a protein binding group. In certain embodiments, the protein binding group is a lipid such as, for example, including but not limited to cholesterol, cholic acid, adamantane acetic acid, 1-pyrene butyric acid, dihydrotestosterone, 1,3-Bis-O(hexadecyl)glycerol, geranyloxyhexyl group, hexadecylglycerol, borneol, menthol, 1,3-propanediol, heptadecyl group, palmitic acid, myristic acid, O3-(oleoyl)lithocholic acid, O3-(oleoyl)cholenic acid, dimethoxytrityl, or phenoxazine), a vitamin (e.g., folate, vitamin A, vitamin E, biotin, pyridoxal), a peptide, a carbohydrate (e.g., monosaccharide, disaccharide, trisaccharide, tetrasaccharide, oligosaccharide, polysaccharide), an endosomolytic component, a steroid (e.g., uvaol, hecigenin, diosgenin), a terpene (e.g., triterpene, e.g., sarsasapogenin, friedelin, epifriedelanol derivatized lithocholic acid), or a cationic lipid. In certain embodiments, the protein binding group is a C₁₆ to C₂₂ long chain saturated or unsaturated fatty acid, cholesterol, cholic acid, vitamin E, adamantane or 1-pentafluoropropyl.

In certain embodiments, a linking group includes, but is not limited to, pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA).

In certain embodiments, a linking group includes, without limitation, those linking groups described in the following references: U.S. Pat. Nos. 5,994,517; 6,300,319; 6,660,720; 6,906,182; 7,262,177; 7,491,805; 8,106,022; 7,723,509; 9,127,276; U.S. 2006/0148740; U.S. 2011/0123520; WO 2013/033230; WO 2012/037254, Biessen et al., J. Med. Chem. 1995, 38, 1846-1852; Lee et al., Bioorganic & Medicinal Chemistry 2011,19, 2494-2500; Rensen et al., J. Biol. Chem. 2001, 276, 37577-37584; Rensen et al., J. Med. Chem. 2004, 47, 5798-5808; Sliedregt et al., J. Med. Chem. 1999, 42, 609-618; Valentijn et al., Tetrahedron, 1997, 53, 759-770; Lee, Carbohydr. Res. 1978, 67, 509-514; Connolly et al., J. Biol. Chem. 1982, 257, 939-945; Pavia et al., Int. J. Pep. Protein Res. 1983, 22, 539-548; Lee et al., Biochem. 1984, 23, 4255-4261; Lee et al., Glycoconjugate J. 1987, 4, 317-328; Toyokuni et al., Tetrahedron Lett. 1990, 31, 2673-2676; Biessen et al., J. Med. Chem. 1995, 38, 1538-1546; Valentijn et al., Tetrahedron, 1997, 53, 759-770; Kim et al., Tetrahedron Lett. 1997, 38, 3487-3490; Lee et al., Bioconjug. Chem. 1997, 8, 762-765; Kato et al., Glycobiol. 2001, 11, 821-829; Rensen et al., J. Biol. Chem. 2001, 276, 37577-37584; Lee et al., Methods Enzymol. 2003, 362, 38-43; Westerlind et al., Glycoconj. J. 2004, 21, 227-241; Lee et al., Bioorg. Med. Chem. Lett. 2006, 16(19), 5132-5135; Maierhofer et al., Bioorg. Med. Chem. 2007, 15, 7661-7676; Khorev et al., Bioorg. Med. Chem. 2008, 16, 5216-5231; Lee et al., Bioorg. Med. Chem. 2011, 19, 2494-2500; Kornilova et al., Analyt. Biochem. 2012, 425, 43-46; Pujol et al., Angew. Chemie Int. Ed. Engl. 2012, 51, 7445-7448; Biessen et al., J. Med. Chem. 1995, 38, 1846-1852; Sliedregt et al., J. Med. Chem. 1999, 42, 609-618; Rensen et al., J. Med. Chem. 2004, 47, 5798-5808; Rensen et al., Arterioscler. Thromh. Vase. Biol. 2006, 26, 169-175; van Rossenberg et al., Gene Ther. 2004, 11, 457-464; Sato et al., J. Am. Chem. Soc. 2004, 126, 14013-14022; Lee et al., J. Org. Chem. 2012, 77, 7564-7571; Biessen et al., FASEB J. 2000, 14, 1784-1792; Rajur et al., Bioconjug. Chem. 1997, 8, 935-940; Duff et al., Methods Enzymol. 2000, 313, 297-321; Maier et al., Bioconjug. Chem. 2003, 14, 18-29; Jayaprakash et al., Org. Lett. 2010, 12, 5410-5413; Manoharan, Antisense Nucleic Acid Drug Dev. 2002, 12, 103-128; Merwin et al., Bioconjug. Chem. 1994, 5, 612-620; Tomiya et al., Bioorg. Med. Chem., 2013, 21, 5275-5281; International Applications WO 1998/013381; WO 2011/038356; WO 1997/046098; WO 2008/098788; WO 2004/101619; WO 2012/037254; WO 2011/120053; WO 2011/100131; WO 2011/163121; WO 2012/177947; WO 2013/033230; WO 2013/075035; WO 2012/083185; WO 2012/083046; WO 2009/082607; WO 2009/134487; WO 2010/144740; WO 2010/148013; WO 1997/020563; WO 2010/088537; WO 2002/043771; WO 2010/129709; WO 2012/068187; WO 2009/126933; WO 2004/024757; WO 2010/054406; WO 2012/089352; WO 2012/089602; WO 2013/166121; WO 2013/165816; U.S. Pat. Nos. 4,751,219; 7,582,744; 8,552,163; 8,137,695; 6,908,903; 6,383,812; 7,262,177; 6,525,031; 5,994,517; 6,660,720; 6,300,319; 7,723,509; 8,106,022; 7,491,805; 7,491,805; 8,541,548; 8,344,125; 8,313,772; 8,349,308; 8,450,467; 8,501,930; 8,158,601; 7,262,177; 6,906,182; 6,620,916; 8,435,491; 8,404,862; 7,851,615; U.S. Patent Application Publications Nos. U.S. 2011/0097264; U.S. 2011/0097265; U.S. 2013/0004427; U.S. 2003/0119724; U.S. 2011/0207799; U.S. 2012/0035115; U.S. 2012/0230938; U.S. 2005/0164235; U.S. 2006/0183886; U.S. 2012/0136042; U.S. 2012/0095075; U.S. 2013/0109817; U.S. 2006/0148740; U.S. 2008/0206869; U.S. 2012/0165393; U.S. 2012/0101148; U.S. 2013/0121954; U.S. 2011/0123520; U.S. 2003/0077829; U.S. 2008/0108801; and U.S. 2009/0203132; each of which is incorporated herein by reference.

In certain embodiments, L₁, L₂, L₃, and L₄ independently comprise or together comprise a structure selected from among:

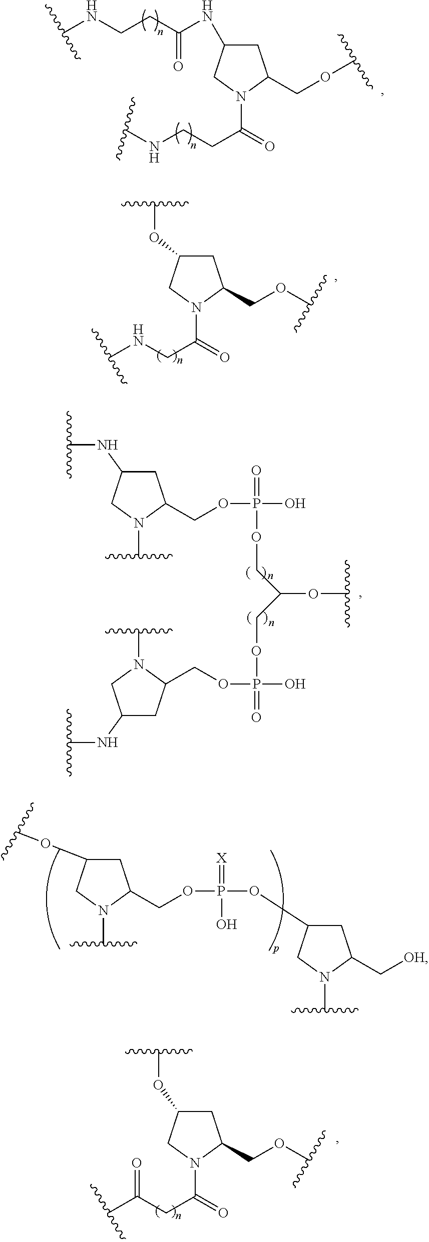

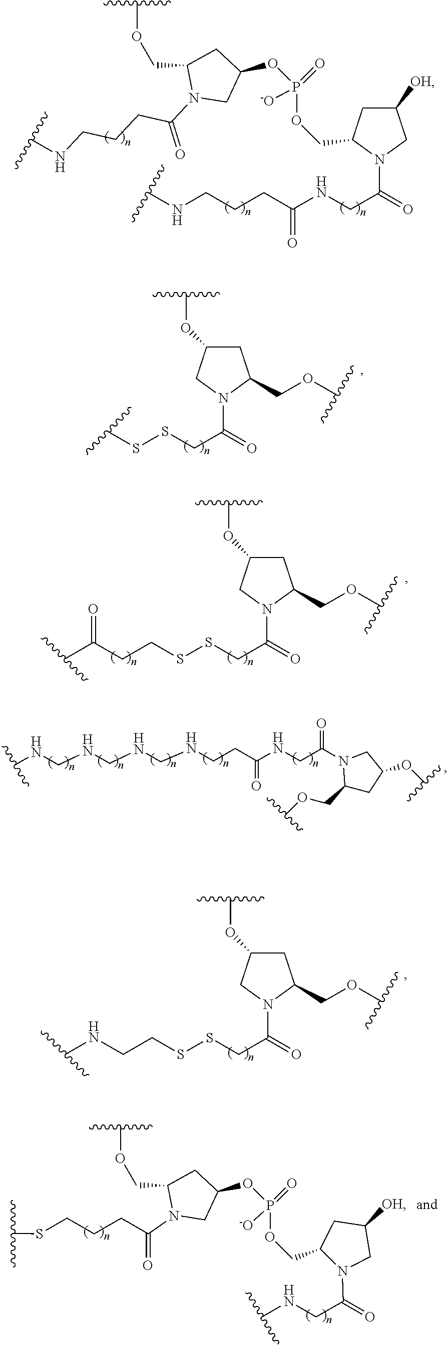

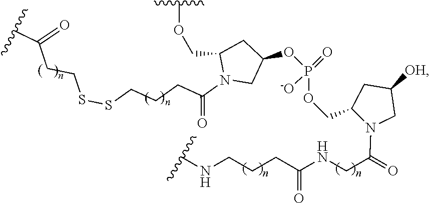

wherein each n is, independently, an integer from 1 to 20 (i.e., 1, 2, 3, 4, 5, 6, 7, 8, 9, 10, 11, 12, 13, 14, 15, 16, 17, 18, 19, or 20); and p is, independently, and integer from 1 to 6 (i. e., 1, 2, 3, 4, 5, or 6).

In certain embodiments, L₁, L₂, L₃, and L₄ independently comprise or together comprise the structure selected from among:

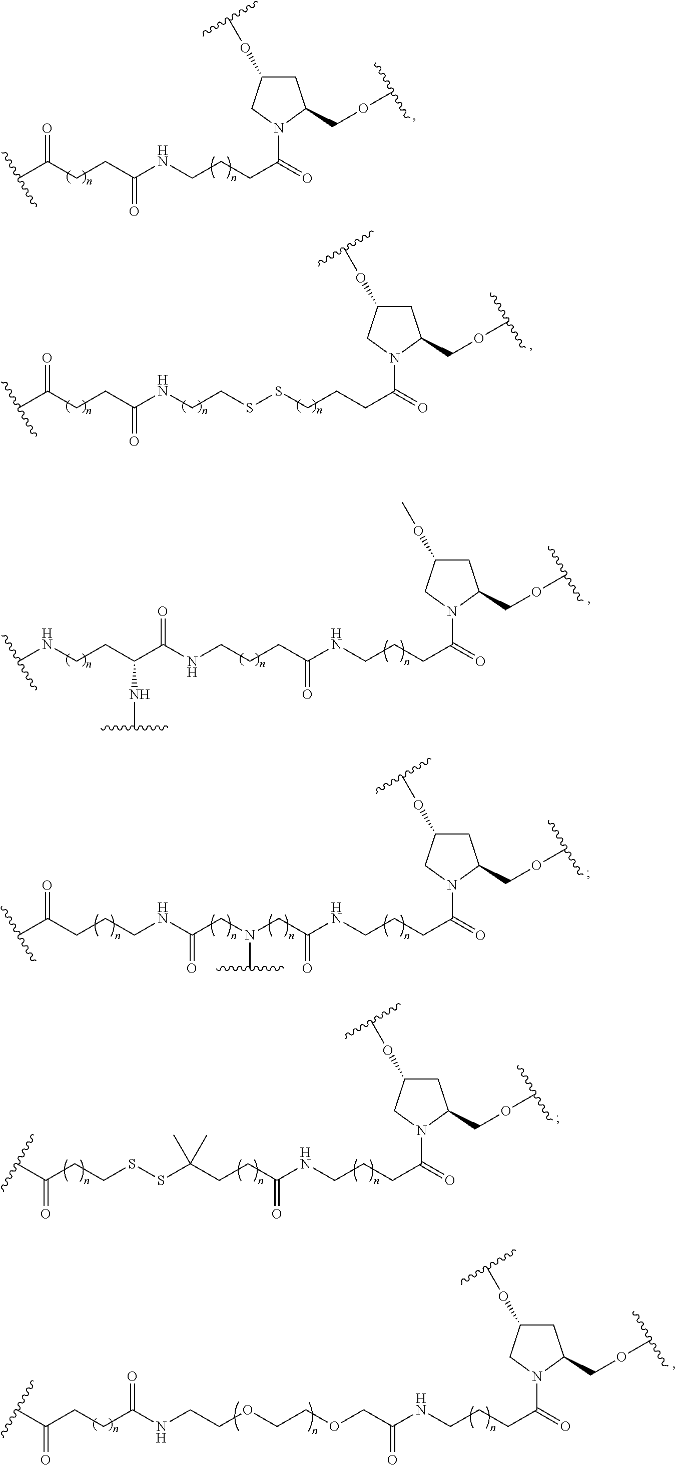

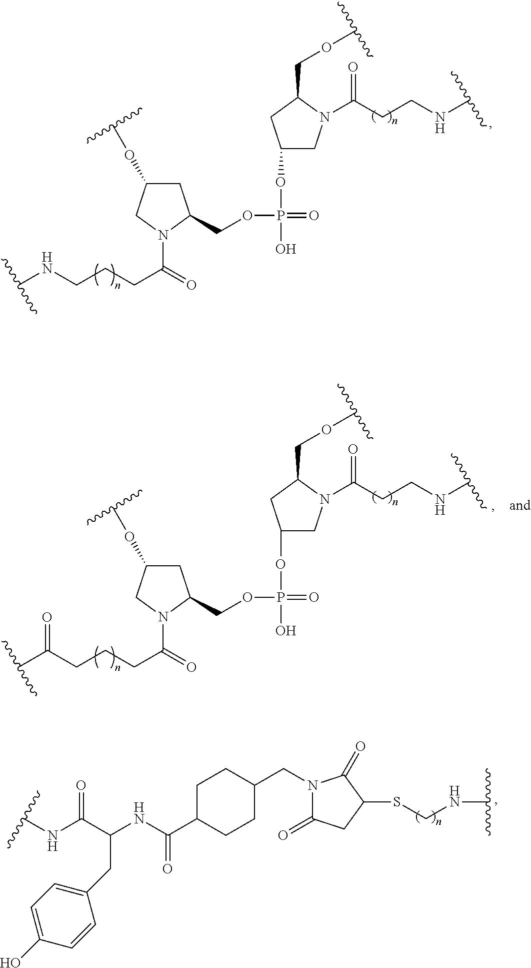

wherein each n is, independently, an integer from 1 to 20 (i.e., 1, 2, 3, 4, 5, 6, 7, 8, 9, 10, 11, 12, 13, 14, 15, 16, 17, 18, 19, or 20).

In certain embodiments, L₁, L₂, L₃, and L₄ independently comprise or together comprise the structure selected from among:

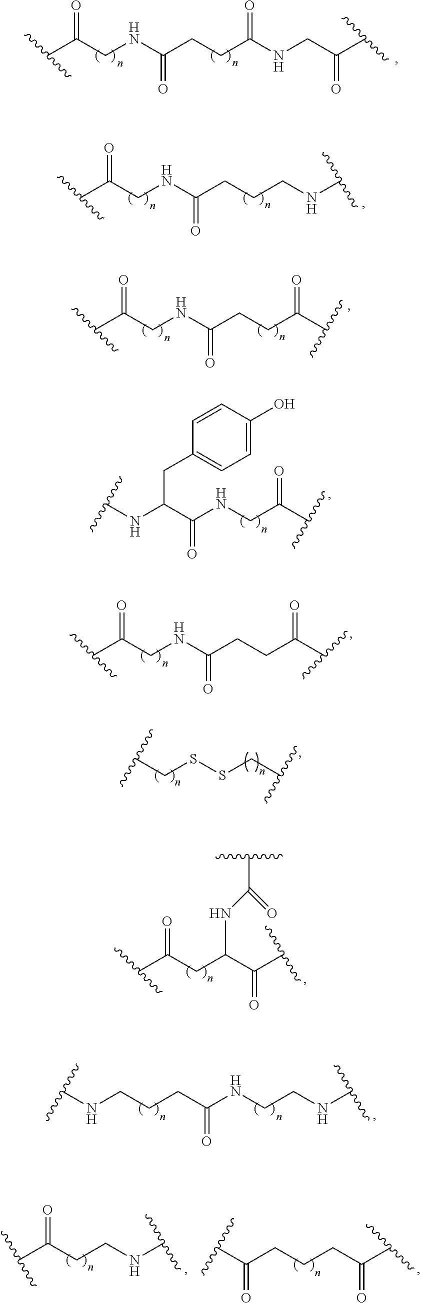

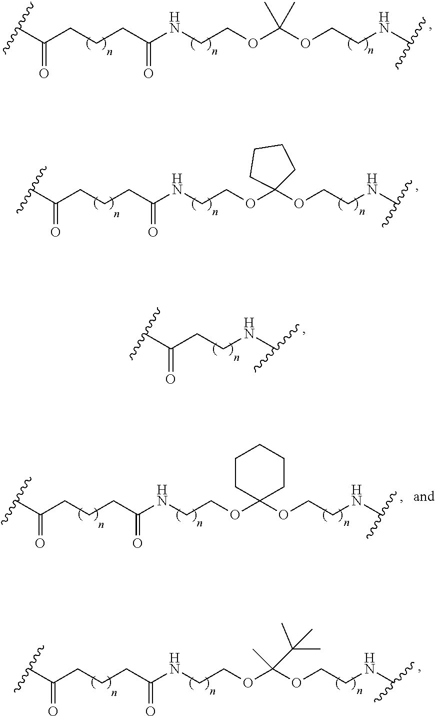

wherein each n is, independently, an integer from 1 to 20 (i.e., 1, 2, 3, 4, 5, 6, 7, 8, 9, 10, 11, 12, 13, 14, 15, 16, 17, 18, 19, or 20).

In certain embodiments, L₁, L₂, L₃, and L₄ independently comprise or together comprise the structure selected from among:

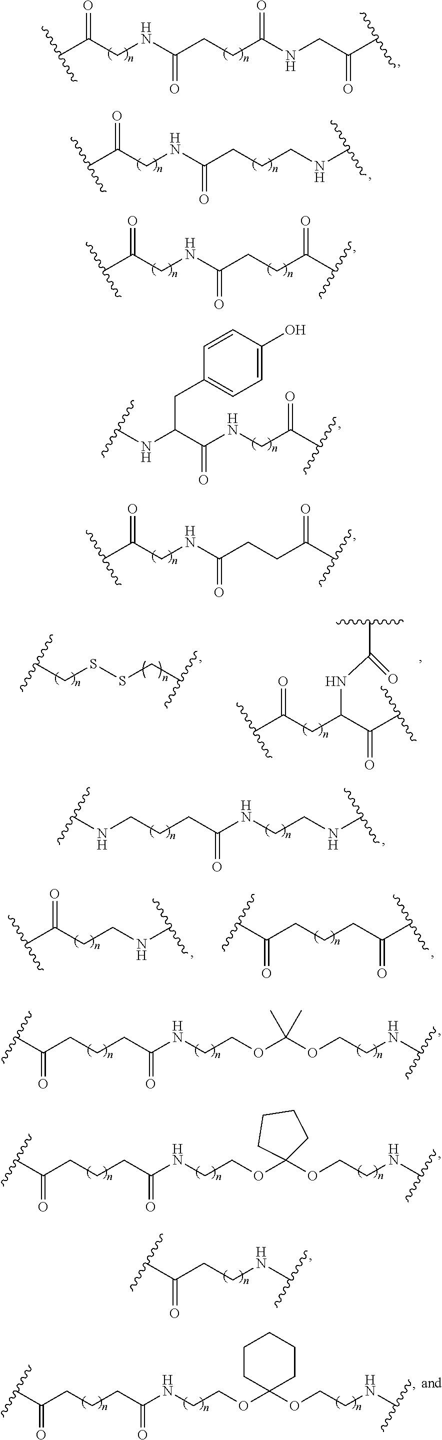

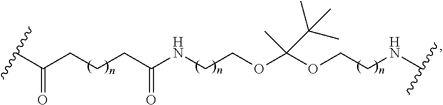

wherein each n is, independently, an integer from 1 to 20 (i.e., 1, 2, 3, 4, 5, 6, 7, 8, 9, 10, 11, 12, 13, 14, 15, 16, 17, 18, 19, or 20).

In certain embodiments, L₁, L₂, L₃, and L₄ independently comprise or together comprise the structure selected from among:

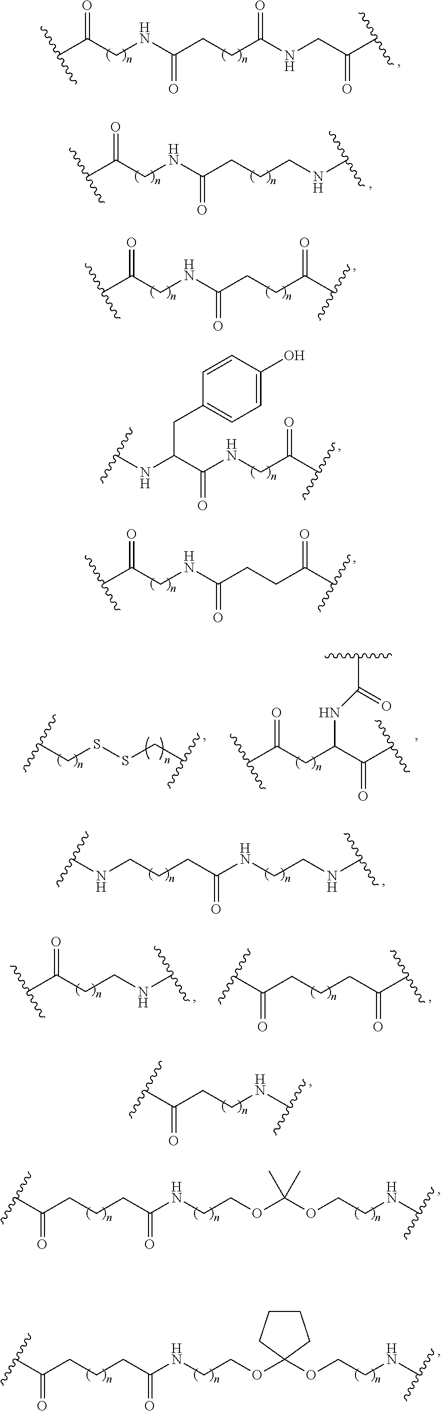

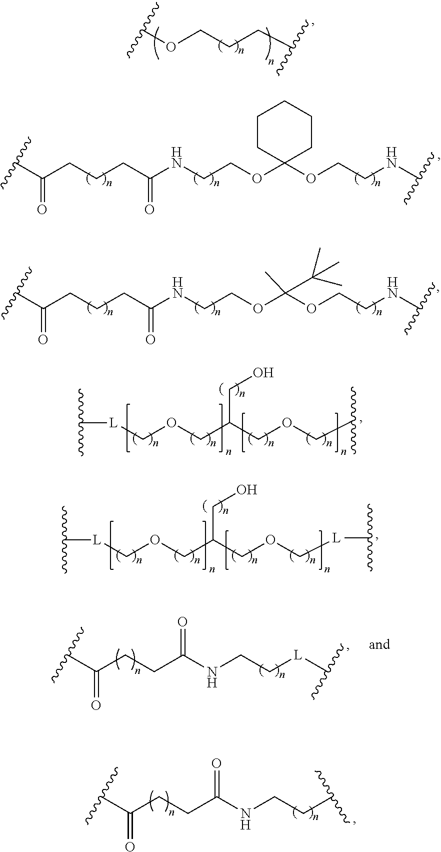

wherein each L is, independently, a phosphorous linking group; and each n is, independently, an integer from 1 to 20 (i.e., 1, 2, 3, 4, 5, 6, 7, 8, 9, 10, 11, 12, 13, 14, 15, 16, 17, 18, 19, or 20).

In certain embodiments, L₁, L₂, L₃, and L₄ independently comprise or together comprise the structure selected from among:

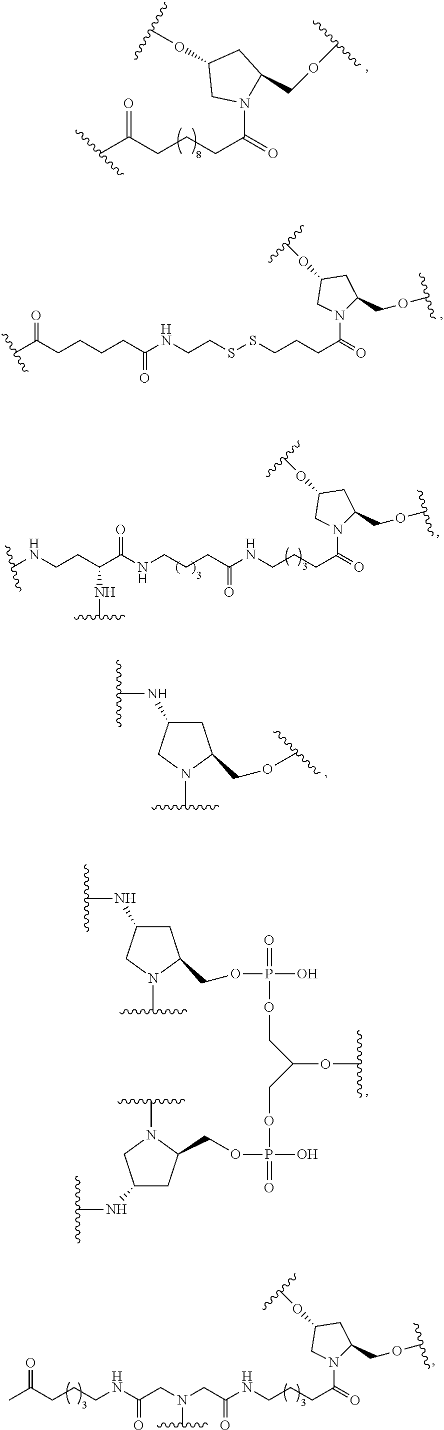

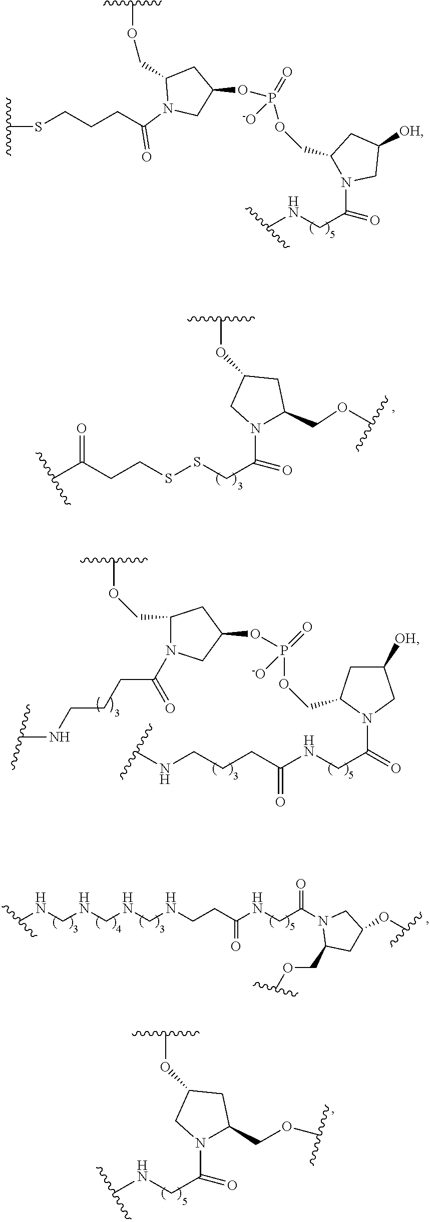

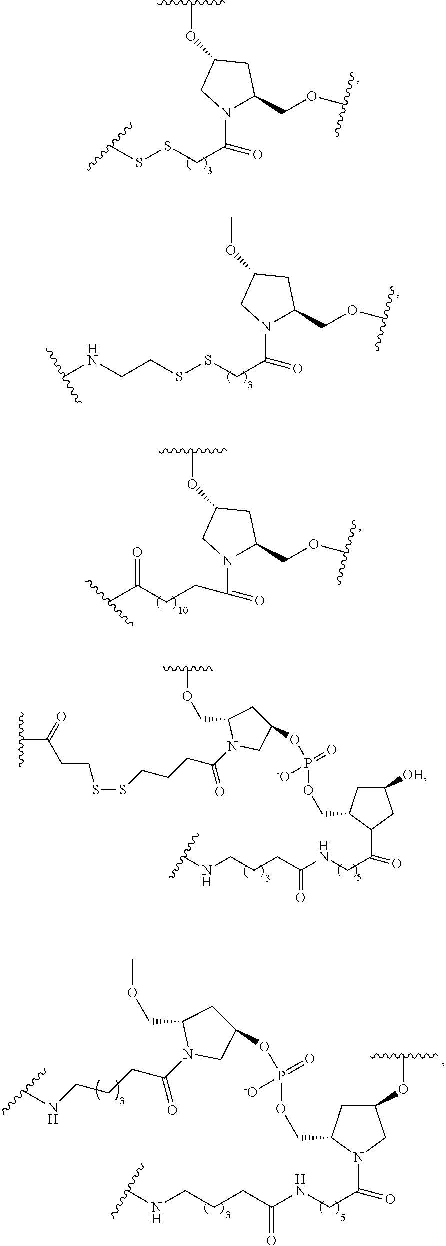

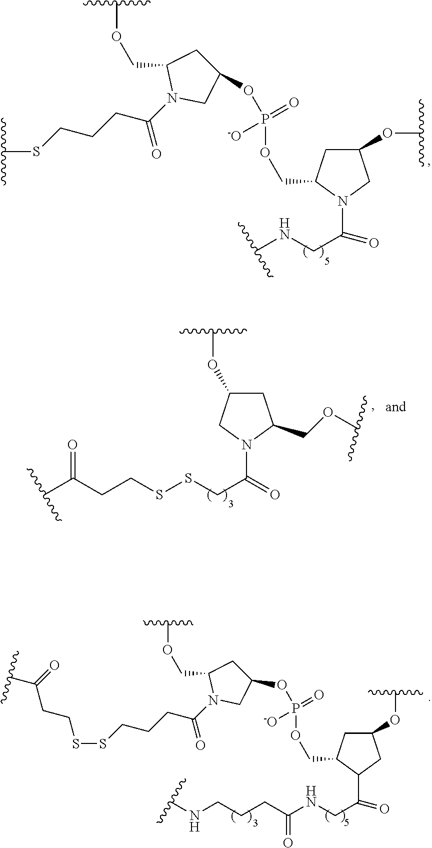

In certain embodiments, L₁, L₂, L₃, and L₄ independently comprise or together comprise the structure selected from among:

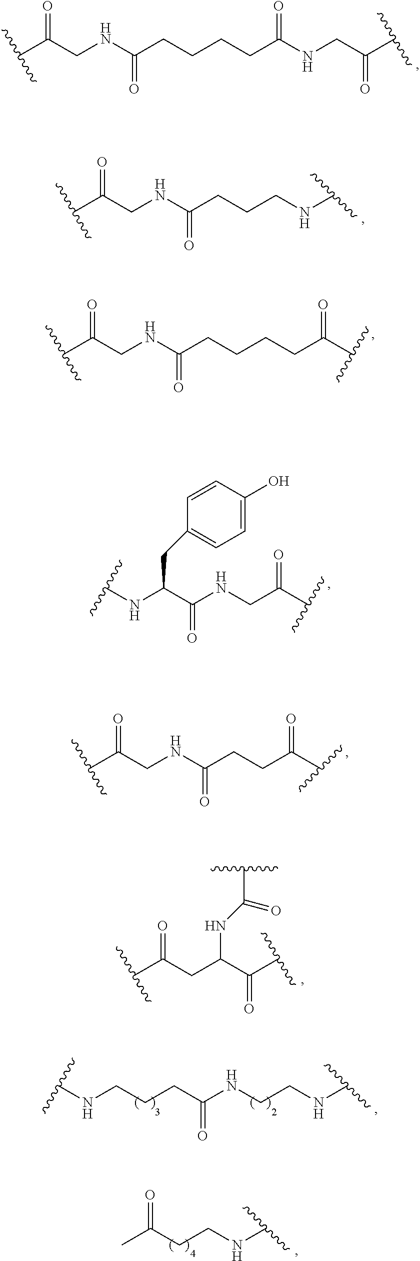

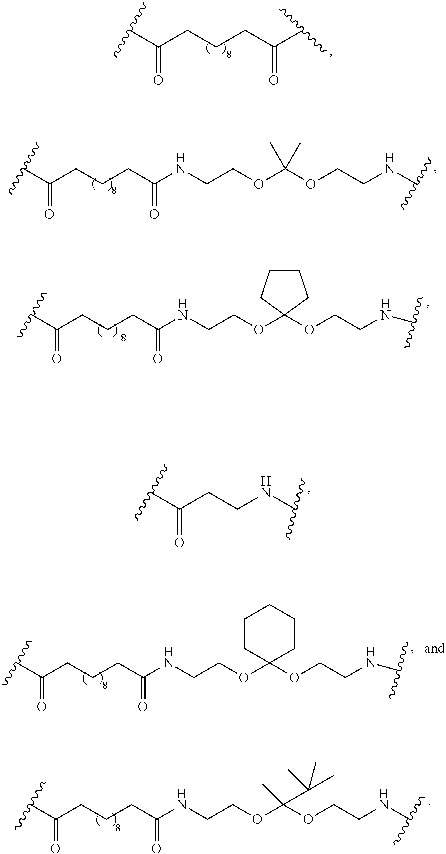

In certain embodiments, L₁, L₂, L₃, and L₄ independently comprise or together comprise the structure selected from among:

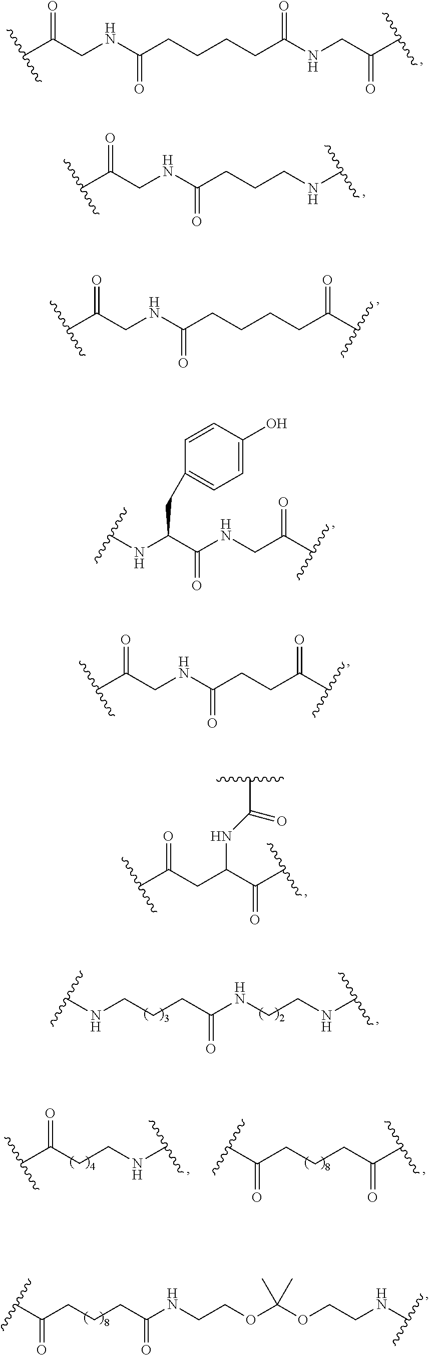

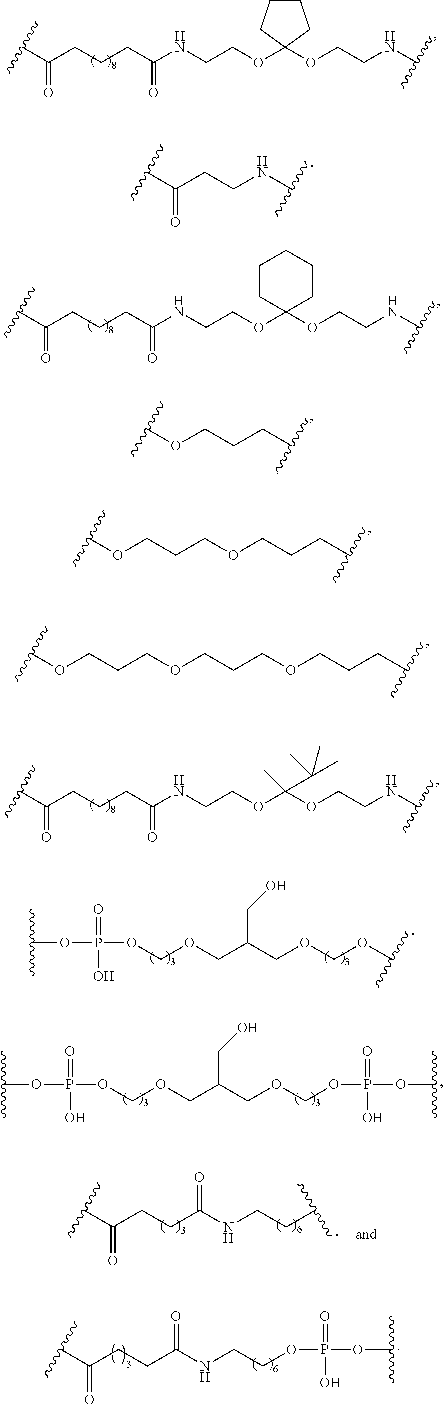

In certain embodiments, L₁, L₂, L₃, and L₄ independently comprise or together comprise the structure selected from among:

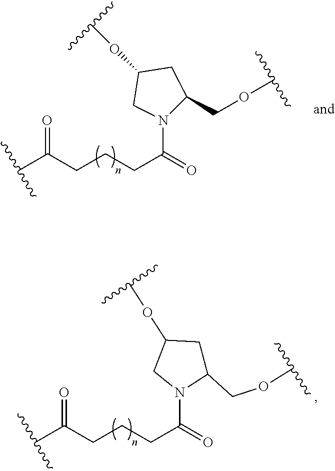

wherein n is from 1 to 20 (i.e., 1, 2, 3, 4, 5, 6, 7, 8, 9, 10, 11, 12, 13, 14, 15, 16, 17, 18, 19, or 20).

In certain embodiments, L₁, L₂, L₃, and L₄ independently comprise or together comprise the structure selected from among:

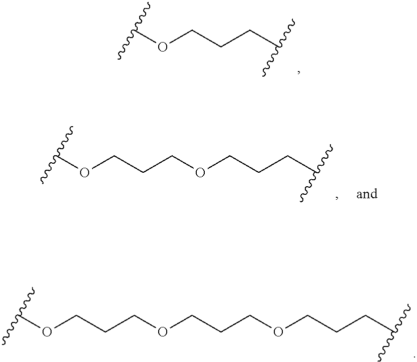 [SMILES: *CCCO* ; *CCCOCCCO* ; *CCCOCCCOCCCO*]

In certain embodiments, L₁, L₂, L₃, and L₄ independently comprise or together comprise the structure selected from among:

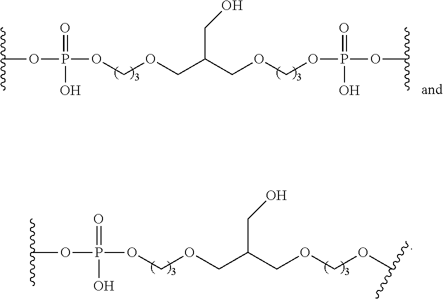

In certain embodiments, L₁, L₂, L₃, and L₄ independently comprise or together comprise the structure selected from among:

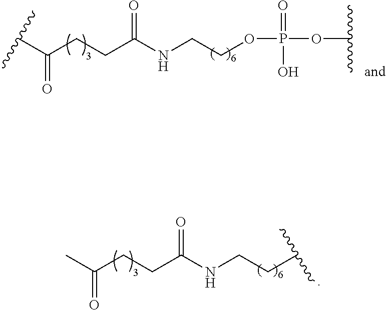

In certain embodiments, L₁, L₂, L₃, and L₄ independently comprise or together have the structure:

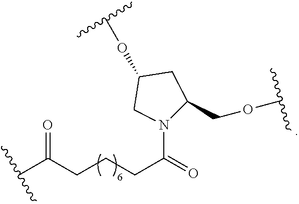

In certain embodiments, L₁, L₂, L₃, and L₄ independently comprise or together have the structure:

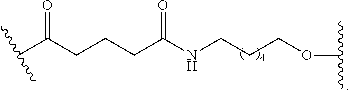

In certain embodiments, L₁, L₂, L₃, and L₄ independently comprise or together comprise the structure selected from among:

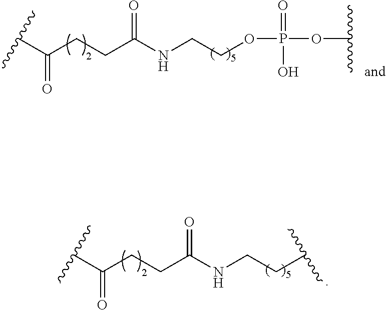

In certain embodiments, L₁, L₂, L₃, and L₄ independently comprise or together comprise the structure selected from among:

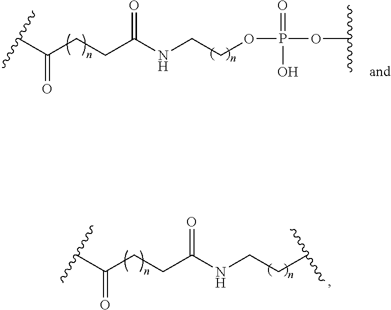

wherein each n is independently 0, 1, 2, 3, 4, 5, 6, or 7.

In some embodiments, any of L₁, L₂, L₃, and L₄ may independently be a linker (e.g., an optionally substituted alkyl linker, an optionally substituted polyethylene glycol (PEG) linker, an optionally substituted heteroalkyl linker, or an optionally substituted heteroaryl linker). In some embodiments, any of L₁, L₂, L₃, and L₄ may independently be a bond (e.g., a carbon-carbon bond, a phosphodiester bond, or a phosphorothioate bond). In some embodiments, any of L₁, L₂, L₃, and L₄ may independently be absent.

In some embodiments, L₁ is a bond. In some embodiments, L₁ is an optionally substituted alkyl linker. In some embodiments, L₁ is an optionally substituted C₁-C₆ alkyl linker. In some embodiments, L₁ is a C₁-C₆ alkyl linker substituted with ═O. In certain embodiments, L₁ comprises the structure

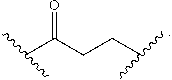 [SMILES: *CCC(*)=O]

In some embodiments, L₂ is an optionally substituted alkyl linker. In some embodiments, L₂ is an optionally substituted C₁-C₁₅ alkyl linker. In some embodiments, L₂ is an optionally substituted C₅-C₁₂ alkyl linker. In certain embodiments, L₂ comprises the structure

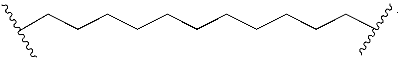 [SMILES: *CCCCCCCCCCC*]

In certain embodiments, L₂ comprises the structure

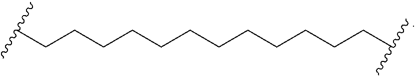 [SMILES: *CCCCCCCCCCCC*]

In some embodiments, L₂ is an optionally substituted PEG linker. In some embodiments, L₂ is an optionally substituted PEG linker comprising one, two, three, four, five, six, seven, or eight PEG units in length, wherein a PEG unit comprises the structure

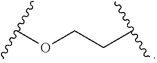 [SMILES: *CCO*]

In certain embodiments, L₂ is an optionally substituted PEG linker comprising three PEG units in length. In certain embodiments, L₂ is an optionally substituted PEG linker comprising four PEG units in length. In certain embodiments, L₂ comprises the structure

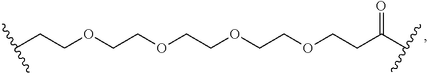 [SMILES: *CCOCCOCCOCCOCCC(*)=O]

In certain embodiments, L₂ comprises the structure

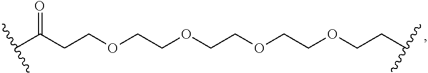 [SMILES: *CCOCCOCCOCCOCCC(*)=O]

In certain embodiments, L₂ comprises the structure

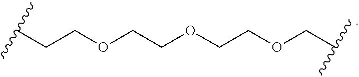 [SMILES: *CCOCCOCCOC*]

In certain embodiments, L₂ comprises the structure

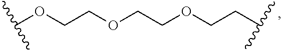 [SMILES: *CCOCCOCCO*]

In certain embodiments, L₂ comprises the structure

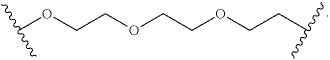 [SMILES: *CCOCCOCCO*]

In certain embodiments, L₂ comprises the structure

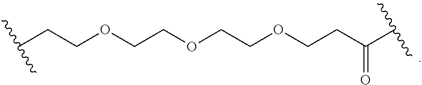 [SMILES: *CCOCCOCCOCCC(*)=O]

In certain embodiments, L₂ comprises the structure

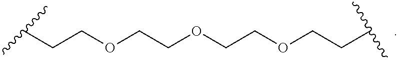 [SMILES: *CCOCCOCCOCC*]

In certain embodiments, L₂ comprises the structure

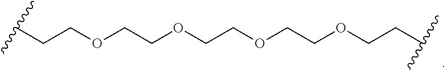 [SMILES: *CCOCCOCCOCCOCC*]

In some embodiments, L₂ is an optionally substituted heteroalkyl linker. In certain embodiments, L₂ comprises the structure

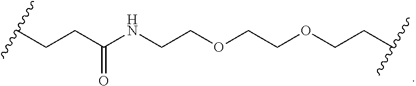 [SMILES: *CCOCCOCCNC(=O)CC*]

In certain embodiments, L₂ comprises the structure

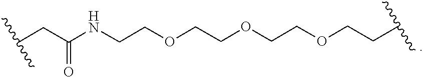 [SMILES: *CCOCCOCCOCCNC(=O)C*]

In some embodiments, L₃ is an optionally substituted heteroaryl linker. In some embodiments, L₃ is an optionally substituted partially unsaturated heterocycloalkyl linker or a heteroaryl linker. In certain embodiments, L₃ comprises the structure

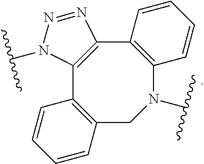 [SMILES: *N1Cc2ccccc2-c2c(nnn2*)-c2ccccc21]

In some embodiments, L₄ is an optionally substituted heteroalkyl linker. In some embodiments, the heteroalkyl linker is substituted with one or more ═O substituents. In certain embodiments, L₄ comprises the structure

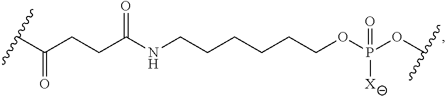

wherein X is O or S. In certain embodiments, L₄ comprises the structure

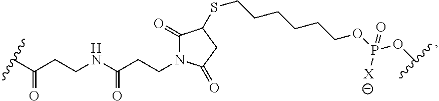

wherein X is O or S.

In some embodiments, L₁, L₂, L₃, and L₄ together comprise the structure

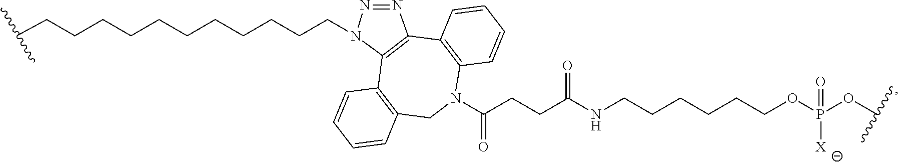

wherein X is O or S. In some embodiments, L₁, L₂, L₃, and L₄ together comprise the structure

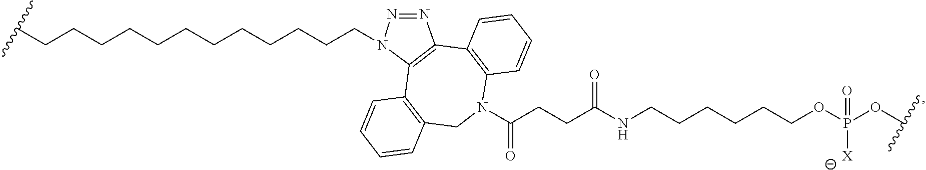

wherein X is O or S. In some embodiments, L₁, L₂, L₃, and L₄ together comprise the structure

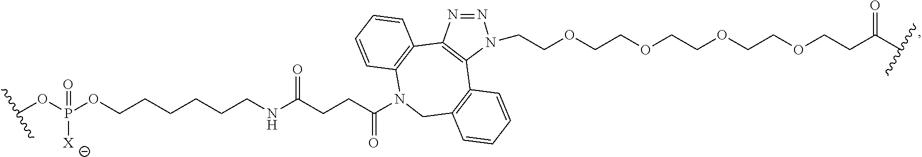

wherein X is O or S. In some embodiments, L₁, L₂, L₃, and L₄ together comprise the structure

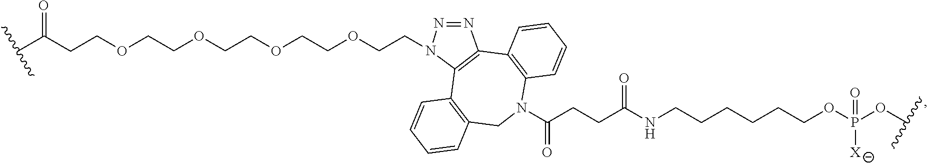

wherein X is O or S. In some embodiments, L₁, L₂, L₃, and L₄ together comprise the structure

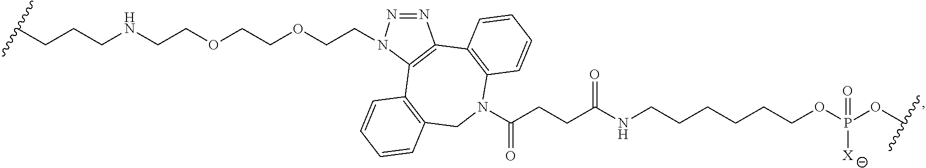

wherein X is O or S. In some embodiments, L₁, L₂, L₃, and L₄ together comprise the structure

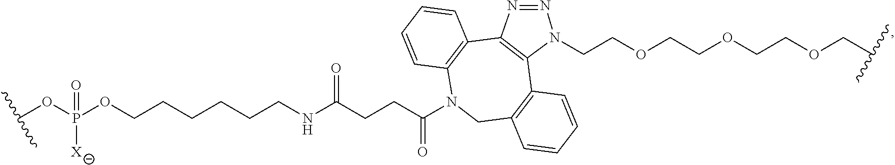

wherein X is O or S. In some embodiments, L₁, L₂, L₃, and L₄ together comprise the structure

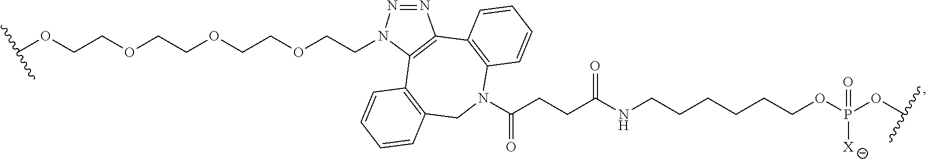

wherein X is O or S. In some embodiments, L₁, L₂, L₃, and L₄ together comprise the structure

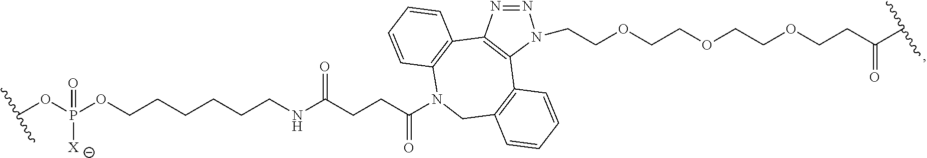

wherein X is O or S. In some embodiments, L₁, L₂, L₃, and L₄ together comprise the structure

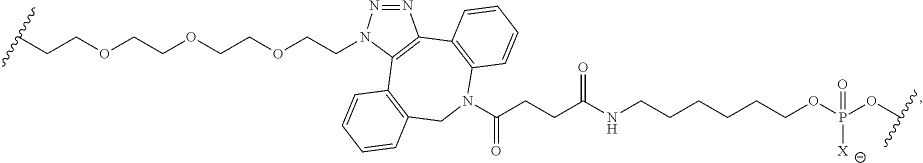

wherein X is O or S. In some embodiments, L₁, L₂, L₃, and L₄ together comprise the structure

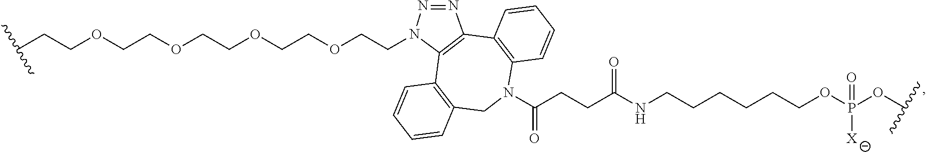

wherein X is O or S. In some embodiments, L₁, L₂, L₃, and L₄ together comprise the structure

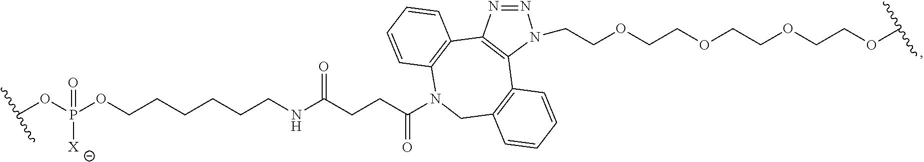

In some embodiments, L₁, L₂, L₃, and L₄ together comprise the structure

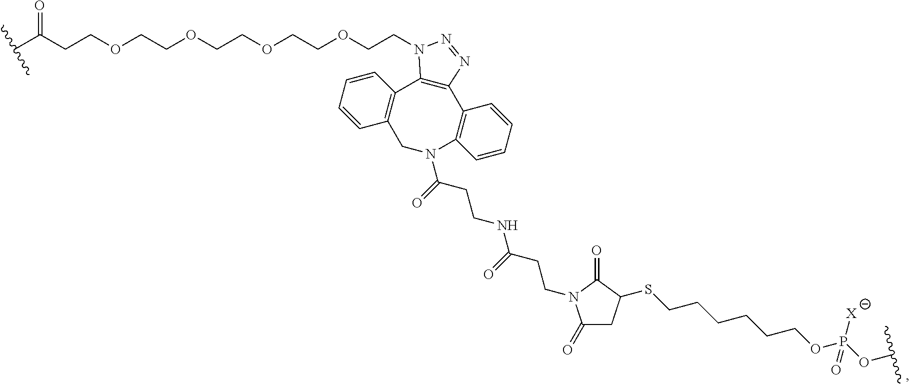

wherein X is O or S.

### Methods of Making Compounds

In some aspects, the disclosure relates to methods of making the compounds and compositions comprising NMDA receptor ligands as disclosed herein.

Compounds of the present disclosure can be made by means known in the art of organic synthesis. Methods for optimizing reaction conditions, and minimizing competing by-products, if necessary, are known in the art. Reaction optimization and scale-up may advantageously utilize high-speed parallel synthesis equipment and computer-controlled microreactors (e.g., Design and Optimization in Organic Synthesis, 2^{nd} Edition, Carlson R, Ed, 2005; Elsevier Science Ltd.; Jahnisch, K et al., Angew. Chem. Int. Ed. Engl. 2004 43: 406; and references therein). Additional reaction schemes and protocols may be determined by the skilled artisan by use of commercially available structure-searchable database software, for instance, SciFinder® (CAS division of the American Chemical Society) and Reaxys® (Elsevier), or by appropriate keyword searching using an internet search engine such as Google® or keyword databases such as the U.S. Patent and Trademark Office text database.

As can be appreciated by the skilled artisan, methods of synthesizing the compounds of the formulae herein will be evident to those of ordinary skill in the art, including in the schemes and examples herein. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired compounds of the present disclosure.

The compounds herein may also contain linkages (e.g., carbon-carbon bonds) wherein bond rotation is restricted about that particular linkage, e.g., restriction resulting from the presence of a ring or double bond. Accordingly, all cis/trans and E/Z isomers are expressly included in the present disclosure. The compounds herein may also be represented in multiple tautomeric forms; in such instances, the present disclosure expressly includes all tautomeric forms of the compounds described herein, even though only a single tautomeric form may be represented. All such isomeric forms of such compounds herein are expressly included in the present disclosure. All crystal forms and polymorphs of the compounds described herein are expressly included in the present disclosure. Also embodied are extracts and fractions comprising compounds of the present disclosure. The term “isomers” is intended to include diastereoisomers, enantiomers, regioisomers, structural isomers, rotational isomers, tautomers, and the like. For compounds which contain one or more stereogenic centers, e.g., chiral compounds, the methods of the present disclosure may be carried out with an enantiomerically enriched compound, a racemate, or a mixture of diastereomers. All isomers of compounds delineated herein are expressly included in the present disclosure.

Preferred enantiomerically enriched compounds have an enantiomeric excess of 50% or more. More preferably, the compound has an enantiomeric excess of 60%, 70%, 80%, 90%, 95%, 98%, 99%, or more. In preferred embodiments, only one enantiomer or diastereomer of a chiral compound of the present disclosure is administered to cells or a subject.

### Methods of Treatment

In one aspect, provided are methods of treating a subject suffering from or susceptible to a disorder or disease, comprising administering to the subject an effective amount of a compound or pharmaceutical composition described herein.

In other aspects, provided are methods of treating a subject suffering from or susceptible to a disorder or disease, wherein the subject has been identified as in need of modulation of the function of a protein, comprising administering to said subject in need thereof, an effective amount of a compound or pharmaceutical composition described herein, such that said subject is treated for said disorder.

In one aspect, provided are methods of delivering a therapeutic oligonucleotide to the brain of a subject, comprising contacting the subject with a compound or pharmaceutical composition described herein, in an amount and under conditions sufficient to target the brain.

In some embodiments, the therapeutic oligonucleotide is delivered to one or more brain regions selected from the group consisting of the striatum, the cerebellum, the brain stem, the hippocampus, the frontal cortex, and the spinal cord.

In certain embodiments, provided are methods of treating a disease, disorder or symptom thereof, wherein the disease is a central nervous system (CNS) disease, disorder, or symptom thereof. In some embodiments, the disease is a neurodegenerative disease, disorder, or symptom thereof. In some embodiments, the disease is Alzheimer's disease, or a symptom thereof.

Exemplary CNS disorders include, but are not limited to, neurotoxicity and/or neurotrauma, stroke, multiple sclerosis, spinal cord injury, epilepsy, a mental disorder, a sleep condition, a movement disorder, nausea and/or emesis, amyotrophic lateral sclerosis, Alzheimer's disease, and substance abuse or substance use disorder (SUD).

In certain embodiments, the CNS disorder is neurotoxicity and/or neurotrauma, e.g., for example, as a result of acute neuronal injury (e.g., traumatic brain injury (TBI), stroke, epilepsy) or a chronic neurodegenerative disorder (e.g., multiple sclerosis, Parkinson's disease, Huntington's disease, amyotrophic lateral sclerosis, Alzheimer's disease). In certain embodiments, the compounds of the present disclosure provide a neuroprotective effect, e.g., against an acute neuronal injury or a chronic neurodegenerative disorder.

In certain embodiments, the CNS disorder is stroke (e.g., ischemic stroke).

In certain embodiments, the CNS disorder is multiple sclerosis.

In certain embodiments, the CNS disorder is spinal cord injury.

In certain embodiments, the CNS disorder is epilepsy.

In certain embodiments, the CNS disorder is a mental disorder, e.g., for example, depression, anxiety or anxiety-related conditions, a learning disability, somatic symptom disorder, schizophrenia, or schizoaffective disorder.

In certain embodiments, the CNS disorder is depression. “Depression” includes, but is not limited to, depressive disorders or conditions, such as, for example, major depressive disorders (e.g., unipolar depression), treatment-resistant depression, dysthymic disorders (e.g., chronic, mild depression), bipolar disorders (e.g., manic-depression), seasonal affective disorder, and/or depression associated with substance abuse or substance abuse disorder (e.g., withdrawal). The depression can be clinical or subclinical depression. The depression can be associated with or premenstrual syndrome and/or premenstrual dysphoric disorder.

In certain embodiments, the CNS disorder is anxiety. “Anxiety” includes, but is not limited to, anxiety and anxiety-related conditions, such as, for example, clinical anxiety, panic disorder, agoraphobia, generalized anxiety disorder (GAD), specific phobia, social phobia, obsessive-compulsive disorder, acute stress disorder, post-traumatic stress disorder, adjustment disorders with anxious features, anxiety disorder associated with depression, anxiety disorder due to general medical conditions, and substance-induced anxiety disorders, anxiety associated with substance abuse or substance use disorder (e.g., withdrawal, dependence, reinstatement) and anxiety associated with nausea and/or emesis. This treatment may also be to induce or promote sleep in a subject (e.g., for example, a subject with anxiety).

In certain embodiments, the CNS disorder is a learning disorder (e.g., attention deficit disorder (ADD)).

In certain embodiments, the CNS disorder is schizophrenia or schizoaffective disorder.

In certain embodiments, the CNS disorder is a sleep condition. “Sleep conditions” include, but are not limited to, insomnia, narcolepsy, sleep apnea, restless legs syndrome (RLS), delayed sleep phase syndrome (DSPS), periodic limb movement disorder (PLMD), hypopnea syndrome, rapid eye movement behavior disorder (RBD), shift work sleep condition (SWSD), and sleep problems (e.g., parasomnias) such as nightmares, night terrors, sleep talking, head banging, snoring, and clenched jaw and/or grinding of teeth (bruxism).

In certain embodiments, the CNS disorder is a movement disorder, e.g., basal ganglia disorders, such as, for example, Parkinson's disease, levodopa-induced dyskinesia, Huntington's disease, Gilles de Ia Tourette's syndrome, tardive dyskinesia, and dystonia.

In certain embodiments, the CNS disorder is Alzheimer's disease.

In certain embodiments, the CNS disorder is amyotrophic lateral sclerosis (ALS).

In certain embodiments, the CNS disorder is nausea and/or emesis.

In certain embodiments, the CNS disorder is substance abuse or substance use disorder (SUD) (e.g., for instance, addiction to opiates, nicotine, cocaine, psychostimulants, or alcohol).

The term “neurological disease” (including, e.g., “neurodegenerative diseases) refers to any disease of the nervous system, including diseases that involve the central nervous system (brain, brainstem and cerebellum), the peripheral nervous system (including cranial nerves), and the autonomic nervous system (parts of which are located in both central and peripheral nervous system). Neurodegenerative diseases refer to a type of neurological disease marked by the loss of nerve cells, including, but not limited to, Alzheimer's disease, Parkinson's disease, amyotrophic lateral sclerosis, tauopathies (including frontotemporal dementia), and Huntington's disease. Examples of neurological diseases include, but are not limited to, headache, stupor and coma, dementia, seizure, sleep disorders, trauma, infections, neoplasms, neuro-ophthalmology, movement disorders, demyelinating diseases, spinal cord disorders, and disorders of peripheral nerves, muscle, and neuromuscular junctions. Substance abuse or substance use disorder (SUD) and mental illness, including, but not limited to, bipolar disorder, schizophrenia and schizoaffective disorder, are also included in the definition of neurological diseases. Further examples of neurological diseases include acquired epileptiform aphasia; acute disseminated encephalomyelitis; adrenoleukodystrophy; agenesis of the corpus callosum; agnosia; Aicardi syndrome; Alexander disease; Alpers' disease; alternating hemiplegia; Alzheimer's disease; amyotrophic lateral sclerosis; anencephaly; Angelman syndrome; angiomatosis; anoxia; aphasia; apraxia; arachnoid cysts; arachnoiditis; Arnold-Chiari malformation; arteriovenous malformation; Asperger syndrome; ataxia telangiectasia; attention deficit hyperactivity disorder; autism; autonomic dysfunction; back pain; Batten disease; Behcet's disease; Bell's palsy; benign essential blepharospasm; benign focal; amyotrophy; benign intracranial hypertension; Binswanger's disease; blepharospasm; Bloch Sulzberger syndrome; brachial plexus injury; brain abscess; brain injury; brain tumors (including glioblastoma multiforme); spinal tumor; Brown-Sequard syndrome; Canavan disease; carpal tunnel syndrome (CTS); causalgia; central pain syndrome; central pontine myelinolysis; cephalic disorder; cerebral aneurysm; cerebral arteriosclerosis; cerebral atrophy; cerebral gigantism; cerebral palsy; Charcot-Marie-Tooth disease; chemotherapy-induced neuropathy and neuropathic pain; Chiari malformation; chorea; chronic inflammatory demyelinating polyneuropathy (CIDP); chronic pain; chronic regional pain syndrome; Coffin Lowry syndrome; coma, including persistent vegetative state; congenital facial diplegia; corticobasal degeneration; cranial arteritis; craniosynostosis; Creutzfeldt-Jakob disease; cumulative trauma disorders; Cushing's syndrome; cytomegalic inclusion body disease (CIBD); cytomegalovirus infection; dancing eyes-dancing feet syndrome; Dandy-Walker syndrome; Dawson disease; De Morsier's syndrome; Dejerine-Klumpke palsy; dementia; dermatomyositis; diabetic neuropathy; diffuse sclerosis; dysautonomia; dysgraphia; dyslexia; dystonias; early infantile epileptic encephalopathy; empty sella syndrome; encephalitis; encephaloceles; encephalotrigeminal angiomatosis; epilepsy; Erb's palsy; essential tremor; Fabry's disease; Fahr's syndrome; fainting; familial spastic paralysis; febrile seizures; Fisher syndrome; Friedreich's ataxia; frontotemporal dementia and other “tauopathies”; Gaucher's disease; Gerstmann's syndrome; giant cell arteritis; giant cell inclusion disease; globoid cell leukodystrophy; Guillain-Barre syndrome; HTLV-1 associated myelopathy; Hallervorden-Spatz disease; head injury; headache; hemifacial spasm; hereditary spastic paraplegia; heredopathia atactica polyneuritiformis; herpes zoster oticus; herpes zoster; Hirayama syndrome; HIV-associated dementia and neuropathy (see also neurological manifestations of AIDS); holoprosencephaly; Huntington's disease and other polyglutamine repeat diseases; hydranencephaly; hydrocephalus; hypercortisolism; hypoxia; immune-mediated encephalomyelitis; inclusion body myositis; incontinentia pigmenti; infantile; phytanic acid storage disease; Infantile Refsum disease; infantile spasms; inflammatory myopathy; intracranial cyst; intracranial hypertension; Joubert syndrome; Kearns-Sayre syndrome; Kennedy disease; Kinsbourne syndrome; Klippel Feil syndrome; Krabbe disease; Kugelberg-Welander disease; kuru; Lafora disease; Lambert-Eaton myasthenic syndrome; Landau-Kleffner syndrome; lateral medullary (Wallenberg) syndrome; learning disabilities; Leigh's disease; Lennox-Gastaut syndrome; Lesch-Nyhan syndrome; leukodystrophy; Lewy body dementia; lissencephaly; locked-in syndrome; Lou Gehrig's disease (also known as motor neuron disease or amyotrophic lateral sclerosis); lumbar disc disease; lyme disease-neurological sequelae; Machado-Joseph disease; macrencephaly; megalencephaly; Melkersson-Rosenthal syndrome; Meniere's disease; meningitis; Menkes disease; metachromatic leukodystrophy; microcephaly; migraine; Miller Fisher syndrome; mini-strokes; mitochondrial myopathies; Mobius syndrome; monomelic amyotrophy; motor neurone disease; moyamoya disease; mucopolysaccharidoses; multi-infarct dementia; multifocal motor neuropathy; multiple sclerosis and other demyelinating disorders; multiple system atrophy with postural hypotension; muscular dystrophy; myasthenia gravis; myelinoclastic diffuse sclerosis; myoclonic encephalopathy of infants; myoclonus; myopathy; myotonia congenital; narcolepsy; neurofibromatosis; neuroleptic malignant syndrome; neurological manifestations of AIDS; neurological sequelae of lupus; neuromyotonia; neuronal ceroid lipofuscinosis; neuronal migration disorders; Niemann-Pick disease; O'Sullivan-McLeod syndrome; occipital neuralgia; occult spinal dysraphism sequence; Ohtahara syndrome; olivopontocerebellar atrophy; opsoclonus myoclonus; optic neuritis; orthostatic hypotension; overuse syndrome; paresthesia; Parkinson's disease; paramyotonia congenita; paraneoplastic diseases; paroxysmal attacks; Parry Romberg syndrome; Pelizaeus-Merzbacher disease; periodic paralyses; peripheral neuropathy; painful neuropathy and neuropathic pain; persistent vegetative state; pervasive developmental disorders; photic sneeze reflex; phytanic acid storage disease; Pick's disease; pinched nerve; pituitary tumors; polymyositis; porencephaly; Post-Polio syndrome; postherpetic neuralgia (PHN); postinfectious encephalomyelitis; postural hypotension; Prader-Willi syndrome; primary lateral sclerosis; prion diseases; progressive; hemifacial atrophy; progressive multifocal leukoencephalopathy; progressive sclerosing poliodystrophy; progressive supranuclear palsy; pseudotumor cerebri; Ramsay-Hunt syndrome (Type I and Type II); Rasmussen's Encephalitis; reflex sympathetic dystrophy syndrome; Refsum disease; repetitive motion disorders; repetitive stress injuries; restless legs syndrome; retrovirus-associated myelopathy; Rett syndrome; Reye's syndrome; Saint Vitus Dance; Sandhoff disease; Schilder's disease; schizencephaly; septo-optic dysplasia; shaken baby syndrome; shingles; Shy-Drager syndrome; Sjogren's syndrome; sleep apnea; Soto's syndrome; spasticity; spina bifida; spinal cord injury; spinal cord tumors; spinal muscular atrophy; stiff-person syndrome; stroke; Sturge-Weber syndrome; subacute sclerosing panencephalitis; subarachnoid hemorrhage; subcortical arteriosclerotic encephalopathy; sydenham chorea; syncope; syringomyelia; tardive dyskinesia; Tay-Sachs disease; temporal arteritis; tethered spinal cord syndrome; Thomsen disease; thoracic outlet syndrome; tic douloureux; Todd's paralysis; Tourette syndrome; transient ischemic attack; transmissible spongiform encephalopathies; transverse myelitis; traumatic brain injury; tremor; trigeminal neuralgia; tropical spastic paraparesis; tuberous sclerosis; vascular dementia (multi-infarct dementia); vasculitis including temporal arteritis; Von Hippel-Lindau Disease (VHL); Wallenberg's syndrome; Werdnig-Hoffman disease; West syndrome; whiplash; Williams syndrome; Wilson's disease; and Zellweger syndrome.

In certain embodiments, the subject is a mammal, preferably a primate or a human.

In another embodiment, provided are methods as described above, wherein the effective amount of the compounds provided herein is as described above.

In another embodiment, provided are methods as described above, wherein the compounds provided herein is administered intrathecally, intravenously, intramuscularly, subcutaneously, intracerebroventricularly, orally, or topically. In certain embodiments, the compound is administered intrathecally.

In other embodiments, provided are methods as described above, wherein the compound of any of the formulae provided herein is administered alone or in combination with one or more other therapeutics. In a further embodiment, the additional therapeutic agent is a central nervous system (CNS) disease agent.

Another object of the present disclosure is the use of a compound as described herein in the manufacture of a medicament for use in the treatment of a disorder or disease. Another object of the present disclosure is the use of a compound as described herein for use in the treatment of a disorder or disease.

### Pharmaceutical Compositions

In one aspect, provided are pharmaceutical compositions comprising any of the compounds described herein, and a pharmaceutically acceptable carrier or pharmaceutically acceptable excipient.

A compound or composition, as described herein, can be administered in combination with one or more additional therapeutic agents (e.g., therapeutically and/or prophylactically active agents). The compounds or compositions can be administered in combination with additional therapeutic agents that improve their activity (e.g., activity (e.g., potency and/or efficacy) in treating a disease in a subject in need thereof, in preventing a disease in a subject in need thereof, and/or in reducing the risk to develop a disease in a subject in need thereof), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional therapeutic agent exhibits a synergistic effect that is absent in a pharmaceutical composition including one of the compounds described herein or the additional therapeutic agent, but not both.

The compound or composition can be administered concurrently with, prior to, or subsequent to one or more additional therapeutic agents, which may be useful as, e.g., combination therapies. Therapeutic agents include therapeutically active agents. Therapeutic agents also include prophylactically active agents. Therapeutic agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional therapeutic agent is a therapeutic agent useful for treating and/or preventing a disease (e.g., CNS disorder). Each additional therapeutic agent may be administered at a dose and/or on a time schedule determined for that therapeutic agent. The additional therapeutic agents may also be administered together with each other and/or with the compound or composition described herein in a single dose or administered separately in different doses. The particular combination to employ in a regimen will take into account compatibility of the compound described herein with the additional therapeutic agent(s) and/or the desired therapeutic and/or prophylactic effect to be achieved. In general, it is expected that the additional therapeutic agent(s) in combination be utilized at levels that do not exceed the levels at which they are utilized individually. In some embodiments, the levels utilized in combination will be lower than those utilized individually.

In one aspect, provided are kits comprising an effective amount of a compound provided herein, in unit dosage form, together with instructions for administering the compound to a subject suffering from or susceptible to a disease or disorder.

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydroiodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present disclosure contain both basic and acidic functionalities that allow the compounds to be converted into either base or acid addition salts. Other pharmaceutically acceptable carriers known to those of skill in the art are suitable for the present disclosure.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

In addition to salt forms, the present disclosure provides compounds which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Certain compounds of the present disclosure can exist in unsolvated forms as well as solvated forms, including hydrated forms. In general, the solvated forms are equivalent to unsolvated forms and are intended to be encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

The present disclosure also provides a pharmaceutical composition, comprising an effective amount of a compound described herein and a pharmaceutically acceptable excipient. In an embodiment, a compound of any of the formulae provided herein is administered to a subject using a pharmaceutically-acceptable formulation, e.g., a pharmaceutically-acceptable formulation that provides sustained delivery of the compound to a subject for at least 12 hours, 24 hours, 36 hours, 48 hours, one week, two weeks, three weeks, or four weeks after the pharmaceutically-acceptable formulation is administered to the subject.

Actual dosage levels and time course of administration of the active ingredients in the pharmaceutical compositions of the disclosure may be varied so as to obtain an amount of the active ingredient which is effective to achieve the desired therapeutic response for a particular subject, composition, and mode of administration, while being acceptably tolerant to the subject.

In use, at least one compound according to the present disclosure is administered in a pharmaceutically effective amount to a subject in need thereof in a pharmaceutical carrier by intravenous, intrathecal, intramuscular, subcutaneous, or intracerebroventricular injection or by oral administration or topical application. In accordance with the present disclosure, a compound of the disclosure may be administered alone or in conjunction with a second, different therapeutic. By “in conjunction with” is meant together, substantially simultaneously, or sequentially. In one embodiment, a compound of the disclosure is administered acutely.

The compound of the disclosure may therefore be administered for a short course of treatment, such as for about 1 day to about 1 week. In another embodiment, the compound of the disclosure may be administered over a longer period of time to ameliorate chronic disorders, such as, for example, for about one week to several months depending upon the condition to be treated.

By “pharmaceutically effective amount,” as used herein, is meant an amount of a compound of the disclosure, high enough to significantly positively modify the condition to be treated but low enough to avoid serious side effects (at a reasonable benefit/risk ratio), within the scope of sound medical judgment. A pharmaceutically effective amount of a compound of the disclosure will vary with the particular goal to be achieved, the age and physical condition of the patient being treated, the severity of the underlying disease, the duration of treatment, the nature of concurrent therapy and the specific compound employed. For example, a therapeutically effective amount of a compound of the disclosure administered to a child or a neonate will be reduced proportionately in accordance with sound medical judgment. The effective amount of a compound of the disclosure will thus be the minimum amount which will provide the desired effect.

A decided practical advantage of the present disclosure is that the compound may be administered in a convenient manner such as by intrathecal, intravenous, intramuscular, subcutaneous, oral, or intra-cerebroventricular injection routes or by topical application, such as in creams or gels. Depending on the route of administration, the active ingredients which comprise a compound of the disclosure may be required to be coated in a material to protect the compound from the action of enzymes, acids and other natural conditions which may inactivate the compound. In order to administer a compound of the disclosure by a mode other than parenteral administration, the compound can be coated by, or administered with, a material to prevent inactivation.

The compound may be administered parenterally or intraperitoneally. Dispersions can also be prepared, for example, in glycerol, liquid polyethylene glycols, and mixtures thereof, and in oils.

Some examples of substances which can serve as pharmaceutical excipients, or pharmaceutical carriers (which terms are used interchangeably herein), are sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil, and oil of theobroma; polyols such as propylene glycol, glycerine, sorbitol, mannitol, and polyethylene glycol; agar; alginic acids; pyrogen-free water; isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, anti-oxidants, and preservatives, can also be present. Solubilizing agents, including for example, cremaphore, and beta-cyclodextrins, can also be used in the pharmaceutical compositions herein.

Pharmaceutical compositions comprising the active compounds of the present disclosure (or derivatives or prodrugs thereof) can be manufactured by means of conventional mixing, dissolving, granulating, dragee-making levigating, emulsifying, encapsulating, entrapping, or lyophilization processes. The compositions can be formulated in conventional manner using one or more physiologically acceptable carriers, diluents, excipients, or auxiliaries, which facilitate processing of the active compounds into preparations that can be used pharmaceutically. The compositions herein can be made by combining (e.g., contacting, mixing, dissolving, granulating, dragee-making, levigating, emulsifying, encapsulating, entrapping, or lyophilizing) a compound delineated herein with one or more suitable carriers, diluents, excipients, or auxiliaries, including those described herein (e.g., for pharmaceutical, agricultural, or veterinary use).

Pharmaceutical compositions of the present disclosure can take a form suitable for virtually any mode of administration, including, for example, intrathecal, topical, ocular, oral, buccal, systemic, nasal, injection, transdermal, rectal, vaginal, and the like, or a form suitable for administration by inhalation or insufflation.

Systemic formulations include those designed for administration by injection, e.g., subcutaneous, intravenous, intramuscular, intrathecal, or intraperitoneal injection, as well as those designed for transdermal, transmucosal, oral, or pulmonary administration.

Useful injectable preparations include sterile suspensions, solutions, or emulsions of the active compound(s) in aqueous or oily vehicles. The compositions also can contain formulating agents, such as suspending, stabilizing and/or dispersing agent. The formulations for injection can be presented in unit dosage form (e.g., in ampules or in multidose containers) and can contain added preservatives.

Alternatively, the injectable formulation can be provided in powder form for reconstitution with a suitable vehicle, including but not limited to, sterile pyrogen free water, buffer, dextrose solution, and the like, before use. To this end, the active compound(s) can be dried by any art-known technique, such as lyophilization, and reconstituted prior to use.

For prolonged delivery, the active compound(s), or prodrug(s) can be formulated as a depot preparation for administration by implantation or intramuscular injection. The active ingredient can be formulated with suitable polymeric or hydrophobic materials (e.g., as an emulsion in an acceptable oil) or ion exchange resins, or as sparingly soluble derivatives, e.g., as a sparingly soluble salt.

Alternatively, other pharmaceutical delivery systems can be employed. Liposomes and emulsions are well-known examples of delivery vehicles that can be used to deliver active compound(s), oligonucleotide(s), or prodrug(s). Certain organic solvents such as dimethylsulfoxide (DMSO) also can be employed.

The pharmaceutical compositions can, if desired, be presented in a pack or dispenser device that can contain one or more unit dosage forms containing the active compound(s). The pack can, for example, comprise metal or plastic foil, such as a blister pack. The pack or dispenser device can be accompanied by instructions for administration.

The active compound(s), or prodrug(s) of the present disclosure, or compositions thereof, will generally be used in an amount effective to achieve the intended result, for example in an amount effective to treat or prevent the particular disease being treated. The compound(s) and oligonucleotide(s) can be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient can still be afflicted with the underlying disorder. Therapeutic benefit also includes halting or slowing the progression of the disease, regardless of whether improvement is realized.

For prophylactic administration, the compound can be administered to a patient at risk of developing one of the previously described diseases. A patient at risk of developing a disease can be a patient having characteristics placing the patient in a designated group of at-risk patients, as defined by an appropriate medical professional or group. A patient at risk may also be a patient that is commonly or routinely in a setting where development of the underlying disease could occur. In other words, an at-risk patient is one who is commonly or routinely exposed to the disease or illness causing conditions or may be acutely exposed for a limited time. Alternatively, prophylactic administration can be applied to avoid the onset of symptoms in a patient diagnosed with the underlying disorder.

The amount of compound administered will depend upon a variety of factors, including, for example, the particular indication being treated, the mode of administration, whether the desired benefit is prophylactic or therapeutic, the severity of the indication being treated, the age and weight of the patient, the bioavailability of the particular active compound, and the like. Determination of an effective dosage is well within the capabilities of those skilled in the art.

Effective dosages can be estimated initially from in vitro assays. For example, an initial dosage for use in animals can be formulated to achieve a circulating blood or serum concentration of active compound that is at or above an IC₅₀₀f the particular compound as measured in an in vitro assay, such as an in vitro fungal MIC or MFC, and other in vitro assays. Calculating dosages to achieve such circulating blood or serum concentrations taking into account the bioavailability of the particular compound is well within the capabilities of skilled artisans. For guidance, see “General Principles,” In: Goodman and Gilman's The Pharmaceutical Basis of Therapeutics, Chapter 1, pp. 1-112, 13th ed., McGraw-Hill, and the references cited therein, which are incorporated herein by reference.

Initial dosages also can be estimated from in vivo data, such as animal models. Animal models useful for testing the efficacy of compounds to treat or prevent the various diseases described above are well-known in the art.

Dosage amounts will typically be in the range of from about 0.0001 or 0.001 or 0.01 mg/kg/day to about 100 mg/kg/day, but can be higher or lower, depending upon, among other factors, the activity of the compound, its bioavailability, the mode of administration, and various factors discussed above. Dosage amount and interval can be adjusted individually to provide plasma levels of the compound(s) that are sufficient to maintain therapeutic or prophylactic effect. In cases of local administration or selective uptake, such as local topical administration, the effective local concentration of active compound(s) cannot be related to plasma concentration. Skilled artisans will be able to optimize effective local dosages without undue experimentation.

Preferably, the compound(s) will provide therapeutic or prophylactic benefit and will have acceptable tolerability. Tolerability of the compound(s) and oligonucleotide(s) can be determined using standard pharmaceutical procedures. The dose ratio between non-tolerable and therapeutic (or prophylactic) effect is the therapeutic index. Compounds(s) that exhibit high therapeutic indices are preferred.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof. The recitation of an embodiment herein includes that embodiment as any single embodiment or in combination with any other embodiments or portions thereof.

### Examples

In order that the embodiments described herein may be more fully understood, the following examples are set forth. The examples described in this application are offered to illustrate the compounds, compositions, and methods provided herein and are not to be construed in any way as limiting their scope.

### General Synthetic Routes

Compounds of Formula (I) may be prepared according to the general synthetic routes described below.

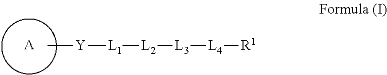

### I Preparation of Ligand Synthetic Intermediates A-Y-L₁-L₂-N₃, where in Y is —C(═O)—

Ligand-linker-azide synthetic intermediates of general formula A-Y-L₁-L₂-N₃ may be prepared according to any of the general procedures outlined below.

In some instances, NMDA receptor ligand A comprises an amino group which is coupled with the acid group of a linker further comprising an azide. In some instances, the amine is a primary amine. In other instances, the amine is a secondary amine forming part of a ring. In some instances, the coupling is achieved using activation groups, such as a carbodiimide-mediated modification of a carboxylic acid (e.g., N-hydroxysuccinimide).

In other instances NMDA receptor ligand A comprises an acid group which is coupled with the amine group of a linker further comprising an azide.

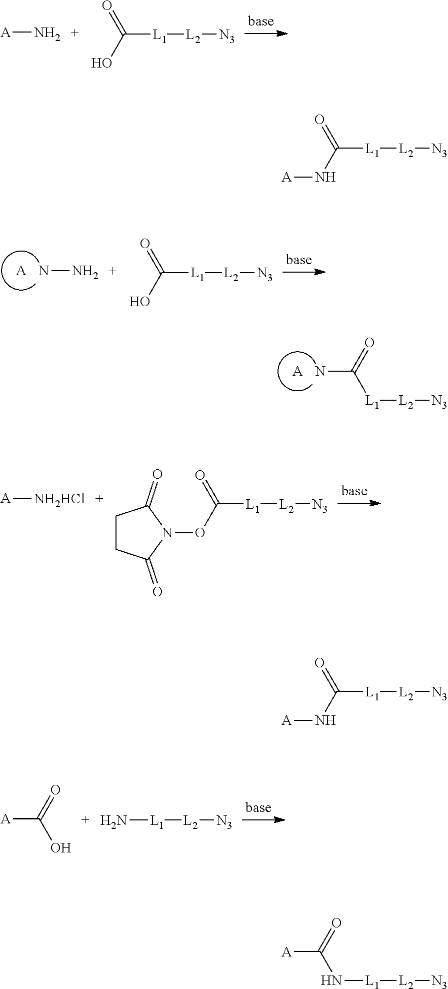

### II Preparation of Ligand Synthetic Intermediates A-Y-L₁-L₂-N₃, where in Y is a Bond

In some instances, NMDA receptor ligand A comprises an amino group which is coupled with a linker comprising a leaving group (e.g., Br, Cl, I, OMs and the like), and wherein the linker further comprises an azide. In other instances, the linker comprises an amino group which is coupled with the NMDA receptor ligand A which comprises a leaving group (e.g., Br, Cl, I, OMs and the like), and wherein the linker further comprises an azide. In other instances, NMDA receptor ligand A comprises an alcohol group which is coupled with a linker comprising a leaving group (e.g., Br, Cl OMs and the like), and wherein the linker further comprises an azide.

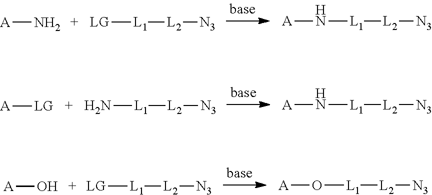

### III Preparation of Synthetic Intermediates L₃′-L₄-R¹ Wherein L₃′ is a Synthetic Precursor to L₃

R¹-Linker synthetic intermediates of general formula L₃′-L₄-R¹, wherein L₃′ is a synthetic precursor to L₃, may be prepared according to any of the general procedures outlined below.

In some instances, an R¹ group is coupled to an L₄′ linker that is a precursor to L₄ and comprises an amino group, to form H₂N-L₄′-R¹. This intermediate is coupled with an L₃′ group comprising an acid group, wherein L₃′ is a precursor to L₃. In some instances, the coupling is achieved using activation groups, such as a carbodiimide-mediated modification of a carboxylic acid (e.g. N-hydroxysuccinimide).

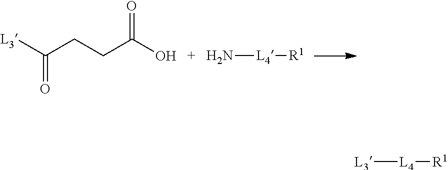

### IV Preparation of Compounds of Formula (I)

Ligand-linker-azide synthetic intermediates of general formula A-Y-L₁-L₂-N₃, prepared as described above are coupled with L₃′-L₄-R¹ intermediates to form compounds of formula (I).

In some instances, L₃′ comprises a triple bond. In some instances, L₃′ comprises a cyclic alkyne.

In some instances, the azide of A-Y-L₁-L₂-N₃ reacts with the triple bond of L₃′-L₄-R¹ to form a triazole.

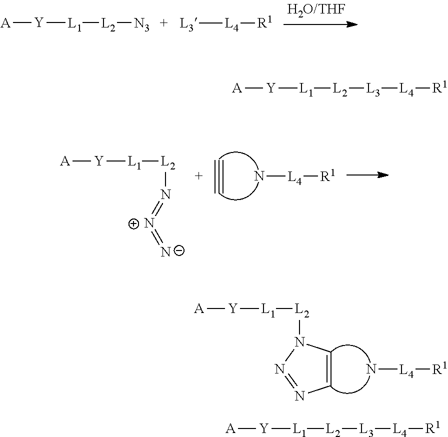

### Synthesis of Nmda Receptor Ligands

Ligands 1-23 were prepared according to the procedures described below

### Example 1: Synthesis of Ligand 1

### (1r,3R^{aa},5S,7r)-N-(12-azidododecyl)-3,5-dimethyladamantan-1-amine

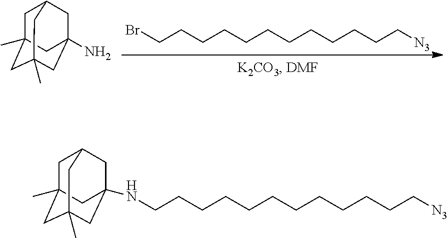 [SMILES: CC12CC3CC(C)(C1)CC(N)(C3)C2 ; [N-]=[N+]=NCCCCCCCCCCCCBr ; CC12CC3CC(C)(C1)CC(NCCCCCCCCCCCCN=[N+]=[N-])(C3)C2]

A mixture of memantine HCl (0.25 g, 1.2 mmol), K₂CO₃ (0.4 g, 2.9 mmol) and 1-azido-12-bromododecane (0.40 g, 1.4 mmol) was stirred at 80° C. in DMF (3 mL) for 48 hours in a sealed vial. The reaction mixture was partitioned between DCM (50 mL) and water (50 mL). The organic phase was separated, and the aqueous phase was extracted with DCM (2×50 mL). The combined organic phases were washed with brine (20 mL), dried with Na₂SO₄, filtered, concentrated, and purified by silica-gel column chromatography (MeOH in DCM, gradient 0-10%) to afford the title compound (144 mg, 32%) as a yellow oil.

MS (ESI), m/z 389.3 [M+H]⁺[0371]¹H NMR (500 MHz, DMSO-d₆) δ 8.39 (s, 1H), 3.31 (t, J=5 Hz, 2H), 2.71-2.74 (m, 2H), 2.15 (s, 1H), 1.40-1.60 (m, 10H), 1.30-1.45 (m, 20H), 1.10-1.20 (m, 2H), 0.60 (s, 6H)

### Example 2: Synthesis of Ligand 2

### (1-azido-15-((5S,10R)-5-methyl-10,11-dihydro-5H-5,10-epiminodibenzo[a,d][7]annulen-12-yl)-3,6,9,12-tetraoxapentadecan-15-one)

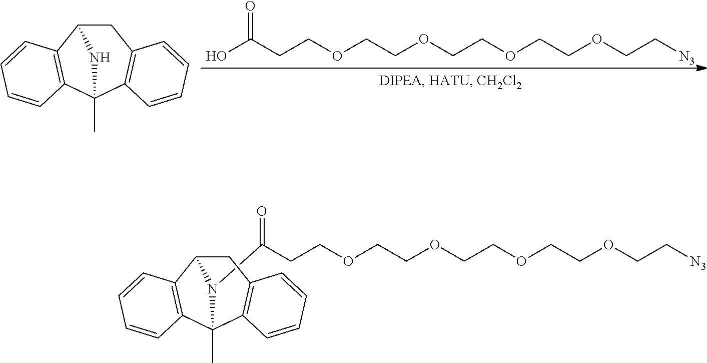

HATU (1.28 g, 3.18 mmol) was added to a solution of (+) MK-801 maleate (0.80 g, 2.37 mmol), DIPEA (2 mL, 10.83 mmol), and 1-azido-3,6,9,12-tetraoxapentadecan-15-oic acid (0.73 g, 2.51 mmol) in DCM (20 mL). The mixture was stirred for 30 minutes and then poured into a saturated NaHCO₃ solution (aq, 100 mL) and extracted with DCM (300 mL×2). The combined organic extracts were dried over Na₂SO₄, filtered, concentrated and purified by silica gel column chromatography (ethyl acetate in hexane, gradient 0-80%) to afford the title compound (0.49 g, 42%) as a yellow oil.

MS (ESI): m/z=495.5 [M+H]⁺; 517.5 [M+Na]⁺

¹H NMR (500 MHz, CDCl₃) δ 7.31-7.34 (m, 2H), 7.15-7.20 (m, 2H), 7.07-7.12 (m, 3H), 6.89-6.90 (m, 1H), 5.41-5.42 (m, 1H), 3.75-3.79 (m, 2H), 3.56-3.66 (m, 15H), 3.35-3.37 (m, 2H), 2.76-2.81 (m, 3H), 2.30-2.40 (m, 3H)

### Example 3: Synthesis of Ligand 3

### (1-azido-N-((5R^{aa},9R^{aa},E)-11-ethylidene-7-methyl-2-oxo-2,6,9,10-tetrahydro-5,9-methanocycloocta[b]pyridin-5(1H)-yl)-3,6,9,12-tetraoxapentadecan-15-amide)

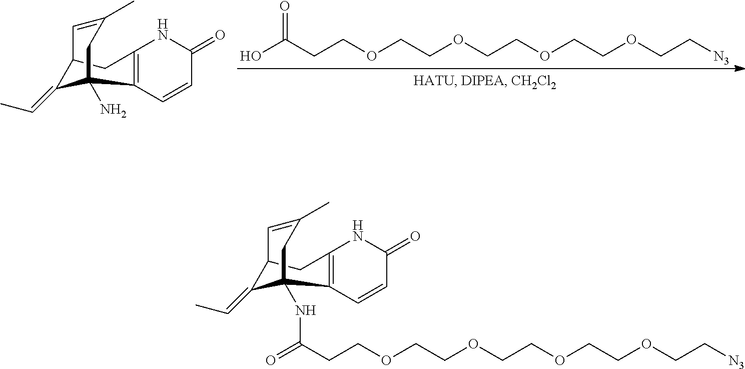

HATU (600 mg, 1.6 mmol) was added to a suspension of 1-azido-3,6,9,12-tetraoxapentadecan-15-oic acid (300 mg, 1.0 mmol), huperzine A (200 mg, 0.8 mmol), and DIPEA (1 mL) in DCM (5 mL). The mixture was stirred for 3 hours, diluted with DCM (100 mL) and washed with water (50 mL). The organic layer was separated, dried over Na₂SO₄, filtered, concentrated and purified with a gradient of EtOH/EtOAc (V/V, 1:3, 0 to 100%) in hexane to afford the title compound (128 mg, 30%) as a yellow oil.

MS (ESI): m/z=516.6 [M+H]⁺; 538.5 [M+Na]⁺

¹H NMR (500 MHz, CDCl₃) δ 8.25 (d, J=10 Hz, 1H), 7.01 (d, J=10 Hz, 1H), 5.54 (q, J=10 Hz, 1H), 5.44-5.45 (m, 1H), 3.82-3.83 (m, 2H), 3.67-3.41 (m, 12H), 3.10-3.41 (m, 2H), 3.00-3.13 (m, 4H), 2.19-2.25 (m, 2H), 1.73 (d, J=5 Hz, 3H), 1.28 (s, 3H), 1.30-1.40 (m, 1H), 1.12-1.22 (m, 2H)

### Example 4: Synthesis of Ligand 4

### ((S)-2-(3-(2-(2-(2-azidoethoxy)ethoxy)ethoxy)propanamido)-N-benzyl-3-methoxypropanamide)

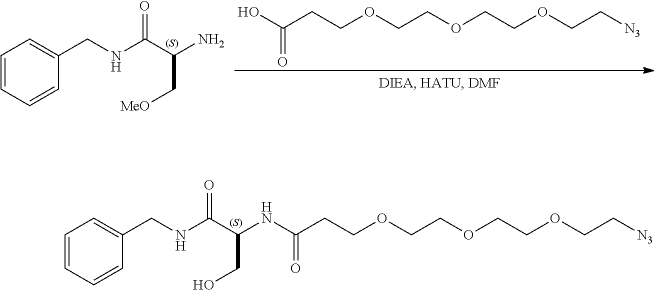

(2S)-2-amino-N-benzyl-3-methoxypropanamide (168 mg, 0.81 mmol), 3-[(8-azido-3,6-dioxaoct-1-yl)oxy]propanoic acid (199 mg, 0.81 mmol), DIEA (208 mg, 1.61 mmol), and HATU (459 mg, 1.21 mmol) were added to DMF (4 mL) at room temperature. The resulting mixture was stirred for 1 h at room temperature. The residue was purified by reverse-phase chromatography (C18 silica gel column; mobile phase, —CH₃CN in water, 0% to 100% gradient in 20 min; detector, UV 254 nm, to afford the title compound (170 mg, 45%) as an off-white solid.

MS (ESI): m/z=460.1 [M+Na]⁺

¹H NMR (CDCl₃, 500 MHz) δ 7.28-7.32 (m, 2H), 7.23-7.26 (m, 3H), 6.90-7.01 (m, 2H), 4.59-4.62 (m, 1H), 4.44-4.49 (m, 2H), 3.89-3.92 (m, 1H), 3.74-3.76 (m, 1H), 3.57-3.69 (m, 9H), 3.45-3.52 (m, 5H), 3.30-3.40 (m, 5H)

### Example 5: Synthesis of Ligand 5

### ((S)-2-acetamido-N-(4-(2-(2-(2-(2-azidoethoxy)ethoxy)ethoxy)ethoxy)benzyl)-3-methoxypropanamide)

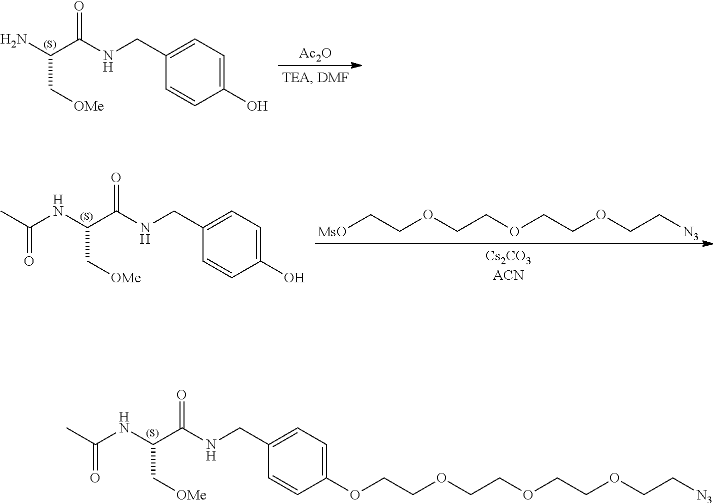

### Step 1: (S)-2-acetamido-N-(4-hydroxybenzyl)-3-methoxypropanamide

To a solution of (2S)-2-amino-N-[(4-hydroxyphenyl)methyl]-3-methoxypropanamide (300 mg, 1.33 mmol) in DCM (5 mL) were added triethylamine (0.30 mL, 2.7 mmol) and Ac₂O (68 mg, 0.67 mmol) dropwise at 0° C. The reaction was stirred at room temperature for 2 hours and then concentrated and purified using C18 column chromatography, eluting with CH₃CN/H₂O to afford the title compound (100 mg, 28%) as a white semi-solid.

MS(ESI) m/z=267.1 [M+H]⁺

### Step 2: (S)-2-acetamido-N-(4-(2-(2-(2-(2-azidoethoxy)ethoxy)ethoxy)ethoxy)benzyl)-3-methoxypropanamide

To a solution of N-[(4S)-7-(4-hydroxyphenyl)-5-oxo-6-aza-2-oxahept-4-yl]acetamide (100 mg, 0.376 mmol) in acetonitrile (1 mL) were added 11-azido-3,6,9-trioxaundec-1-yl methanesulfonate (106 mg, 0.357 mmol), and Cs₂CO₃ (244 mg, 0.751 mmol). The mixture was stirred 60° C. for 2 hours and then quenched by the addition of water (5 mL) at room temperature and extracted with ethyl acetate (100 mL). The organic layer was washed with water (10 mL) and brine, dried over Na₂SO₄, filtered, concentrated and purified with silica gel column chromatography eluting with a gradient of MeOH (0 to 5%) in DCM to afford the title compound (84.3 mg, 47%) as a white solid.

MS(ESI): m/z=468.2 [M+H]⁺

¹H NMR (CDCl₃, 500 MHz) δ 7.10-7.20 (m, 2H), 6.80-6.90 (m, 2H), 6.59-6.68 (m, 1H), 6.38-6.42 (m, 1H), 4.49-4.53 (m, 1H), 4.39-4.40 (m, 2H), 4.10 (t, J=5 Hz, 2H), 3.85 (t, J=5 Hz, 2H), 3.78-3.82 (m, 1H), 3.70-3.74 (m, 1H), 3.65-3.70 (m, 9H), 3.32-3.42 (m, 6H), 3.03 (s, 3H)

### Example 6: Synthesis of Ligand 6

### ((S)—N-((2S,3R)-1-amino-3-hydroxy-1-oxobutan-2-yl)-1-(3-(2-(2-(2-azidoethoxy) ethoxy)ethoxy)propanoyl)pyrrolidine-2-carboxamide)

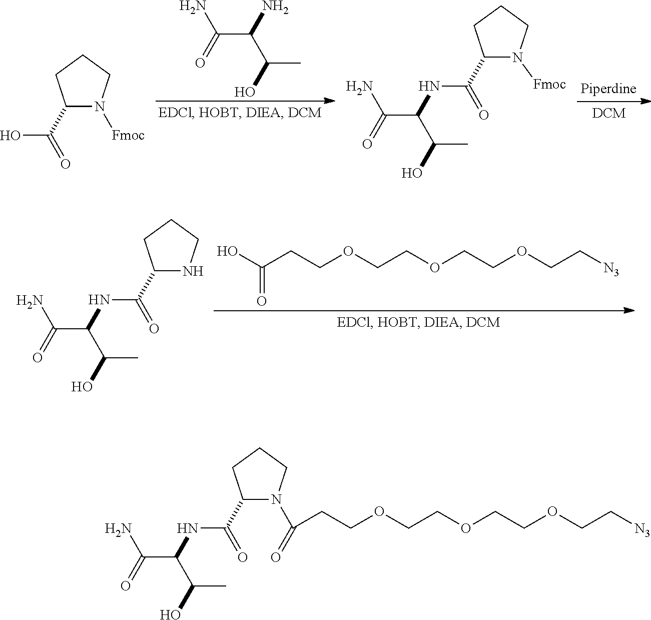

### Step 1: (9H-fluoren-9-yl)methyl (S)-2-(((2S,3R)-1-amino-3-hydroxy-1-oxobutan-2-yl)carbamoyl)pyrrolidine-1-carboxylate

To a stirred solution of N-{[(9H-fluoren-9-ylmethyl)oxy]carbonyl}-L-proline (2.0 g, 5.9 mmol) in DCM (20 mL) were added (2S,3R)-2-amino-3-hydroxybutanamide (0.70 g, 5.9 mmol) and HATU (2.7 g, 7.1 mmol) dropwise at room temperature. To the above mixture was added DIEA (1.15 g, 8.89 mmol) in portions at room temperature. The resulting mixture was stirred overnight and then extracted with ethyl acetate (3×250 mL). The combined organic layers were washed with saturated NaHCO₃ solution (3×250 mL) and brine (100 mL), dried over Na₂SO₄, filtered, concentrated and purified by silica gel column chromatography, eluted with a gradient of MeOH (0 to 10%) in DCM to afford the title compound (1.57 g, 60%) as a light yellow solid.

MS (ESI) m/z=438.0 [M+H]⁺.

### Step 2: (S)—N-((2S,3R)-1-amino-3-hydroxy-1-oxobutan-2-yl)pyrrolidine-2-carboxamide

To a stirred solution of 9H-fluoren-9-ylmethyl (2S)-2-({[(1S,2R)-1-carbamoyl-2-hydroxy propyl]amino}carbonyl)tetrahydropyrrole-1-carboxylate (400 mg, 0.91 mmol) in DCM (4 mL) was added piperidine (0.8 mL) in portions at room temperature. The resulting mixture was stirred for 2 hours. After filtration, the filtrate was concentrated under reduced pressure to afford the crude product as a light yellow solid which was used directly in the next step without further purification.

### Step 3: (S)—N-((2S,3R)-1-amino-3-hydroxy-1-oxobutan-2-yl)-1-(3-(2-(2-(2-azidoethoxy) ethoxy)ethoxy)propanoyl)pyrrolidine-2-carboxamide

To a stirred solution of (2S,3R)-3-hydroxy-2-({[(2S)-tetrahydro-1H-pyrrol-2-yl]carbonyl}amino) butanamide (220 mg, 1.02 mmol) and in DCM (4 mL) were added 3-[(8-azido-3,6-dioxaoct-1-yl)oxy]propanoic acid (252 mg, 1.02 mmol) and HATU (583 mg, 1.53 mmol) dropwise at room temperature. To the above mixture was added DIEA (396 mg, 3.07 mmol) in portions. The resulting mixture was stirred for an additional 1 hour. After filtration, the filtrate was concentrated under reduced pressure. The crude product was purified by reversed-phase chromatography (C18 silica gel column; mobile phase, ACN in water, 10% to 90% gradient in 20 min; detector, UV 254 nm) to afford the title compound (247 mg, 51%) as a yellow oil.

MS (ESI): m/z=445.4 [M+H]⁺, 467.4 [M+Na]⁺

¹H NMR (CDCl₃, 500 MHz) δ 4.50-4.55 (m, 2H), 4.40-4.45 (m, 1H), 3.86-3.91 (m, 2H), 3.50-3.60 (m, 12H), 3.30-3.40 (m, 2H), 3.25 (s, 2H, br), 2.80-2.90 (m, 1H), 2.48-2.51 (m, 1H), 2.20-2.30 (m, 2H), 2.00-2.10 (m, 2H), 1.16 (d, J=5.0 Hz, 3H)

### Example 7: Synthesis of

### Ligand 7

### (2-((2-(2-(2-(2-azidoethoxy)ethoxy)ethoxy)ethyl)amino)-2-(2-chlorophenyl) cyclohexan-1-one (second eluted) and

### Ligand 8

### N-(2-(2-(2-(2-azidoethoxy)ethoxy)ethoxy)ethyl)-2-(2-chlorophenyl)cyclopentane-1-carboxamide (first eluted))

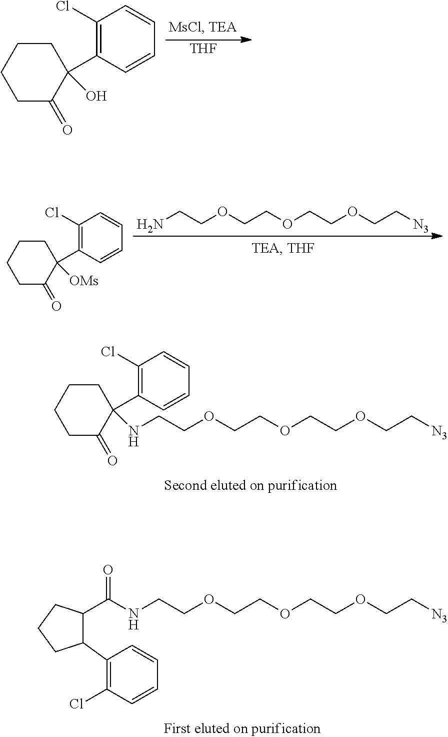

### Step 1: 1-(2-chlorophenyl)-2-oxocyclohexyl methanesulfonate

To a stirred solution of 2-(2-chlorophenyl)-2-hydroxycyclohexan-1-one (0.80 g, 3.5 mmol, prepared as described in J. Org. Chem. 2020, 85(13), 8656-8664) in THF (0.4 mL) were added triethylamine (1.7 mL, 12 mmol) and methanesulfonyl chloride (1.22 g, 10.7 mmol) in portions at 0° C. The resulting mixture was stirred for 1 hour at 0° C. under nitrogen atmosphere. The reaction was quenched by the addition of water (25 mL) at room temperature and extracted with ethyl acetate (2×250 mL). The combined organic layers were washed with water (2×25 mL) and brine (25 mL), dried over Na₂SO₄, filtered, and concentrated under reduced pressure. The crude product was used directly in the next step without further purification.

MS (ESI): m/z=301.0 [M−H]⁻.

### Step 2: 2-((2-(2-(2-(2-azidoethoxy)ethoxy)ethoxy)ethyl)amino)-2-(2-chlorophenyl)cyclohexan-1-one and N-(2-(2-(2-(azidomethoxy)ethoxy)ethoxy)ethyl)-2-(2-chlorophenyl)cyclopentane-1-carboxamide

To a stirred solution of 1-(2-chlorophenyl)-2-oxocyclohexyl methanesulfonate (0.80 g, 2.64 mmol) in THF (1 mL) was added triethylamine (1.84 mL, 13.2 mmol) dropwise at 0° C. 2-[(8-azido-3,6-dioxaoct-1-yl)oxy]ethan-1-amine (1.73 g, 7.93 mmol) was added dropwise at 0° C. and the resulting mixture was stirred overnight at room temperature. The reaction was quenched by the addition of water (10 mL) and extracted with ethyl acetate (3×100 mL). The combined organic layers were washed with water (100 mL) and brine (100 mL), dried over Na₂SO₄, filtered, concentrated and purified by reverse-phase flash chromatography (C18 silica gel column; mobile phase, ACN in water, 10% to 90% gradient in 20 min; detector, UV 254 nm) to afford:

first eluted compound: N-(2-(2-(2-(2-azidoethoxy)ethoxy)ethoxy)ethyl)-2-(2-chlorophenyl)cyclopentane-1-carboxamide (ligand 8; 181.9 mg, 15%) as a light yellow solid

MS (ESI): m/z=425.2 [M+H]⁺

¹H NMR (DMSO-d₆, 400 MHz) δ 7.80-7.90 (m, 1H), 7.15-7.45 (m, 4H), 3.20-3.70 (m, 15H), 3.05-3.15 (m, 2H), 2.80-2.95 (m, 1H), 2.10-2.20 (m, 1H), 1.90-2.05 (m, 1H), 1.70-1.80 (m, 3H), 1.35-1.50 (m, 1H)

second eluted compound: 2-[(11-azido-3,6,9-trioxaundec-1-yl)amino]-2-(2-chlorophenyl)cyclohexan-1-one (ligand 7; 199.9 mg, 16%) as a light yellow solid

MS (ESI): m/z=425.2 [M+H]⁺

¹H NMR (DMSO-d₆, 400 MHz) δ 7.65-7.70 (m, 1H), 7.25-7.40 (m, 3H), 3.42-3.62 (m, 11H), 3.34-3.42 (m, 2H), 2.65-2.75 (m, 1H), 2.50-2.60 (m, 1H), 2.25-2.45 (m, 3H), 2.05-2.15 (m, 1H), 1.80-1.90 (m, 3H), 1.60-1.75 (m, 2H)

### Example 8: Synthesis of Ligand 9

### (1-azido-N-((1r,3R^{aa},5S,7r)-3,5-dimethyladamantan-1-yl)-3,6,9,12-tetraoxapentadecan-15-amide)

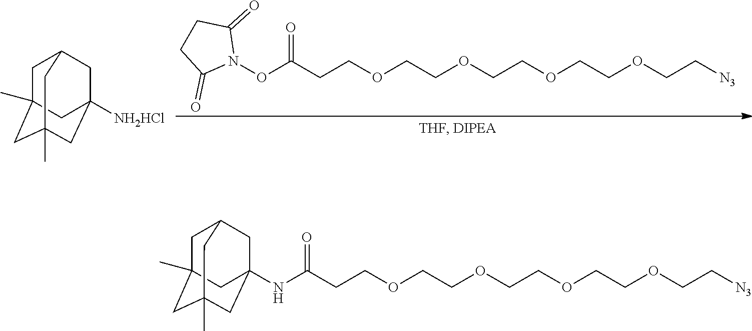

A suspension of azido-PEG4-NHS ester (541 mg, 1.395 mmol), memantine hydrochloride (300 mg, 1.395 mmol) and DIPEA (0.43 ml, 3.1 mmol) in THF (10 mL) was stirred for 3 h at 60° C. The reaction mixture was concentrated, and the residue purified by flash chromatography (10 g 20-micron Biotage column) using hexane and EtOAc (0-100%) to afford the title compound (293 mg, 46%) as a beige solid.

MS (ESI): m/z=453 [M+H]⁺

¹H NMR (499 MHz, DMSO-d₆) δ 7.29 (s, 1H), 3.63-3.56 (m, 2H), 3.56-3.52 (m, 6H), 3.52-3.43 (m, 8H), 3.39 (dd, J=5.6, 4.3 Hz, 2H), 2.24 (t, J=6.6 Hz, 2H), 2.05 (p, J=3.1 Hz, 1H), 1.73 (d, J=3.2 Hz, 2H), 1.60-1.50 (m, 4H), 1.30 (dd, J=12.3, 3.1 Hz, 2H), 1.23 (d, J=12.1 Hz, 2H), 1.09 (s, 2H), 0.80 (s, 6H)

### Example 9: Synthesis of Ligand 10

### (1-azido-15-((5R^{aa},10S)-5-methyl-10,11-dihydro-5H-5,10-epiminodibenzo[a,d][7]annulen-12-yl)-3,6,9,12-tetraoxapentadecan-15-one)

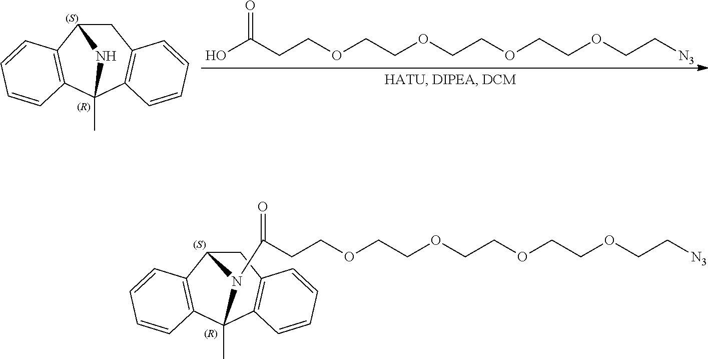

DIPEA (3.0 mL, 17 mmol) was added to a suspension of (−) MK801 maleate (0.86 g, 2.31 mmol) in DCM (20 mL) to obtain a clear solution. N₃—PEG₄-COOH (0.9 g, 3.09 mmol) and HATU (1.2 g, 3.16 mmol) were added in one portion. The initial suspension turned into a yellow solution. The reaction was stirred for 1 h and then poured into a saturated aqueous NaHCO₃ solution (200 mL) and diluted with DCM (500 mL). The organic layer was separated, dried over anhydrous sodium sulfate, filtered, concentrated and purified by column chromatography (silica gel column 80 g, eluted with 30% EtOH in EtOAc, gradient from 0 to 80% in hexane) to afford the title compound (836 mg, 58%) as a light yellow oil.

MS (ESI): m/z=495.3 [M+H]⁺, 517.5 [M+Na]⁺

¹H NMR (499 MHz, CDCl₃) δ 7.37-7.29 (m, 2H), 7.21-7.14 (m, 2H), 7.13-7.03 (m, 3H), 6.93-6.84 (m, 1H), 5.43 (s, 1H), 3.85-3.72 (m, 2H), 3.70-3.52 (m, 16H), 3.40-3.33 (m, 2H), 2.89-2.70 (m, 2H), 2.37 (s, 3H)

### Example 10: Synthesis of Ligand 11

### (1-azido-15-(3-chloro-5-methyl-10,11-dihydro-5H-5,10-epiminodibenzo[a,d][7]annulen-12-yl)-3,6,9,12-tetraoxapentadecan-15-one)

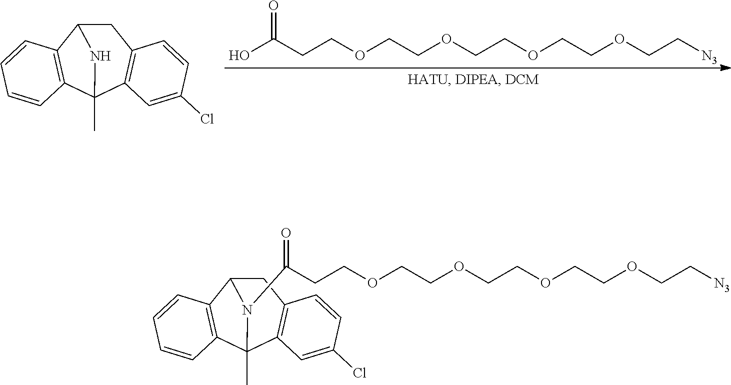 [SMILES: CC12NC(Cc3ccc(Cl)cc31)c1ccccc12 ; [N-]=[N+]=NCCOCCOCCOCCOCCC(=O)O ; CC12c3cc(Cl)ccc3CC(c3ccccc31)N2C(=O)CCOCCOCCOCCOCCN=[N+]=[N-]]

Compound 13 was prepared according to the procedure described for compound 12 using 3-chloro-5-methyl-10,11-dihydro-5H-5,10-epiminodibenzo[a,d][7]annulene as starting material (prepared as described in J. Med. Chem. 1996, 39, 5257-5266) to afford the title compound as yellow oil.

MS (ESI): m/z=529.2 [M+H]⁺, 551.2 [M+Na]⁺

¹H NMR (400 MHz, DMSO-d₆) δ 7.48-7.32 (m, 2H), 7.26-7.14 (m, 4H), 6.96 (d, J=8.2 Hz, 1H), 5.65 (d, J=5.4 Hz, 1H), 3.66-3.43 (m, 17H), 3.39-3.36 (m, 2H), 2.79-2.61 (m, 3H), 2.24 (s, 3H)

### Example 11: Synthesis of Ligand 12

### ((4aR,5R^{aa},10bR)-1-(1-azido-3,6,9,12-tetraoxapentadecan-15-oyl)-12-methyl-2,3,4,4a,5,6-hexahydro-1H-5,10b-prop[1]eno-1,7-phenanthrolin-8(7H)-one)

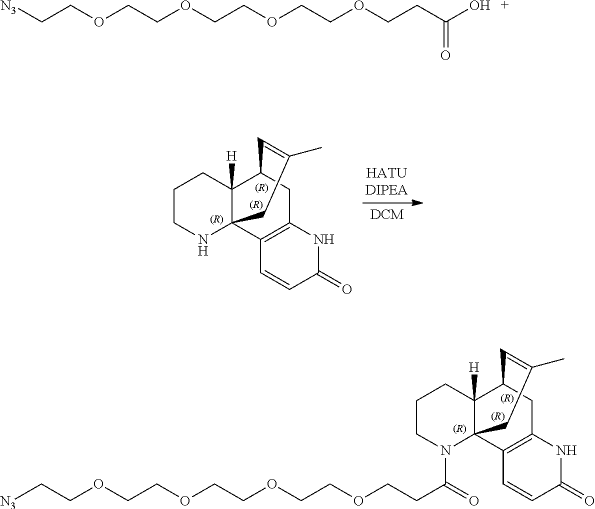

Huperzine B (486 mg, 1.90 mmol) was dissolved in DCM (20 mL), and DIPEA (1 mL) was added. Azido-PEG4-acid (694 mg, 2.38 mmol) and HATU (786 mg, 2.07 mmol) were added, and the solution stirred for 2 hours. The reaction mixture was then poured into saturated sodium bicarbonate solution (100 mL) and diluted with DCM (500 mL). The organic layer was separated, dried over anhydrous sodium sulfate, filtered, concentrated and purified with silica gel RediSepGold column (80 g, eluted with 0 to 80%, hexane and green solvent) to afford the title compound (466 mg, 48%) as a light-yellow oil.

MS (ESI): m/z=530.2 [M+H]⁺, 552.6 [M+Na]⁺

¹H NMR (499 MHz, CDCl₃) δ 8.07 (d, J=8.4 Hz, 1H), 6.99 (d, J=8.3 Hz, 1H), 5.50 (d, J=5.6 Hz, 1H), 3.86 (t, J=6.3 Hz, 2H), 3.68-3.62 (m, 13H), 3.60 (t, J=5.1 Hz, 2H), 3.37-3.23 (m, 2H), 3.02 (dd, J=18.1, 5.7 Hz, 1H), 2.90 (t, J=6.3 Hz, 2H), 2.85-2.75 (m, 1H), 2.70-2.56 (m, 1H), 2.51-2.38 (m, 1H), 2.34-2.14 (m, 2H), 1.90 (d, J=16.9 Hz, 1H), 1.84-1.77 (m, 1H), 1.67-1.57 (m, 2H), 1.57-1.51 (m, 1H), 1.51 (s, 3H)

### Example 12: Synthesis of Ligand 13

### ((S)-1-((2S,4R)-1-(L-threonyl)-4-(2-(2-(2-(2-azidoethoxy)ethoxy)ethoxy)ethoxy)pyrrolidine-2-carbonyl)-N-((2S,3R)-1-amino-3-hydroxy-1-oxobutan-2-yl)pyrrolidine-2-carboxamide)

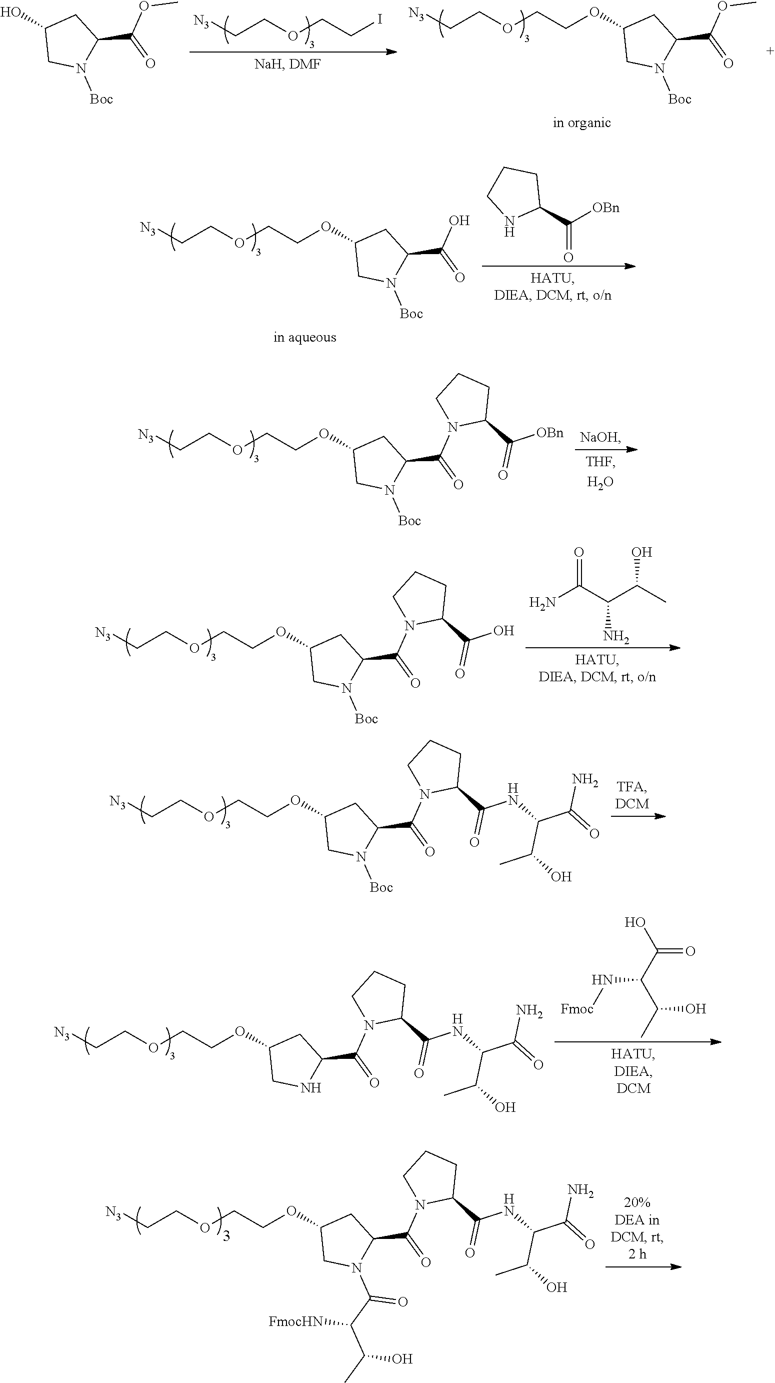

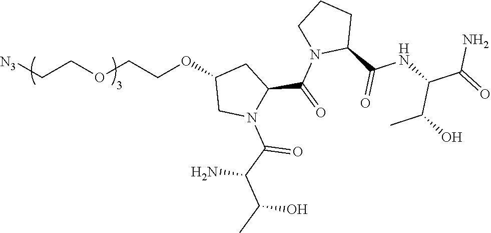

### Step 1: (2S,4R)-4-(2-(2-(2-(2-azidoethoxy)ethoxy)ethoxy)ethoxy)-1-(tert-butoxycarbonyl) pyrrolidine-2-carboxylic acid

To 1-tert-butyl 2-methyl (2S,4R)-4-hydroxypyrrolidine-1,2-dicarboxylate (2.0 g, 8.2 mmol) in DMF (20 mL) was added NaH (0.65 g, 16.3 mmol, 60% in oil) in portions at 0° C. The resulting mixture was stirred for 30 min at 0° C. under nitrogen atmosphere. 11-azido-1-iodo-3,6,9-trioxaundecane (2.68 g, 8.15 mmol) was added dropwise at 0° C. and the resulting mixture stirred for 2 hours at room temperature. The reaction was quenched by the addition of ice-water, extracted with ethyl acetate (500 mL), and the layers separated. The organic layer was concentrated to afford the methyl ester (3.4 g, 10% purity) as a yellow oil.

MS(ESI) m/z=447.3 [M+H]⁺.

The aqueous layer was acidified with aqueous HCl (1N), extracted with ethyl acetate (500 mL) and the organic layer dried over anhydrous sodium sulfate, filtered, and concentrated to afford the title compound (4.6 g, crude) containing DMF, which was used in the next step without further purification.

MS (ESI, negative mode) m/z=431.0 [M−H]⁻.

### Step 2: tert-butyl (2S,4R)-4-(2-(2-(2-(2-azidoethoxy)ethoxy)ethoxy)ethoxy)-2-((S)-2-((benzyloxy)carbonyl)pyrrolidine-1-carbonyl)pyrrolidine-1-carboxylate

To a stirred solution of (2S,4R)-4-(2-(2-(2-(2-azidoethoxy)ethoxy)ethoxy)ethoxy)-1-(tert-butoxycarbonyl) pyrrolidine-2-carboxylic acid (4.6 g, crude from step 1) and benzyl L-prolinate hydrochloride (1.54 g, 6.38 mmol) in DCM (46 mL) was added HATU (2.43 g, 6.38 mmol) in portions at room temperature. DIEA (3.30 g, 25.5 mmol) was added dropwise at room temperature and the resulting mixture stirred overnight at room temperature. The reaction was quenched by the addition of water (10 mL) and diluted with ethyl acetate (100 mL). The combined organic layers were washed with water and brine, dried over anhydrous sodium sulfate, filtered, concentrated and purified by reversed-phase flash chromatography (C18 silica gel column; mobile phase, ACN in water, 10% to 90% gradient in 20 min; detector, UV 254 nm), to afford the title compound (540 mg, 50% purity, 26.6%) as a brown oil.

MS (ESI): m/z=620.5 [M+H]⁺

### Step 3: ((2S,4R)-4-(2-(2-(2-(2-azidoethoxy)ethoxy)ethoxy)ethoxy)-1-(tert-butoxycarbonyl) pyrrolidine-2-carbonyl)-L-proline

To a stirred solution of tert-butyl (2S,4R)-4-(2-(2-(2-(2-azidoethoxy)ethoxy)ethoxy)ethoxy)-2-((S)-2-((benzyloxy)carbonyl)pyrrolidine-1-carbonyl)pyrrolidine-1-carboxylate (500 mg, 0.807 mmol) in THF (5 mL) was added NaOH (1N, 4.03 mL, 4.03 mmol) dropwise at room temperature. The resulting mixture was stirred overnight at room temperature under nitrogen atmosphere and then acidified (pH 2) with HCl solution (1N). The resulting mixture was extracted with ethyl acetate and the combined organic layers were washed with water, dried over anhydrous sodium sulfate, filtered and concentrated to obtain the title compound (500 mg, crude) as a yellow oil which was used directly in the next step without further purification.

MS(ESI) m/z=530.4 [M+H]⁺

### Step 4: tert-butyl (2S,4R)-2-((S)-2-(((2S,3R)-1-amino-3-hydroxy-1-oxobutan-2-yl)carbamoyl) pyrrolidine-1-carbonyl)-4-(2-(2-(2-(2-azidoethoxy)ethoxy)ethoxy)ethoxy)pyrrolidine-1-carboxylate

To a stirred solution of ((2S,4R)-4-(2-(2-(2-(2-azidoethoxy)ethoxy)ethoxy)ethoxy)-1-(tert-butoxycarbonyl) pyrrolidine-2-carbonyl)-L-proline (500 mg, 0.944 mmol) in DCM (5 mL) was added (2S,3R)-2-amino-3-hydroxybutanamide hydrochloride (291.91 mg, 1.888 mmol) in portions at room temperature. DIEA (366 mg, 2.83 mmol) was added dropwise at room temperature and the mixture stirred for 2 h at room temperature. The mixture was concentrated and purified by reverse-phase flash chromatography (C18 silica gel column; mobile phase, ACN in water, 10% to 90% gradient in 20 min; detector, UV 254 nm), to afford the title compound (550 mg, 93%) as a light-yellow oil.

MS (ESI) m/z=630.5 [M+H]⁺

### Step 5: (S)—N-((2S,3R)-1-amino-3-hydroxy-1-oxobutan-2-yl)-1-((2S,4R)-4-(2-(2-(2-(2-azidoethoxy)ethoxy)ethoxy)ethoxy)pyrrolidine-2-carbonyl)pyrrolidine-2-carboxamide

To a solution of tert-butyl (2S,4R)-2-((S)-2-(((2S,3R)-1-amino-3-hydroxy-1-oxobutan-2-yl)carbamoyl) pyrrolidine-1-carbonyl)-4-(2-(2-(2-(2-azidoethoxy)ethoxy)ethoxy)ethoxy) pyrrolidine-1-carboxylate (550 mg, 0.873 mmol) in DCM (5 mL) was added TFA (5 mL, 65 mmol) dropwise at room temperature. The resulting mixture was stirred for 1 h at room temperature under nitrogen atmosphere and concentrated to obtain the title compound (1.00 g, crude) as a yellow oil, which was used directly in the next step without further purification.

MS (ESI) m/z=530.3 [M+H]⁺

### Step 6: (9H-fluoren-9-yl)methyl ((2S,3R)-1-((2S,4R)-2-((S)-2-(((2S,3R)-1-amino-3-hydroxy-1-oxobutan-2-yl)carbamoyl)pyrrolidine-1-carbonyl)-4-(2-(2-(2-(2-azidoethoxy)ethoxy)ethoxy) ethoxy)pyrrolidin-1-yl)-3-hydroxy-1-oxobutan-2-yl)carbamate

To a stirred solution of (S)—N-((2S,3R)-1-amino-3-hydroxy-1-oxobutan-2-yl)-1-((2S,4R)-4-(2-(2-(2-(2-azidoethoxy)ethoxy)ethoxy)ethoxy)pyrrolidine-2-carbonyl)pyrrolidine-2-carboxamide (500 mg, 0.944 mmol) in DCM (5 mL) was added N-{[(9H-fluoren-9-ylmethyl)oxy]carbonyl}-L-threonine (322 mg, 0.944 mmol) in portions at room temperature. HATU (538 mg, 1.42 mmol) and DIEA (732 mg, 5.67 mmol) were added in portions and the resulting mixture was stirred overnight at room temperature. The resulting solution was concentrated and purified with C18 chromatography (ACN/H₂O) to afford the title compound (300 mg, 37%) as a yellow solid.

MS(ESI) m/z=853.4 [M+H]⁺

### Step 7: (S)-1-((2S,4R)-1-(L-threonyl)-4-(2-(2-(2-(2-azidoethoxy)ethoxy)ethoxy)ethoxy) pyrrolidine-2-carbonyl)-N-((2S,3R)-1-amino-3-hydroxy-1-oxobutan-2-yl)pyrrolidine-2-carboxamide

To a stirred solution of (9H-fluoren-9-yl)methyl ((2S,3R)-1-((2S,4R)-2-((S)-2-(((2S,3R)-1-amino-3-hydroxy-1-oxobutan-2-yl)carbamoyl)pyrrolidine-1-carbonyl)-4-(2-(2-(2-(2-azido-ethoxy)ethoxy)ethoxy) ethoxy)pyrrolidin-1-yl)-3-hydroxy-1-oxobutan-2-yl)carbamate (260 mg, 0.305 mmol) in DCM (3 mL) was added Et₂NH (0.8 mL) dropwise at room temperature. The resulting mixture was stirred for 2 h at room temperature under nitrogen atmosphere and then concentrated and extracted with water. The aqueous layer was washed with ethyl acetate and the combined aqueous layers were lyophilized overnight, then co-evaporated with ACN and further lyophilized to obtain the title compound (150.6 mg, 73.8%) as a light yellow solid.

MS (ESI) m/z=631.5 [M+H]⁺

¹H NMR (499 MHz, DMSO-d₆) δ 7.42 (d, J=8.4 Hz, 1H), 7.10-7.04 (m, 2H), 4.86 (s, 1H), 4.58 (t, J=8.0 Hz, 1H), 4.43-4.34 (m, 1H), 4.22-4.15 (m, 1H), 4.10-3.98 (m, 2H), 3.85-3.76 (m, 1H), 3.75-3.65 (m, 1H), 3.67-3.61 (m, 1H), 3.64-3.57 (m, 3H), 3.60-3.48 (m, 12H), 3.50-3.42 (m, 1H), 3.42-3.36 (m, 2H), 3.29-3.23 (m, 1H), 2.34-2.26 (m, 1H), 2.07-1.97 (m, 1H), 1.97-1.87 (m, 3H), 1.90 (s, 1H), 1.15-0.97 (m, 6H)

### Example 13: Synthesis of

### Ligand 14

### (N-(2-(2-(2-(2-azidoethoxy)ethoxy)ethoxy)ethyl)-2-(1-((1R^{aa},2S)-1-hydroxy-1-(4-hydroxyphenyl)propan-2-yl)piperidin-4-yl)acetamide and

### Ligand 15

### N-(2-(2-(2-(2-azidoethoxy)ethoxy)ethoxy)ethyl)-2-(1-((1R^{aa},2R)-1-hydroxy-1-(4-hydroxyphenyl)propan-2-yl)piperidin-4-yl)acetamide)

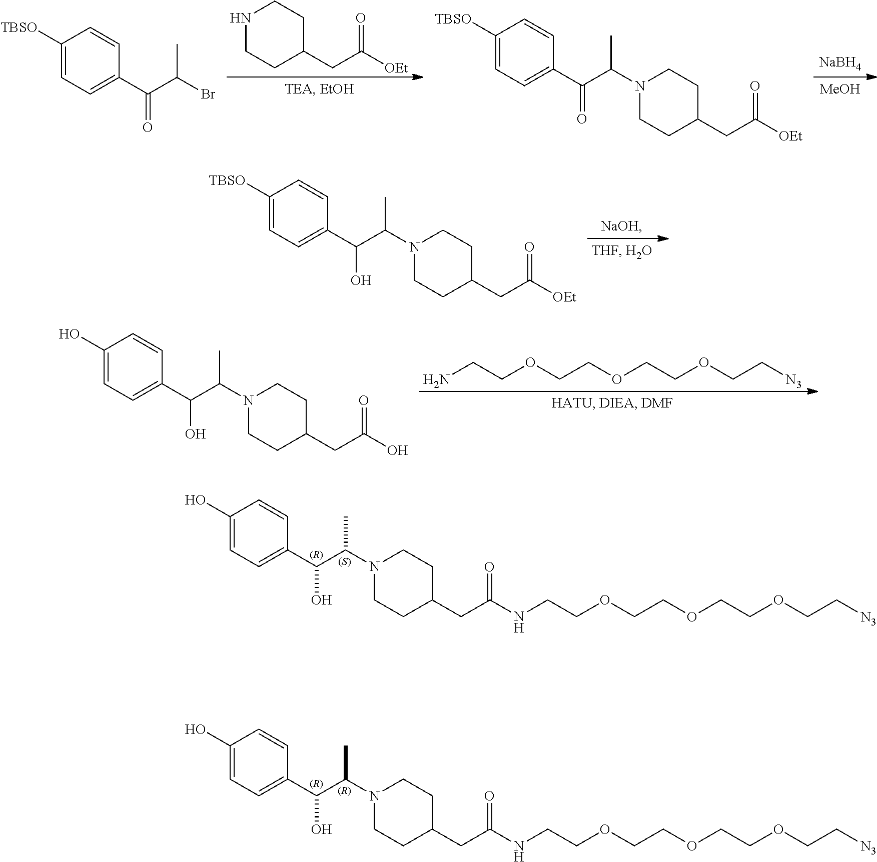

### Step 1: ethyl 2-(1-(1-(4-((tert-butyldimethylsilyl)oxy)phenyl)-1-oxopropan-2-yl)piperidin-4-yl)acetate

To a stirred solution of 2-bromo-1-(4-((tert-butyldimethylsilyl)oxy)phenyl)propan-1-one (1.2 g, 3.5 mmol, prepared as described in Bioorg. Med. Chem. Lett. 2007, 5558) in ethanol (20 mL) were added TEA (0.53 g, 5.24 mmol) and ethyl hexahydropyridin-4-ylacetate (0.90 g, 5.2 mmol) in portions at room temperature. The resulting mixture was heated at 80° C. for 3 h under nitrogen and then quenched with water (5 mL) at room temperature. The mixture was extracted with DCM (200 mL) and the combined organic layers were washed with brine, dried over anhydrous sodium sulfate, filtered, concentrated and purified by silica gel column chromatography, eluted with PE:EA (2:1, v/v), to afford the title compound (1.0 g, 66%) as a colorless oil.

MS(ESI) m/z=434.2 [M+H]⁺

¹H NMR (300 MHz, CDCl₃) δ 8.02 (d, 2H), 6.85 (d, J=8.3 Hz, 2H), 4.18-4.05 (m, 2H), 4.02 (m, 1H), 2.89 (m, 1H), 2.79 (m, 1H), 2.39 (m, 1H), 2.20 (m, 3H), 1.68 (m, 5H), 1.31-1.19 (m, 6H), 0.99 (s, 9H), 0.24 (s, 6H)

### Step 2: ethyl 2-(1-(1-(4-((tert-butyldimethylsilyl)oxy)phenyl)-1-hydroxypropan-2-yl)piperidin-4-yl)acetate

To a solution of ethyl 2-(1-(1-(4-((tert-butyldimethylsilyl)oxy)phenyl)-1-oxopropan-2-yl)piperidin-4-yl)acetate (900 mg, 2.075 mmol) in methanol (10 mL) was added NaBH₄ (157 mg, 4.15 mmol) batchwise at 0° C. The resulting mixture was stirred overnight at room temperature under nitrogen atmosphere and then quenched by the addition of water and extracted with ethyl acetate (200 mL). The combined organic layers were washed with water and brine, dried over anhydrous sodium sulfate, filtered and concentrated to afford the title compound (1.0 g, crude) as a yellow oil, which was used directly without further purification.

MS(ESI) m/z=436.3 [M+H]⁺

### Step 3: 2-(1-(1-hydroxy-1-(4-hydroxyphenyl)propan-2-yl)piperidin-4-yl)acetic acid

To a stirred solution of ethyl 2-(1-(1-(4-((tert-butyldimethylsilyl)oxy)phenyl)-1-hydroxypropan-2-yl)piperidin-4-yl)acetate (1.0 g, 2.3 mmol) in THF (10 mL) was added aqueous NaOH (1N, 13.8 mL, 13.8 mmol) dropwise at 25° C. The resulting mixture was stirred overnight at room temperature and then quenched by the dropwise addition of acetic acid (0.92 mL, 16 mmol) at room temperature. The mixture was concentrated and purified by reverse phase column chromatography (combi-flash C18 column, ACN/H₂O) to afford the trans/cis mixture of the title compound (450 mg, 67%) as a white solid.

MS(ESI): m/z=294.2 [M+H]⁺

¹H NMR (300 MHz, DMSO-d₆) δ 7.14-7.02 (m, 2H), 6.73-6.61 (m, 2H), 4.57 (d, J=4.8 Hz, 1H, corresponds to cis-isomer), 4.12 (d, J=9.4 Hz, 1H, corresponds to trans isomer), 2.80 (t, J=12.7 Hz, 1H), 2.65 (d, J=13.5 Hz, 1H), 2.34 (d, J=11.5 Hz, OH), 2.10 (m, J=24.1, 6.4 Hz, 2H), 1.70 (d, J=12.4 Hz, 2H), 1.56 (d, J=11.5 Hz, 1H), 1.36-1.25 (m, OH),0.88 (d, J=6.7 Hz, 1H), 0.63 (d, J=6.6 Hz, 2H) NMR indicated a mixture of cis:trans (5:7) isomers

### Step 4: N-(2-(2-(2-(2-azidoethoxy)ethoxy)ethoxy)ethyl)-2-(1-((1R^{aa},2S)-1-hydroxy-1-(4-hydroxyphenyl)propan-2-yl)piperidin-4-yl)acetamide (Compound 14) and N-(2-(2-(2-(2-azidoethoxy)ethoxy)ethoxy)ethyl)-2-(1-((1R^{aa},2R)-1-hydroxy-1-(4-hydroxyphenyl)propan-2-yl)piperidin-4-yl)acetamide (Compound 15)

To a solution of 2-(1-(1-hydroxy-1-(4-hydroxyphenyl)propan-2-yl)piperidin-4-yl)acetic acid (270 mg, 0.920 mmol) in DMF (3 mL) were added HOBt (187 mg, 1.38 mmol), EDCI (265 mg, 1.38 mmol), and DIEA (1070 mg, 8.283 mmol) and the mixture stirred for 5 min at room temperature under nitrogen atmosphere. 2-[(8-azido-3,6-dioxaoct-1-yl)oxy]ethan-1-amine (301 mg, 1.38 mmol) was added dropwise at room temperature and the resulting mixture was stirred for 36 h at 30° C. The mixture was concentrated and purified by reverse-phase flash chromatography (C18 silica gel column; mobile phase, ACN in water, 10% to 90% gradient in 20 min; detector, UV 254 nm) and further purified by Prep-HPLC (Column: XBridge Prep OBD C18 Column 19*150 mm, 5m; Mobile Phase A: Water(10 mmol/L NH₄HCO₃), Mobile Phase B: ACN; Flow rate: 25 mL/min mL/min; Gradient: isocratic 18-28; Wave Length: 254 nm/210 nm nm), to afford:

Ligand 14: (Rt=4.9 min, 80.3 mg, 17%, racemic cis) as a yellow oil.

MS(ESI): m/z=494.4 [M+H]⁺

¹H NMR (499 MHz, DMSO-d₆) δ 9.13 (s, 1H), 7.80 (t, J=5.7 Hz, 1H), 7.09-7.00 (m, 2H), 6.68-6.59 (m, 2H), 4.79 (d, J=4.5 Hz, 1H), 4.52 (t, J=4.7 Hz, 1H), 3.60 (dd, J=5.6, 4.3 Hz, 2H), 3.57-3.47 (m, 8H), 3.41-3.35 (m, 4H), 3.16 (q, J=5.8 Hz, 2H), 2.77 (d, J=11.0 Hz, 1H), 2.65 (d, J=11.3 Hz, 1H), 2.53 (t, J=6.7 Hz, 1H), 2.28 (td, J=11.5, 2.3 Hz, 1H), 2.12 (td, J=11.5, 2.3 Hz, 1H), 1.92 (d, J=7.0 Hz, 2H), 1.40-1.60 (m, 3H), 0.90-1.00 (m, 2H), 0.88 (d, J=6.7 Hz, 3H)

Ligand 15: (Rt=7.3 min, 119.6 mg, 25%, racemic trans) as yellow oil.

MS (ESI): m/z=494.45 [M+H]⁺

¹H NMR (499 MHz, DMSO-d₆) δ 9.27 (s, 1H), 7.86 (t, J=5.7 Hz, 1H), 7.13-7.07 (m, 2H), 6.72-6.66 (m, 2H), 4.81 (s, 1H), 4.11 (d, J=9.4 Hz, 1H), 3.63-3.55 (m, 2H), 3.59-3.47 (m, 8H), 3.43-3.36 (m, 3H), 3.31 (s, 1H), 3.19 (q, J=5.8 Hz, 2H), 2.76 (d, J=11.3 Hz, 1H), 2.63 (d, J=11.5 Hz, 1H), 2.51-2.41 (m, 2H), 2.11-2.03 (m, 1H), 2.01 (d, J=6.7 Hz, 2H), 1.69-1.61 (m, 3H), 1.32-1.21 (m, 1H), 1.18-1.07 (m, 1H), 0.63 (d, J=6.6 Hz, 3H)

### Example 14: Synthesis of Ligand 16

### 1-azido-15-(3-chloro-5-methyl-10,11-dihydro-5H-5,10-epiminodibenzo[a,d][7]-annulen-12-yl)-3,6,9,12-tetraoxapentadecan-15-one

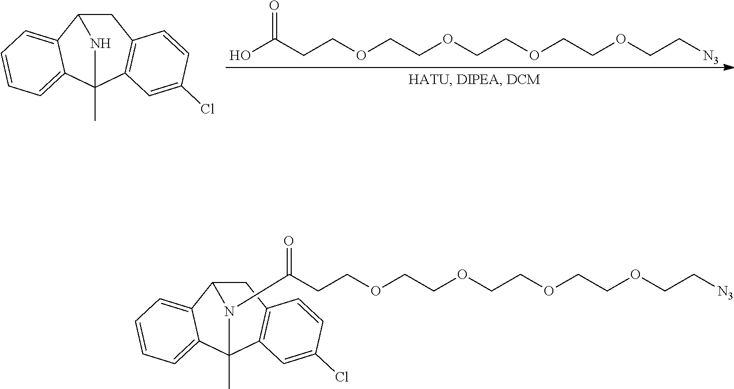 [SMILES: CC12NC(Cc3ccc(Cl)cc31)c1ccccc12 ; [N-]=[N+]=NCCOCCOCCOCCOCCC(=O)O ; CC12c3cc(Cl)ccc3CC(c3ccccc31)N2C(=O)CCOCCOCCOCCOCCN=[N+]=[N-]]

DIPEA (1.0 mL, 5.74 mmol) was added to a suspension of 3-chloro-5-methyl-10,11-dihydro-5H-5,10-epiminodibenzo[a,d][7]annulene (prepared as described in J. Med. Chem. 1990, 33, 789-808, 90 mg, 0.35 mmol) in DCM (2 mL). HATU (201 mg, 0.53 mmol) and N₃PEG₄COOH (0.9 g, 3.09 mmol) were then added in one portion and the mixture stirred for 1 h. The mixture was poured into a solution of saturated aqueous NaHCO₃ (20 mL) and diluted with DCM (50 mL). The organic layer was separated, dried over Na₂SO₄, filtered, concentrated and purified by column chromatography (silica gel column 25 g, eluted with 30% EtOH in EtOAc, with a gradient of 0 to 80% in hexane) to afford the titled compound (90 mg, 49%) as a light-yellow oil.

MS (ESI) m/z=529.4 [M+H]⁺, 551.4 [M+Na]⁺

¹H NMR (499 MHz, CDCl₃) δ 7.36-7.28 (m, 2H), 7.24-7.16 (m, 2H), 7.11-6.99 (m, 2H), 6.83 (d, J=8.1 Hz, 1H), 5.43 (s, 1H), 3.79 (dt, J=9.5, 6.9 Hz, 2H), 3.71-3.46 (m, 15H), 3.37 (t, J=5.0 Hz, 2H), 2.90-2.62 (m, 3H), 2.35 (s, 3H)

### Example 15: Synthesis of Ligand 17

### (5S,10R)-12-(12-azidododecyl)-5-methyl-10,11-dihydro-5H-5,10-epiminodibenzo-[a,d][7]annulene

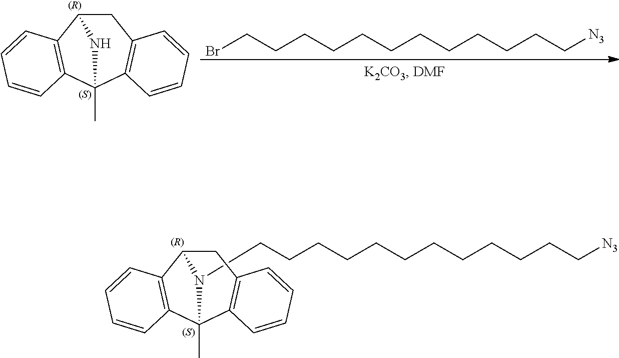

K₂CO₃ (1.3 g, 9.4 mmol) was added to (+)-MK801 maleate (0.51 g, 1.5 mmol) and 1-azido-12-bromododecane (0.6 g, 1.37 mmol) in DMF (5 mL) the mixture heated to 80° C. A second portion of 1-azido-12-bromododecane (0.3 g, 0.69 mmol) was added and the mixture was heated to 90° C. for 5 hours. After cooling, the mixture was extracted with ethyl acetate (300 mL), washed with lithium chloride solution (aq, 10%) and brine, dried over Na₂SO₄, filtered, concentrated and purified via flash column chromatography to afford the title compound (340 mg, 53%) as a yellow oil.

MS (ESI): m/z=431.4 [M+H]⁺

¹H NMR (499 MHz, CDCl₃) δ 7.31-7.28 (m, 1H), 7.24-7.20 (m, 1H), 7.13-7.01 (m, 5H), 6.93-6.87 (m, 1H), 4.56 (d, J=5.2 Hz, 1H), 3.37-3.19 (m, 3H), 2.63-2.52 (m, 1H), 2.50-2.45 (m, 1H), 2.40-2.29 (m, 1H), 1.86 (s, 3H), 1.73-1.54 (m, 4H), 1.41-1.18 (m, 16H)

### Example 16: Synthesis of Ligand 18

### (S)—N-(((1R^{aa},3S,5S,7S)-adamantan-2-yl)methyl)-2-(3-(2-(2-(2-azidoethoxy)ethoxy)-ethoxy)propanamido)-3-methoxypropanamide

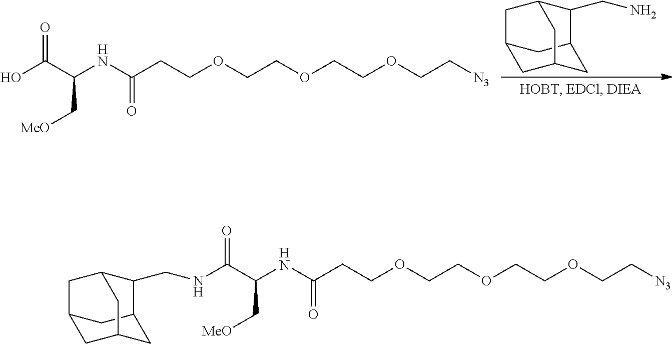

To a stirred solution of (2S)-2-[(1-azido-12-oxo-3,6,9-trioxadodec-12-yl)amino]-3-methoxypropanoic acid (200 mg, 0.574 mmol) in DMF (3 mL) were added adamantan-2-ylmethanamine (95 mg, 0.57 mmol), HOBt (116 mg, 0.861 mmol) and EDCI (165 mg, 0.861 mmol) batchwise at room temperature. DIEA (371 mg, 2.87 mmol) was added dropwise, and the mixture stirred at room temperature overnight under nitrogen atmosphere. The reaction was quenched with water and extracted with ethyl acetate. The combined organic extracts were washed with saturated aqueous NaHCO₃ solution and brine, dried over Na₂SO₄, filtered, concentrated and purified by reversed-phase flash chromatography (SunFire C18 OBD Prep Column 19*150 mm, 5m; Mobile Phase A: Water (0.1% FA), Mobile Phase B: CH₃CN; Flow rate: 25 mL/min mL/min; Gradient: 52% B to 62% B in 8 min; Wave Length: 254 nm/220 nm nm; RT1(min) 4.37) to afford the title compound (123 mg, 42.8%) as a yellow oil.

MS(ESI) m/z=496.3 [M+H]⁺

¹H NMR (400 MHz, CDCl₃) δ 7.01 (d, J=7.2 Hz, 1H), 6.51 (s, 1H), 4.56-4.47 (m, 1H), 3.87-3.68 (m, 3H), 3.72-3.59 (m, 10H), 3.46-3.30 (m, 8H), 2.62-2.43 (m, 2H), 1.94-1.79 (m, 7H), 1.69 (s, 6H), 1.60-1.50 (m, 2H)

### Example 17: Synthesis of Ligand 19

### (S)-2-(3-(2-(2-(2-azidoethoxy)ethoxy)ethoxy)propanamido)-N-(4-fluorobenzyl)-3-methoxypropanamide

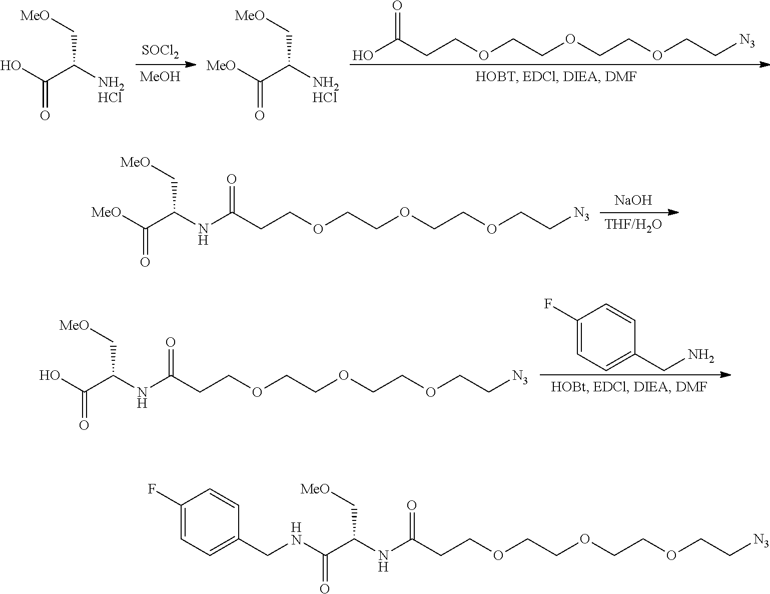

### Step 1: (2S)-2-amino-3-methoxypropanoate hydrochloride

To a stirred solution of (2S)-2-amino-3-methoxypropanoic acid hydrochloride (5.0 g, 32 mmol) in methanol (50 mL) was added thionyl chlordie (7.65 g, 64.3 mmol) dropwise at 0° C. The resulting mixture was stirred for 2 hours at 60° C. under nitrogen atmosphere. The mixture was cooled to RT and then concentrated to afford the title compound (6.0 g, 100%) an off-white solid which was directly used without further purification.

MS(ESI) m/z=134.1 [M+H]⁺

¹H NMR (300 MHz, DMSO-d₆) δ 8.83 (s, 3H), 4.70 (s, 2H), 4.27 (s, 1H), 3.81 (d, J=3.5 Hz, 2H), 3.75 (s, 3H), 3.29 (s, 3H)

### Step 2: (2S)-2-[(1-azido-12-oxo-3,6,9-trioxadodec-12-yl)amino]-3-methoxypropanoate

To a stirred solution of methyl (2S)-2-amino-3-methoxypropanoate hydrochloride (1.17 g, 6.88 mmol) in DMF (17 mL) were added 3-[(8-azido-3,6-dioxaoct-1-yl)oxy]propanoic acid (1.70 g, 6.88 mmol), HOBt (1.39 g, 10.3 mmol) and EDCI (1.98 g, 10.3 mmol) batchwise at room temperature. DIEA (2.67 g, 20.627 mmol) was then added dropwise, and the mixture stirred overnight under nitrogen atmosphere. The reaction was quenched by the addition of water and then extracted with ethyl acetate. The combined organic extracts were washed with water, saturated NaHCO₃ solution and brine, dried over Na₂SO₄, filtered and concentrated to afford the title compound (1.6 g, 64%) as a yellow oil which was used directly in the next step without further purification.

MS(ESI) m/z=349.2 [M+H]⁺

¹H NMR (400 MHz, CDCl₃) δ 7.04 (d, J=8.2 Hz, 1H), 4.78-4.70 (m, 1H), 3.86-3.73 (m, 3H), 3.76 (s, 3H), 3.77-3.61 (m, 11H), 3.64-3.57 (m, 1H), 3.39 (t, J=5.1 Hz, 2H), 3.34 (s, 3H), 2.66-2.50 (m, 2H)

### Step 3: N-(3-(2-(2-(2-azidoethoxy)ethoxy)ethoxy)propanoyl)-O-methyl-L-serine

To a stirred solution of methyl (2S)-2-[(1-azido-12-oxo-3,6,9-trioxadodec-12-yl)amino]-3-methoxypropanoate (1.6 g, 4.4 mmol) in THF (16 mL) was added sodium hydroxide solution (17.7 mL, 17.7 mmol, 1 mol/L) dropwise at room temperature and the resulting mixture stirred for 2 hours. The mixture was then acidified to pH 3 with Dowex resin (Beijing innochem science & Technology Co. Ltd), washed with acetonitrile, filtered and the filtrate concentrated to afford the title compound (1.2 g, 78%) as a yellow oil which was used directly in the next step without further purification.

MS(ESI) m/z=349.2 [M+H]⁺

¹H NMR (300 MHz, CDCl₃) δ 7.23 (d, J=7.6 Hz, 1H), 5.37 (s, 3H), 4.76-4.65 (m, 1H), 3.91-3.81 (m, 1H), 3.82-3.70 (m, 3H), 3.70-3.55 (m, 13H), 3.44-3.39 (m, 2H), 3.38 (s, 3H), 2.69-2.40 (m, 2H)

### Step 4: (S)-2-(3-(2-(2-(2-azidoethoxy)ethoxy)ethoxy)propanamido)-N-(4-fluorobenzyl)-3-methoxypropanamide

To a stirred solution of N-(3-(2-(2-(2-azidoethoxy)ethoxy)ethoxy)propanoyl)-O-methyl-L-serine (200 mg, 0.574 mmol) in DMF (3 mL) were added (4-fluorophenyl)methanamine (71 mg, 0.57 mmol), HOBt (116 mg, 0.861 mmol) and EDCI (165 mg, 0.861 mmol) batchwise at room temperature. DIEA (371 mg, 2.87 mmol) was then added dropwise, and the mixture stirred for 3 hours under nitrogen atmosphere. The reaction was quenched with water and extracted with ethyl acetate. The combined organic extracts were washed with saturated NaHCO₃ solution and brine, dried over Na₂SO, filtered, concentrated and purified by reversed-phase flash chromatography (SunFire C18 OBD Prep Column 19*150 mm, 5m; Mobile Phase A: Water(0.1% FA), Mobile Phase B: CH₃CN; Flow rate: 25 mL/min mL/min; Gradient: 33% B to 43% B in 8 min; Wave Length: 254 nm/220 nm nm; Rt(min): 5.18), to afford the title compound (99.5 mg, 38%) as a white solid.

MS(ESI) m/z=456.1 [M+H]⁺

¹H NMR (400 MHz, CDCl₃) δ 7.27-7.18 (m, 2H), 7.05-6.95 (m, 3H), 4.64-4.56 (m, 1H), 4.52-4.43 (m, 1H), 4.43-4.35 (m, 1H), 3.96-3.88 (m, 1H), 3.82-3.72 (m, 2H), 3.71-3.53 (m, 6H), 3.57-3.43 (m, 5H), 3.38 (s, 5H), 2.61-2.38 (m, 2H)

### Example 18: Synthesis of Ligand 20

### (S)-2-(3-(2-(2-(2-azidoethoxy)ethoxy)ethoxy)propanamido)-3-methoxy-N-(4-methylbenzyl)propanamide

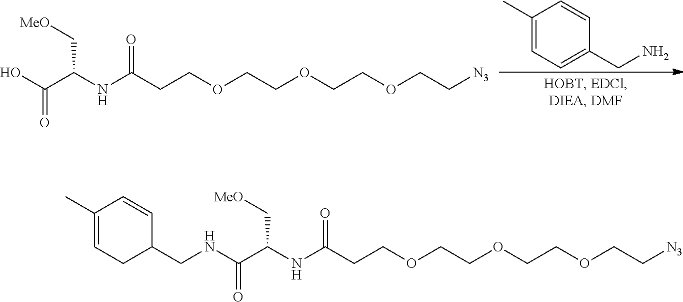

(S)-2-(3-(2-(2-(2-azidoethoxy)ethoxy)ethoxy)propanamido)-3-methoxy-N-(4-methylbenzyl) propenamide was prepared according to similar procedures as described for (S)-2-(3-(2-(2-(2-azidoethoxy)ethoxy)ethoxy)propanamido)-N-(4-fluorobenzyl)-3-methoxypropanamide, using p-tolylmethanamine in place of (4-fluorophenyl)methanamine, to afford the title compound as a white solid, 95% pure.

MS (ESI) m/z=452.10 [M+H]⁺, 474.10 [M+Na]⁺

### Example 19: Synthesis of Ligand 21

### N-(2-(2-(2-(2-azidoethoxy)ethoxy)ethoxy)ethyl)-1-(2-chlorophenyl)pyrrolidine-2-carboxamide

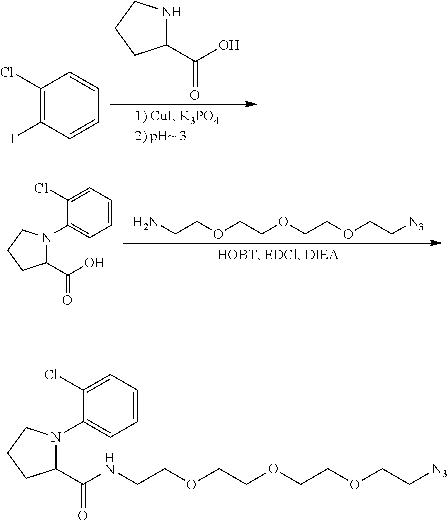

### Step 1: (2-chlorophenyl)proline

To a stirred solution of 1-chloro-2-iodobenzene (1.0 g, 4.194 mmol) in isopropanol (10 mL) were added DL-proline (0.58 g, 5.033 mmol), copper(I) iodide (0.16 g, 0.839 mmol), tripotassium phosphate (1.78 g, 8.388 mmol) and ethylene glycol (0.468 mL, 8.388 mmol) at room temperature. The resulting mixture was stirred for 20 hours at 85° C. under nitrogen atmosphere. The reaction was quenched with water, adjusted pH to 3 with HCl (6M), extracted with ethyl acetate, washed with brine, dried over sodium sulfate, filtered, concentrated, and purified by silica gel chromatography (MeOH/DCM) to afford the title compound (200 mg, 21%) as a yellow oil.

MS(ESI) m/z=226.1, [M+H]⁺

### Step 2: N-(2-(2-(2-(2-azidoethoxy)ethoxy)ethoxy)ethyl)-1-(2-chlorophenyl)pyrrolidine-2-carboxamide

To a stirred solution of (2-chlorophenyl)proline (0.22 g, 0.975 mmol) in N,N-dimethylmethanamide (2.2 mL) were added HOBT (0.20 g, 1.462 mmol), EDCI (0.28 g, 1.462 mmol), 2-(2-(2-(2-azidoethoxy)ethoxy)ethoxy)ethan-1-amine (0.21 g, 0.975 mmol) and DIEA (0.50 g, 3.900 mmol) at room temperature. The resulting mixture was stirred for 2 hours at room temperature under nitrogen atmosphere. The reaction was quenched with water, extracted with ethyl acetate, washed with brine, dried over sodium sulfate, filtered concentrated, and purified by Prep-HPLC to afford the title compound (234.2 mg, 54%) as a yellow oil.

MS(ESI) m/z=426.3 [M+H]⁺

¹H NMR (300 MHz, Chloroform-d) δ 7.40-7.27 (m, 2H), 7.24-7.12 (m, 1H), 7.12-7.02 (m, 1H), 7.01-6.90 (m, 1H), 4.32-4.21 (m, 1H), 4.01-3.88 (m, 1H), 3.71-3.59 (m, 6H), 3.59-3.52 (m, 2H), 3.53-3.44 (m, 2H), 3.44-3.34 (m, 4H), 3.35-3.25 (m, 2H), 3.06-2.92 (m, 1H), 2.51-2.34 (m, 1H), 2.21-1.62 (m, 4H)

### Example 20: Synthesis of Ligand 22

### 3-(2-(2-(2-azidoethoxy)ethoxy)ethoxy)-1-(2-(2-chlorophenyl)pyrrolidin-1-yl)propan-1-one

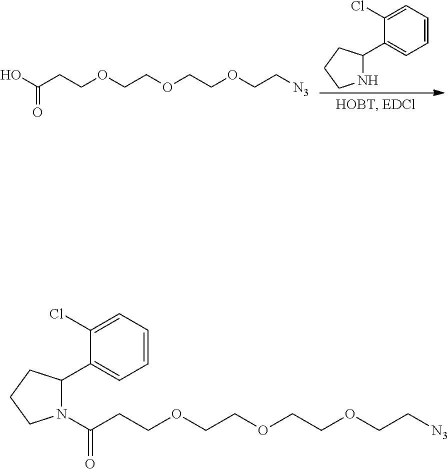

To a stirred solution of 3-[(8-azido-3,6-dioxaoct-1-yl)oxy]propanoic acid (300 mg, 1.213 mmol) in DMF (3 mL) were added 2-(2-chlorophenyl)tetrahydropyrrole (220.42 mg, 1.213 mmol), HOBt (245.94 mg, 1.820 mmol) and EDCI (348.90 mg, 1.820 mmol) batchwise at room temperature. DIEA (784.13 mg, 6.067 mmol) was added dropwise and the resulting mixture stirred overnight at room temperature under nitrogen atmosphere. The reaction was quenched by the addition of saturated aqueous NaHCO₃ solution and then extracted with ethyl acetate. The combined organic extracts were washed with saturated NaHCO₃ solution and brine, dried over Na₂SO₄, filtered, concentrated and purified by reversed-phase flash chromatography (SunFire C18 OBD Prep Column 19*150 mm, 5m; Mobile Phase A: Water (0.1% FA), Mobile Phase B: CH₃CN; Flow rate: 25 mL/min mL/min; Gradient: isocratic 38-61; Wave Length: 254 nm/220 nm nm; RT1(min): 6.83) to afford the title compound (0.2491 g, 49%) as a light yellow oil.

MS(ESI) m/z=411.1 [M+H]⁺

¹H NMR (300 MHz, Chloroform-d) δ 7.43-7.29 (m, 1H), 7.28-7.14 (m, 2H), 7.11-6.99 (m, 1H), 5.35 (m, 1H), 3.83-3.63 (m, 13H), 3.55-3.53 (m, 1H), 3.39-3.34 (m, 2H), 2.72-2.36 (m, 2H), 2.18-2.10 (m, 1H), 2.01-1.72 (m, 3H)

### Example 21: Synthesis of Ligand 23

### N-(2-(2-(2-(2-azidoethoxy)ethoxy)ethoxy)ethyl)-2-(2-chlorophenyl)pyrrolidine-1-carboxamide

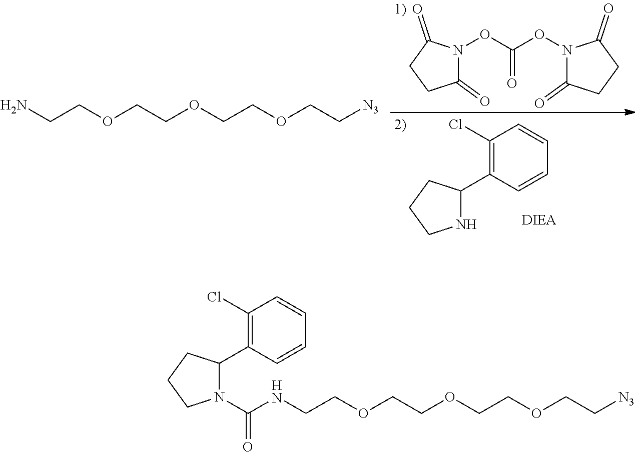

To a stirred solution of 2-[(8-azido-3,6-dioxaoct-1-yl)oxy]ethan-1-amine (300 mg, 1.375 mmol) in DCM (6 mL) was added disuccinimidyl carbonate (422.53 mg, 1.649 mmol) in portions at 0° C. and the mixture stirred for 4 h at room temperature under nitrogen atmosphere. DIEA (44.06 mg, 0.341 mmol) and 2-(2-chlorophenyl)tetrahydropyrrole (247.71 mg, 1.364 mmol) were then added dropwise at 0° C. and the resulting mixture stirred overnight at room temperature under nitrogen atmosphere. The reaction was quenched by the addition of saturated NaHCO₃ solution at 0° C. and extracted with ethyl acetate. The combined organic extracts were washed with water and brine, dried over Na₂SO₄, filtered, concentrated and purified by prep-HPLC (SunFire C18 OBD Prep Column 19*150 mm, 5m; Mobile Phase A: Water (0.1% FA), Mobile Phase B: ACN; Flow rate: 25 mL/min mL/min; Gradient: isocratic 37-47; Wave Length: 254 nm/220 nm nm; RT1(min): 8.26) to afford the title compound (180 mg, 31%) as a colorless oil.

MS(ESI) m/z=426.1 [M+H]⁺

¹H NMR (300 MHz, Chloroform-d) δ 7.36-7.24 (m, 1H), 7.20-7.15 (m, 1H), 7.15-7.07 (m, 2H), 5.16-5.00 (m, 1H), 4.01 (s, 1H), 3.71-3.51 (m, 8H), 3.50-3.38 (m, 4H), 3.37-3.23 (m, 5H), 2.43-2.26 (m, 1H), 1.89-1.71 (m, 3H)

In summary the following NMDA ligands were prepared:

| Li- | | |
|---|---|---|
| gand | Name | Structure |
| 1 | (1r,3R,5S,7r)- N-(12-azidododecyl)- 3,5-dimethyl adamantan-1-amine | 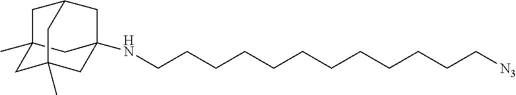 [SMILES: CC12CC3CC(C)(C1)CC(NCCCCCCCCCCCCN=[N+]=[N-])(C3)C2] |
| 2 | (1-azido-15- ((5S,10R)-5-methyl- 10,11-dihydro-5H- 5,10-epiminodibenzo [a,d][7]annulen- 12-yl)-3,6,9,12-tetra- oxapentadecan-15-one) | 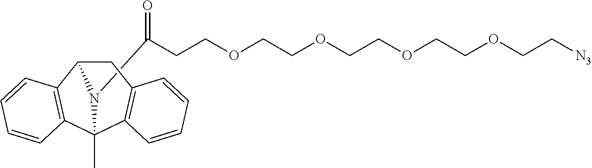 [SMILES: C[C@]12c3ccccc3C[C@H](c3ccccc31)N2C(=O)CCOCCOCCOCCOCCN=[N+]=[N-]] |
| 3 | (1-azido-N- ((5R,9R,E)-11- ethylidene-7-methyl- 2-oxo-2,6,9,10- tetrahydro-5,9- methanocycloocta [b]pyridin- 5(1H)-yl)-3,6,9,12- tetraoxapentadecan- 15-amide) | 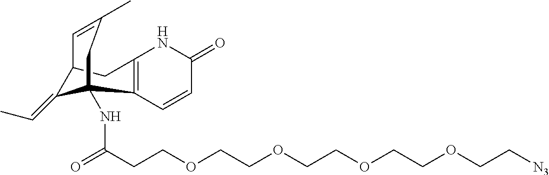 |
| 4 | ((S)-2-(3-(2-(2-(2- azidoethoxy)ethoxy) ethoxy)propanamido)- N-benzyl-3- methoxypropanamide) | 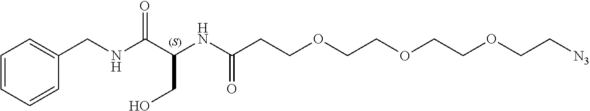 |
| 5 | ((S)-2-acetamido- N-(4-(2-(2-(2- (2-azidoethoxy) ethoxy)ethoxy) ethoxy)benzyl)-3- methoxypropanamide) | 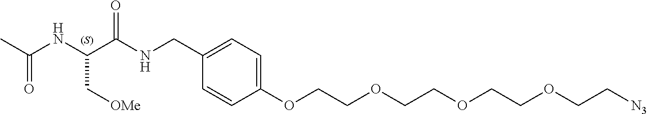 |
| 6 | ((S)-N-((2S,3R)- 1-amino-3-hydroxy-1- oxobutan-2-yl)-1-(3- (2-(2-(2-azidoethoxy) ethoxy)ethoxy) propanoyl)pyrrolidine- 2-carboxamide) | 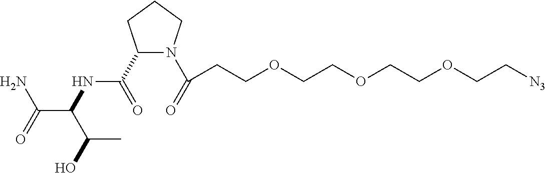 |
| 7 | (2-((2-(2-(2-(2- azidoethoxy) ethoxy)ethoxy) ethyl)amino)-2- (2-chlorophenyl) cyclohexan-1- one | 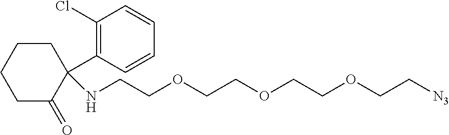 [SMILES: [N-]=[N+]=NCCOCCOCCOCCNC1(c2ccccc2Cl)CCCCC1=O] |
| 8 | N-(2-(2-(2-(2- azidoethoxy) ethoxy)ethoxy) ethyl)-2-(2- chlorophenyl) cyclopentane-1- carboxamide | 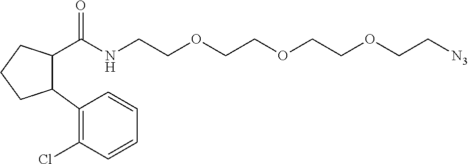 [SMILES: [N-]=[N+]=NCCOCCOCCOCCNC(=O)C1CCCC1c1ccccc1Cl] |
| 9 | (1-azido-N- ((1r,3R,5S,7r)-3,5- dimethyladamantan- 1-yl)-3,6,9,12- tetraoxapentadecan- 15-amide) | 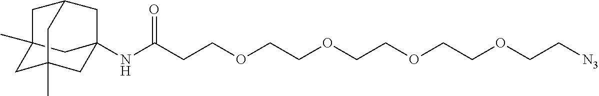 [SMILES: CC12CC3CC(C)(C1)CC(NC(=O)CCOCCOCCOCCOCCN=[N+]=[N-])(C3)C2] |
| 10 | (1-azido-15- ((5R,10S)-5-methyl- 10,11-dihydro-5H- 5,10-epiminodibenzo [a,d][7]annulen- 12-yl)-3,6,9,12- tetraoxapentadecan- 15-one) | 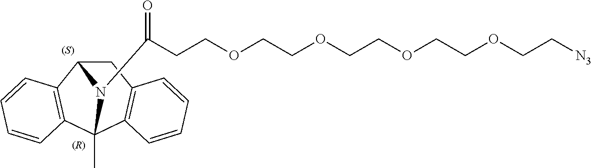 |
| 11 | (1-azido-15- (3-chloro-5-methyl- 10,11-dihydro-5H- 5,10-epiminodibenzo [a,d][7]annulen- 12-yl)-3,6,9,12- tetraoxapentadecan- 15-one) | 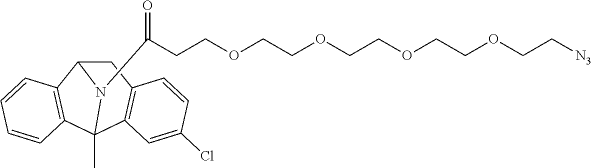 [SMILES: CC12c3cc(Cl)ccc3CC(c3ccccc31)N2C(=O)CCOCCOCCOCCOCCN=[N+]=[N-]] |
| 12 | ((4aR,5R,10bR)-1- (1-azido-3,6,9,12- tetraoxapentadecan- 15-oyl)-12-methyl- 2,3,4,4a,5,6- hexahydro-1H-5,10b- prop[1]eno-1,7- phenanthrolin- 8(7H)-one) | 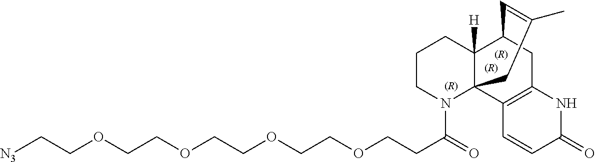 |
| 13 | ((S)-1-((2S,4R)- 1-(L-threonyl)- 4-(2-(2-(2-(2- azidoethoxy) ethoxy)ethoxy) ethoxy)pyrrolidine- 2-carbonyl)-N- ((2S,3R)-1-amino- 3-hydroxy-1- oxobutan-2-yl) pyrrolidine-2- carboxamide) | 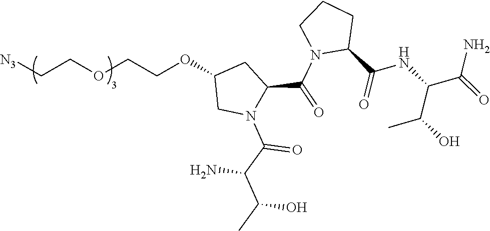 |
| 14 | (N-(2-(2-(2-(2- azidoethoxy) ethoxy)ethoxy)ethyl)- 2-(1-((1R,2S)-1- hydroxy-1-(4- hydroxyphenyl)propan- 2-yl)piperidin- 4-yl)acetamide | 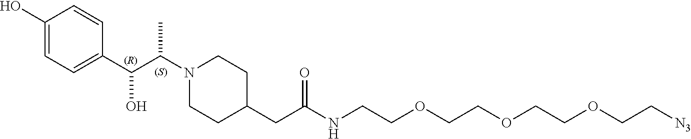 |
| 15 | N-(2-(2-(2-(2- azidoethoxy)ethoxy) ethoxy)ethyl)- 2-(1-((1R,2R)- 1-hydroxy-1-(4- hydroxyphenyl)propan- 2-yl)piperidin- 4-yl)acetamide) | 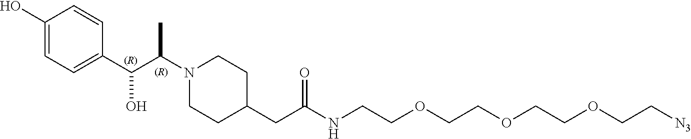 |
| 16 | 1-azido-15- (3-chloro-5-methyl- 10,11-dihydro-5H- 5,10-epimino- dibenzo[a,d][7]- annulen-12-yl)- 3,6,9,12- tetraoxapentadecan- 15-one | 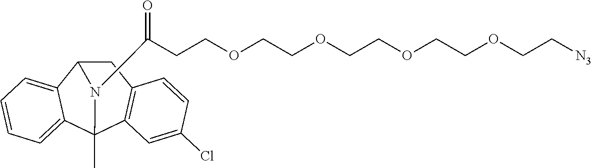 [SMILES: CC12c3cc(Cl)ccc3CC(c3ccccc31)N2C(=O)CCOCCOCCOCCOCCN=[N+]=[N-]] |
| 17 | (5S,10R)-12- (12-azidododecyl)-5- methyl-10,11- dihydro-5H- 5,10-epiminodibenzo- [a,d][7]annulene | 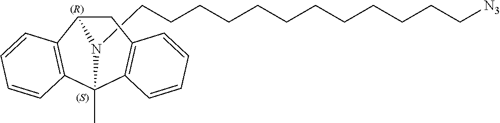 |
| 18 | (S)-N-(((1R,3S,5S,7S)- adamantan-2-yl) methyl)-2-(3-(2- (2-(2-azidoethoxy) ethoxy)-ethoxy) propanamido)-3- methoxypropanamide | 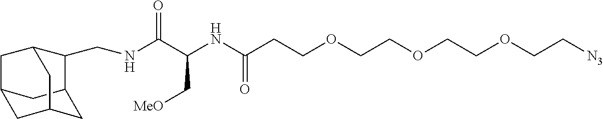 [SMILES: COC[C@H](NC(=O)CCOCCOCCOCCN=[N+]=[N-])C(=O)NCC1C2CC3CC(C2)CC1C3] |
| 19 | (S)-2-(3-(2-(2-(2- azidoethoxy)ethoxy) ethoxy) propanamido)-N- (4-fluorobenzyl)- 3-methoxy- propanamide | 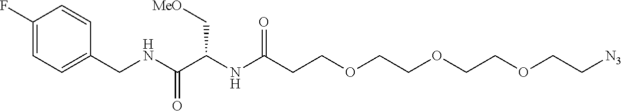 [SMILES: COC[C@H](NC(=O)CCOCCOCCOCCN=[N+]=[N-])C(=O)NCc1ccc(F)cc1] |
| 20 | (S)-2-(3-(2-(2-(2- azidoethoxy)ethoxy) ethoxy)propanamido)- 3-methoxy-N- (4-methylbenzyl) propanamide | 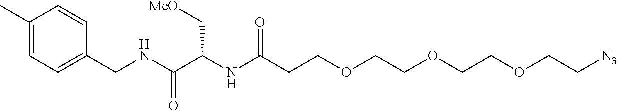 [SMILES: COC[C@H](NC(=O)CCOCCOCCOCCN=[N+]=[N-])C(=O)NCc1ccc(C)cc1] |
| 21 | N-(2-(2-(2-(2- azidoethoxy) ethoxy)ethoxy)ethyl)- 1-(2-chlorophenyl) pyrrolidine- 2-carboxamide | 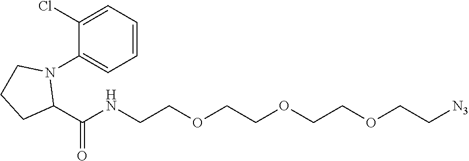 [SMILES: [N-]=[N+]=NCCOCCOCCOCCNC(=O)C1CCCN1c1ccccc1Cl] |
| 22 | 3-(2-(2-(2- azidoethoxy) ethoxy)ethoxy)-1- (2-(2-chlorophenyl) pyrrolidin-1- yl)propan-1-one | 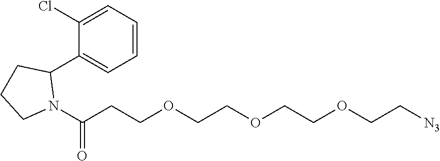 [SMILES: [N-]=[N+]=NCCOCCOCCOCCC(=O)N1CCCC1c1ccccc1Cl] |
| 23 | N-(2-(2-(2-(2- azidoethoxy) ethoxy)ethoxy)ethyl)- 2-(2-chlorophenyl) pyrrolidine- 1-carboxamide | 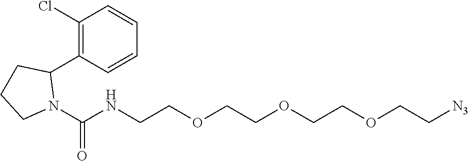 [SMILES: [N-]=[N+]=NCCOCCOCCOCCNC(=O)N1CCCC1c1ccccc1Cl] |

### Preparation of Ligand-Conjugated Oligonucleotides

Exemplary ligand-conjugated oligonucleotides falling within the scope of the present disclosure, may be synthesized according to the following general procedures.

In some instances, one Ligand (Ligand A in the general procedures described below) is conjugated to the 5′ end of an oligonucleotide.

In some instances, two identical Ligands (Ligand A and Ligand A) are conjugated to the 5′ and 3′ ends of an oligonucleotide.

In some instances, two different Ligands (Ligand A and Ligand B) are conjugated to the 5′ and 3′ ends of an oligonucleotide.

In some instances, one Ligand (Ligand A) is conjugated to the 3′ end of an oligonucleotide.

### Example 22: Ligand Conjugated to 5′ End of Sense Strand

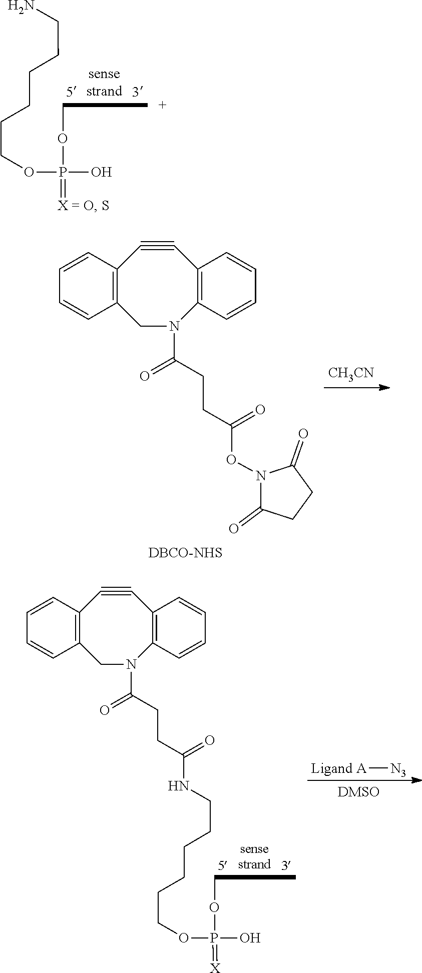

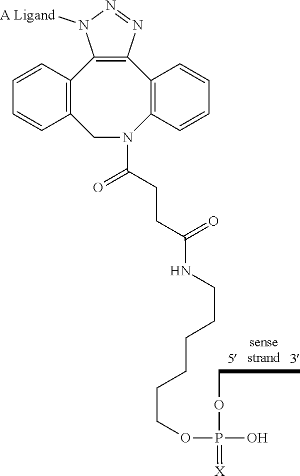

### Step 1: 5′-DBCO Functionalized Sense Strand

Sodium Phosphate buffer (10% V/V, 1M, pH7) and acetonitrile (20%-50% V/V) were added to an aqueous solution of 5′-amine functionalized sense strand. A solution of DBCO-NHS (1.5-3 eq) in DMSO or acetonitrile was then added and the reaction monitored by LCMS and HPLC. Upon completion, any precipitate was removed via centrifugation and the aqueous solution purified by reverse phase HPLC, dried by lyophilization and the dried 5′-DBCO functionalized sense strand reconstituted in RNase free water.

### Step 2: 5′-Ligand Conjugated Sense Strand

A solution of Ligand A-N3 (2 eq) in DMSO or THF was added to a solution of 5′-DBCO modified sense strand (1 eq) and the reaction monitored by HPLC and LCMS. Upon completion, the 5′-conjugated sense strand was purified by reverse phase HPLC or molecular weight cut-off with Amicon® Ultra-15 Centrifugal filter (3K, 5 times).

### Example 23: Ligand Conjugated to 5′ End of Sense Strand

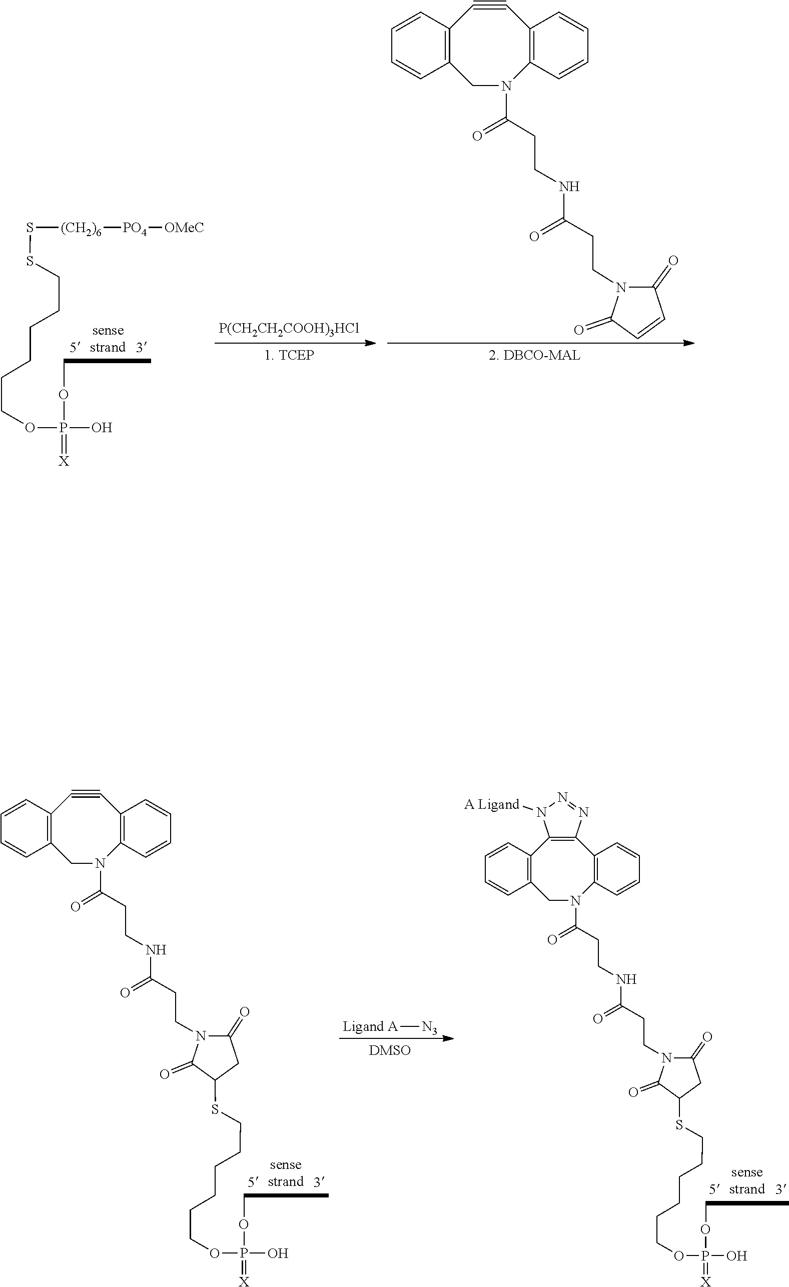

### Step 1: 5′-DBCO Functionalized Sense Strand

Sodium Phosphate buffer (10% V/V 1M, pH7) is added to an aqueous solution of 5′-(C6-SS—C6)-mC functionalized sense strand. Tris(2-carboxyethyl)phosphine hydrochloride (TCEP) (25 eq) in water (pH7) is added and the reaction monitored by HPLC and LCMS. Upon completion, excess TCEP is removed by molecular weight cut-off with sodium phosphate buffer (100 mM, pH7, 3×). A solution of DBCO-MAL (3 eq) in DMSO is added and the reaction monitored by LCMS and HPLC. Upon completion, any solids are removed via centrifugation, the solution purified by reverse phase HPLC, dried by lyophilization and the dried product reconstituted in RNase free water.

### Step 2: 5′-Ligand Conjugated Sense Strand

To a solution of 5′-DBCO functionalized sense strand (1 eq) is added a solution of Ligand A-N3 (3 eq) in DMSO or THF and the reaction monitored by HPLC and LCMS. Upon completion, the 5′-conjugated sense strand is purified by reverse phase HPLC or molecular weight cut-off with Amicon® Ultra-15 Centrifugal filter (3K, 5 times).

### Example 24: Ligand Conjugated to 5′ End of Sense Strand

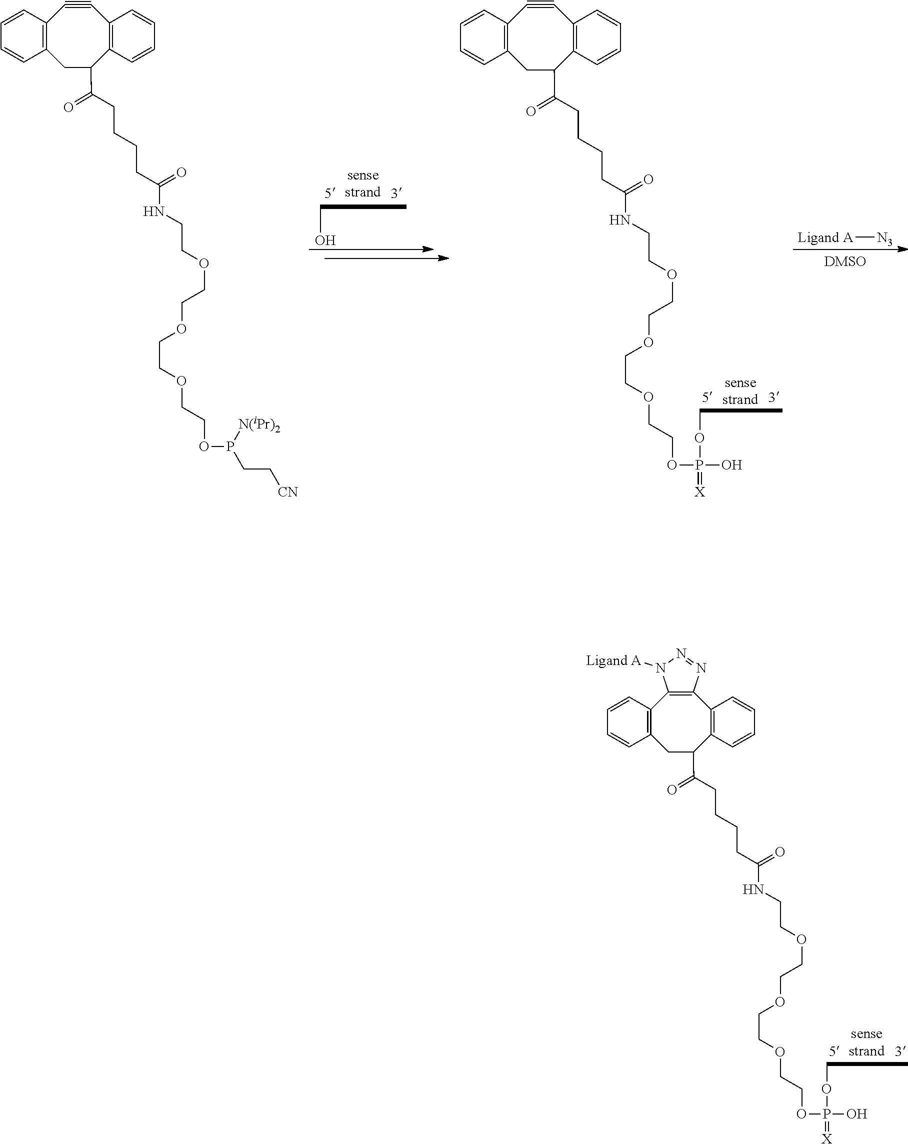

### Step 1: 5′-DBCO Functionalized Sense Strand

10-(6-oxo-6-(dibenzo[b,f]azacyclooct-4-yn-1-yl)-capramido-N-ethyl)-O-triethyleneglycol-1-[(2-cyanoethyl)-(N,N-diisopropyl)]-phosphoramidite (5′-DBCO-TEG Phosphoramidite, purchased from Glen research, 10-1941-02E) is coupled to the 5′ end of sense strand, under standard Phosphoramidite coupling conditions.

### Step 2: 5′-Ligand Conjugated Sense Strand

A solution of Ligand A-N3 (2 eq) in DMSO is added to a solution of 5′-DBCO modified sense strand (1 eq) and the reaction monitored by HPLC and LCMS. Upon completion, the 5′-conjugated sense strand is purified by reverse phase HPLC or molecular weight cut-off with Amicon® Ultra-15 Centrifugal filter.

### Example 25: Bis-homo-3′,5′-Ligand Conjugated Sense Strand

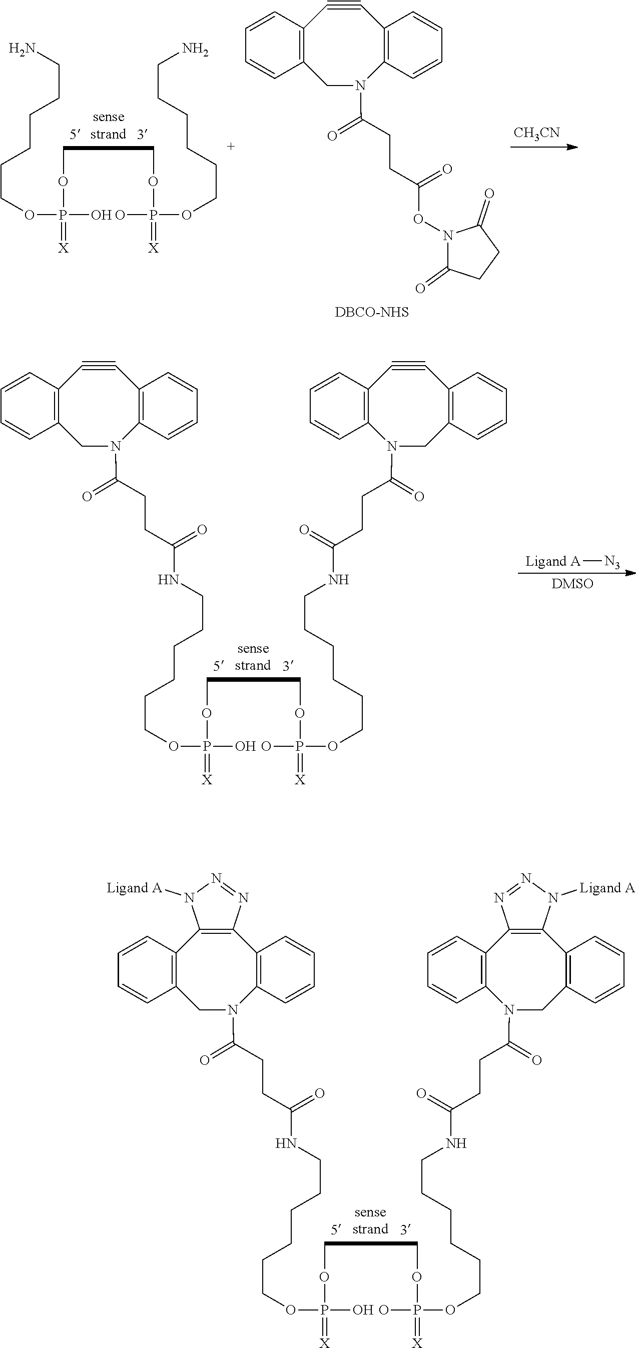

### Step 1: 3′,5′-Bis-DBCO Modified Sense Strand

Sodium Phosphate buffer (10% V/V 1M, pH7) and acetonitrile (20%-50% V/V) were added to an aqueous solution of 3′,5′ amine functionalized sense strand. A solution of DBCO-NHS (3 eq) in DMSO or CH₃CN was then added and the reaction monitored by LCMS and HPLC. Upon completion, the product was purified by reverse phase HPLC, dried by lyophilization and reconstituted in RNase free water.

### Step 2: 3′,5′-Bis-Conjugated Sense Strand

A solution of Ligand A-N3 (3 eq) in DMSO or CH₃CN was added to a solution of 3′,5′-bis-DBCO modified sense strand (1 eq) and the reaction monitored by HPLC and LCMS. Upon completion, the 3′,5′-bis conjugated sense strand was purified by reverse phase HPLC, dried by lyophilization, reconstituted in RNase free water and desalted using Amicon® Ultra-15 Centrifugal filter (3K, 5 times).

### Example 26: Bis-homo-3′,5′-Ligand Conjugated Sense Strand

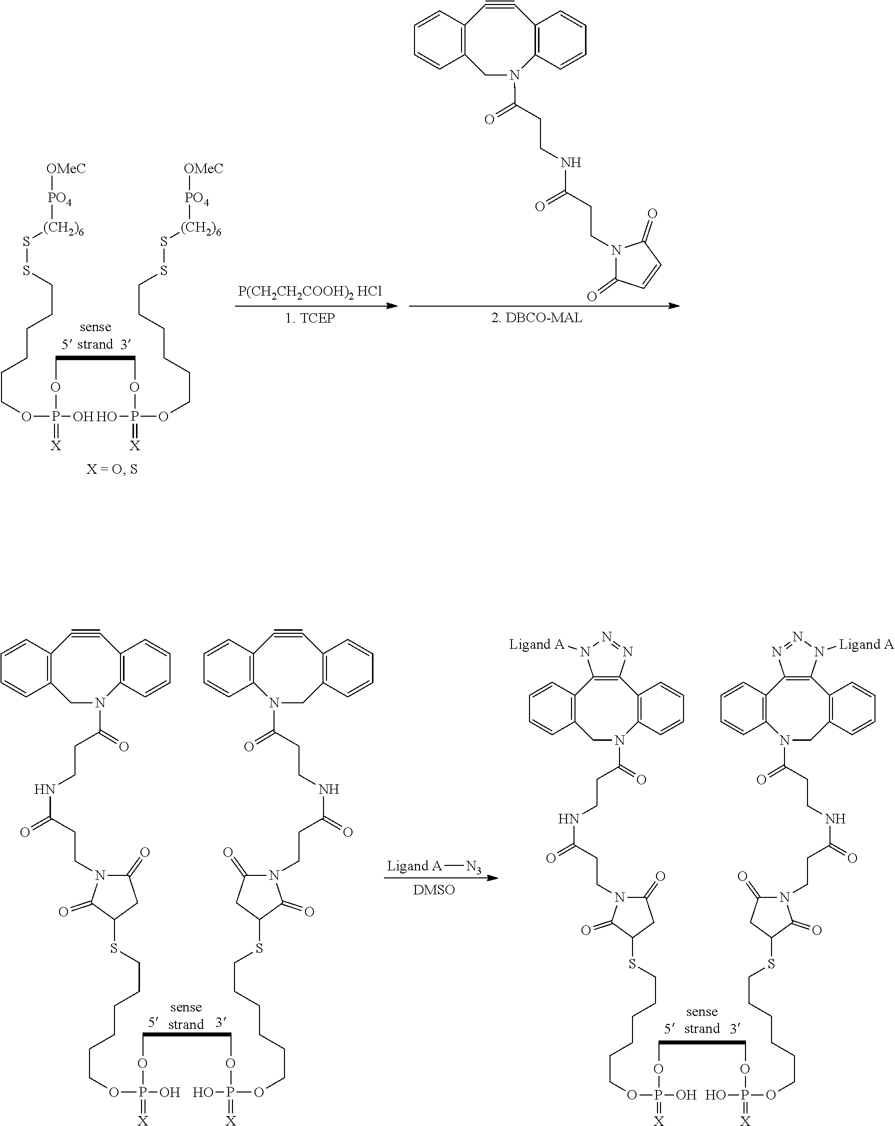

### Step 1: 3′,5′-Bis-DBCO Modified Sense Strand

Sodium Phosphate buffer (10% V/V 1M, pH7) is added to an aqueous solution of 5′, 3′-Bis (C6-SS—C6)-mC functionalized sense strand. Tris(2-carboxyethyl)phosphine hydrochloride (TCEP) (25 eq) in water (pH7) is added and the reaction monitored by HPLC and LCMS. Upon completion, excess TCEP is removed by MWCO with sodium phosphate buffer (100 mM, pH7, 3×). A solution of DBCO-MAL (3 eq) in DMSO is added and the reaction monitored by LCMS and HPLC. Upon completion, any solids are removed via centrifugation and the solution purified by reverse phase HPLC, dried by lyophilization and the dried bis-DBCO modified sense strand reconstituted in RNase free water.

### Step 2: 3′,5′-Bis-Conjugated Sense Strand

To a solution of 5′,3′-Bis-DBCO functionalized sense strand (1 eq) is added a solution of Ligand A-N₃ (3 eq) in DMSO or THF and the reaction monitored by HPLC and LCMS. Upon completion, the bis-homo-5′-, 3′conjugated sense strand is purified by reverse phase HPLC or molecular weight cut-off with Amicon® Ultra-15 Centrifugal filter (3K, 5 times).

### Example 27: Bis-homo-3′,5′-Ligand Conjugated Sense Strand

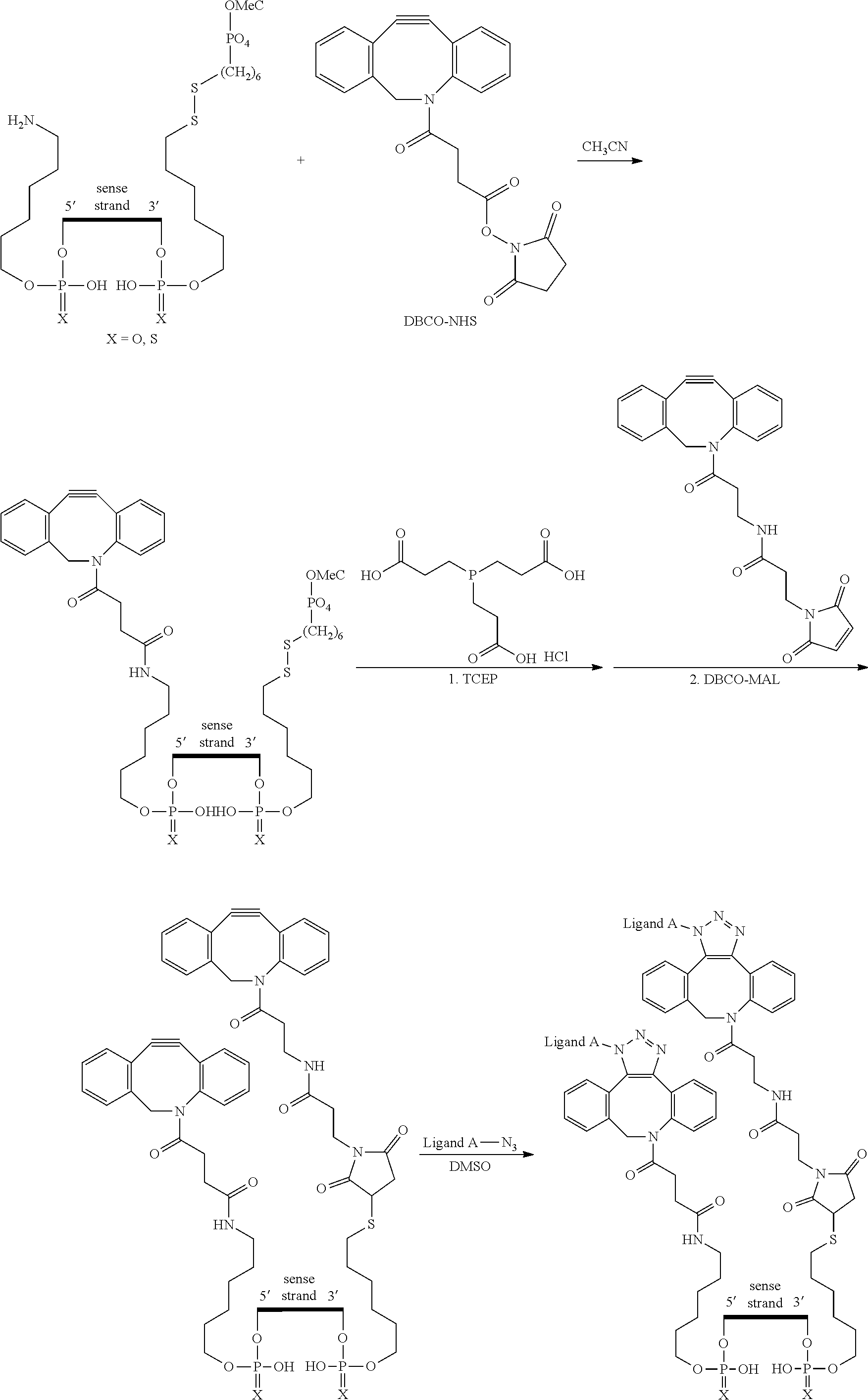

### Step 1: 5′-DBCO/3′-(C₆—SS—C₆)-mC Functionalized Sense Strand

Sodium Phosphate buffer (10% V/V 1M, pH7) and acetonitrile (20%-50% V/V) were added to an aqueous solution of 5′-amine functionalized sense strand. A solution of DBCO-NHS (1.5-3 eq) in DMSO or acetonitrile was then added and the reaction was monitored by LCMS and HPLC. Upon completion, any precipitate was removed via centrifugation and the aqueous solution purified by reverse phase HPLC. The product fractions were combined, dried by lyophilization and the dried N-DBCO modified sense strand reconstituted in RNase free water for step 2.

### Step 2: 5′,3′-Bis DBCO Functionalized Sense Strand

Sodium Phosphate buffer (10% V/V 1M, pH7) was added to an aqueous solution of 5′-DBCO/3′-(C₆—SS—C₆)-mC functionalized sense strand. Tris(2-carboxyethyl)phosphine hydrochloride (TCEP) (25 eq) in water (pH7) was added and the reaction was monitored by HPLC and LCMS. Upon completion, excess TCEP was removed by MWCO with sodium phosphate buffer (100 mM, pH7, 3×). A solution of DBCO-MAL (3 eq) in DMSO was added and the reaction was monitored by LCMS and HPLC. Upon completion, any solids were removed via centrifugation and the solution was purified by reverse phase HPLC, dried by lyophilization and the dried bis-DBCO modified sense strand reconstituted in RNase free water for step 3.

### Step 3: Bis-homo-5′,3′-Ligand Conjugated Sense Strand

To a solution of 5′-, 3′-Bis-DBCO functionalized sense strand (1 eq) was added a solution of Ligand A-N₃ (3 eq) in DMSO or THF and the reaction was monitored by HPLC and LCMS. Upon completion, the bis-homo-5′-, 3′conjugated sense strand was purified by reverse phase HPLC or molecular weight cut-off with Amicon® Ultra-15 Centrifugal filter (3K, 5 times). The product was confirmed by HPLC and LCMS.

### Example 28: Bis-homo-3′,5′-Ligand Conjugated Sense Strand

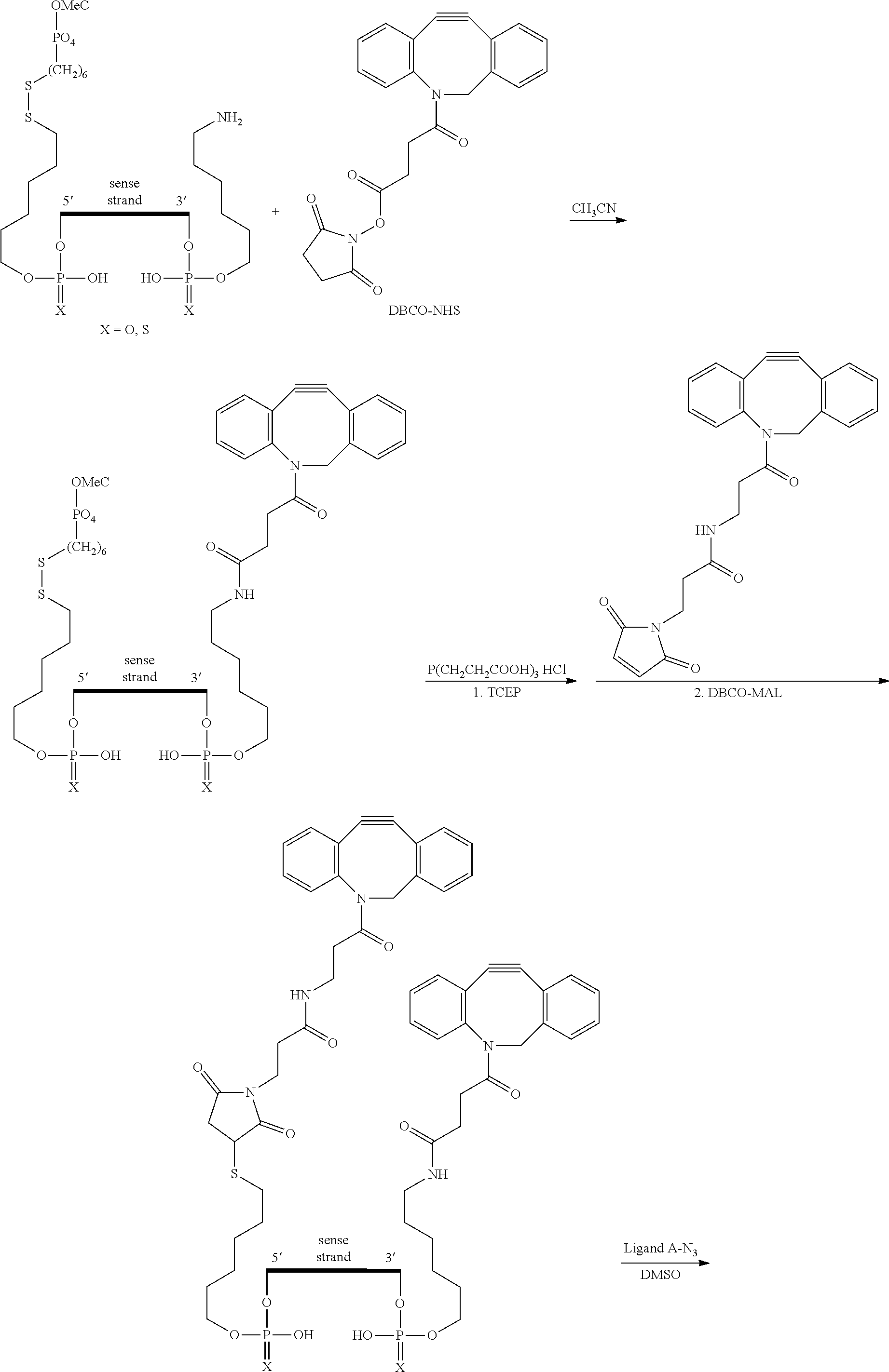

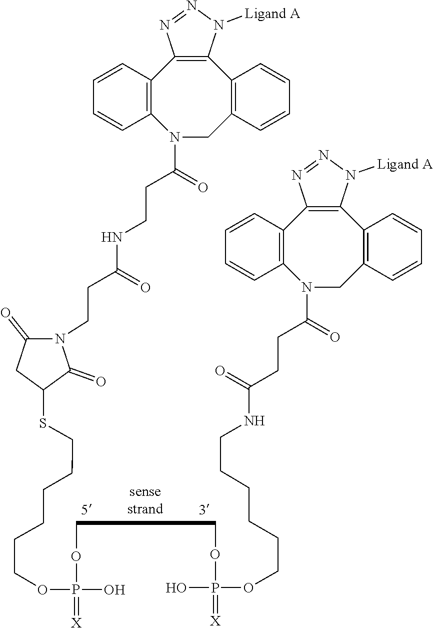

### Step 1: 5′-(C6-SS—C6)-mC/3′-DBCO Functionalized Sense Strand

Sodium Phosphate buffer (10% V/V 1M, pH7) and acetonitrile (20%-50% V/V) were added to an aqueous solution of 5′-amine functionalized sense strand. A solution of DBCO-NHS (1.5-3 eq) in DMSO or acetonitrile was then added and the reaction was monitored by LCMS and HPLC. Upon completion, any precipitate was removed via centrifugation and the aqueous solution purified by reverse phase HPLC. The product fractions were combined, dried by lyophilization and the dried N-DBCO modified sense strand reconstituted in RNase free water for step 2.

### Step 2: 5′,3′-Bis DBCO Functionalized Sense Strand

Sodium Phosphate buffer (10% V/V 1M, pH7) was added to an aqueous solution of 5′-DBCO/3′-(C₆—SS—C₆)-mC functionalized sense strand. Tris(2-carboxyethyl)phosphine hydrochloride (TCEP) (25 eq) in water (pH7) was added and the reaction was monitored by HPLC and LCMS. Upon completion, excess TCEP was removed by MWCO with sodium phosphate buffer (100 mM, pH7, 3×). A solution of DBCO-MAL (3 eq) in DMSO was added and the reaction was monitored by LCMS and HPLC. Upon completion, any solids were removed via centrifugation and the solution was purified by reverse phase HPLC, dried by lyophilization and the dried bis-DBCO modified sense strand reconstituted in RNase free water for step 3.

### Step 3: Bis-homo-5′,3′-Ligand Conjugated Sense Strand

To a solution of 5′-, 3′-Bis-DBCO functionalized sense strand (1 eq) was added a solution of Ligand A-N₃ (3 eq) in DMSO or THF and the reaction was monitored by HPLC and LCMS. Upon completion, the bis-homo-5′-, 3′conjugated sense strand was purified by reverse phase HPLC or molecular weight cut-off with Amicon® Ultra-15 Centrifugal filter (3K, 5 times). The product was confirmed by HPLC and LCMS.

### Example 29: Bis-hetero-3′,5′-Ligand Conjugated Sense Strand

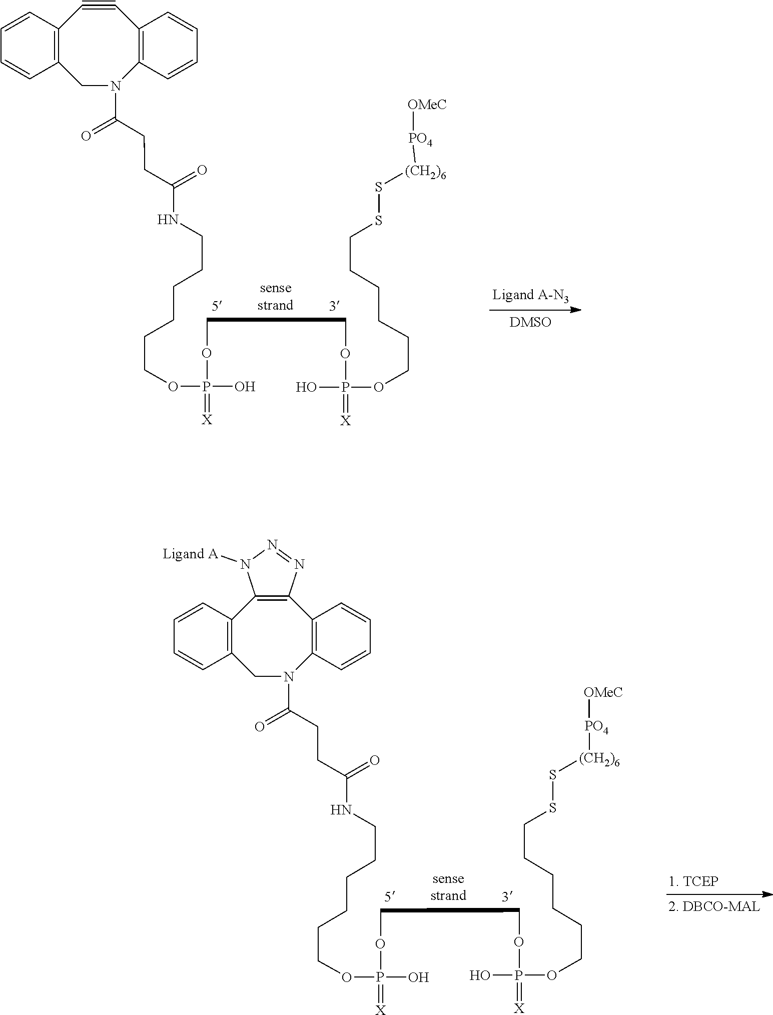

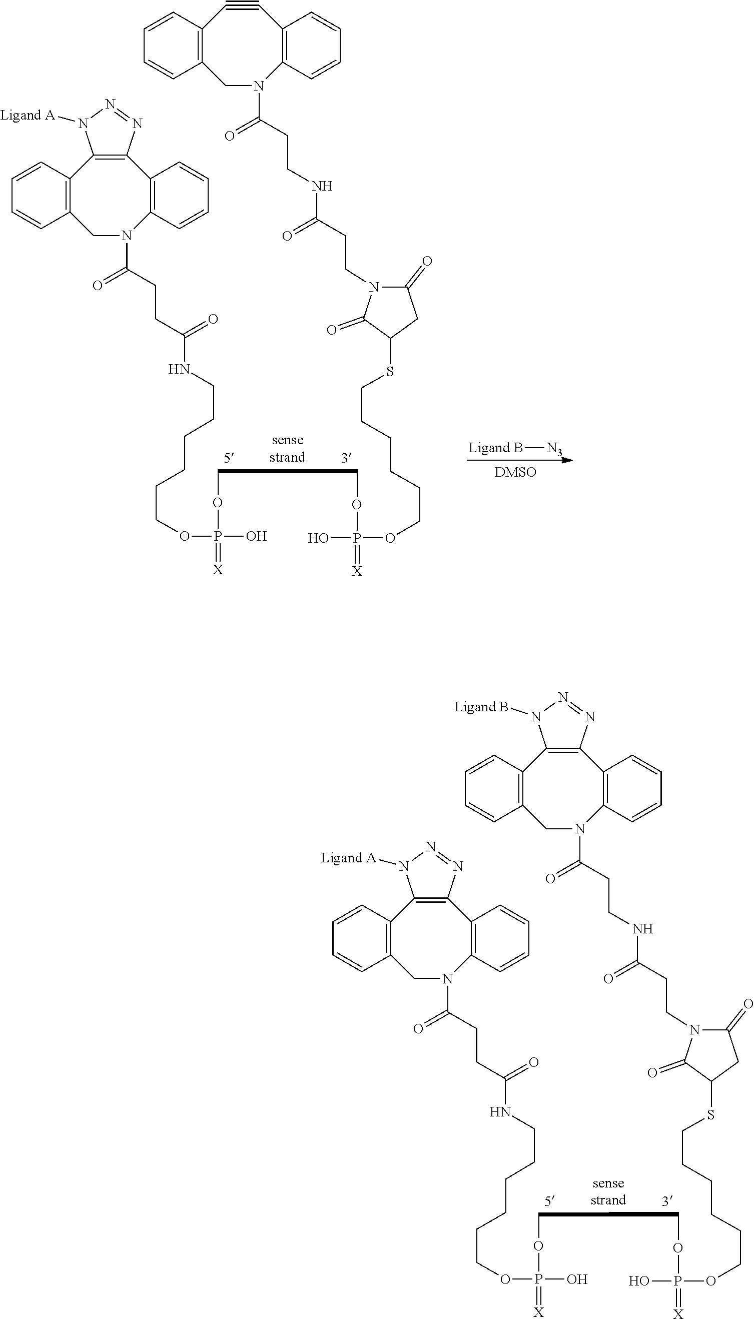

### Step 1: 5′-Conjugated, 3′-(C₆—SS—C₆)-mC Functionalized Sense Strand

A solution of Ligand A-N3 (2 eq) in DMSO was added to an aqueous solution of 5′-DBCO modified sense strand (1 eq, see above for preparation) and the reaction was monitored by HPLC and LCMS. Upon completion, the 5′-conjugated sense strand was purified by reverse phase HPLC or molecular weight cut-off with Amicon® Ultra-15 Centrifugal filter (3K, 5 times).

### Step 2: The 5′-Conjugated, 3′-DBCO Modified Sense Strand

Sodium phosphate buffer (10% V/V 1M, pH7) was added to a solution of 5′-conjugated, 3′-(C6-SS—C6)-mC functionalized sense strand (1 eq) in water. Tris(2-carboxyethyl)phosphine hydrochloride (TCEP, 25 eq) in water (pH7) was added and the reaction was monitored by HPLC and LCMS. Upon completion, excess TCEP was removed by MWCO with sodium phosphate buffer (100 mM, pH7, 3×). A solution of DBCO-MAL (3 eq) in DMSO was added and the reaction monitored by HPLC and LCMS. Upon completion, the aqueous solution was purified by reverse phase HPLC, dried by lyophilization and the dried 5′-conjugated, 3′-DBCO modified sense strand was reconstituted in Sodium Phosphate buffer (100 mM) for step 3.

### Step 3: Bis-hetero-3′,5′-Ligand Conjugated Sense Strand

A solution of Ligand B—N3 (2 eq) in DMSO was added to an aqueous solution of 5′-conjugated, 3′-DBCO functionalized sense strand (1 eq) and the reaction was monitored by HPLC and LCMS. Upon completion, the 5′-, 3′-conjugated sense strand was purified by reverse phase HPLC or molecular weight cut-off with Amicon® Ultra-15 Centrifugal filter (3K, 5 times).

### Example 30: Bis-hetero-3′,5′-Ligand Conjugated Sense Strand

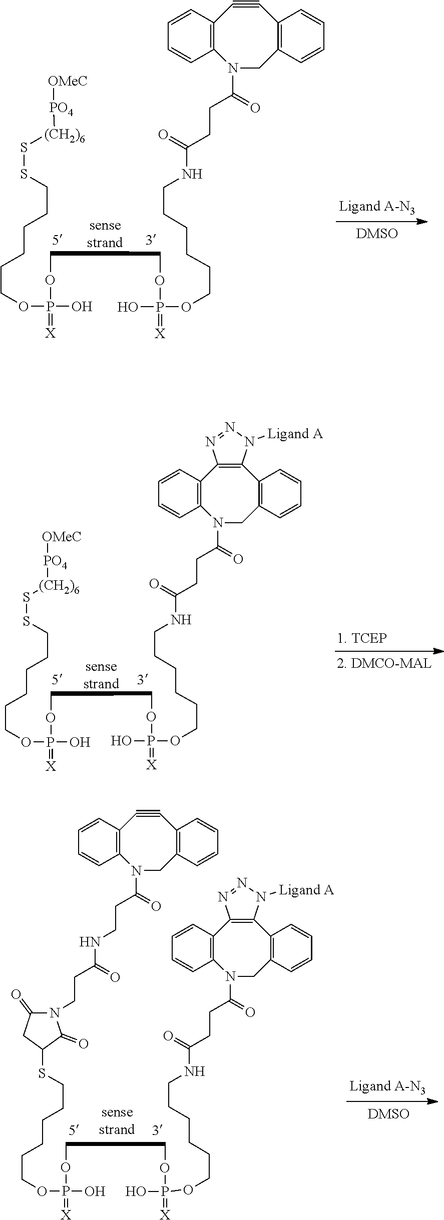

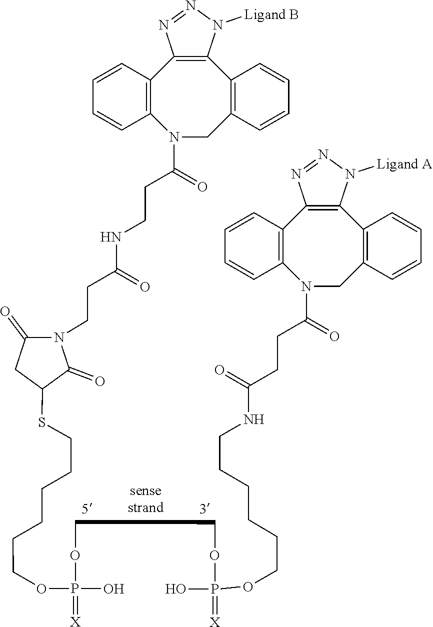

### Step 1: 5′-(C6-SS—C6)-mC, 3′-Conjugated Sense Strand

A solution of Ligand A-N3 (2 eq) in DMSO was added to an aqueous solution of 3′-DBCO modified sense strand (1 eq, see above for preparation) and the reaction was monitored by HPLC and LCMS. Upon completion, the 3′-conjugated sense strand was purified by reverse phase HPLC or molecular weight cut-off with Amicon® Ultra-15 Centrifugal filter (3K, 5 times).

### Step 2: The 5′-DBCO, 3′-Conjugated Sense Strand

Sodium phosphate buffer (10% V/V 1M, pH7) was added to a solution of 5′-conjugated, 3′-(C6-SS—C6)-mC functionalized sense strand (1 eq) in water. Tris(2-carboxyethyl)phosphine hydrochloride (TCEP, 25 eq) in water (pH7) was added and the reaction was monitored by HPLC and LCMS. Upon completion, excess TCEP was removed by MWCO with sodium phosphate buffer (100 mM, pH7, 3×). A solution of DBCO-MAL (3 eq) in DMSO was added and the reaction monitored by HPLC and LCMS. Upon completion, the aqueous solution was purified by reverse phase HPLC, dried by lyophilization and the dried 5′-conjugated, 3′-DBCO modified sense strand was reconstituted in Sodium Phosphate buffer (100 mM) for step 3.

### Step 3: Bis-hetero-3′,5′-Ligand Conjugated Sense Strand

A solution of Ligand B—N3 (2 eq) in DMSO was added to an aqueous solution of 5′-conjugated, 3′-DBCO functionalized sense strand (1 eq) and the reaction was monitored by HPLC and LCMS. Upon completion, the 5′-, 3′-conjugated sense strand was purified by reverse phase HPLC or molecular weight cut-off with Amicon® Ultra-15 Centrifugal filter (3K, 5 times).

### Example 31: Ligand Conjugated to 3′ End of Sense Strand

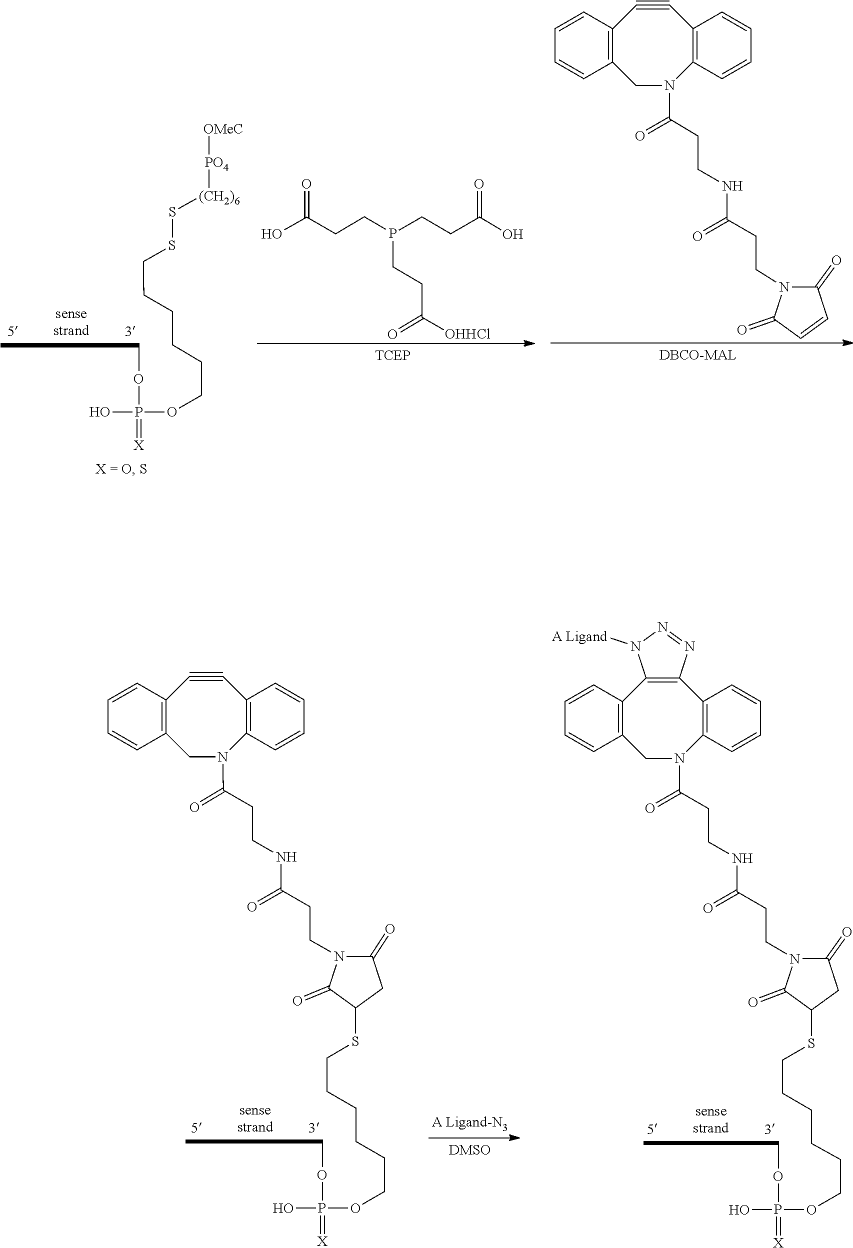

### Step 1: 3′-DBCO Modified Sense Strand

Sodium Phosphate buffer (10% V/V 1M, pH7) was added to an aqueous solution of 3′-(C6-SS—C6)-mC functionalized sense strand. Tris(2-carboxyethyl)phosphine hydrochloride TCEP, 25 eq) in water (pH7) was added and the reaction monitored by HPLC and LCMS. Upon completion, excess TCEP was removed by MWCO with sodium phosphate buffer (100 mM, pH=7, 3×). A solution of DBCO-MAL (3 eq) in DMSO was added and the reaction was monitored by HPLC and LCMS. Upon completion, any solids were removed via centrifugation and the solution was purified by reverse phase HPLC, dried by lyophilization and the dried 3′-DBCO modified sense strand was reconstituted in 100 mM Sodium Phosphate buffer for step 2.

### Step 2: 3′-Ligand Conjugated Sense Strand

A solution of Ligand A-N3 (3 eq) in DMSO was added to an aqueous solution of 3′-DBCO functionalized sense strand (1 eq) and the reaction was monitored by HPLC and LCMS. Upon completion, the 3′-conjugated sense strand was purified by reverse phase HPLC or molecular weight cut-off with Amicon® Ultra-15 Centrifugal filter (3K, 5×).

### Example 32: Ligand Conjugated to 3′ End of Sense Strand

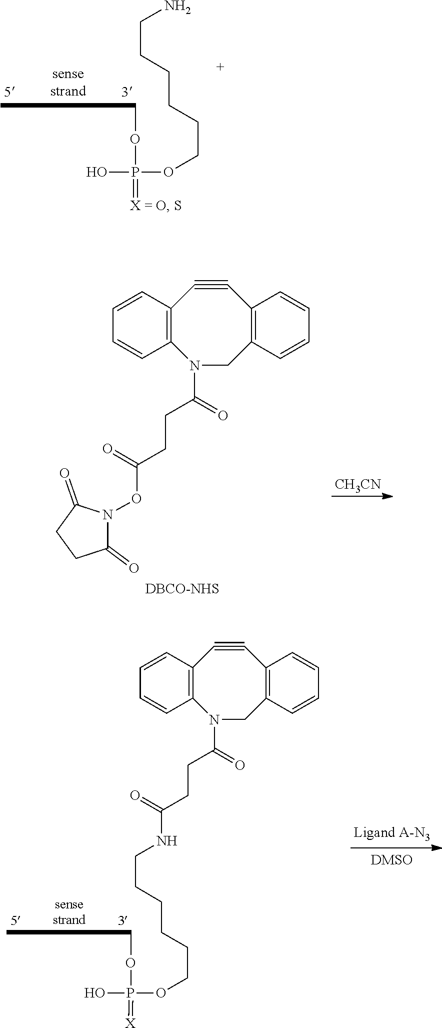

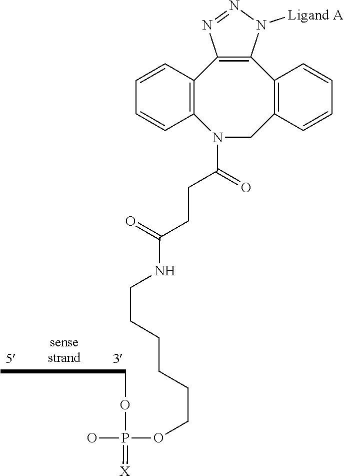

### Step 1: 3′-DBCO Functionalized Sense Strand

Sodium Phosphate buffer (10% V/V, 1M, pH7) and acetonitrile (20%-50% V/V) are added to an aqueous solution of 3′-amine functionalized sense strand. A solution of DBCO-NHS (1.5-3 eq) in DMSO or acetonitrile is then added and the reaction monitored by LCMS and HPLC. Upon completion, any precipitate is removed via centrifugation, the aqueous solution purified by reverse phase HPLC, dried by lyophilization and the dried DBCO modified sense strand reconstituted in RNase free water.

### Step 2: 3′-Ligand Conjugated Sense Strand

A solution of Ligand A-N3 (2 eq) in DMSO or THF is added to a solution of 3′-DBCO modified sense strand (1 eq) and the reaction monitored by HPLC and LCMS. Upon completion, the 3′-conjugated sense strand was purified by reverse phase HPLC or molecular weight cut-off with Amicon® Ultra-15 Centrifugal filter (3K, 5 times).

### General Procedures for Strand Preparation

### Example 33: Strand Synthesis

Strands were synthesized on solid phase using an oligonucleotide synthesizer Oligopilot100 (Cytiva Life Sciences). Solid support (CPG, 80-90 μmol/g, 500 A, from LGC-Biosearch Technologies, Petaluma, CA) was loaded to 150-300 μmol scales. RNA and 2′ modified RNA phosphoramidites were purchased from Hongene Biotech (Union City, CA).
2′-O-methyl phosphoramidites used were:
5′-O-(4,4′-Dimethoxytrityl)-N6-benzoyl-2′-O-methyl-adenosine-3′-[(2-cyanoethyl)-(N,N-diisopropyl)]-phosphoramidite
5′-O-(4,4′-Dimethoxytrityl)-N4-acetyl-2′-O-methyl-cytidine-3′-O-[(2-cyanoethyl)-(N,N-diisopropyl)]-phosphoramidite
5′-O-(4,4′-Dimethoxytrityl)-N2-isobutyryl-2′-O-methyl-guanosine-3′-O-[(2-cyanoethyl)-(N,N-diisopropyl)]-phosphoramidite
5′-O-(4,4′-Dimethoxytrityl)-2′-O-methyl-uridine-3′-O-[(2-cyanoethyl)-(N,N-diisopropyl)]-phosphoramidite.
2′-Fluoro phosphoramidites used were:
5′-O-(4,4′-Dimethoxytrityl)-N6-benzoyl-2′-fluoroadenosine-3′-O-[(2-cyanoethyl)-(N,N-diisopropyl)]-phosphoramidite
5′-O-(4,4′-Dimethoxytrityl)-N4-acetyl-2′-fluorocytidine-3′-O-[(2-cyanoethyl)-(N,N-diisopropyl)]-phosphoramidite
5′-O-(4,4′-Dimethoxytrityl)-N2-isobutyryl-2′-fluoroguanosine-3′-O-[(2-cyanoethyl)-(N,N-diisopropyl)]-phosphoramidite
5′-O-(4,4′-Dimethoxytrityl)-2′-fluorouridine-3′-O-[(2-cyanoethyl)-(N,N-diisopropyl)]-phosphoramidite.

To create phosphorohioate linkages 3-((Dimethylamino-methylidene)amino)-3H-1,2,4-dithiazole-3-thione (DDTT. 0.1M solution from Chemgenes, Wilmington, MA) was used for 4-6 minutes. To create phosphodiester linkage a solution of 120 in Pyridine/Water (0.05M from Sigma Aldrich, St Louis, MO) was used. Following the oxidation/sulfurization a mixture of 20% n-Methylimidazole in Acetonitrile, and 40% Acetic Anhydride in 60% Lutidine in Acetonitrile (Sigma Aldrich, St Louis, MO) were used to acetylate any unreacted chain attached to the CPG.

Phosphoramidites were dissolved in anhydrous acetonitrile (0.2M) and molecular sieves (4 A) were added and set overnight (Sigma Aldrich, St. Louis, MO). For the oligonucleotide chain 5-(Ethylthio)-1H-Tetrazole (ETT, 0.6M in acetonitrile, from Sigma Aldrich) was used as activator solution. Coupling times were 6 minutes carried out at 3.0 equivalents for each step. Prior to coupling the support bound oligonucleotide is treated with a solution of Dichloroacetic Acid in Dichloromethane (3% Deblock, Sigma Aldrich) and washed with Anhydrous Acetonitrile.

### Example 34: Cleavage and Deprotection of Support Bound Oligomer

After completion of the solid phase synthesis, the support was treated with AMA solution, a 1:1 volume solution of NH₄OH:CH₃NH₂ (Fisher Scientific, Spectrum Chemicals), for 20 minutes at 65° C. The solution was then evaporated. Prior to purification, in-process analysis is performed on analytical HPLC and LCMS to determine crude purity, identify the target mass and monitor the deprotection for completion.

### Example 35: LCMS Method

A Waters XBridge Oligonucleotide BEH C18 Column, 130A, 2.5 μm, 2.1 mm×50 mm (P/N 186003952) column was used with buffer solutions: 400 mM HFIP+15 mM TEA (buffer A) and 100% methanol (buffer B) at a gradient of 15-40% or 50-75% Buffer B over 15 CV at 70° C. with a flowrate of 1 mL/minute

### Example 36: Concentration by Tangential Flow Filtration (TFF)

The crude oligos are concentrated using Pall Minimate EVO System (Product ID: OAPMPUNV), using a Pall Minimate TFF cassette capsule with 3k Omega membrane.

### Example 37: Purification

Purification was performed using reverse phase HPLC. Waters XBridge Prep C18 5 μm OBD, 250×19 mm (P/N: 186004021). Buffer solution mixtures are 100 mM TEAA, 5% ACN at pH of 7.0 (buffer A) and 1:1 acetonitrile:methanol (buffer B). Gradient was 5-30% or 30-60% Buffer B over 60 minutes at 60° C. with a flowrate of 20 mL/minute. After purification, fractions are analyzed by reverse phase UPLC. The column used is a Waters ACQUITY UPLC Oligonucleotide BEH C18 1.7 μm, 2.1×50 mm (P/N: 186003949). Buffer solution mixtures are 100 mM TEAA, 5% ACN at pH of 7.0 (buffer A) and 1:1 acetonitrile:methanol (buffer B). Gradient was set at 5-30% or 30-60% Buffer B over 5 minutes at 70° C. with a flowrate of 1.0 mL/minute. The minimum spec of the purified pool is 85%.

### Example 38: Desalting

Once a pool has been established, the oligos are desalted using Pall Minimate EVO System (Product ID: OAPMPUNV). Cassette used is the Pall Minimate TFF capsule with 3k Omega membrane (Product ID: OA003C12). Retentate is collected for lyophilization or annealing directly.

### Example 39: General Procedure for Annealing

The concentrations of both sense strand and antisense strand were determined by Nanodrop. The double-stranded siRNA was prepared by mixing equimolar of sense stand and antisense strand. The annealing process was monitored by RP-HPLC, non-denaturing method. After annealing, no more that 5% of antisense strand was in the duplex mixture. Duplex concentration was determined by measuring the solution absorbance on Nanodrop.

### Preparation of Nmda Ligand—Sense Strand Conjugates

Ligands 1-23, as described above, were conjugated to sense strands targeting Target A (regions I and II), Target B (regions I, II and III) and Target C, according to the processes described above.

### Example 40: Preparation of Conjugate 1

Ligand 1 was conjugated to the 5′ end of an oligo sense strand targeting Target A-Region I, according to the general procedures above.

The product was prepared with 99% purity, confirmed by HPLC.

LCMS: m/z: 8098.6 (calc. 8100.0 g/mol)

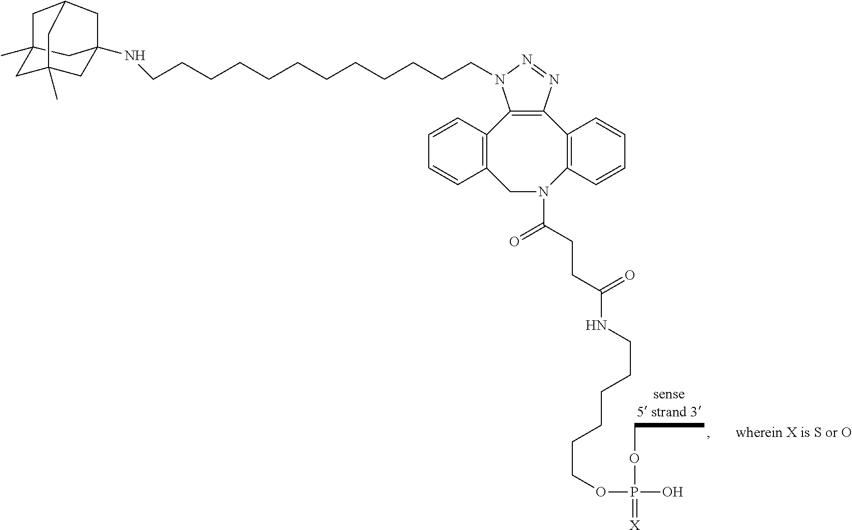

### Example 41: Preparation of Conjugate 2

Ligand 2 was conjugated to the 5′ end of an oligo sense strand targeting Target A-Region I, according to the general procedures above.

The product was prepared with 98% purity, confirmed by HPLC.

LCMS: m/z: 8204.4 (calc. 8206.0 g/mol)

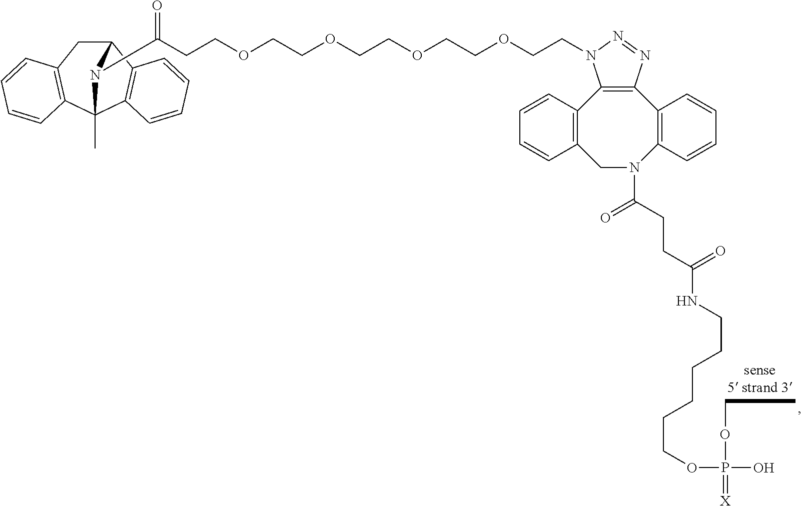

wherein X is S or O

### Example 42: Preparation of Conjugate 3

Ligand 4 was conjugated to the 5′ end of an oligo sense strand targeting Target A-Region I, according to the general procedures above.

The product was prepared with 92% purity, confirmed by HPLC.

LCMS: m/z: 8147.2 (calc. 8148.9 g/mol)

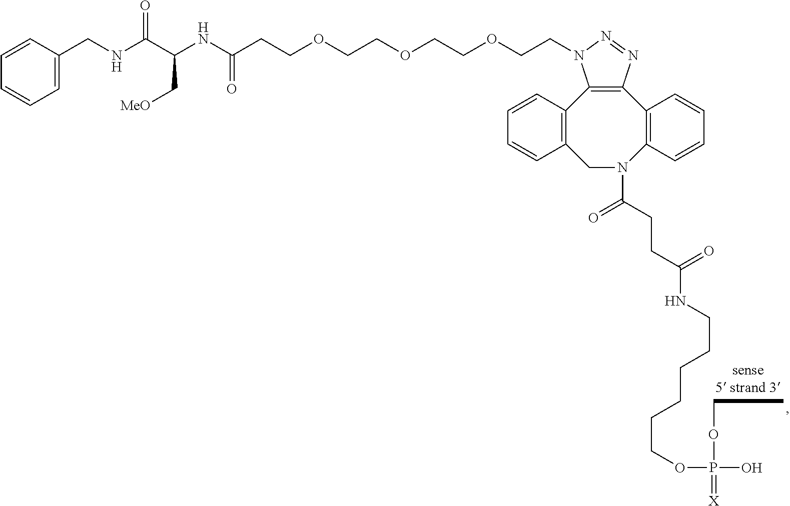

### Example 43: Preparation of Conjugate 4

Ligand 5 was conjugated to the 5′ end of an oligo sense strand targeting Target A-Region I, according to the general procedures above.

The product was prepared with 98% purity, confirmed by HPLC.

LCMS: m/z: 8177.3 (calc. 8178.9 g/mol)

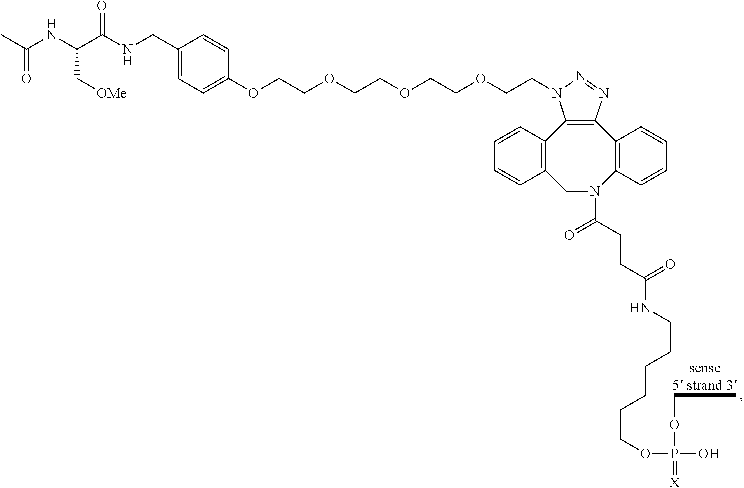

wherein X is S or

### Example 44: Preparation of Conjugate 5

Ligand 6 was conjugated to the 5′ end of an oligo sense strand targeting Target A-Region I, according to the general procedures above.

The product was prepared with 98% purity, confirmed by HPLC.

LCMS: m/z: 8154.2 (calc. 8155.9 g/mol)

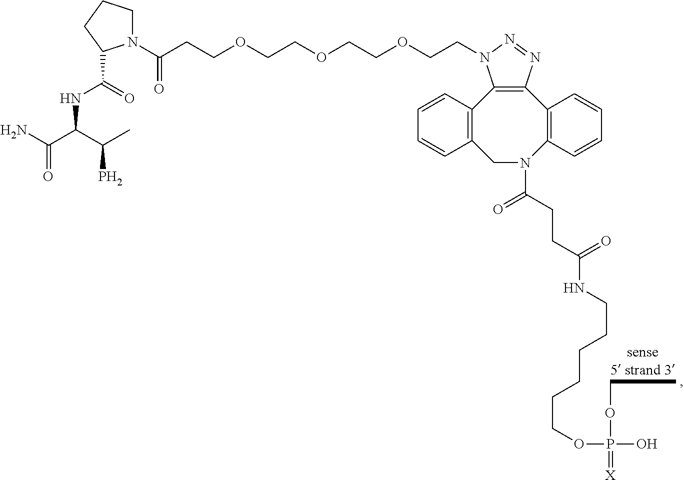

wherein X is S or O

### Example 45: Preparation of Conjugate 6

Ligand 9 was conjugated to the 5′ end of an oligo sense strand targeting Target A-Region I, according to the general procedures above.

The product was prepared with 97% purity, confirmed by HPLC.

LCMS: m/z: 8162.3 (calc. 8164.0 g/mol)

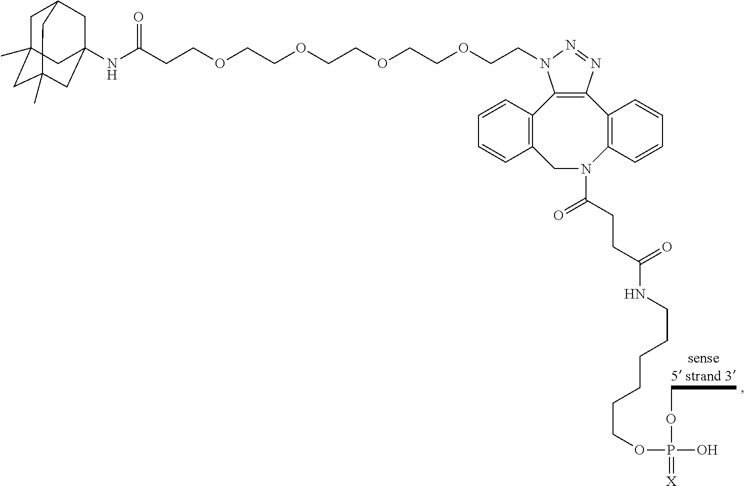

wherein X is S or

### Example 46: Preparation of Conjugate 7

Ligand 1 was conjugated to the 5′ end of an oligo sense strand targeting Target A-Region II, according to the general procedures above.

The product was prepared with 95% purity, confirmed by HPLC.

LCMS: m/z: 8555.7 (calc. 8557.4 g/mol)

### Example 47: Preparation of Conjugate 8

Ligand 2 was conjugated to the 5′ end of an oligo sense strand targeting Target A-Region II, according to the general procedures above.

The product was prepared with 98% purity, confirmed by HPLC.

LCMS: m/z: 8661.6 (calc. 8663.4 g/mol)

### Example 48: Preparation of Conjugate 9

Ligand 2 was conjugated to the 5′ end of an oligo sense strand targeting Target B-Region I, according to the general procedures above.

The product was prepared with 92% purity, confirmed by HPLC.

LCMS: m/z: 8406.6 (calc. 8408.1 g/mol)

### Example 49: Preparation of Conjugate 10

Ligand 2 was conjugated to the 5′ end of an oligo sense strand targeting Target B-Region II, according to the general procedures above.

The product was prepared with 86% purity, confirmed by HPLC.

LCMS: m/z: 8440.4 (calc. 8442.5 g/mol)

### Example 50: Preparation of Conjugate 11

Ligand 2 was conjugated to the 5′ end of an oligo sense strand targeting Target B-Region III, according to the general procedures above.

The product was prepared with 94% purity, confirmed by HPLC.

LCMS: m/z: 8356.4 (calc. 8358.1 g/mol)

### Example 51: Preparation of Conjugate 12

Ligand 1 was conjugated to the 5′ end of an oligo sense strand targeting Target C-Region I, according to the general procedures above.

The product was prepared with 95% purity, confirmed by HPLC.

LCMS: m/z: 7687.6 (calc. 7688.7 g/mol)

### Example 52: Preparation of Conjugate 13

Ligand 13 was conjugated to the 5′ end of an oligo sense strand targeting Target A-Region I, according to the general procedures above.

The product was prepared with 89% purity, confirmed by HPLC.

LCMS: m/z: 8797.7 (calc. 8799.5 g/mol)

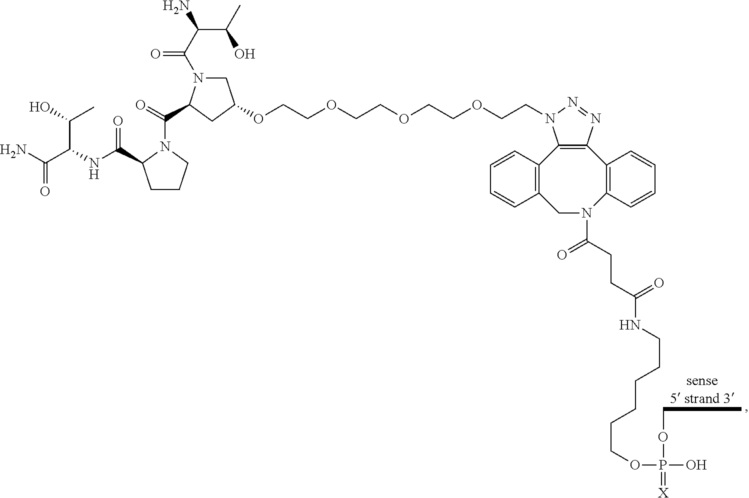

wherein X is S or

### Example 53: Preparation of Conjugate 14

Ligand 14 was conjugated to the 5′ end of an oligo sense strand targeting Target A-Region II, according to the general procedures above.

The product was prepared with 90% purity, confirmed by HPLC.

LCMS: m/z: 8660.8 (calc. 8662.4 g/mol)

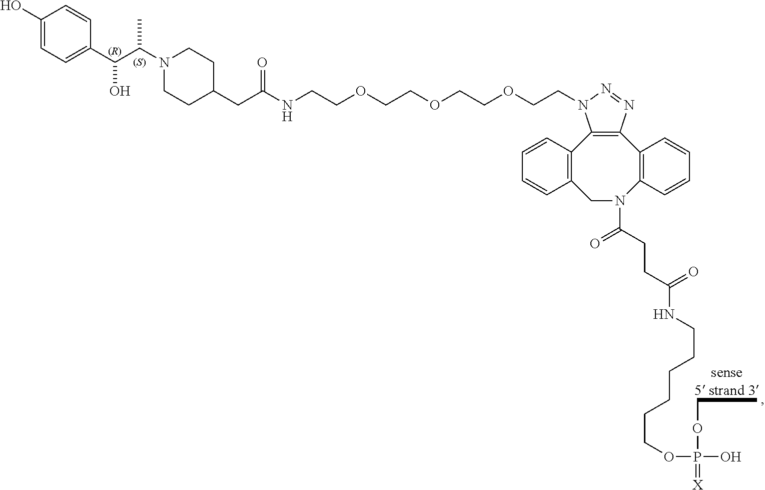

wherein X is S or

### Example 54: Preparation of Conjugate 15

Ligand 15 was conjugated to the 5′ end of an oligo sense strand targeting Target A-Region II, according to the general procedures above.

The product was prepared with 94% purity, confirmed by HPLC.

LCMS: m/z: 8660.9 (calc. 8662.4 g/mol)

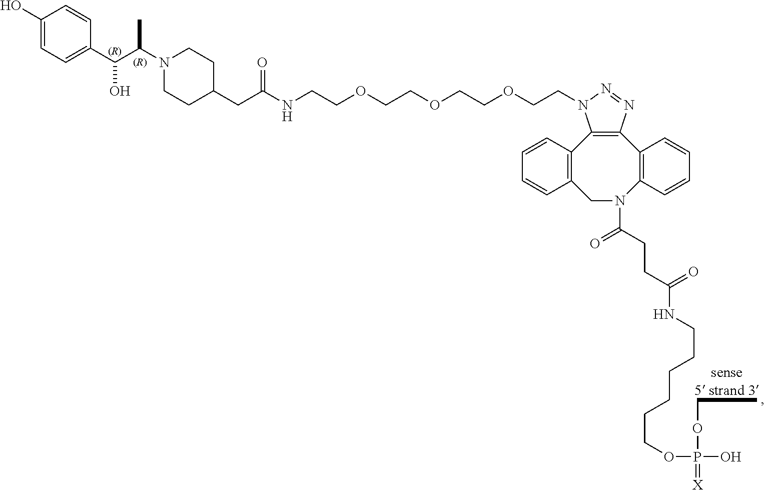

wherein X is S or

### Example 55: Preparation of Conjugate 16

Ligand 10 was conjugated to the 5′ end of an oligo sense strand targeting Target A-Region II, according to the general procedures above.

The product was prepared with 90% purity, confirmed by HPLC.

LCMS: m/z: 8661.6 (calc. 8663.1 g/mol)

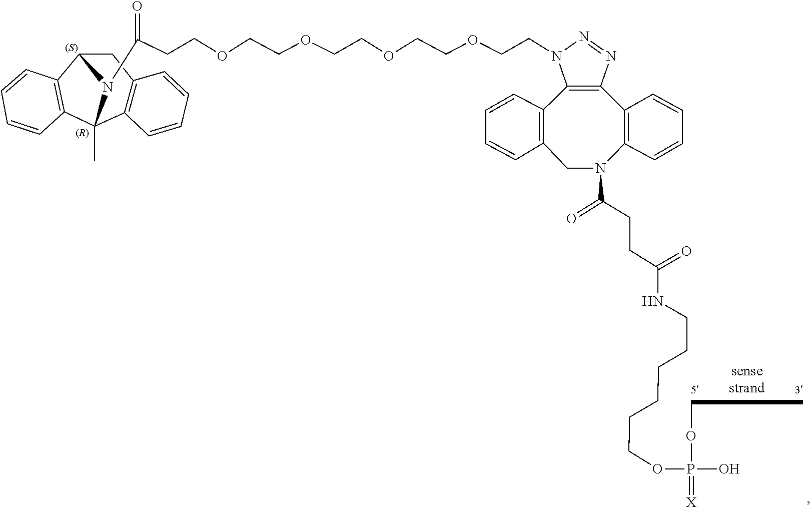

wherein X is S or

### Example 56: Preparation of Conjugate 17

Ligand 7 was conjugated to the 5′ end of an oligo sense strand targeting Target A-Region II, according to the general procedures above.

The product was prepared with 89% purity, confirmed by HPLC.

LCMS: m/z: 8592.0 (calc. 8593.7 g/mol)

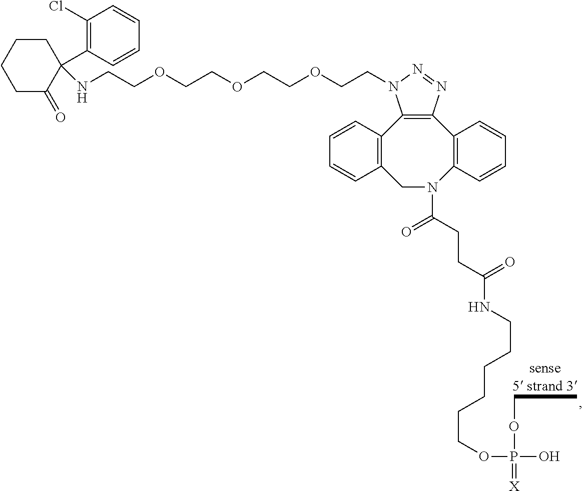

wherein X is S or O

### Example 57: Preparation of Conjugate 18

Ligand 8 was conjugated to the 5′ end of an oligo sense strand targeting Target A-Region II, according to the general procedures above.

The product was prepared with 93% purity, confirmed by HPLC.

LCMS: m/z: 8592.0 (calc. 8593.7 g/mol)

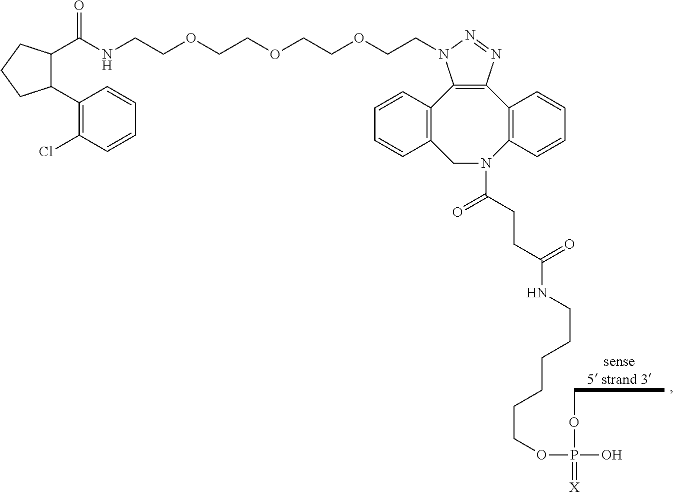

wherein X is S or O

### Example 58: Preparation of Conjugate 19

Ligand 1 was conjugated to the 5′ end of an oligo sense strand targeting Target A-Region II, according to the general procedures above.

The product was prepared with 98% purity, confirmed by HPLC.

LCMS: m/z: 8696.2 (calc. 8697.8 g/mol)

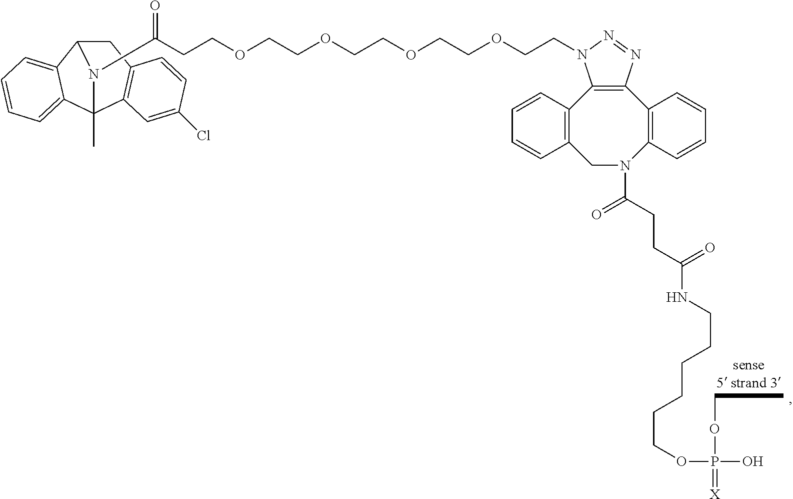

wherein X is S or O

### Example 59: Preparation of Conjugate 20

Ligand 2 was conjugated to the 3′ end of an oligo sense strand targeting Target A-Region I, according to the general procedures above.

The product was prepared with 97% purity, confirmed by HPLC.

LCMS: m/z: 8560.1 (calc. 8561.8 g/mol)

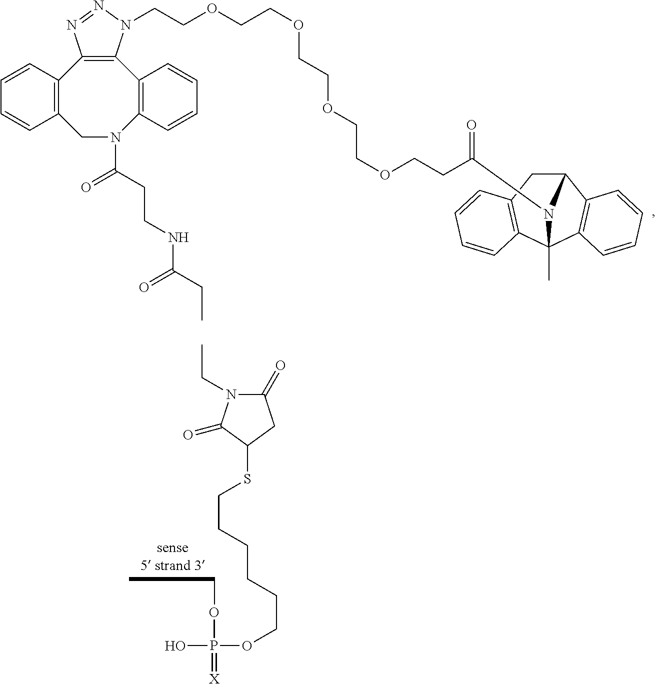

wherein X is S or O

### Example 60: Preparation of Conjugate 21

Ligand 2 was conjugated to the 5′ and 3′ ends of an oligo sense strand targeting Target A-Region II, according to the general procedures above.

The product was prepared with 85% purity, confirmed by HPLC.

LCMS: m/z: 9132.2 (calc. 9134.0 g/mol)

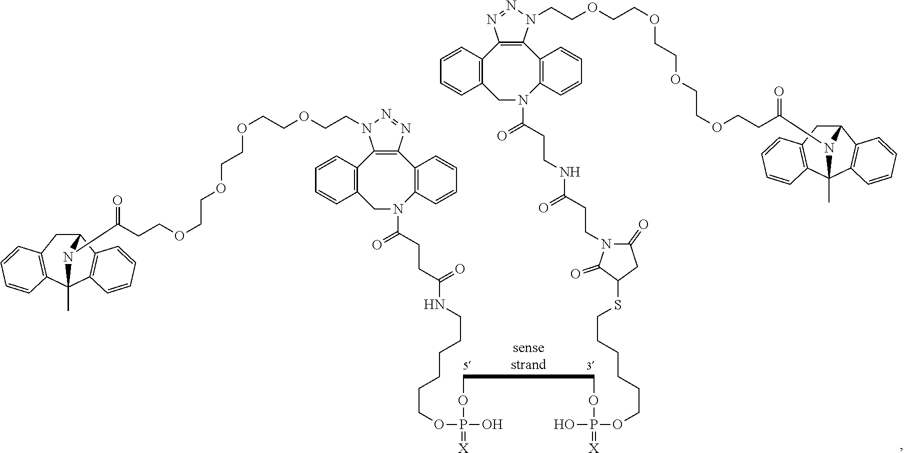

wherein each X is independently S or O

### Example 61: Preparation of Conjugate 22

Ligand 19 was conjugated to the 5′ and 3′ ends of an oligo sense strand targeting Target A-Region I, according to the general procedures above.

The product was prepared with 85% purity, confirmed by HPLC.

LCMS: m/z: 9087.5 (calc. 9088.9 g/mol)

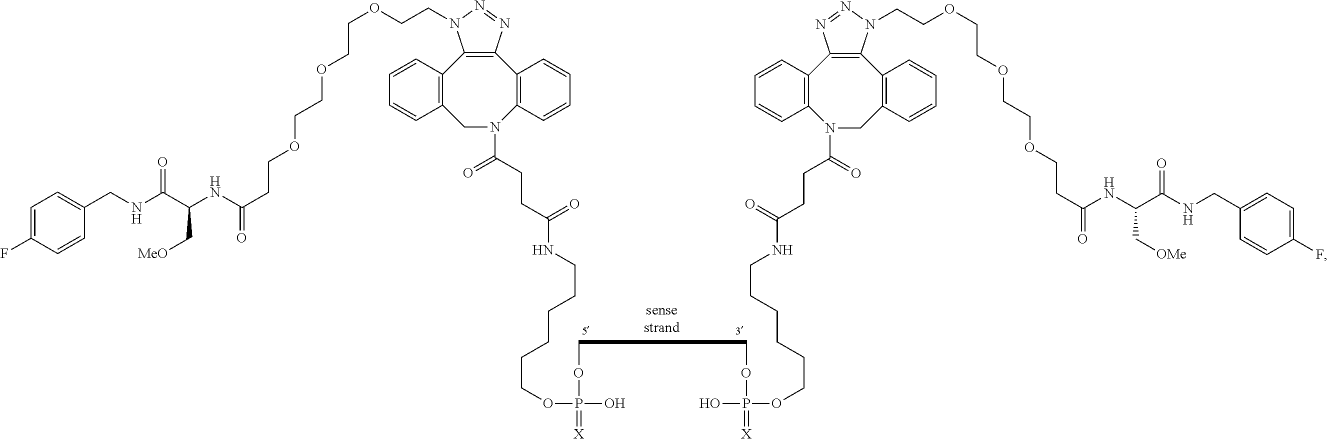

wherein each X is independently S or O

### Example 62: Preparation of Conjugate 23

Ligand 20 was conjugated to the 5′ and 3′ ends of an oligo sense strand targeting Target A-Region I, according to the general procedures above.

The product was prepared with 95% purity, confirmed by HPLC.

LCMS: m/z: 9079.4 (calc. 9081.0 g/mol)

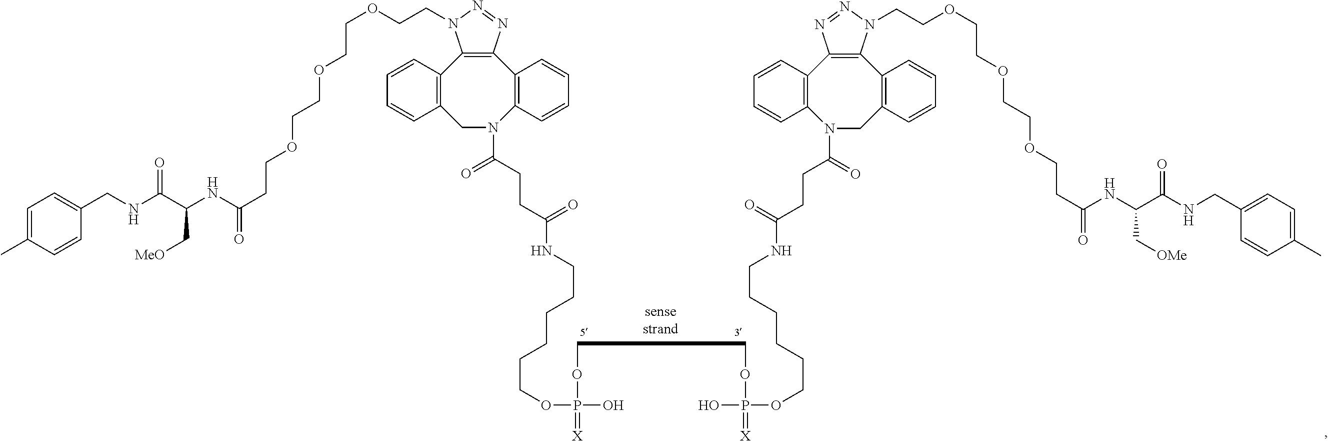

wherein each X is independently S or O

### Example 63: Preparation of Conjugate 24

Ligand 18 was conjugated to the 5′ and 3′ ends of an oligo sense strand targeting Target A-Region I, according to the general procedures above.

The product was prepared with 85% purity, confirmed by HPLC.

LCMS: m/z: 9167.6 (calc. 9169.2 g/mol)

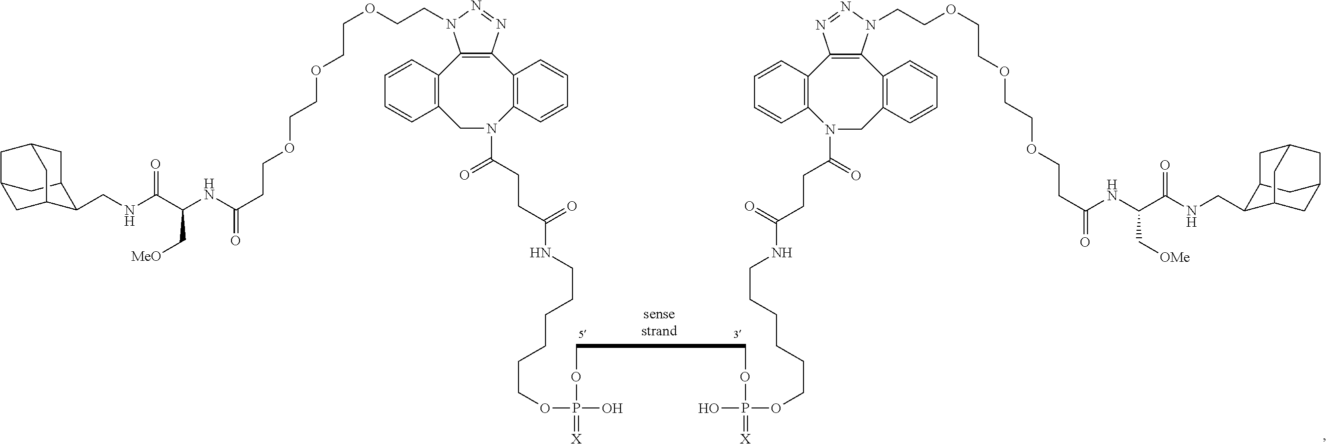

wherein each X is independently S or O

### Example 64: Preparation of Conjugate 25

Ligand 17 was conjugated to the 5′ and 3′ ends of an oligo sense strand targeting Target A-Region I, according to the general procedures above.

The product was prepared with 93% purity, confirmed by HPLC.

LCMS: m/z: 9037.4 (calc. 9039.2 g/mol)

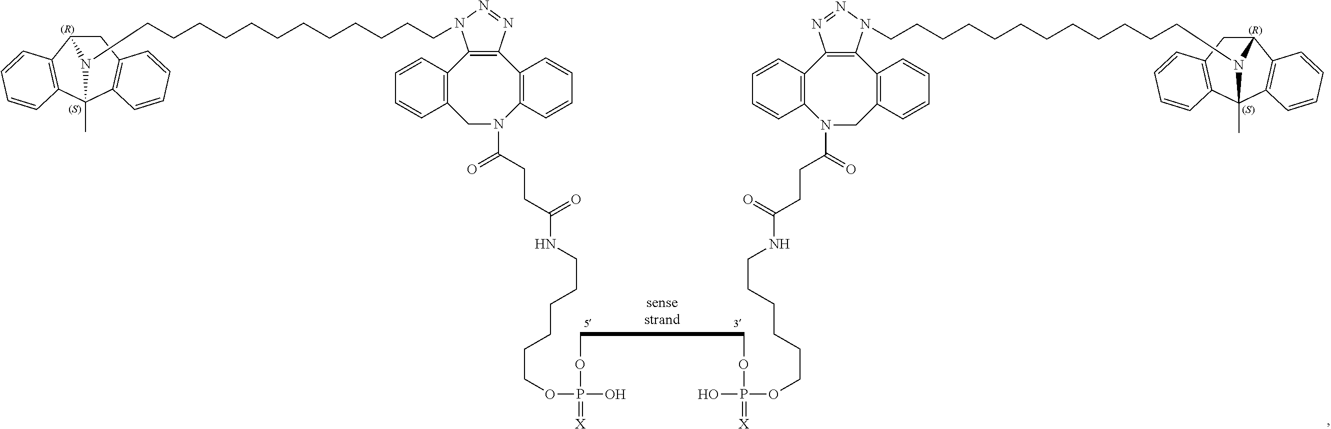

wherein each X is independently S or O

### Example 65: Preparation of Conjugate 26

Ligand 21 was conjugated to the 5′ and 3′ ends of an oligo sense strand targeting Target A-Region I, according to the general procedures above.

The product was prepared with 96% purity, confirmed by HPLC.

LCMS: m/z: 9028.4 (calc. 9029.4 g/mol)

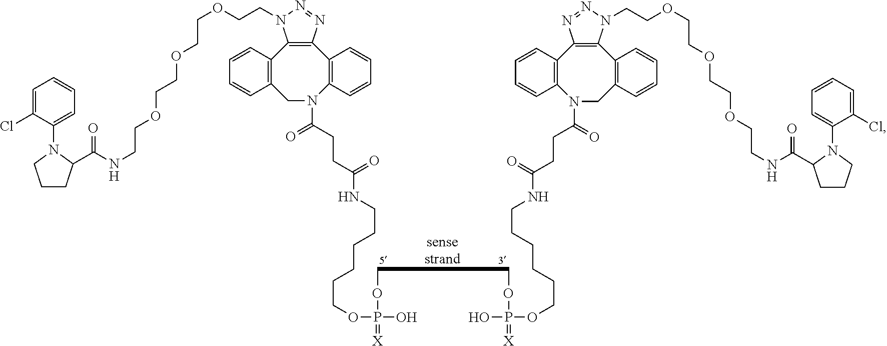

wherein each X is independently S or 0

### Example 66: Preparation of Conjugate 27

Ligand 22 was conjugated to the 5′ and 3′ ends of an oligo sense strand targeting Target A-Region I, according to the general procedures above.

The product was prepared with 93% purity, confirmed by HPLC.

LCMS: m/z: 8998.7 (calc. 8999.7 g/mol)

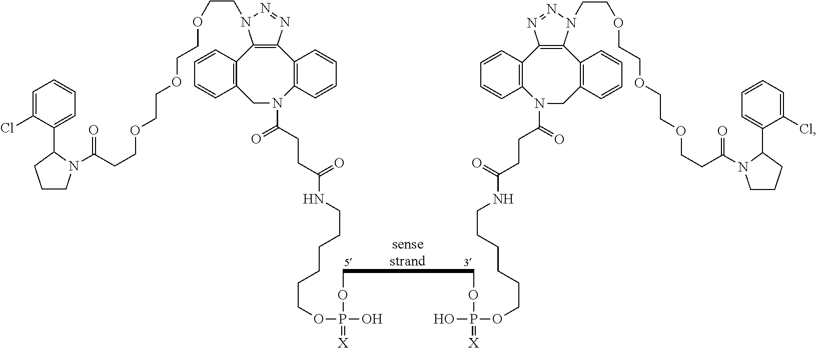

wherein each X is independently S or O

### Example 67: Preparation of Conjugate 28

Ligand 23 was conjugated to the 5′ and 3′ ends of an oligo sense strand targeting Target A-Region I, according to the general procedures above.

The product was prepared with 93% purity, confirmed by HPLC.

LCMS: m/z: 9028.5 (calc. 9029.8 g/mol)

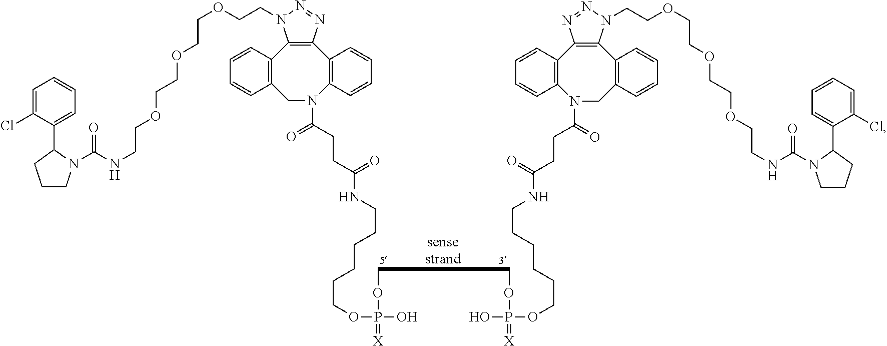

wherein each X is independently S or O

### Example 68: Preparation of Conjugate 29

Ligand 2 was conjugated to the 5′ and 3′ ends of an oligo sense strand targeting Target C-Region II, according to the general procedures above.

The product was prepared with 99% purity, confirmed by HPLC.

LCMS: m/z: 9405.2 (calc. 9406.9 g/mol)

### Example 69: Preparation of Conjugate 30

Ligand 2 was conjugated to the 5′ and 3′ ends of an oligo sense strand targeting Target C-Region III, according to the general procedures above.

The product was prepared with 99% purity, confirmed by HPLC.

LCMS: m/z: 9444.3 (calc. 9445.9 g/mol)

### Example 70: Preparation of Conjugate 31

Ligand 2 was conjugated to the 5′ and 3′ ends of an oligo sense strand targeting Target C-Region IV, according to the general procedures above.

The product was prepared with 97% purity, confirmed by HPLC.

LCMS: m/z: 9459.3 (calc. 9460.8 g/mol)

### Example 71: Preparation of Conjugate 32

Ligand 2 was conjugated to the 5′ and 3′ ends of an oligo sense strand targeting Target C-Region V, according to the general procedures above.

The product was prepared with 98% purity, confirmed by HPLC.

LCMS: m/z: 9451.3 (calc. 9453.0 g/mol)

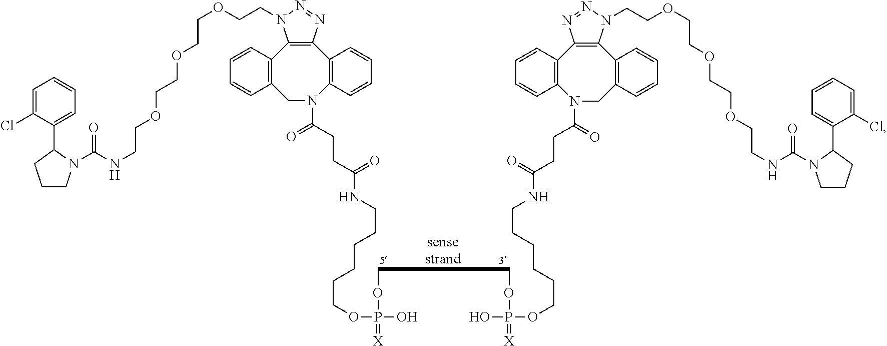

wherein each X is independently S or O

### Example 72: Preparation of Conjugate 33

Ligand 2 was conjugated to the 5′ and 3′ ends of an oligo sense strand targeting Target B-Region I, according to the general procedures above.

The product was prepared with 76% purity, confirmed by HPLC.

LCMS: m/z: 9524.4 (calc. 9526.3 g/mol)

### Example 73: Preparation of Conjugate 34

Ligand 2 was conjugated to the 5′ and 3′ ends of an oligo sense strand targeting Target B-Region II, according to the general procedures above.

The product was prepared with 83% purity, confirmed by HPLC.

LCMS: m/z: 9558.4 (calc. 9560.2 g/mol)

### Example 74: Preparation of Conjugate 35

Ligand 2 was conjugated to the 5′ and 3′ ends of an oligo sense strand targeting Target B-Region VI, according to the general procedures above.

The product was prepared with 88% purity, confirmed by HPLC.

LCMS: m/z: 9620.6 (calc. 9622.3 g/mol)

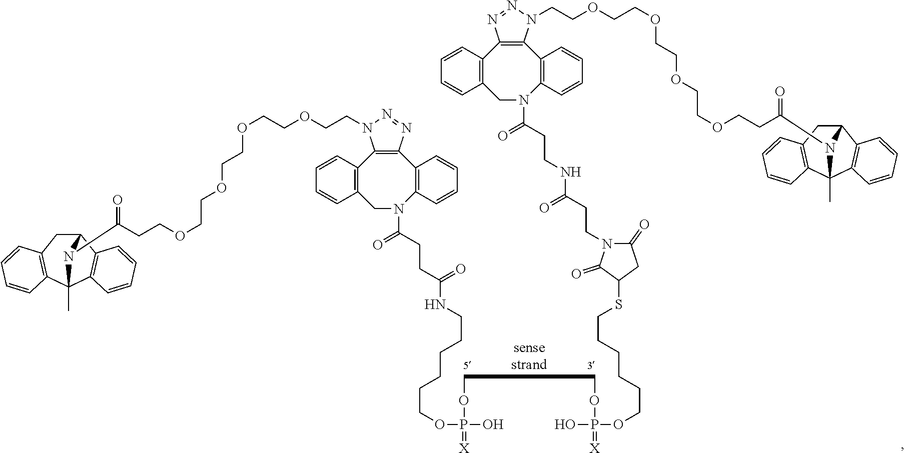

wherein each X is independently S or O

In summary the following conjugates were prepared:

| Conj | Ligand | Target | purity | MW (Obs/Calc) |
|---|---|---|---|---|
| 1 | Ligand 1 | A-I | 99% | 8098.6/8100.0 |
| 2 | Ligand 2 | A-I | 98% | 8206.0/8206.0 |
| 3 | Ligand 4 | A-I | 92% | 8147.2/8148.9 |
| 4 | Ligand 5 | A-I | 98% | 8177.3/8178.9 |
| 5 | Ligand 6 | A-I | 98% | 8154.2/8155.9 |
| 6 | Ligand 9 | A-I | 97% | 8162.3/8164.0 |
| 7 | Ligand 1 | A-II | 95% | 8555.7/8557.4 |
| 8 | Ligand 2 | A-II | 98% | 8661.6/8663.4 |
| 9 | Ligand 2 | B-I | 92% | 8406.6/8408.1 |
| 10 | Ligand 2 | B-II | 86% | 8440.4/8442.5 |
| 11 | Ligand 2 | B-III | 94% | 8356.4/8358.1 |
| 12 | Ligand 1 | C-I | 95% | 7687.6/7688.7 |
| 13 | Ligand 13 | A-I | 89% | 8797.7/8799.5 |
| 14 | Ligand 14 | A-II | 90% | 8660.8/8662.4 |
| 15 | Ligand 15 | A-II | 94% | 8660.9/8662.4 |
| 16 | Ligand 10 | A-II | 90% | 8661.6/8663.1 |
| 17 | Ligand 7 | A-II | 89% | 8592.0/8593.7 |
| 18 | Ligand 8 | A-II | 93% | 8592.0/8593.7 |
| 19 | Ligand 16 | A-II | 98% | 8696.2/8697.8 |
| 20 | Ligand 2 | A-I | 97% | 8560.1/8561.8 |
| 21 | Ligand 2 | A-II | 85% | 9132.2/9134.0 |
| 22 | Ligand 19 | A-I | 85% | 9087.5/9088.9 |
| 23 | Ligand 20 | A-I | 95% | 9079.4/9081.0 |
| 24 | Ligand 18 | A-I | 85% | 9167.6/9169.2 |
| 25 | Ligand 17 | A-I | 93% | 9037.4/9039.2 |
| 26 | Ligand 21 | A-I | 96% | 9028.4/9029.4 |
| 27 | Ligand 22 | A-I | 93% | 8998.7/8999.7 |
| 28 | Ligand 23 | A-I | 93% | 9028.5/9029.8 |
| 29 | Ligand 23 | C-II | 99% | 9405.2/9406.9 |
| 30 | Ligand 23 | C-III | 99% | 9444.3/9445.9 |
| 31 | Ligand 23 | C-IV | 97% | 9459.3/9460.8 |
| 32 | Ligand 23 | C-V | 98% | 9451.3/9453.0 |
| 33 | Ligand 2 | B-I | 76% | 9524.4/9526.3 |
| 34 | Ligand 2 | B-II | 83% | 9558.4/9560.2 |
| 35 | Ligand 2 | B-II | 88% | 9620.6/9622.3 |

### In Vivo Animal Studies

NMDA conjugate compounds were evaluated in vivo. RNA was collected from various brain regions as indicated below, and Target inhibition measured.

### Brain Region Abbrev.

Striatum: STR Hippocampus: HPC Frontal Cortex: CTX Cerebellum: CBM Cervical spinal cord: CERV Midbrain: MB

### Example 75: In Vivo Human Target a Transgenic Mice PD Study

NMDA conjugate compounds were evaluated in an in vivo human Target A transgenic mice PD study. The animals received a single vehicle or 0.2 mg (10 mg/kg) dose via intracerebroventricular injection on day 1 (n=3/group). Animals were observed every day for behavioral changes. Brain regions were collected on day 15, and tissue was immediately placed in homogenizing tube, snap frozen, then kept at −80° C. for gene expression analysis.

RNA Isolation was performed according to the RNeasy Micro Kit (Qiagen Cat #74004) instructions. Following RNA isolation, a 96-well plate was placed on ice while the qRT-PCR reaction was prepared. 2 μl of RNA was added to the reaction mixture containing 5 μl TaqMan Fast Virus 1-Step Master Mix (Thermo Fisher #44444432), 1 μl Target A TaqMan Gene Expression Assay (Thermo Fisher), 1 μl mouse GAPDH (VIC) TaqMan Gene Expression Assay (Thermo Fisher:Mm99999915_g1, VIC), and 11 μl RT-PCR grade nuclease-free water in a MicroAmp Optical 96-well plate (0.2 mL). qPCR was performed using a QuantStudio3 qPCR machine with the following cycles: 50° C. for 1 minute, 95° C. for 20 seconds, 40 cycles at 95° C. for 15 seconds, and 60° C. for 1 minute. Results are presented in the table below as percent inhibition of Target A, relative to vehicle control.

| | Target A | | | | | |
|---|---|---|---|---|---|---|
| Conjugate | Region | CTX | STR | HPC | CBM | CERV |
| 1 | I | 56 | 74 | 74 | 53 | 48 |
| 2 | I | 73 | 77 | 79 | 47 | 51 |
| 3 | I | 61 | 68 | 73 | 51 | 72 |
| 4 | I | 47 | 55 | 54 | 32 | 44 |
| 5 | I | 52 | 62 | 61 | 53 | 61 |
| 6 | I | 53 | 47 | 49 | 37 | 50 |
| 7 | II | 54 | 43 | 57 | 47 | 42 |
| 8 | II | 65 | 49 | 62 | 51 | 49 |
| 13 | II | 47 | 39 | 54 | 41 | 48 |
| 14 | II | 47 | 39 | 54 | 38 | 44 |
| 15 | II | 45 | 39 | 51 | 42 | 45 |
| 16 | II | 65 | 52 | 65 | 46 | 51 |
| 17 | II | 55 | 49 | 62 | 47 | 50 |
| 18 | II | 64 | 56 | 68 | 46 | 50 |
| 19 | II | 44 | 25 | 59 | 7 | 35 |
| 20 | I | 58 | 71 | 77 | 70 | 76 |
| 21 | II | 56 | 42 | 58 | 21 | 48 |
| 22 | I | 54 | 65 | 67 | 57 | 84 |
| 23 | I | 61 | 71 | 71 | 62 | 87 |
| 24 | I | 66 | 74 | 76 | 63 | 72 |
| 25 | I | 70 | 59 | 74 | 69 | 77 |
| 26 | I | 63 | 67 | 75 | 62 | 72 |
| 27 | I | 65 | 69 | 76 | 63 | 71 |
| 28 | I | 67 | 71 | 74 | 62 | 74 |

### Example 76: In Vivo Human Target B Transgenic Mice PD Study

NMDA conjugate compounds were evaluated in an in vivo human Target B transgenic mice PD study. The animals received a single vehicle or 0.2 mg (10 mg/kg) dose via intracerebroventricular injection on day 1 (n=3/group). Animals were observed every day for behavioral changes. Brain regions were collected on day 15, and tissue was immediately placed in homogenizing tube, snap frozen, then kept at −80° C. for gene expression analysis.

RNA Isolation was performed according to the RNeasy Micro Kit (Qiagen Cat #74004) instructions. Following RNA isolation, a 96-well plate was placed on ice while the qRT-PCR reaction was prepared. 2 μl of RNA was added to the reaction mixture containing 5 μl TaqMan Fast Virus 1-Step Master Mix (Thermo Fisher #44444432), 1 μl Target B TaqMan Gene Expression Assay (Thermo Fisher), 1 μl mouse GAPDH (VIC) TaqMan Gene Expression Assay (Thermo Fisher:Mm99999915_g1, VIC), and 11 μl RT-PCR grade nuclease-free water in a MicroAmp Optical 96-well plate (0.2 mL). qPCR was performed using a QuantStudio3 qPCR machine with the following cycles: 50° C. for 1 minute, 95° C. for 20 seconds, 40 cycles at 95° C. for 15 seconds, and 60° C. for 1 minute. Results are presented in the table below as percent inhibition of Target B, relative to vehicle control.

| | Target B | | | | | | |
|---|---|---|---|---|---|---|---|
| Conjugate | Region | CTX | STR | MB | CERV | CERV | MB |
| 9 | I | 6 | 41 | | | 29 | 23 |
| 10 | II | 56 | 64 | 46 | 62 | | |
| 11 | III | 19 | 9 | 6 | 31 | | |
| 33 | I | 29 | 14 | | | 30 | 30 |
| 35 | IV | 39 | 9 | | | 45 | 30 |
| 34 | II | 33 | 23 | | | 33 | 38 |

### Example 77: In Vivo Rat PD Study

NMDA conjugate compounds were evaluated in an in vivo rat PD study. The animals received a single vehicle or 0.9 mg (3 mg/kg) dose via intracisternal magna (ICM) injection on day 1 (n=3/group). Animals were observed every day for behavioral changes. Brain regions were collected on day 15 and tissue was immediately placed in a homogenizing tube, snap frozen, and maintained at −80° C. for gene expression analysis.

RNA Isolation was performed according to the RNeasy Micro Kit (Qiagen Cat #74004) instructions. Following RNA isolation, a 96-well plate was placed on ice while the qRT-PCR reaction was prepared. 2 μl of RNA was added to the reaction mixture containing 5 μl TaqMan Fast Virus 1-Step Master Mix (Thermo Fisher #44444432), 1 μl rat Target C TaqMan Gene Expression Assay (Thermo Fisher), 1 μl ACTB (VIC) TaqMan Gene Expression Assay (Thermo Fisher:Rn00667869_m1, VIC), and 11 μl RT-PCR grade nuclease-free water in a MicroAmp Optical 96-well plate (0.2 mL). qPCR was performed using a QuantStudio3 qPCR machine with the following cycles: 50° C. for 1 minute, 95° C. for 20 seconds, 40 cycles at 95° C. for 15 seconds, and 60° C. for 1 minute. Results are presented in the table below as percent inhibition of Target C, relative to vehicle control.

| Conjugate | CTX | STR | HPC |
|---|---|---|---|
| 12 | 83 | 69 | 54 |

### INCORPORATION BY REFERENCE

The contents of all references (including literature references, issued patents, published patent applications, and co-pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments described herein. Such equivalents are intended to be encompassed by the following claims.

### Embodiments

Additional embodiments include:

Embodiment P1. A compound comprising the structure of Formula (I), or a salt thereof:

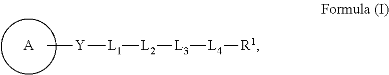

wherein

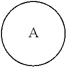

is an N-methyl-D-aspartate (NMDA) receptor ligand;
each of L₁, L₂, L₃, and L₄ is independently a linker, a bond, or absent;
Y is a bond or —C(═O)—; and
R¹ is one or more oligonucleotides, protecting groups, small molecules, proteins, antibodies, peptides, or combinations thereof.

Embodiment P2. The compound, or salt thereof, of embodiment P1, wherein the NMDA receptor ligand is an NMDA receptor agonist.

Embodiment P3. The compound, or salt thereof, of embodiment P1, wherein the NMDA receptor ligand is an NMDA receptor antagonist.

Embodiment P4. The compound, or salt thereof, of embodiment P1, wherein the NMDA receptor ligand is selected from the group consisting of:

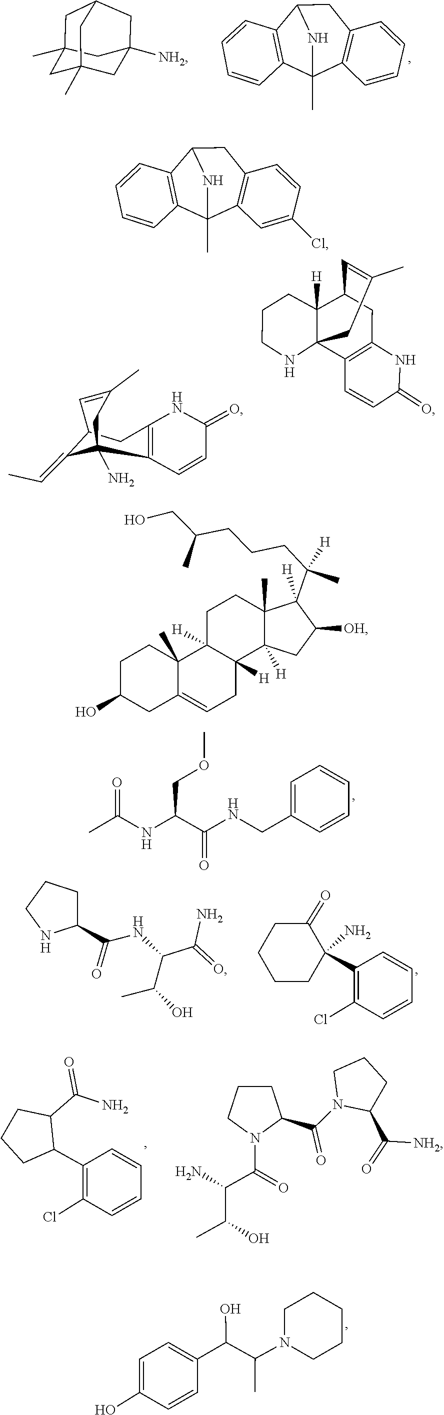

an anti-NMDA receptor antibody, and derivatives thereof.

Embodiment P5. The compound, or salt thereof, of embodiment P1, wherein the compound comprises the structure of Formula (II):

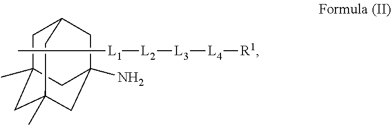

or a salt thereof.

Embodiment P6. The compound, or salt thereof, of embodiment P5, wherein the compound comprises the structure of Formula (II-a):

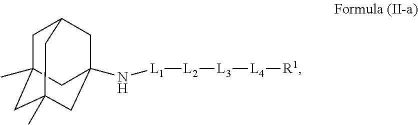

or a salt thereof.

Embodiment P7. The compound, or salt thereof, of embodiment P1, wherein the compound comprises the structure of Formula (III) and R² is hydrogen, halogen, —OH, or —OMe:

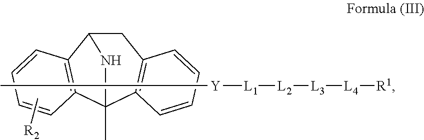

or a salt thereof.

Embodiment P8. The compound, or salt thereof, of embodiment P7, wherein the compound comprises the structure of Formula (III-e):

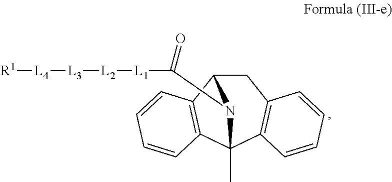

or a salt thereof.

Embodiment P9. The compound, or salt thereof, of embodiment P7, wherein the compound comprises the structure of Formula (III-f):

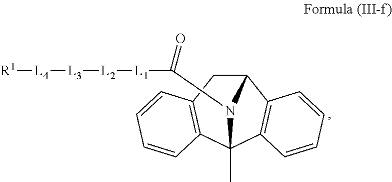

or a salt thereof.

Embodiment P10. The compound, or salt thereof, of embodiment P7, wherein the compound comprises the structure of Formula (III-g):

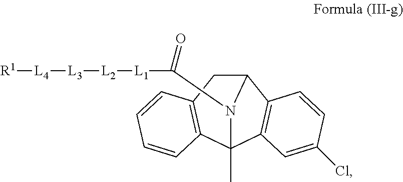

or a salt thereof.

Embodiment P11. The compound, or salt thereof, of embodiment P1, wherein the compound comprises the structure of Formula (IV):

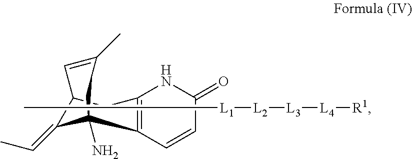

or a salt thereof.

Embodiment P12. The compound, or salt thereof, of embodiment P11, wherein the compound comprises the structure of Formula (IV-a):

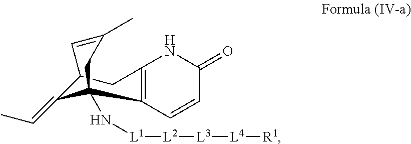

or a salt thereof.

Embodiment P13. The compound, or salt thereof, of embodiment P11, wherein the compound comprises the structure of Formula (IV-b):

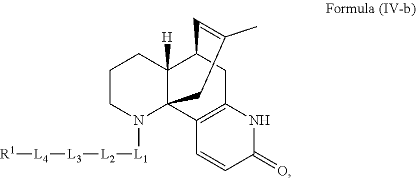

or a salt thereof.

Embodiment P14. The compound, or salt thereof, of embodiment P1, wherein the compound comprises the structure of Formula (V):

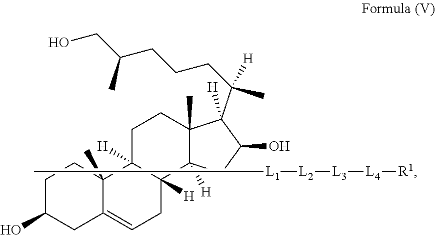

or a salt thereof.

Embodiment P15. The compound, or salt thereof, of embodiment P14, wherein the compound comprises the structure of Formula (V-a):

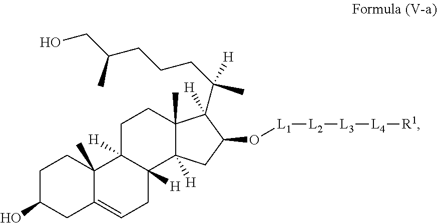

or a salt thereof.

Embodiment P16. The compound, or salt thereof, of embodiment P1, wherein the compound comprises the structure of Formula (VI):

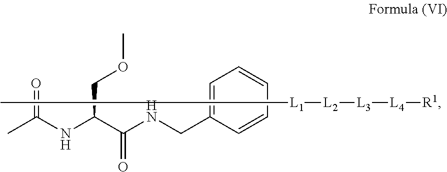

or a salt thereof.

Embodiment P17. The compound, or salt thereof, of embodiment P16, wherein the compound comprises the structure of Formula (VI-a):

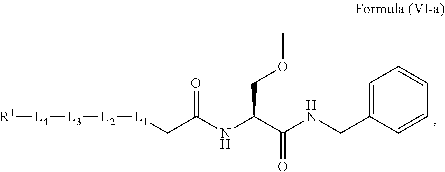

or a salt thereof.

Embodiment P18. The compound, or salt thereof, of embodiment P16, wherein the compound comprises the structure of Formula (VI-b):

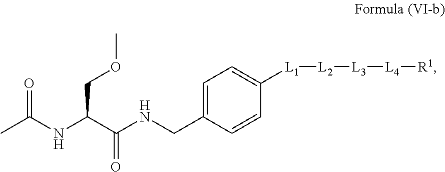

or a salt thereof.

Embodiment P19. The compound, or salt thereof, of embodiment P1, wherein the compound comprises the structure of Formula (VII):

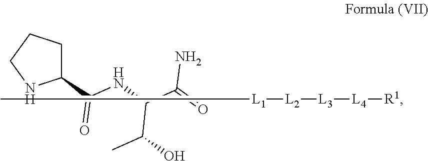

or a salt thereof.

Embodiment P20. The compound, or salt thereof, of embodiment P19, wherein the compound comprises the structure of Formula (VII-a):

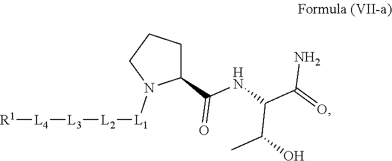

or a salt thereof.

Embodiment P21. The compound, or salt thereof, of embodiment P1, wherein the compound comprises the structure of Formula (VIII):

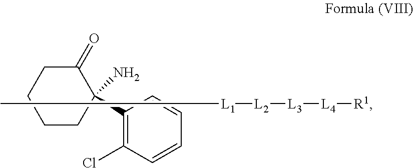

or a salt thereof.

Embodiment P22. The compound, or salt thereof, of embodiment P21, wherein the compound comprises the structure of Formula (VIII-a):

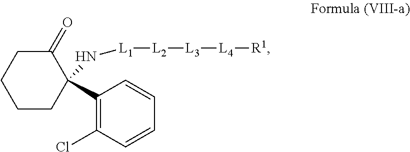

or a salt thereof.

Embodiment P23. The compound, or salt thereof, of embodiment P1, wherein the compound comprises the structure of Formula (IX):

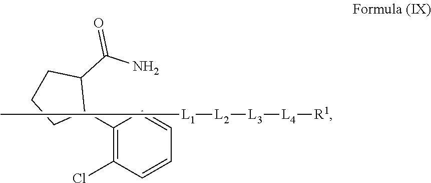

or a salt thereof.

Embodiment P24. The compound, or salt thereof, of embodiment P23, wherein the compound comprises the structure of Formula (IX-a):

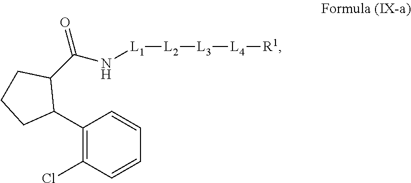

or a salt thereof.

Embodiment P25. The compound, or salt thereof, of embodiment P1, wherein the compound comprises the structure of Formula (X):

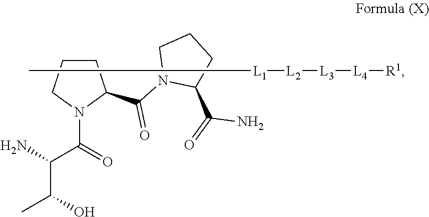

or a salt thereof.

Embodiment P26. The compound, or salt thereof, of embodiment P25, wherein the compound comprises the structure of Formula (X-a):

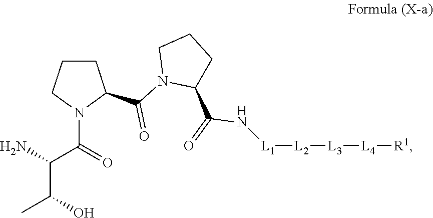

or a salt thereof.

Embodiment P27. The compound, or salt thereof, of embodiment P25, wherein the compound comprises the structure of Formula (X-b):

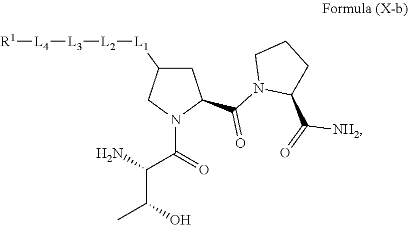

or a salt thereof.

Embodiment P28. The compound, or salt thereof, of embodiment P1, wherein the compound comprises the structure of Formula (XI):

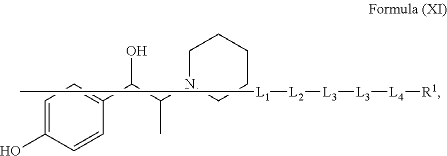

or a salt thereof.

Embodiment P29. The compound, or salt thereof, of embodiment P28, wherein the compound comprises the structure of Formula (XI-a):

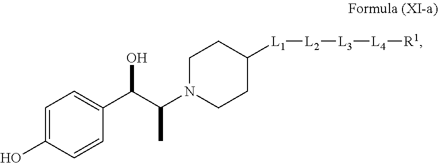

or a salt thereof.

Embodiment P30. The compound, or salt thereof, of embodiment P28, wherein the compound comprises the structure of Formula (XI-b):

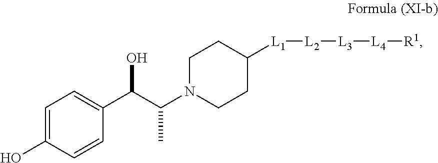

or a salt thereof.

Embodiment P31. The compound, or salt thereof, of any one of embodiments P1-P30, wherein each of L₁, L₂, L₃, and L₄ is independently absent, a bond, an optionally substituted alkyl linker, an optionally substituted polyethylene glycol (PEG) linker, an optionally substituted heteroalkyl linker, an optionally substituted heteroaryl linker, a phosphodiester bond, or a phosphorothioate bond.

Embodiment P32. The compound, or salt thereof, of any one of embodiments P5-P31, wherein L₁ is a bond.

Embodiment P33. The compound, or salt thereof, of embodiment P5 or P6, wherein L₂ is an optionally substituted alkyl linker or an optionally substituted PEG linker.

Embodiment P34. The compound, or salt thereof, of embodiment P33, wherein L₂ comprises the structure

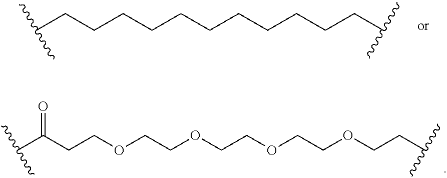 [SMILES: *CCCCCCCCCCC* ; *CCOCCOCCOCCOCCC(*)=O]

Embodiment P35. The compound, or salt thereof, of any one of embodiments P7-P10, wherein L₂ is an optionally substituted PEG linker.

Embodiment P36. The compound, or salt thereof, of embodiment P35, wherein L₂ comprises the structure or

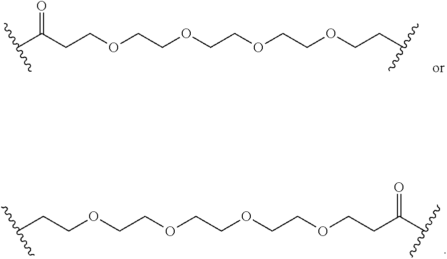 [SMILES: *CCOCCOCCOCCOCCC(*)=O ; *CCOCCOCCOCCOCCC(*)=O]

Embodiment P37. The compound, or salt thereof, of any one of embodiments P11-P13, wherein L₂ is an optionally substituted PEG linker.

Embodiment P38. The compound, or salt thereof, of embodiment P37, wherein L₂ comprises the structure

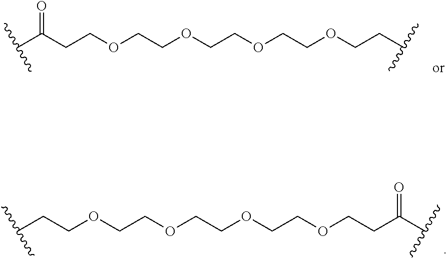 [SMILES: *CCOCCOCCOCCOCCC(*)=O ; *CCOCCOCCOCCOCCC(*)=O]

Embodiment P39. The compound, or salt thereof, of embodiment P14 or P15, wherein L₂ is an optionally substituted heteroalkyl linker.

Embodiment P40. The compound, or salt thereof, of embodiment P39, wherein L₂ comprises the structure

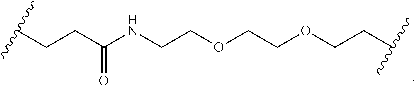 [SMILES: *CCOCCOCCNC(=O)CC*]

Embodiment P41. The compound, or salt thereof, of any one of embodiments P16-P18, wherein L₂ is an optionally substituted PEG linker.

Embodiment P42. The compound, or salt thereof, of embodiment P41, wherein L₂ comprises the structure

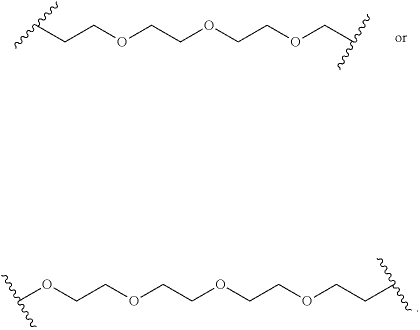 [SMILES: *CCOCCOCCOC* ; *CCOCCOCCOCCO*]

Embodiment P43. The compound, or salt thereof, of embodiment P19 or P20, wherein L₂ is an optionally substituted PEG linker.

Embodiment P44. The compound, or salt thereof, of embodiment P43, wherein L₂ comprises the structure

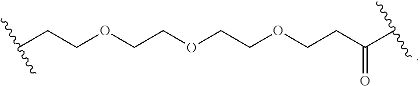 [SMILES: *CCOCCOCCOCCC(*)=O ; "."]

Embodiment P45. The compound, or salt thereof, of any one of embodiments P21-P24, wherein L₂ is an optionally substituted PEG linker.

Embodiment P46. The compound, or salt thereof, of embodiment P45, wherein L₂ comprises the structure

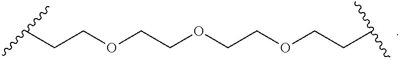 [SMILES: *CCOCCOCCOCC*]

Embodiment P47. The compound, or salt thereof, of any one of embodiments P25-P27, wherein L₂ is an optionally substituted PEG linker.

Embodiment P48. The compound, or salt thereof, of embodiment P47, wherein L₂ comprises the structure

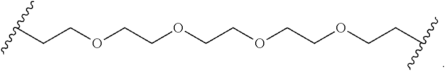 [SMILES: *CCOCCOCCOCCOCC*]

Embodiment P49. The compound, or salt thereof, of any one of embodiments P28-P30, wherein L₂ is an optionally substituted PEG linker.

Embodiment P50. The compound, or salt thereof, of embodiment P49, wherein L₂ comprises the structure

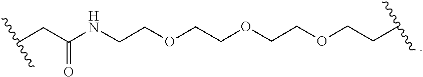 [SMILES: *CCOCCOCCOCCNC(=O)C*]

Embodiment P51. The compound, or salt thereof, of any one of embodiments P5-P50, wherein L₃ is an optionally substituted heteroaryl linker.

Embodiment P52. The compound, or salt thereof, of embodiment P51, wherein L₃ is an optionally substituted partially unsaturated heterocycloalkyl or heteroaryl linker.

Embodiment P53. The compound, or salt thereof, of embodiment P51 or P52, wherein L₃ comprises the structure

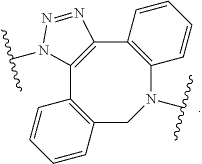 [SMILES: *N1Cc2ccccc2-c2c(nnn2*)-c2ccccc21]

Embodiment P54. The compound, or salt thereof, of any one of embodiments P5-P53, wherein L₄ is an optionally substituted heteroalkyl linker.

Embodiment P55. The compound, or salt thereof, of embodiment P54, wherein the heteroalkyl linker is substituted with one or more ═O substituents.

Embodiment P56. The compound, or salt thereof, of embodiment P54 or P55, wherein L₄ comprises the structure

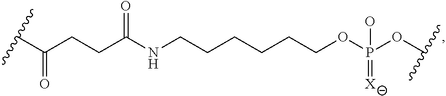

wherein X is O or S.

Embodiment P57. The compound, or salt thereof, of embodiment P5 or P6, wherein L₁, L₂, L₃, and L₄ together comprise the structure

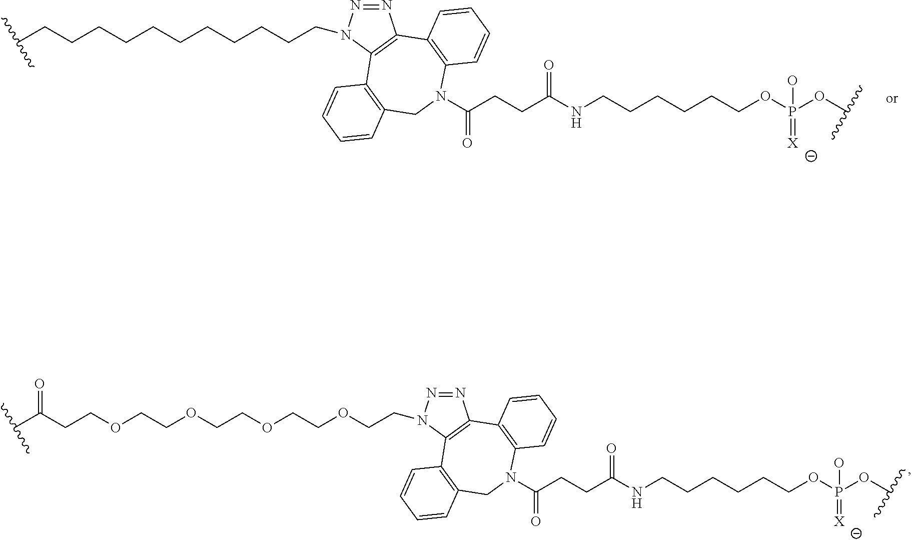

wherein X is O or S.

Embodiment P58. The compound, or salt thereof, of any one of embodiments P7-P10, wherein L₁, L₂, L₃, and L₄ together comprise the structure

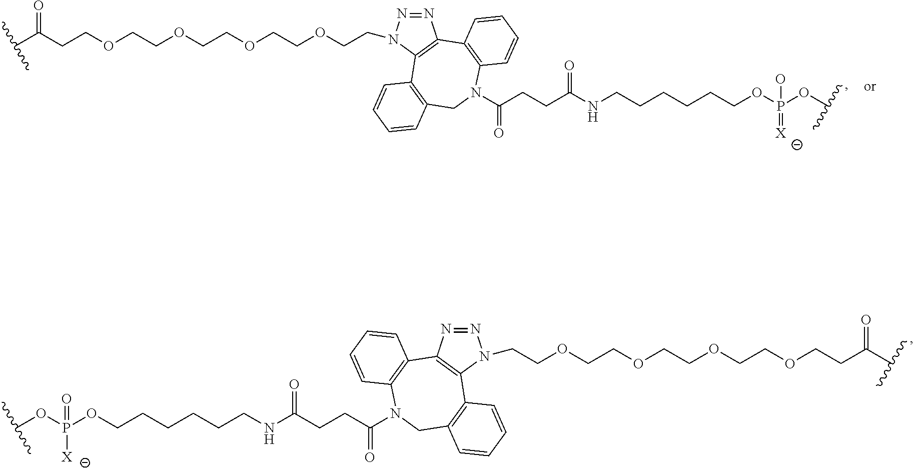

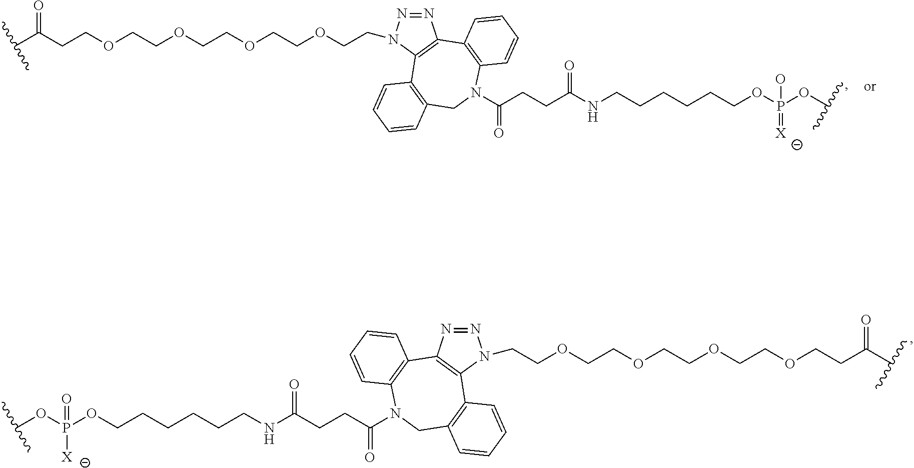

wherein X is O or S.

Embodiment P60. The compound, or salt thereof, of embodiment P14 or P15, wherein L₁, L₂, L₃, and L₄ together comprise the structure

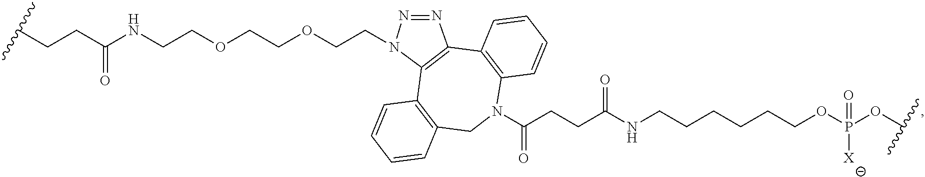

wherein X is O or S.

Embodiment P61. The compound, or salt thereof, of any one of embodiments P16-P18, wherein L₁, L₂, L₃, and L₄ together comprise the structure

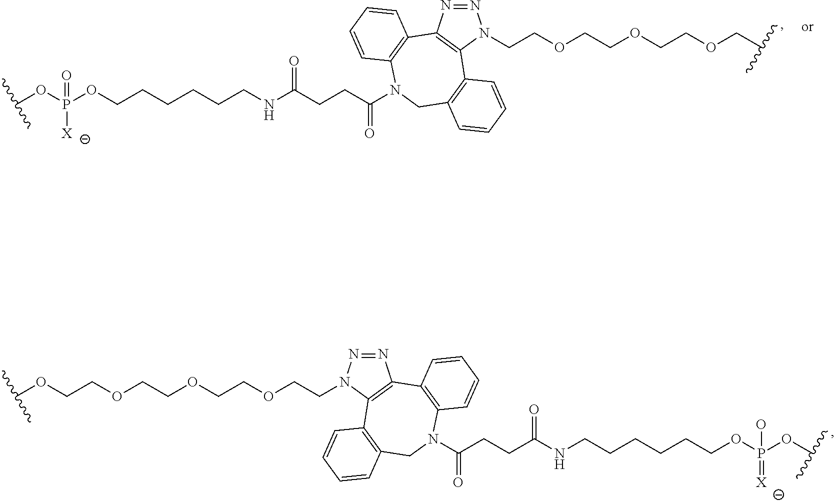

wherein X is O or S.

Embodiment P62. The compound, or salt thereof, of embodiment P19 or P20, wherein L₁, L₂, L₃, and L₄ together comprise the structure

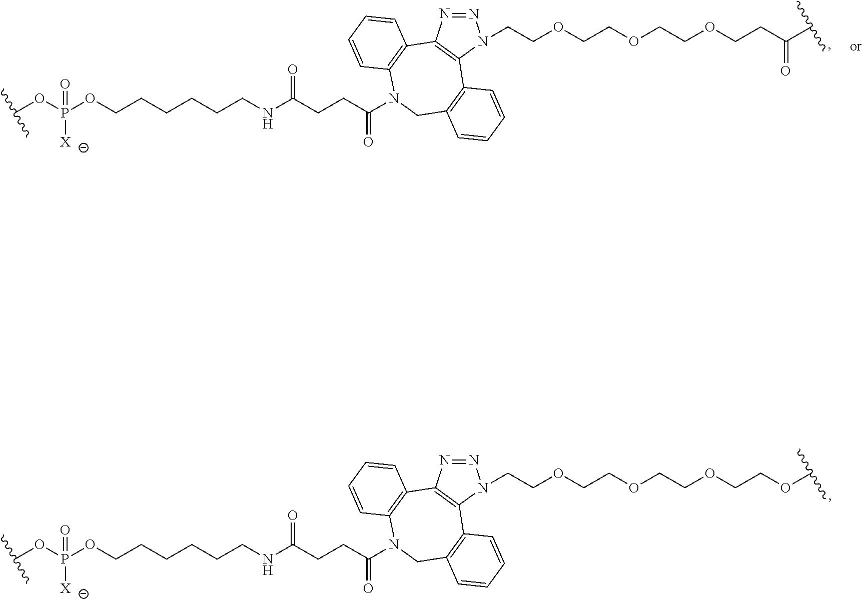

wherein X is O or S.

Embodiment P63. The compound, or salt thereof, of any one of embodiments P21-P24, wherein L₁, L₂, L₃, and L₄ together comprise the structure

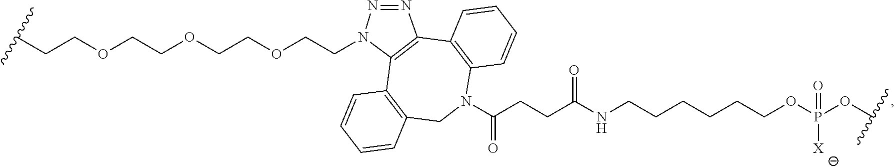

wherein X is O or S.

Embodiment P64. The compound, or salt thereof, of embodiment P25 or P26, wherein L₁, L₂, L₃, and L₄ together comprise the structure

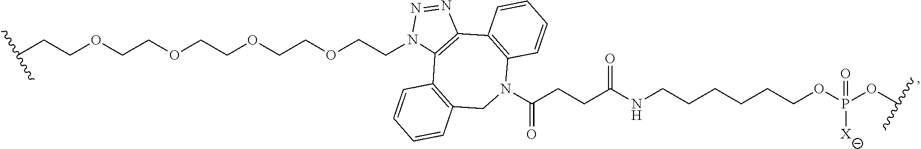

wherein X is O or S.

Embodiment P65. The compound, or salt thereof, of any one of embodiments P1-P64, wherein the compound comprises the structure

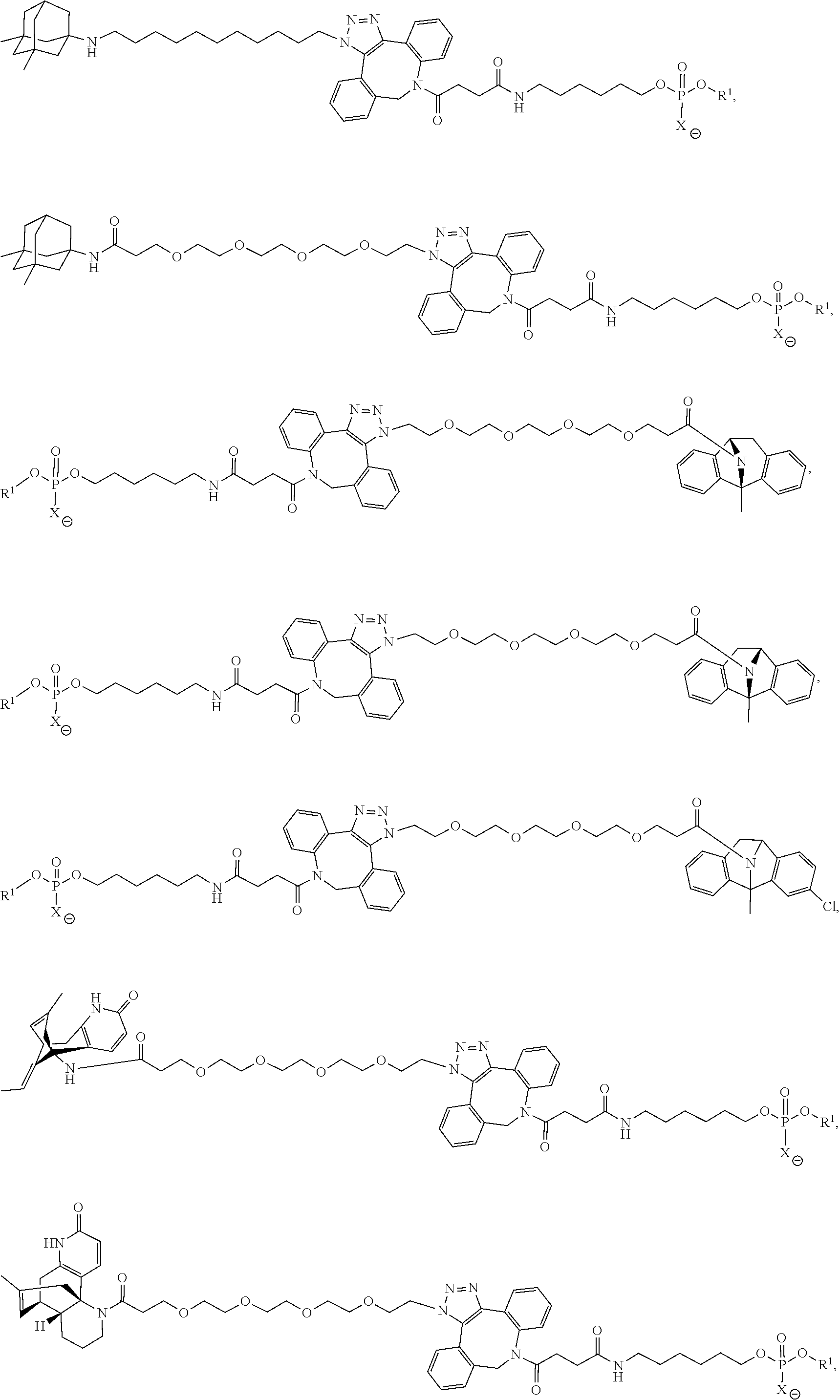

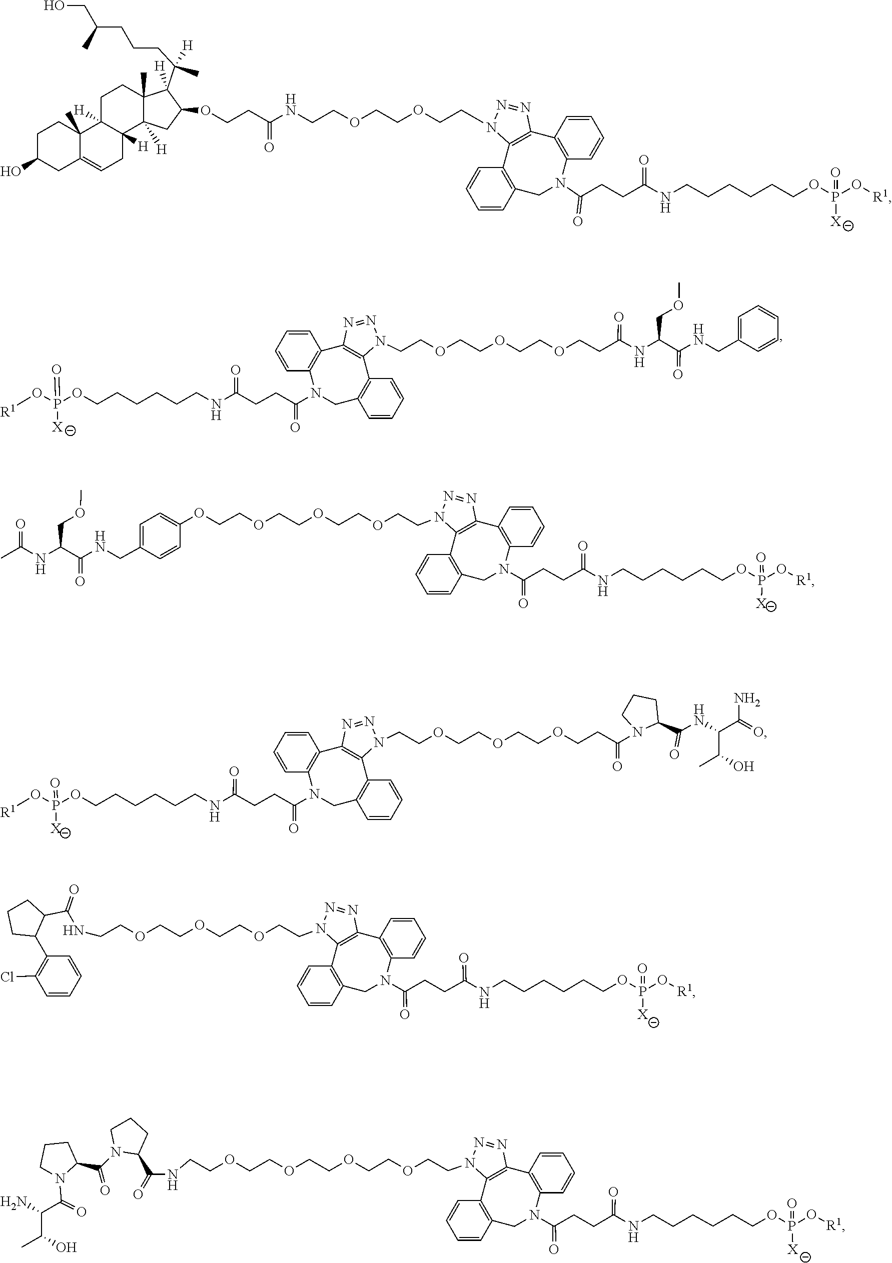

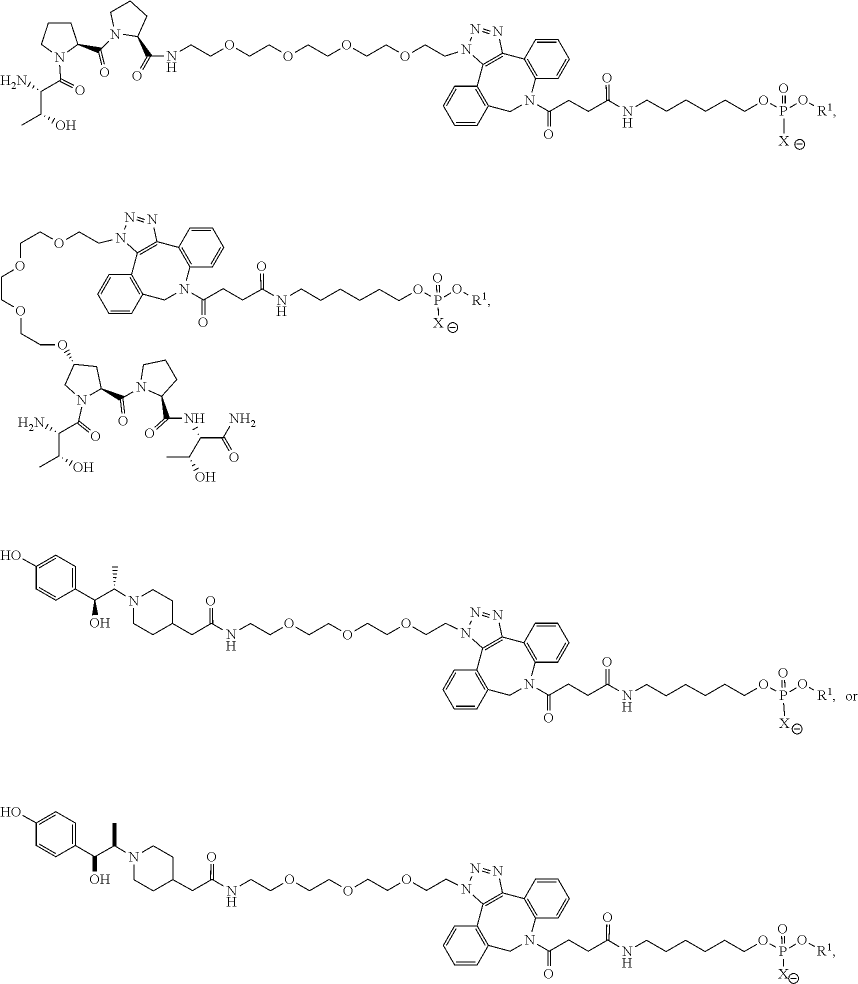

wherein X is O or S.

Embodiment P66. The compound, or salt thereof, of any one of embodiments P56-P65, wherein X is O.

Embodiment P67. The compound, or salt thereof, of any one of embodiments P56-P65, wherein X is S.

Embodiment P68. The compound, or salt thereof, of any one of embodiments P1-P67, wherein R¹ comprises an oligonucleotide.

Embodiment P69. The compound, or salt thereof, of embodiment P68, wherein the oligonucleotide is attached at its 5′ end.

Embodiment P70. The compound, or salt thereof, of embodiment P68, wherein the oligonucleotide is attached at its 3′ end.

Embodiment P71. The compound, or salt thereof, of embodiment P68, wherein the oligonucleotide is attached at an internal position on the oligonucleotide.

Embodiment P72. The compound, or salt thereof, of embodiment P71, wherein the internal position is an internucleoside linkage.

Embodiment P73. The compound, or salt thereof, of any one of embodiments P1-P72, wherein R¹ comprises an oligonucleotide conjugated to one or more additional NMDA receptor ligands.

Embodiment P74. The compound, or salt thereof, of embodiment P73, wherein the oligonucleotide is conjugated to two, three, four, five, or more than five additional NMDA receptor ligands.

Embodiment P75. The compound, or salt thereof, of embodiment P73 or P74, wherein the additional NMDA receptor ligands are conjugated to the oligonucleotide at the 5′ end of the oligonucleotide, the 3′ end of the oligonucleotide, one or more internal positions on the oligonucleotide, or any combination thereof.

Embodiment P76. The compound, or salt thereof, of any one of embodiments P68-P75, wherein the oligonucleotide is a modified oligonucleotide.

Embodiment P77. A composition comprising a compound, or salt thereof, of any one of embodiments P1-P76, and a pharmaceutically acceptable excipient.

Embodiment P78. A method for delivering a therapeutic oligonucleotide to the brain of a subject, comprising administration of a compound, or salt thereof, of any one of embodiments P1-P76, or a composition of embodiment P77, to the subject.

Embodiment P79. The method of embodiment P78, wherein the therapeutic oligonucleotide is delivered to one or more brain regions selected from the group consisting of the striatum, the cerebellum, the brain stem, the hippocampus, the frontal cortex, and the spinal cord.

Embodiment P80. A method for treating or ameliorating a disease, disorder, or symptom thereof in a subject, comprising administration of a compound, or salt thereof, of any one of embodiments P1-P76, or a composition of embodiment P77, to the subject.

Embodiment P81. The method of embodiment P80, wherein the disease, disorder, or symptom thereof is a central nervous system (CNS) disease, disorder, or symptom thereof.

Embodiment P82. The method of embodiment P80 or P81, wherein the disease, disorder, or symptom thereof is Alzheimer's disease, or a symptom thereof.

Embodiment P83. The method of any one of embodiments P80-P82, wherein the compound, or salt thereof, is administered to the subject intrathecally.

Embodiment P84. A method for making a compound, or salt thereof, of any one of embodiments P1-P76, comprising one or more compounds and chemical transformations described herein, including Example 1.

Embodiment 1. A compound comprising the structure of Formula (I), or a salt thereof:

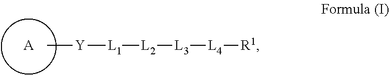

wherein

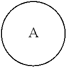

is an N-methyl-D-aspartate (NMDA) receptor ligand;
each of L₁, L₂, L₃, and L₄ is independently a linker, a bond, or absent;
Y is a bond or —C(═O)—; and
R¹ is one or more oligonucleotides, protecting groups, small molecules, proteins, antibodies, peptides, or combinations thereof.

Embodiment 2. The compound, or salt thereof, of embodiment 1, wherein the NMDA receptor ligand is an NMDA receptor agonist.

Embodiment 3. The compound, or salt thereof, of embodiment 1, wherein the NMDA receptor ligand is an NMDA receptor antagonist.

Embodiment 4. The compound, or salt thereof, of embodiment 1, wherein the NMDA receptor ligand is selected from the group consisting of:

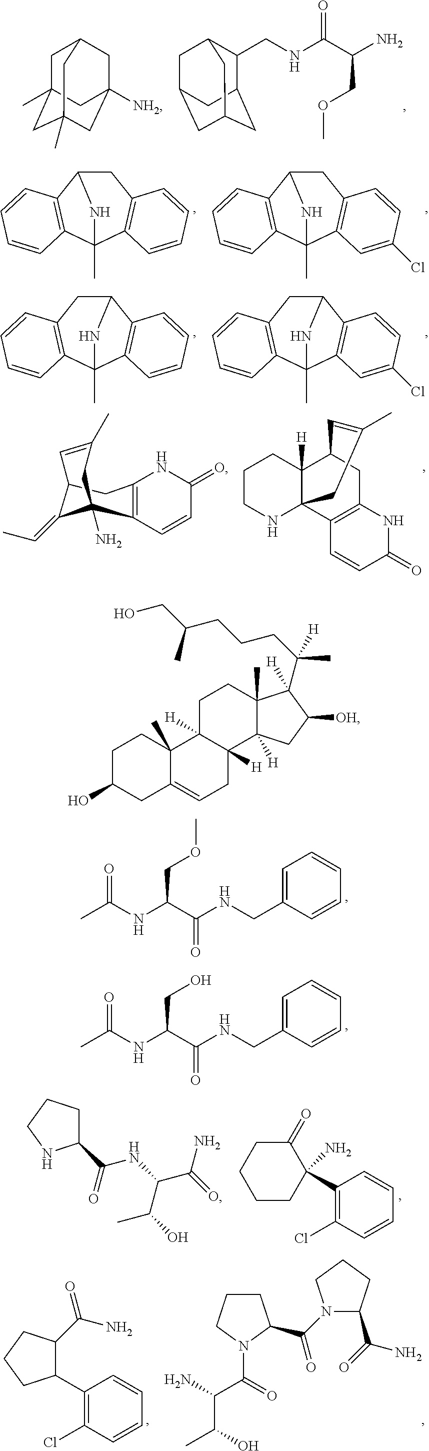

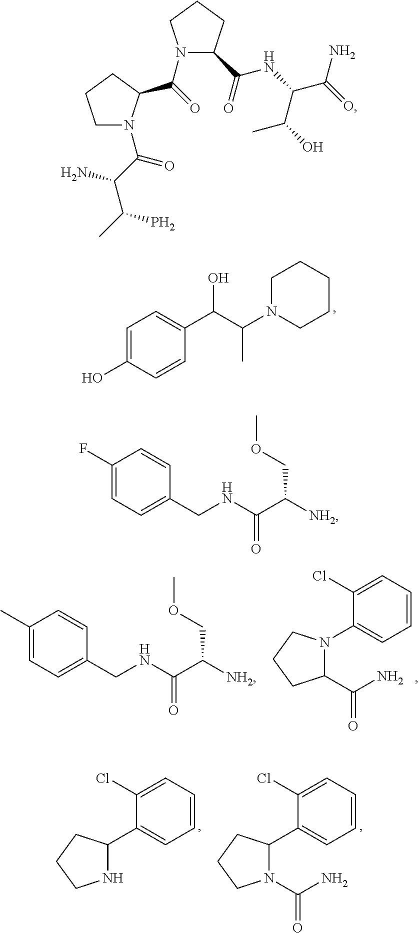

an anti-NMDA receptor antibody, and derivatives thereof.

Embodiment 5. The compound, or salt thereof, of embodiment 1, wherein the compound comprises the structure of Formula (II):

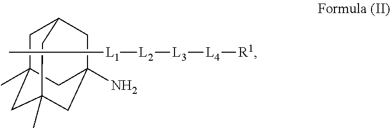

or a salt thereof.

Embodiment 6. The compound, or salt thereof, of embodiment 5, wherein the compound comprises the structure of Formula (II-a):

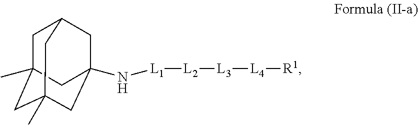

or a salt thereof.

Embodiment 7. The compound, or salt thereof, of embodiment 1, wherein the compound comprises the structure of Formula (III):

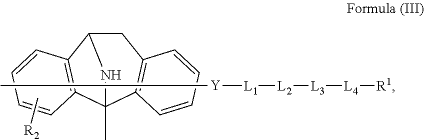

wherein R² is hydrogen, halogen, —OH, or —OMe;
or a salt thereof.

Embodiment 8. The compound, or salt thereof, of embodiment 7, wherein the compound comprises the structure of Formula (III-e):

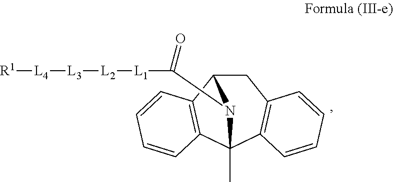

or a salt thereof.

Embodiment 9. The compound, or salt thereof, of embodiment 7, wherein the compound comprises the structure of Formula (III-f):

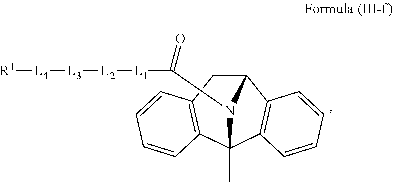

or a salt thereof.

Embodiment 10. The compound, or salt thereof, of embodiment 7, wherein the compound comprises the structure of Formula (III-g):

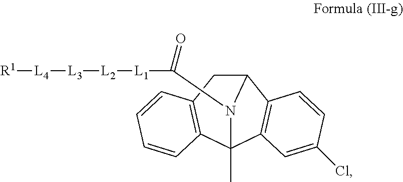

or a salt thereof.

Embodiment 11. The compound, or salt thereof, of embodiment 7, wherein the compound comprises the structure of Formula (III-h):

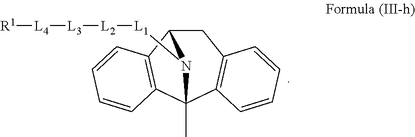

Embodiment 12. The compound, or salt thereof, of embodiment 7, wherein the compound comprises the structure of Formula (III-i):

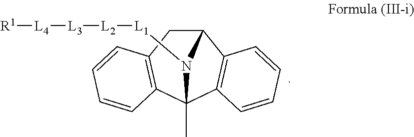

Embodiment 13. The compound, or salt thereof, of embodiment 7, wherein the compound comprises the structure of Formula (III-j):

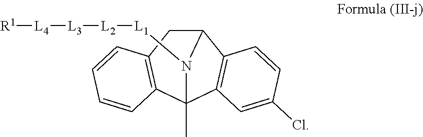

Embodiment 14. The compound, or salt thereof, of embodiment 7, wherein the compound comprises the structure of Formula (III-k):

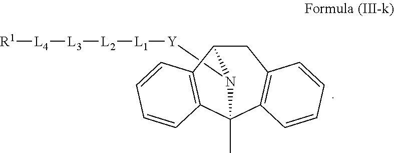

Embodiment 15. The compound, or salt thereof, of embodiment 7, wherein the compound comprises the structure of Formula (III-l):

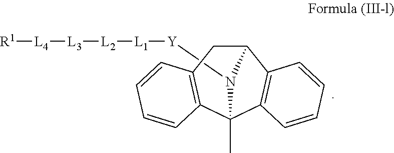

Embodiment 16. The compound, or salt thereof, of embodiment 7, wherein the compound comprises the structure of Formula (III-m):

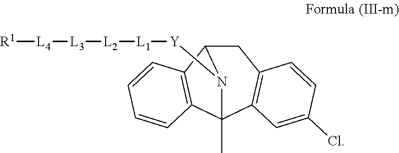

Embodiment 17. The compound, or salt thereof, of embodiment 7, wherein the compound comprises the structure of Formula (III-n):

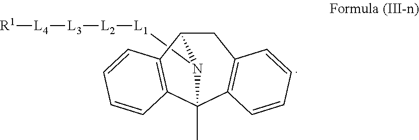

Embodiment 18. The compound, or salt thereof, of embodiment 7, wherein the compound comprises the structure of Formula (III-o):

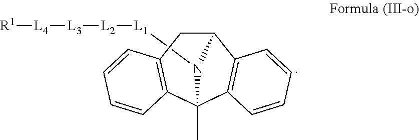

Embodiment 19. The compound, or salt thereof, of embodiment 7, wherein the compound comprises the structure of Formula (III-p):

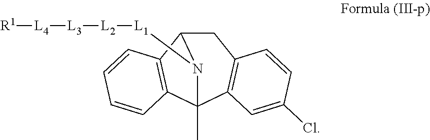

Embodiment 20. The compound, or salt thereof, of embodiment 1, wherein the compound comprises the structure of Formula (IV):

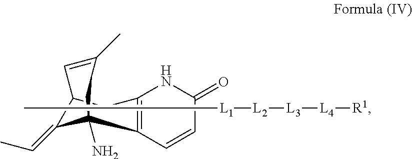

or a salt thereof.

Embodiment 21. The compound, or salt thereof, of embodiment 20, wherein the compound comprises the structure of Formula (IV-a):

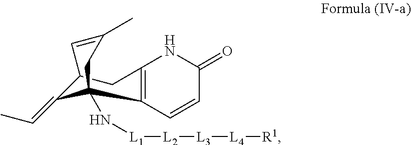

or a salt thereof.

Embodiment 22. The compound, or salt thereof, of embodiment 1, wherein the compound comprises the structure of Formula (XIX):

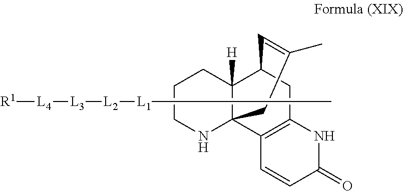

or a salt thereof.

Embodiment 23. The compound, or salt thereof, of embodiment 22, wherein the compound comprises the structure of Formula (XIX-a):

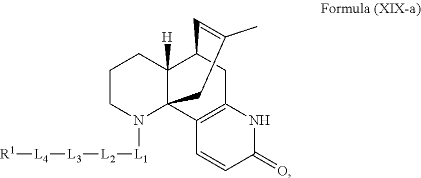

or a salt thereof.

Embodiment 24. The compound, or salt thereof, of embodiment 1, wherein the compound comprises the structure of Formula (V):

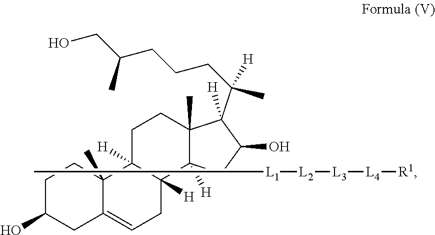

or a salt thereof.

Embodiment 25. The compound, or salt thereof, of embodiment 24, wherein the compound comprises the structure of Formula (V-a):

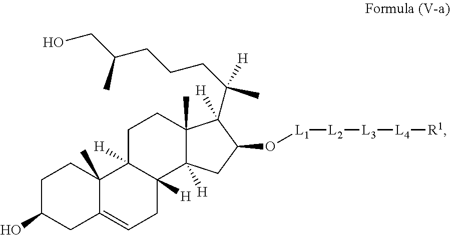

or a salt thereof.

Embodiment 26. The compound, or salt thereof, of embodiment 1, wherein the compound comprises the structure of Formula (VI):

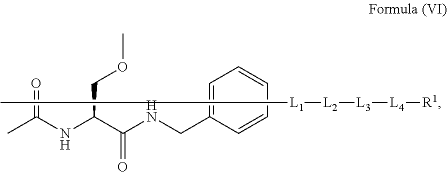

or a salt thereof.

Embodiment 27. The compound, or salt thereof, of embodiment 26, wherein the compound comprises the structure of Formula (VI-a):

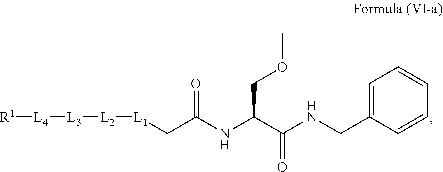

or a salt thereof.

Embodiment 28. The compound, or salt thereof, of embodiment 26, wherein the compound comprises the structure of Formula (VI-b):

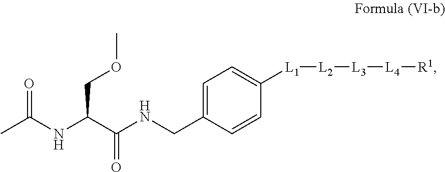

or a salt thereof.

Embodiment 29. The compound, or salt thereof, of embodiment 1, wherein the compound comprises the structure of Formula (VII):

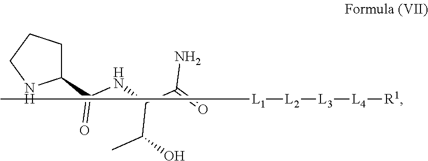

or a salt thereof.

Embodiment 30. The compound, or salt thereof, of embodiment 29, wherein the compound comprises the structure of Formula (VII-a):

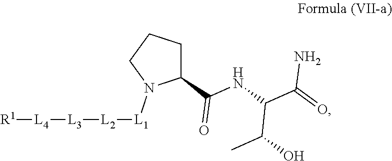

or a salt thereof.

Embodiment 31. The compound, or salt thereof, of embodiment 1, wherein the compound comprises the structure of Formula (VIII):

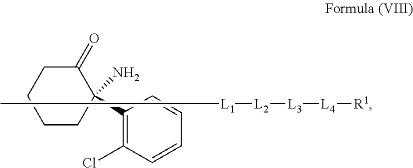

or a salt thereof.

Embodiment 32. The compound, or salt thereof, of embodiment 31, wherein the compound comprises the structure of Formula (VIII-a):

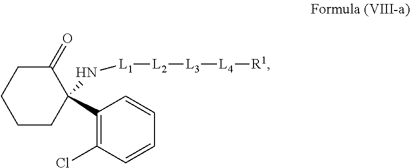

or a salt thereof.

Embodiment 33. The compound, or salt thereof, of embodiment 1, wherein the compound comprises the structure of Formula (IX):

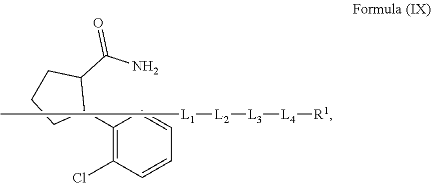

or a salt thereof.

Embodiment 34. The compound, or salt thereof, of embodiment 33, wherein the compound comprises the structure of Formula (IX-a):

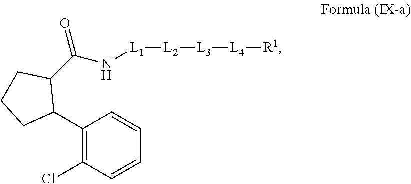

or a salt thereof.

Embodiment 35. The compound, or salt thereof, of embodiment 1, wherein the compound comprises the structure of Formula (X):

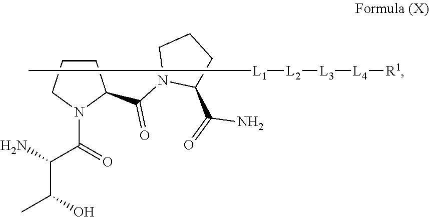

or a salt thereof.

Embodiment 36. The compound, or salt thereof, of embodiment 35, wherein the compound comprises the structure of Formula (X-a):

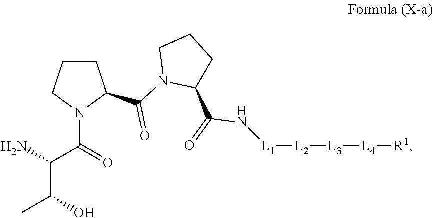

or a salt thereof.

Embodiment 37. The compound, or salt thereof, of embodiment 35, wherein the compound comprises the structure of Formula (X-b):

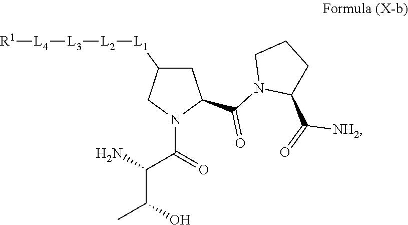

or a salt thereof.

Embodiment 38. The compound, or salt thereof, of embodiment 1, wherein the compound comprises the structure of Formula (XI):

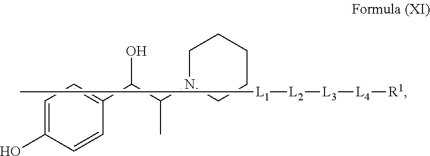

or a salt thereof.

Embodiment 39. The compound, or salt thereof, of embodiment 38, wherein the compound comprises the structure of Formula (XI-a):

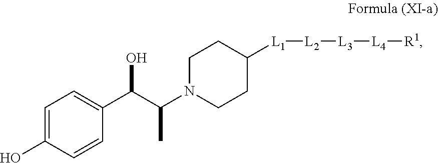

or a salt thereof.

Embodiment 40. The compound, or salt thereof, of embodiment 38, wherein the compound comprises the structure of Formula (XI-b):

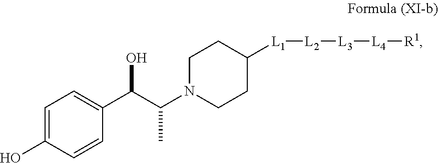

or a salt thereof.

Embodiment 41. The compound, or salt thereof, of embodiment 38, wherein the compound comprises the structure of Formula (XI-c):

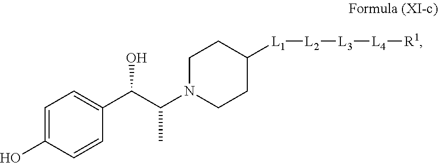

or a salt thereof.

Embodiment 42. The compound, or salt thereof, of embodiment 1, wherein the compound comprises the structure of Formula (XII):

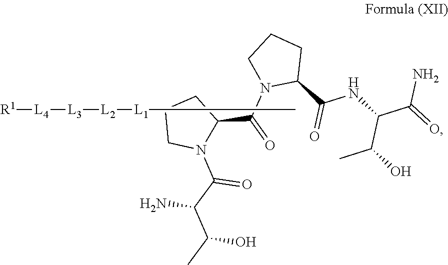

or a salt thereof.

Embodiment 43. The compound, or salt thereof, of embodiment 42, wherein the compound comprises the structure of Formula (XII-a):

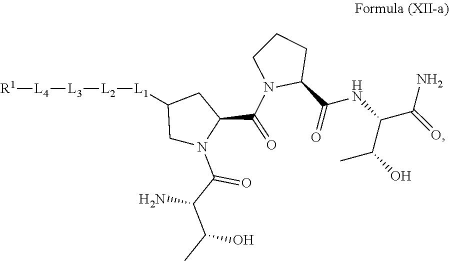

or a salt thereof.

Embodiment 44. The compound, or salt thereof, of embodiment 42, wherein the compound comprises the structure of Formula (XII-b):

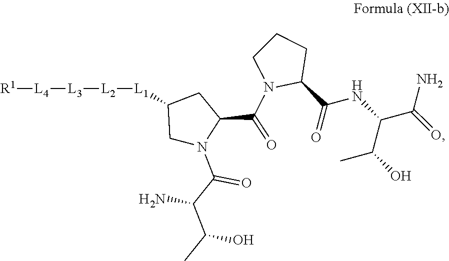

or a salt thereof.

Embodiment 45. The compound, or salt thereof, of embodiment 1, wherein the compound comprises the structure of Formula (XIII):

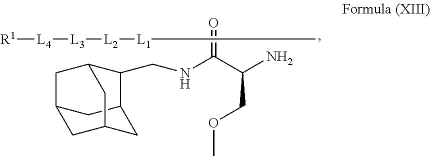

or a salt thereof.

Embodiment 46. The compound, or salt thereof, of embodiment 45, wherein the compound comprises the structure of Formula (XIII-a):

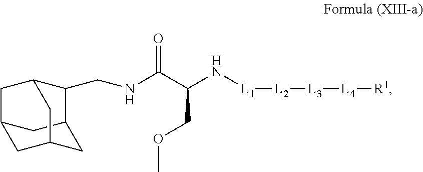

or a salt thereof.

Embodiment 47. The compound, or salt thereof, of embodiment 1, wherein the compound comprises the structure of Formula (XIV):

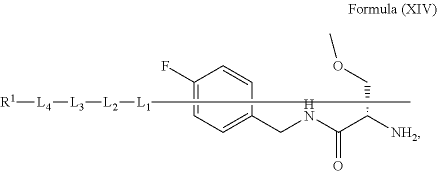

or a salt thereof.

Embodiment 48. The compound, or salt thereof, of embodiment 47, wherein the compound comprises the structure of Formula (XIV-a):

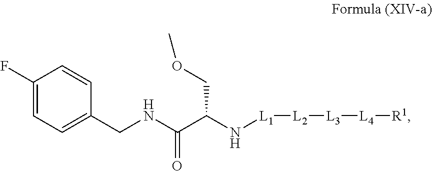

or a salt thereof.

Embodiment 49. The compound, or salt thereof, of embodiment 1, wherein the compound comprises the structure of Formula (XV):

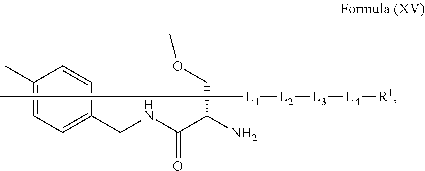

or a salt thereof.

Embodiment 50. The compound, or salt thereof, of embodiment 49, wherein the compound comprises the structure of Formula (XV-a):

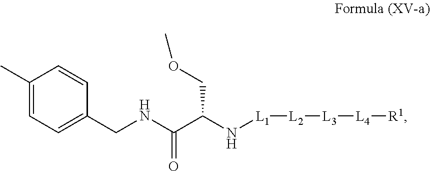

or a salt thereof.

Embodiment 51. The compound, or salt thereof, of embodiment 1, wherein the compound comprises the structure of Formula (XVI):

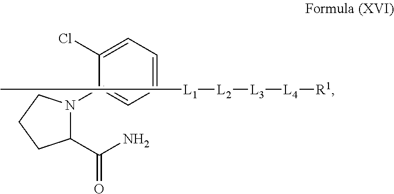

or a salt thereof.

Embodiment 52. The compound, or salt thereof, of embodiment 51, wherein the compound comprises the structure of Formula (XVI-a):

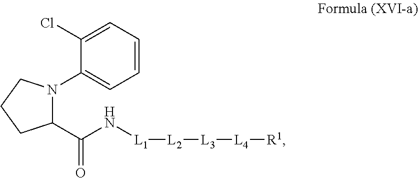

or a salt thereof.

Embodiment 53. The compound, or salt thereof, of embodiment 1, wherein the compound comprises the structure of Formula (XVII):

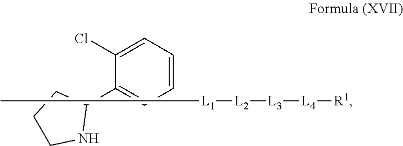

or a salt thereof.

Embodiment 54. The compound, or salt thereof, of embodiment 53, wherein the compound comprises the structure of Formula (XVII-a):

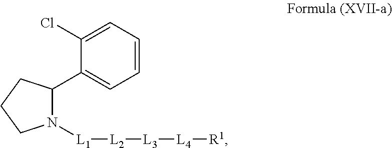

or a salt thereof.

Embodiment 55. The compound, or salt thereof, of embodiment 1, wherein the compound comprises the structure of Formula (XVIII):

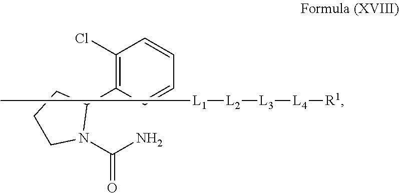

or a salt thereof.

Embodiment 56. The compound, or salt thereof, of embodiment 55, wherein the compound comprises the structure of Formula (XVIII-a):

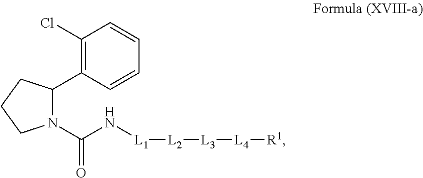

or a salt thereof.

Embodiment 57. The compound, or salt thereof, of any one of embodiments 1-56, wherein each of L₁, L₂, L₃, and L₄ is independently absent, a bond, an optionally substituted alkyl linker, an optionally substituted polyethylene glycol (PEG) linker, an optionally substituted heteroalkyl linker, an optionally substituted heteroaryl linker, a phosphodiester bond, or a phosphorothioate bond.

Embodiment 58. The compound, or salt thereof, of any one of embodiments 1-57, wherein L₁ is a bond.

Embodiment 59. The compound, or salt thereof, of any one of embodiments 1-57, wherein L₁ is an optionally substituted alkyl linker.

Embodiment 60. The compound, or salt thereof, of any one of embodiments 1-57, wherein L₁ is an optionally substituted C1-C6 alkyl linker.

Embodiment 61. The compound, or salt thereof, of any one of embodiments 1-57, wherein L₁ is a C1-C6 alkyl linker substituted with ═O.

Embodiment 62. The compound, or salt thereof, of any one of embodiments 1-57, wherein L₁ comprises the structure

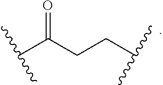 [SMILES: *CCC(*)=O]

Embodiment 63. The compound, or salt thereof, of any one of embodiments 1-62, wherein L₂ is an optionally substituted alkyl linker.

Embodiment 64. The compound, or salt thereof, of any one of embodiments 1-62, wherein L₂ is an optionally substituted C1-C15 alkyl linker.

Embodiment 65. The compound, or salt thereof, of any one of embodiments 1-62, wherein L₂ is an optionally substituted C5-C12 alkyl linker.

Embodiment 66. The compound, or salt thereof, of any one of embodiments 1-62, wherein L₂ comprises the structure

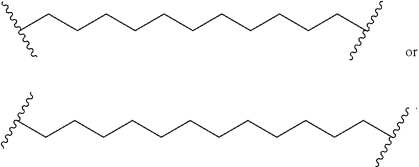 [SMILES: *CCCCCCCCCCC* ; *CCCCCCCCCCCC*]

Embodiment 67. The compound, or salt thereof, of any one of embodiments 1-62, wherein L₂ is an optionally substituted PEG linker

Embodiment 68. The compound, or salt thereof, of any one of embodiments 1-62, wherein L₂ is an optionally substituted PEG linker comprising one, two, three, four, five, six, seven, or eight PEG units in length, wherein a PEG unit comprises the structure

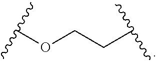 [SMILES: *CCO*]

Embodiment 69. The compound, or salt thereof, of any one of embodiments 1-62, wherein L₂ is an optionally substituted PEG linker comprising three PEG units in length, wherein a PEG unit comprises the structure

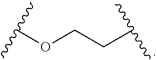 [SMILES: *CCO*]

Embodiment 70. The compound, or salt thereof, of any one of embodiments 1-62, wherein L₂ is an optionally substituted PEG linker comprising four PEG units in length, wherein a PEG unit comprises the structure

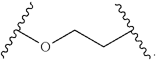 [SMILES: *CCO*]

Embodiment 71. The compound, or salt thereof, of any one of embodiments 1-62, wherein L₂ comprises the structure

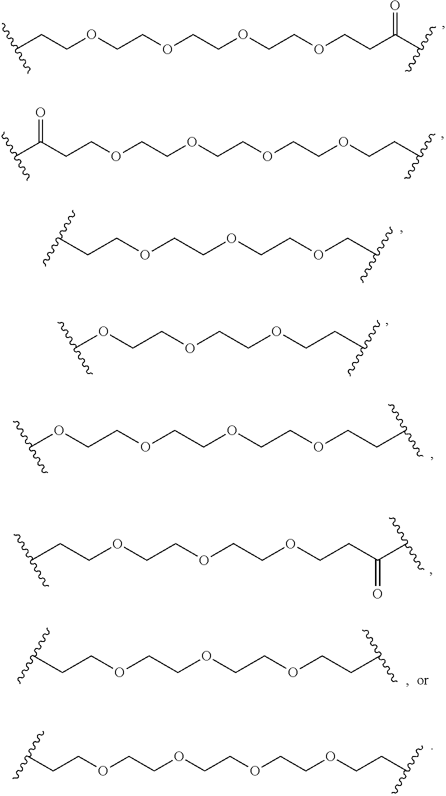

Embodiment 72. The compound, or salt thereof, of any one of embodiments 1-62, wherein L₂ is an optionally substituted heteroalkyl linker.

Embodiment 73. The compound, or salt thereof, of embodiment 72, wherein L₂ comprises the structure

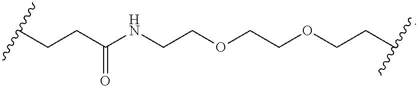 [SMILES: *CCOCCOCCNC(=O)CC*]

Embodiment 74. The compound, or salt thereof, of any one of embodiments 1-73, wherein L₃ is an optionally substituted heteroaryl linker.

Embodiment 75. The compound, or salt thereof, of any one of embodiments 1-73, wherein L₃ is an optionally substituted partially unsaturated heterocycloalkyl or optionally unsubstituted heteroaryl linker.

Embodiment 76. The compound, or salt thereof, of embodiment 74 or 75, wherein L₃ comprises the structure

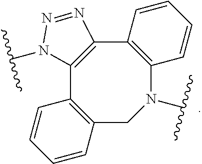 [SMILES: *N1Cc2ccccc2-c2c(nnn2*)-c2ccccc21]

Embodiment 77. The compound, or salt thereof, of any one of embodiments 1-76, wherein L₄ is an optionally substituted heteroalkyl linker.

Embodiment 78. The compound, or salt thereof, of embodiment 77, wherein the heteroalkyl linker is substituted with one or more ═O substituents.

Embodiment 79. The compound, or salt thereof, of embodiment 77 or 78, wherein L₄ comprises the structure

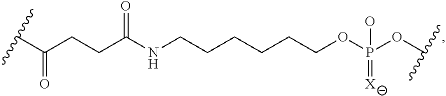

wherein X is O or S.

Embodiment 80. The compound, or salt thereof, of embodiment 77 or 78, wherein L₄ comprises the structure

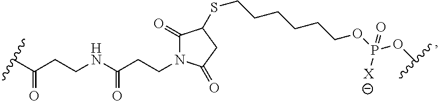

wherein X is S or O

Embodiment 81. The compound, or salt thereof, of any one of embodiments 1-80, wherein L₁, L₂, L₃, and L₄ together comprise the structure

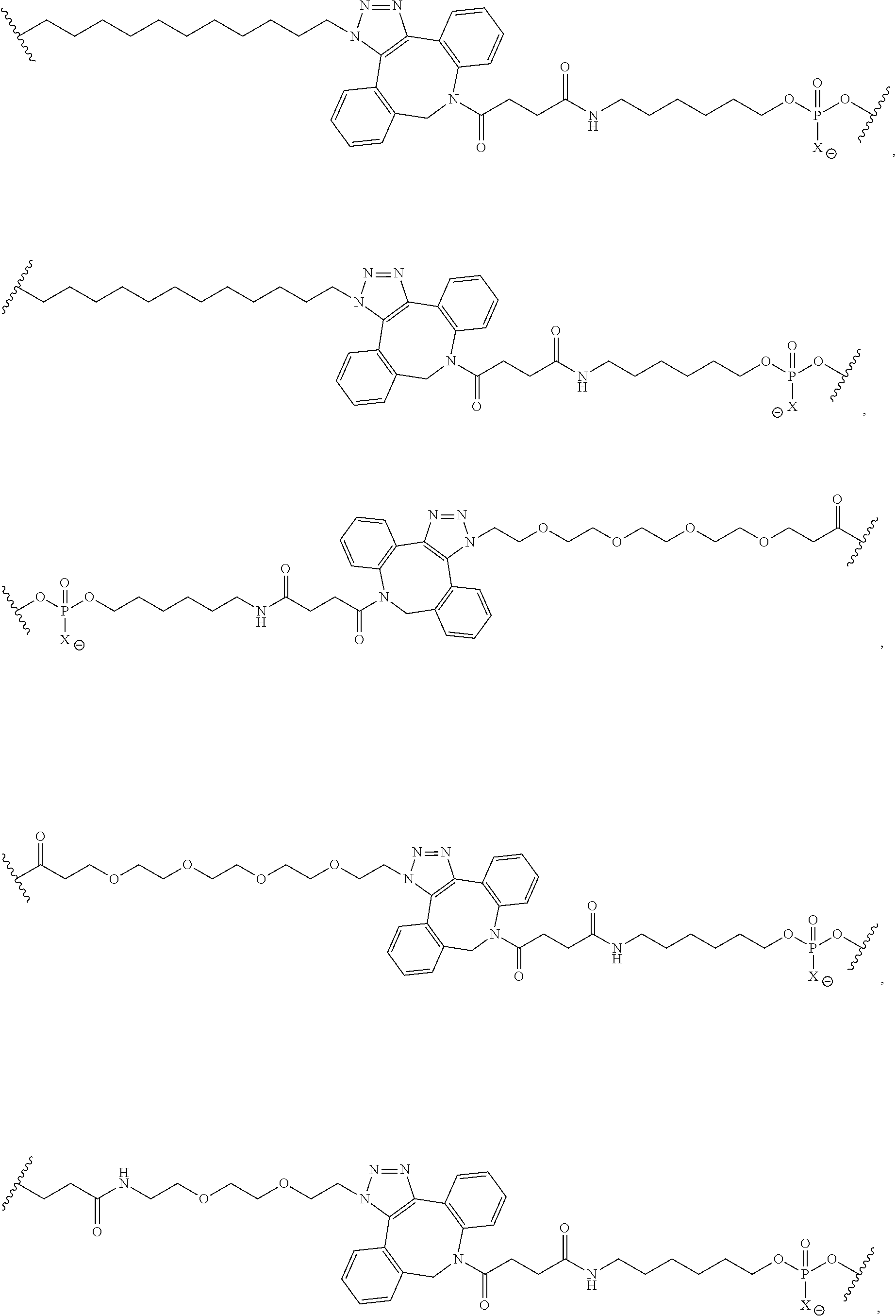

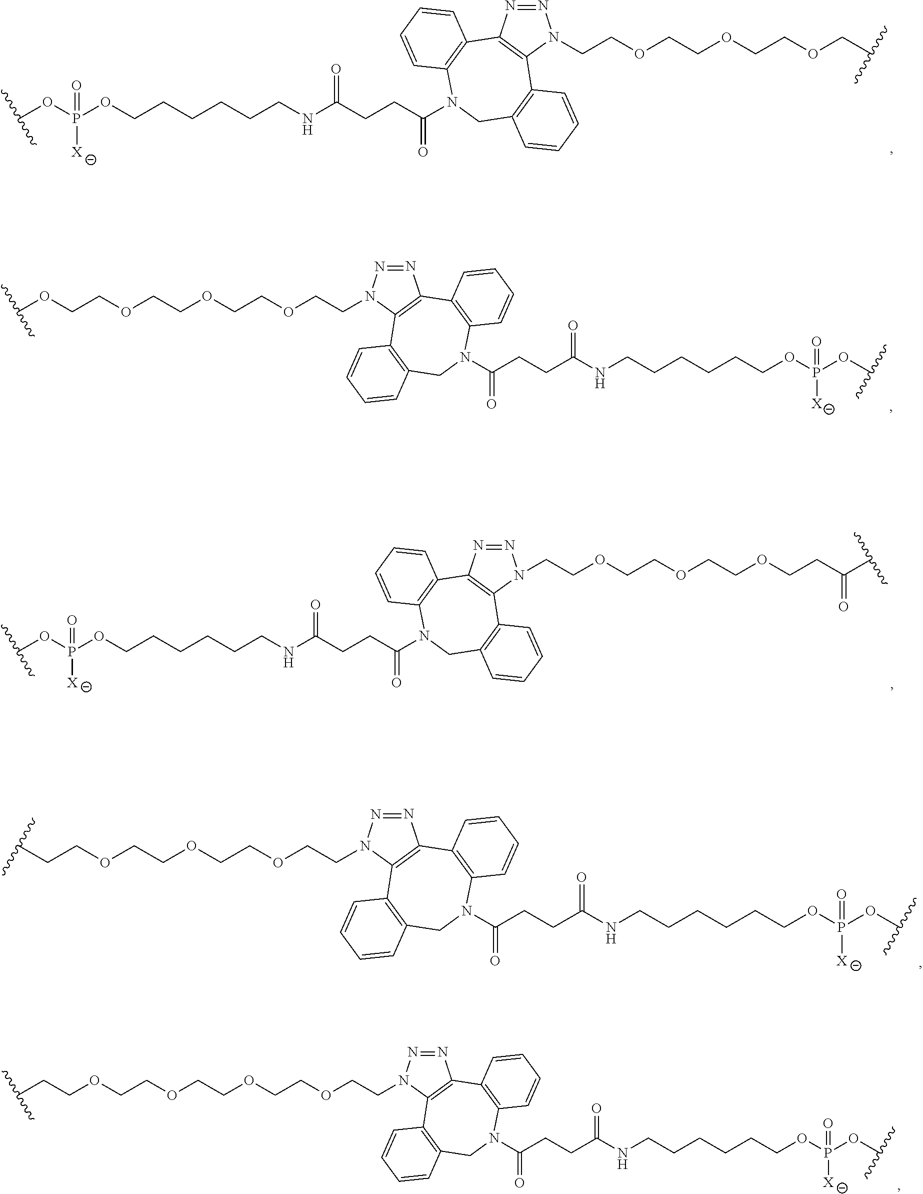

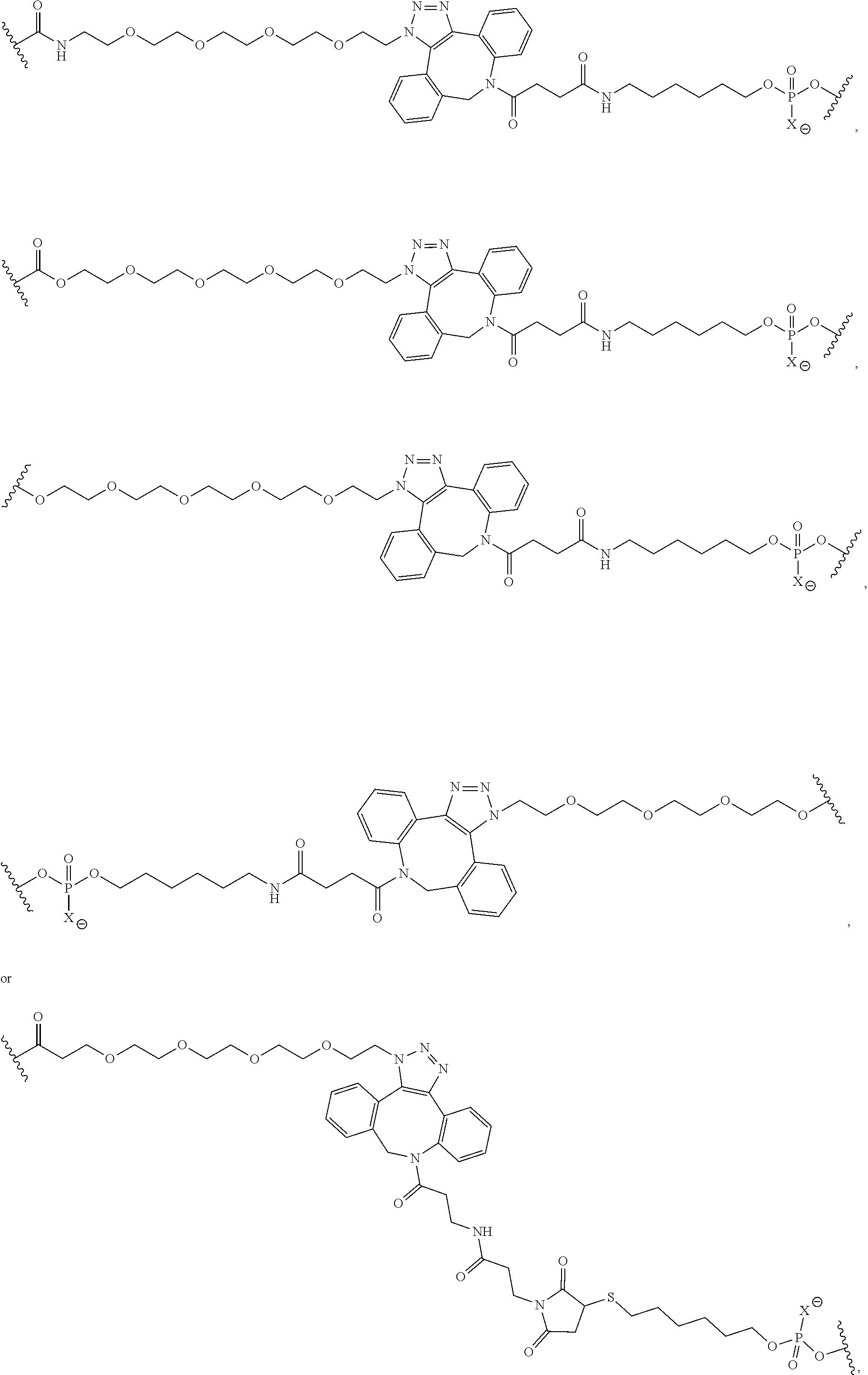

wherein X is O or S.

Embodiment 82. The compound, or salt thereof, of any one of embodiments 1-81, wherein the compound comprises the structure

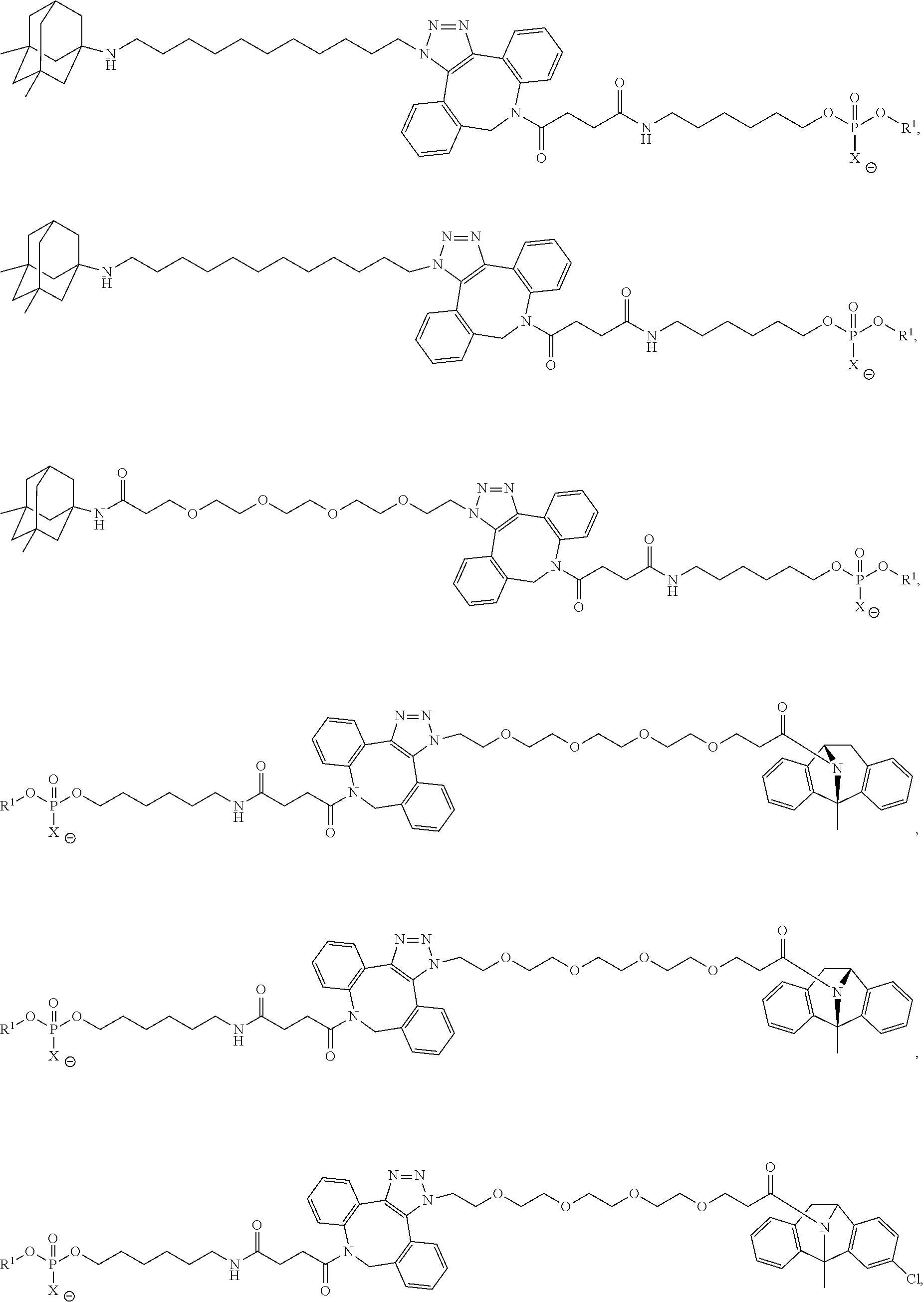

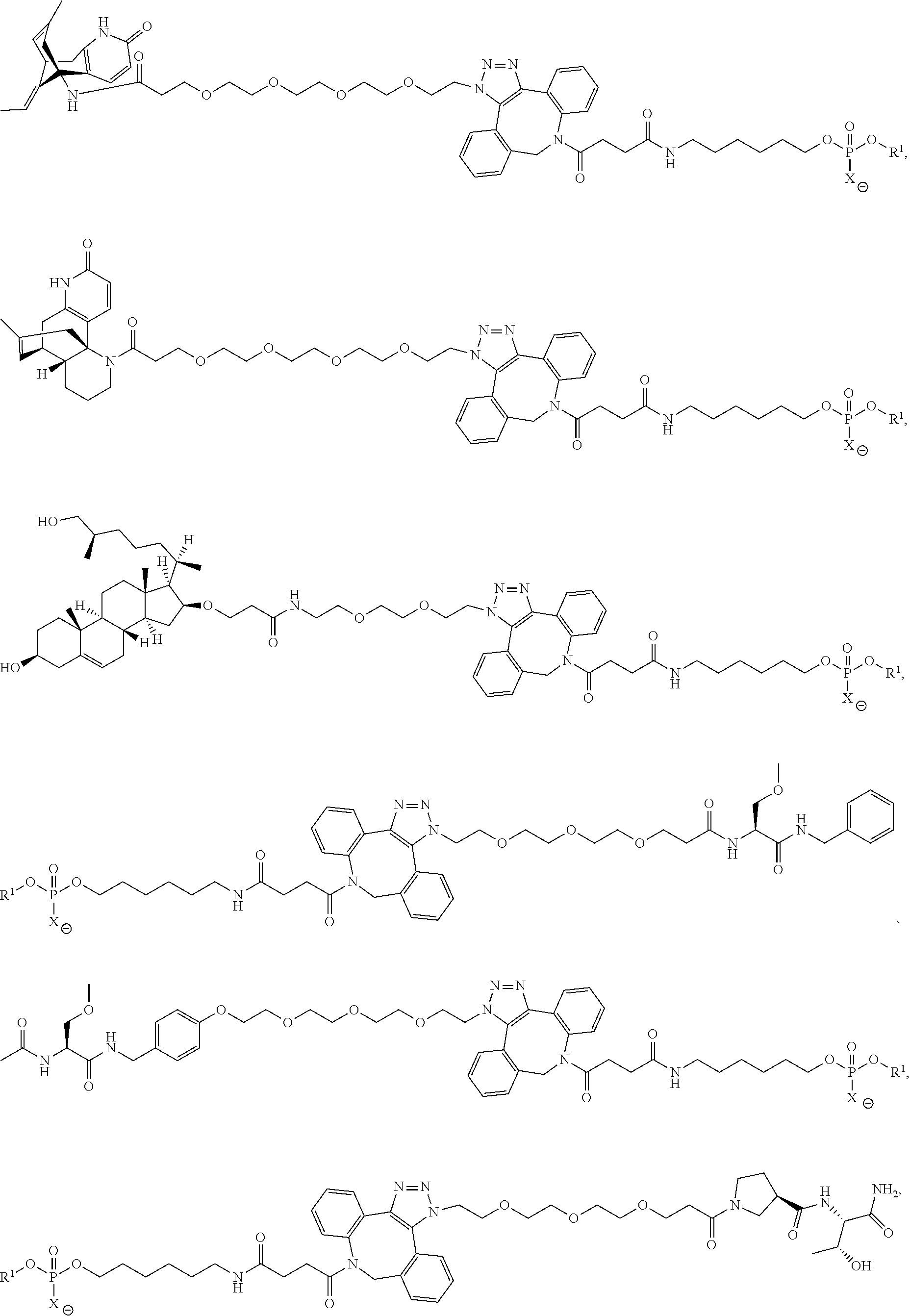

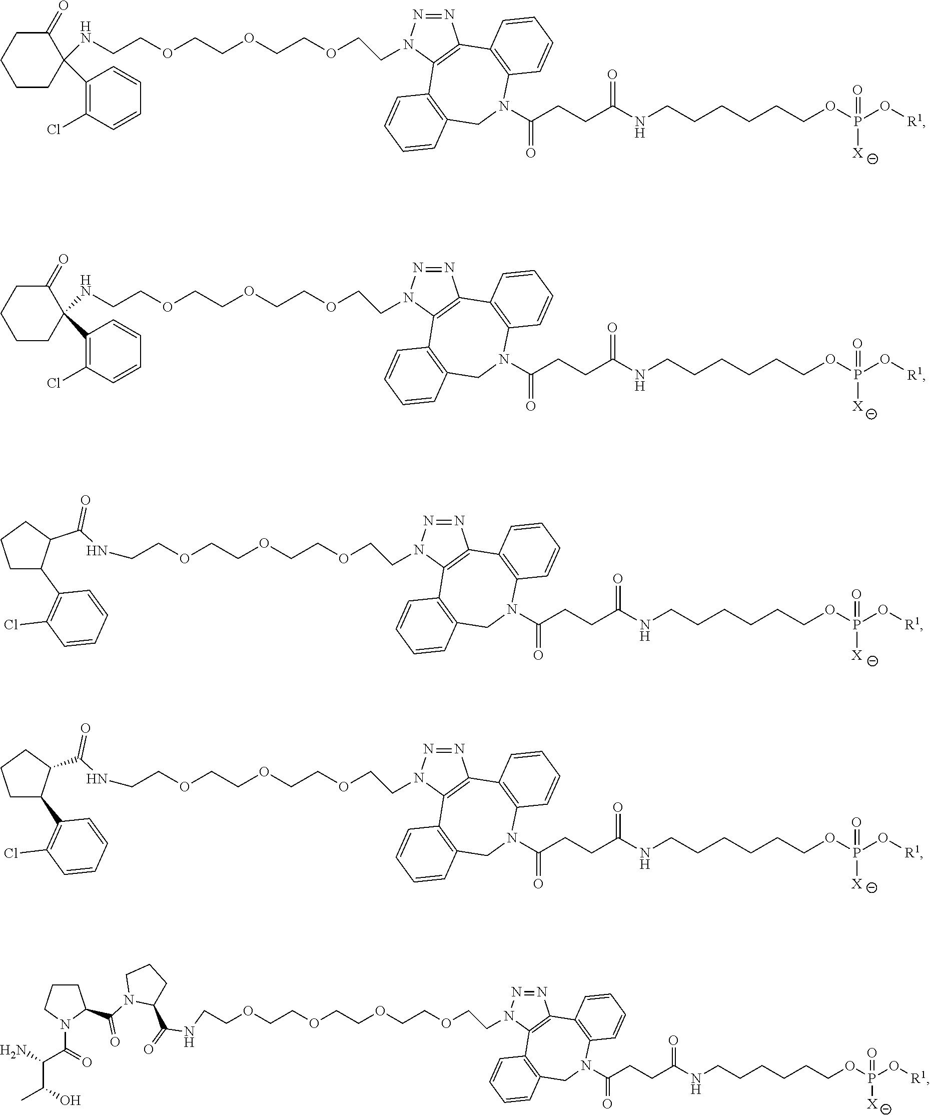

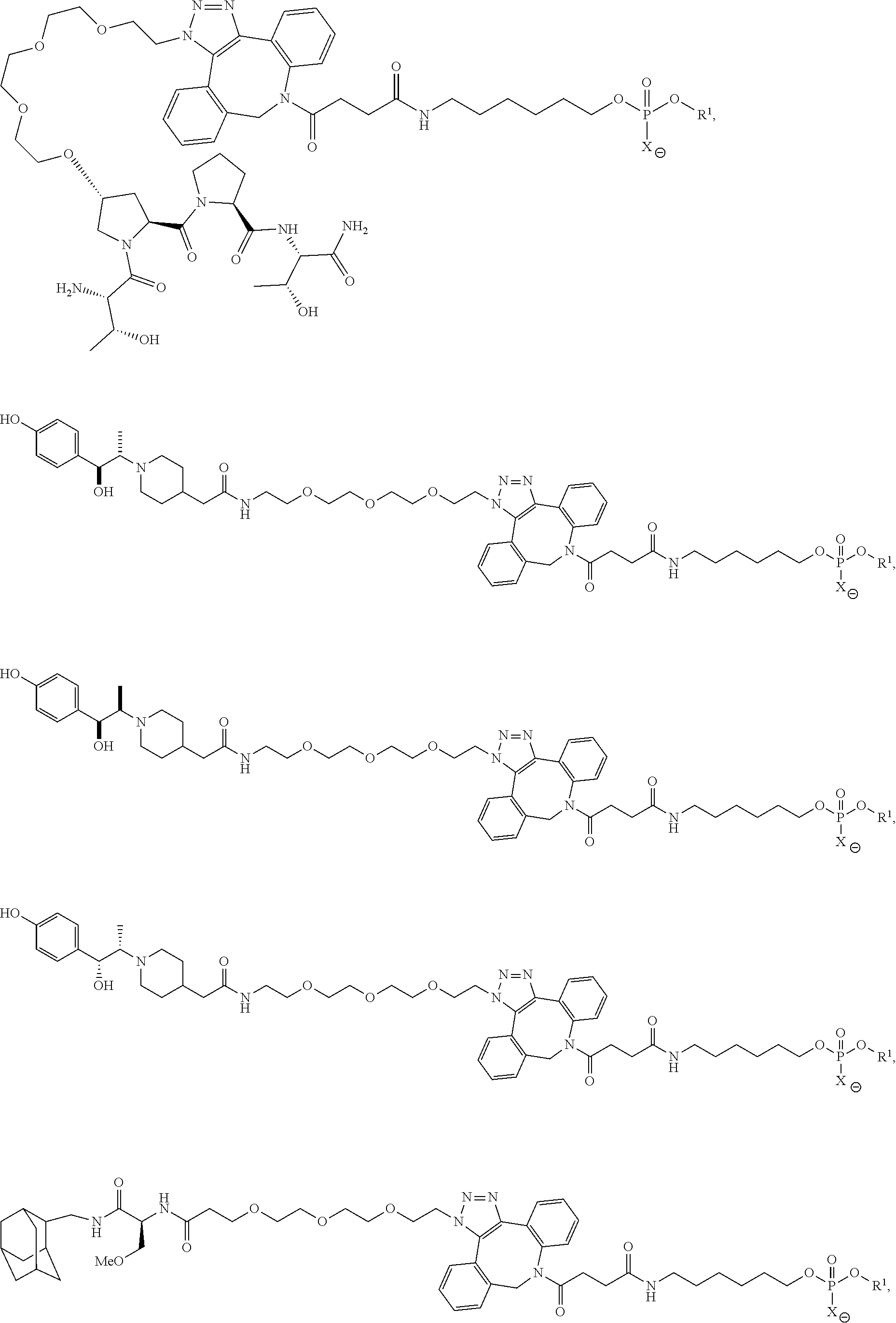

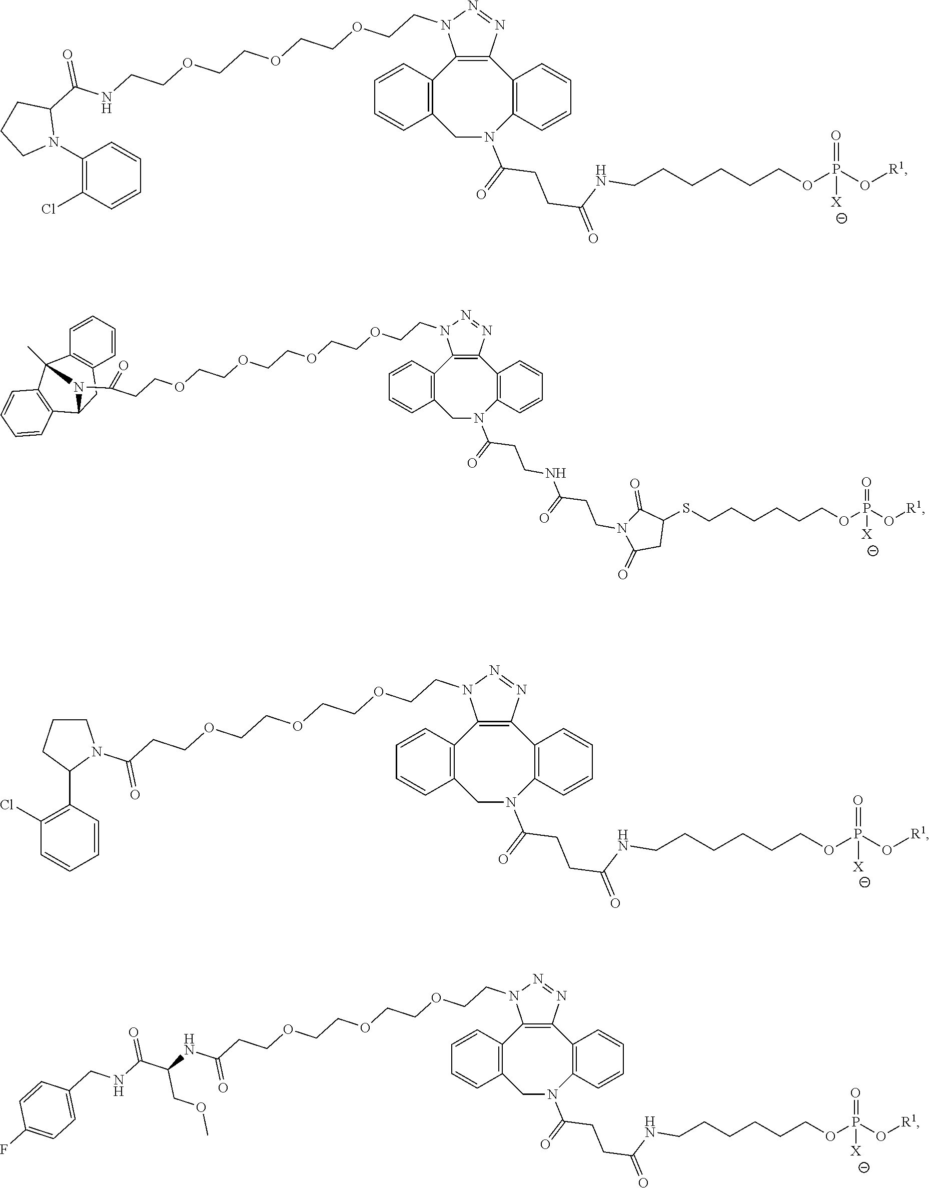

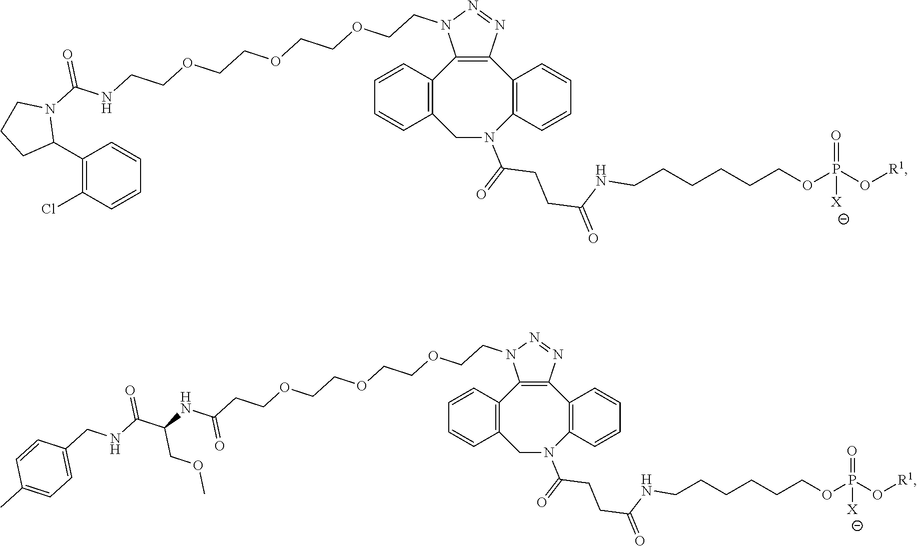

wherein X is O or S.

Embodiment 83. The compound, or salt thereof, of any one of embodiments 79-82, wherein X is O.

Embodiment 84. The compound, or salt thereof, of any one of embodiments 79-82, wherein X is S.

Embodiment 85. The compound, or salt thereof, of any one of embodiments 1-84, wherein R¹ comprises an oligonucleotide.

Embodiment 86. The compound, or salt thereof, of embodiment 85, wherein the oligonucleotide is attached at its 5′ end.

Embodiment 87. The compound, or salt thereof, of embodiment 85, wherein the oligonucleotide is attached at its 3′ end.

Embodiment 88. The compound, or salt thereof, of embodiment 85, wherein the oligonucleotide is attached at an internal position on the oligonucleotide.

Embodiment 89. The compound, or salt thereof, of embodiment 88, wherein the internal position is an internucleoside linkage.

Embodiment 90. The compound, or salt thereof, of any one of embodiments 1-89, wherein R¹ comprises an oligonucleotide conjugated to one or more additional NMDA receptor ligands.

Embodiment 91. The compound, or salt thereof, of embodiment 90, wherein the oligonucleotide is conjugated to two, three, four, five, or more than five additional NMDA receptor ligands.

Embodiment 92. The compound, or salt thereof, of embodiment 90 or 91, wherein the additional NMDA receptor ligands are conjugated to the oligonucleotide at the 5′ end of the oligonucleotide, the 3′ end of the oligonucleotide, one or more internal positions on the oligonucleotide, or any combination thereof.

Embodiment 93. The compound, or salt thereof, of any one of embodiments 85-92, wherein the oligonucleotide is a modified oligonucleotide.

Embodiment 94. A composition comprising a compound, or salt thereof, of any one of embodiments 1-93, and a pharmaceutically acceptable excipient.

Embodiment 95. A method for delivering a therapeutic oligonucleotide to the brain of a subject, comprising administration of a compound, or salt thereof, of any one of embodiments 1-93, or a composition of embodiment 94, to the subject.

Embodiment 96. The method of embodiment 95, wherein the therapeutic oligonucleotide is delivered to one or more brain regions selected from the group consisting of the striatum, the cerebellum, the brain stem, the hippocampus, the frontal cortex, and the spinal cord.

Embodiment 97. A method for treating or ameliorating a disease, disorder, or symptom thereof in a subject, comprising administration of a compound, or salt thereof, of any one of embodiments 1-93, or a composition of embodiment 94, to the subject.

Embodiment 98. The method of embodiment 97, wherein the disease, disorder, or symptom thereof is a central nervous system (CNS) disease, disorder, or symptom thereof.

Embodiment 99. The method of embodiment 97 or 98, wherein the disease, disorder, or symptom thereof is Alzheimer's disease, or a symptom thereof.

Embodiment 100. The method of any one of embodiments 97-99, wherein the compound, or salt thereof, is administered to the subject intrathecally.

Embodiment 101. A method for making a compound, or salt thereof, of any one of embodiments 1-93, comprising one or more compounds and chemical transformations described herein, including Examples 1-77.

What is claimed is:

1. A compound comprising the structure of Formula (I), or a salt thereof: 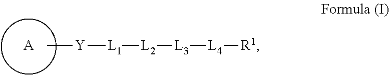
wherein 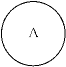
is an N-methyl-D-aspartate (NMDA) receptor ligand;
each of L₁, L₂, L₃, and L₄ is independently a linker, a bond, or absent;
Y is a bond or —C(═O)—; and
R¹ is one or more oligonucleotides, protecting groups, small molecules, proteins, antibodies, peptides, or combinations thereof.

2. The compound, or salt thereof, of claim 1, wherein the NMDA receptor ligand is an NMDA receptor agonist.

3. The compound, or salt thereof, of claim 1, wherein the NMDA receptor ligand is an NMDA receptor antagonist.

4. The compound, or salt thereof, of claim 1, wherein the NMDA receptor ligand is selected from the group consisting of: 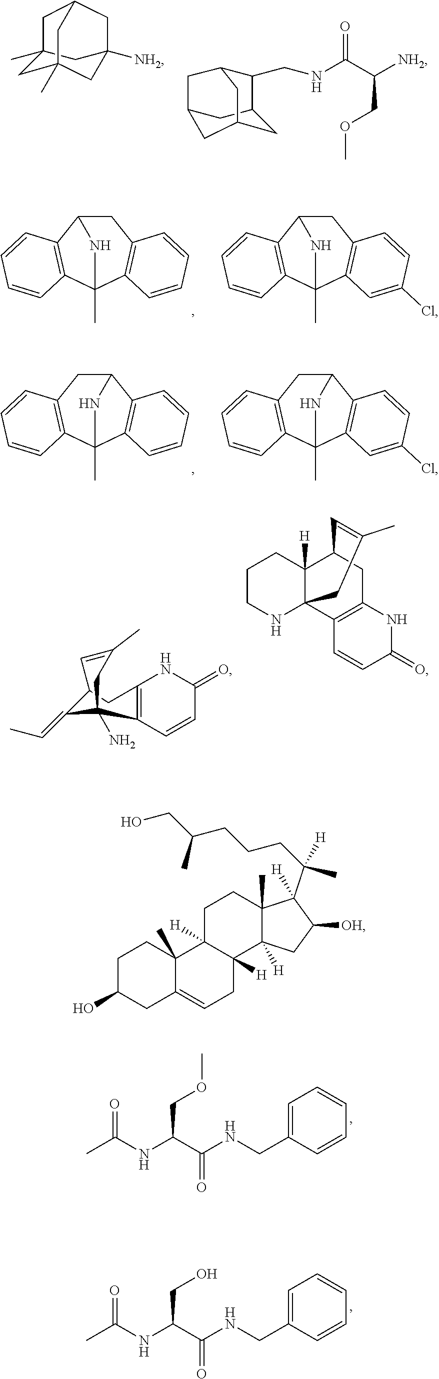 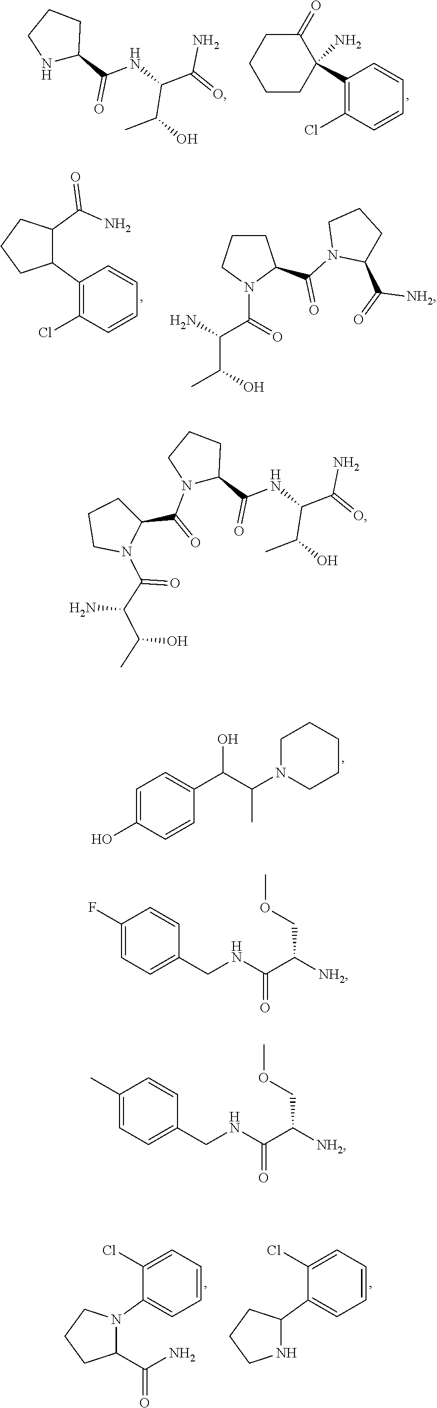 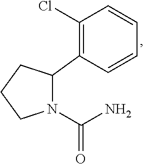 [SMILES: NC(=O)N1CCCC1c1ccccc1Cl]
an anti-NMDA receptor antibody, and derivatives thereof.

5. The compound, or salt thereof, of claim 1, wherein the compound comprises the structure of Formula (II): 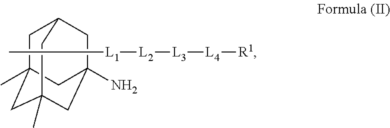
or a salt thereof.

6. The compound, or salt thereof, of claim 5, wherein the compound comprises the structure of Formula (II-a): 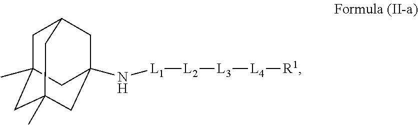
or a salt thereof.

7. The compound, or salt thereof, of claim 1, wherein the compound comprises the structure of Formula (III): 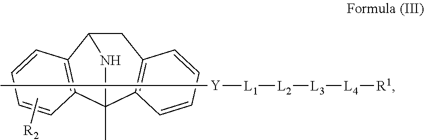
wherein R² is hydrogen, halogen, —OH, or —OMe;
or a salt thereof.

8. The compound, or salt thereof, of claim 7, wherein the compound comprises the structure of Formula (III-e): 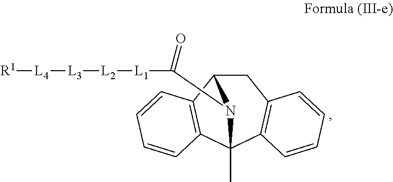
or a salt thereof.

9. The compound, or salt thereof, of claim 7, wherein the compound comprises the structure of Formula (III-f): 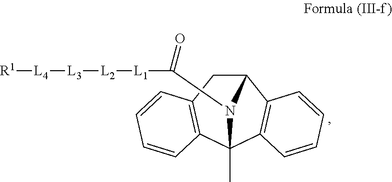
or a salt thereof.

10. The compound, or salt thereof, of claim 7, wherein the compound comprises the structure of Formula (III-g): 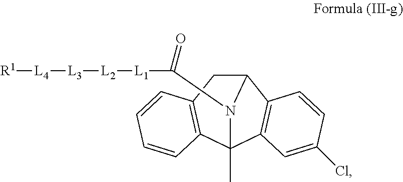
or a salt thereof.

11. The compound, or salt thereof, of claim 7, wherein the compound comprises the structure of Formula (III-h):

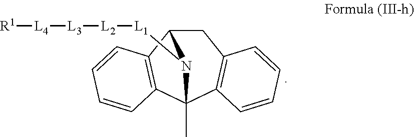

12. The compound, or salt thereof, of claim 7, wherein the compound comprises the structure of Formula (III-i):

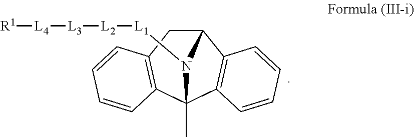

13. The compound, or salt thereof, of claim 7, wherein the compound comprises the structure of Formula (III-j):

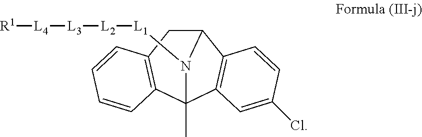

14. The compound, or salt thereof, of claim 7, wherein the compound comprises the structure of Formula (III-k):

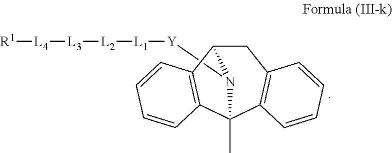

15. The compound, or salt thereof, of claim 7, wherein the compound comprises the structure of Formula (III-l):

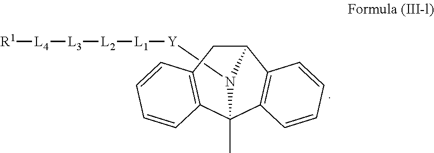

16. The compound, or salt thereof, of claim 7, wherein the compound comprises the structure of Formula (III-m):

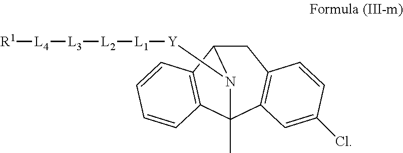

17. The compound, or salt thereof, of claim 7, wherein the compound comprises the structure of Formula (III-n):

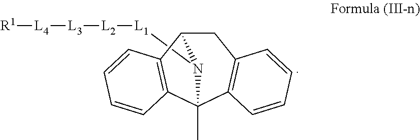

18. The compound, or salt thereof, of claim 7, wherein the compound comprises the structure of Formula (III-o):

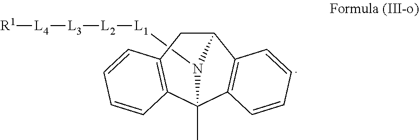

19. The compound, or salt thereof, of claim 7, wherein the compound comprises the structure of Formula (III-p):

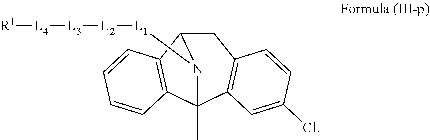

20. The compound, or salt thereof, of claim 1, wherein the compound comprises the structure of Formula (IV): 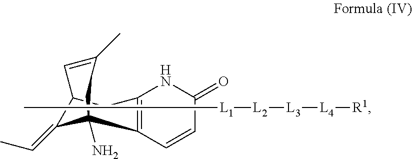
or a salt thereof.

21. The compound, or salt thereof, of claim 20, wherein the compound comprises the structure of Formula (IV-a): 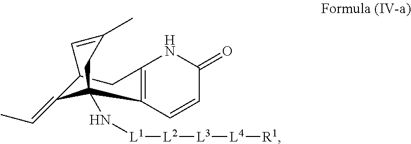
or a salt thereof.

22. The compound, or salt thereof, of claim 1, wherein the compound comprises the structure of Formula (XIX): 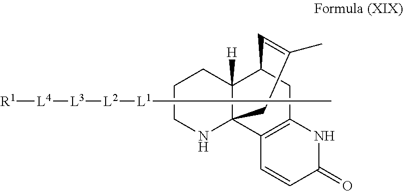
or a salt thereof.

23. The compound, or salt thereof, of claim 22, wherein the compound comprises the structure of Formula (XIX-a): 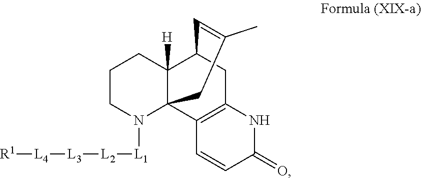
or a salt thereof.

24. The compound, or salt thereof, of claim 1, wherein the compound comprises the structure of Formula (V): 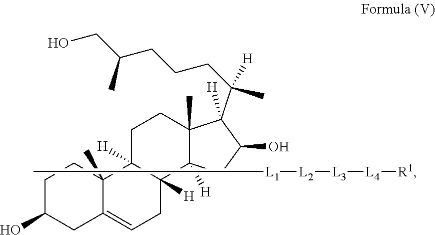
or a salt thereof.

25. The compound, or salt thereof, of claim 24, wherein the compound comprises the structure of Formula (V-a): 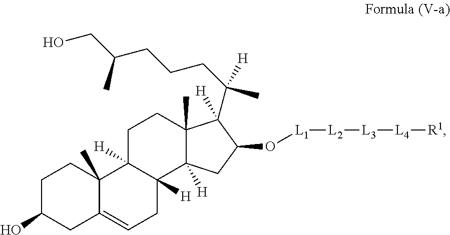
or a salt thereof.

26. The compound, or salt thereof, of claim 1, wherein the compound comprises the structure of Formula (VI): 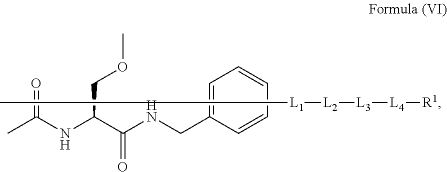
or a salt thereof.

27. The compound, or salt thereof, of claim 26, wherein the compound comprises the structure of Formula (VI-a): 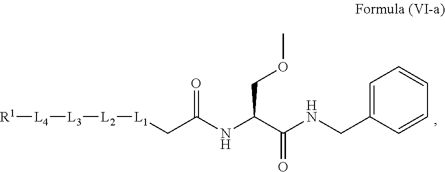
or a salt thereof.

28. The compound, or salt thereof, of claim 26, wherein the compound comprises the structure of Formula (VI-b): 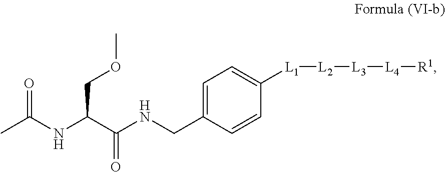
or a salt thereof.

29. The compound, or salt thereof, of claim 1, wherein the compound comprises the structure of Formula (VII): 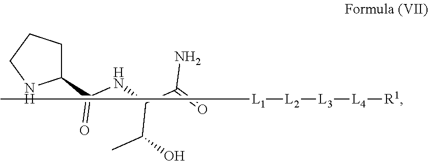
or a salt thereof.

30. The compound, or salt thereof, of claim 29, wherein the compound comprises the structure of Formula (VII-a): 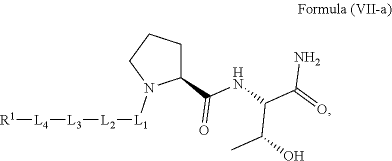
or a salt thereof.

31. The compound, or salt thereof, of claim 1, wherein the compound comprises the structure of Formula (VIII): 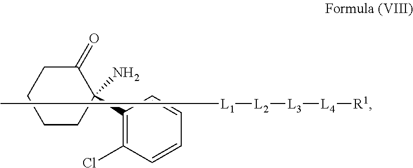
or a salt thereof.

32. The compound, or salt thereof, of claim 31, wherein the compound comprises the structure of Formula (VIII-a): 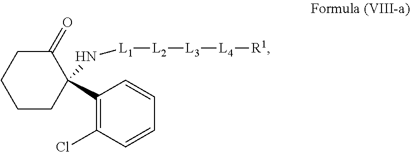
or a salt thereof.

33. The compound, or salt thereof, of claim 1, wherein the compound comprises the structure of Formula (IX): 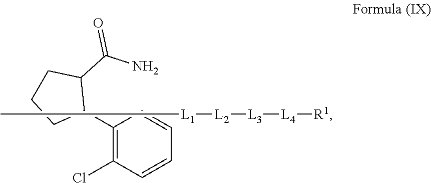
or a salt thereof.

34. The compound, or salt thereof, of claim 33, wherein the compound comprises the structure of Formula (IX-a): 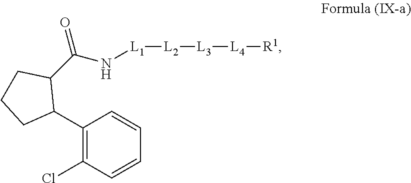
or a salt thereof.

35. The compound, or salt thereof, of claim 1, wherein the compound comprises the structure of Formula (X): 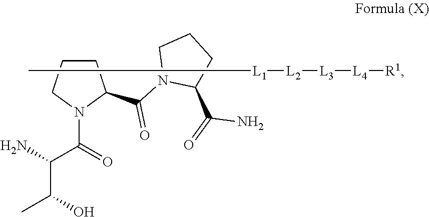
or a salt thereof.

36. The compound, or salt thereof, of claim 35, wherein the compound comprises the structure of Formula (X-a): 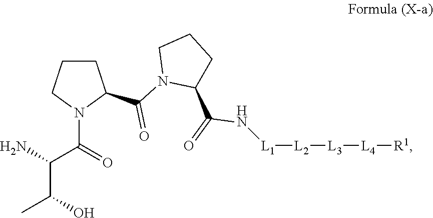
or a salt thereof.

37. The compound, or salt thereof, of claim 35, wherein the compound comprises the structure of Formula (X-b): 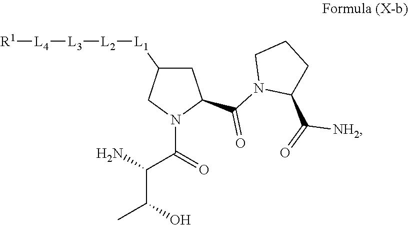
or a salt thereof.

38. The compound, or salt thereof, of claim 1, wherein the compound comprises the structure of Formula (XI): 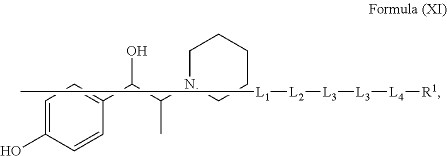
or a salt thereof.

39. The compound, or salt thereof, of claim 38, wherein the compound comprises the structure of Formula (XI-a): 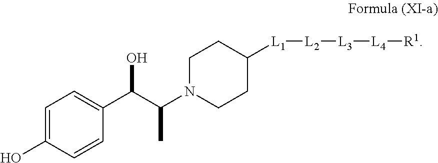
or a salt thereof.

40. The compound, or salt thereof, of claim 38, wherein the compound comprises the structure of Formula (XI-b): 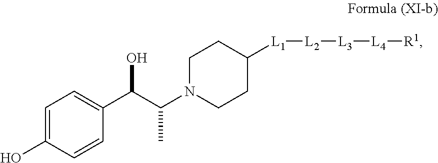
or a salt thereof.

41. The compound, or salt thereof, of claim 38, wherein the compound comprises the structure of Formula (XI-c): 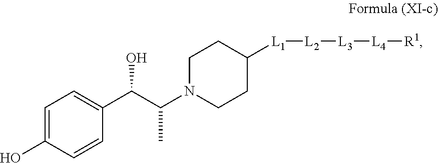
or a salt thereof.

42. The compound, or salt thereof, of claim 1, wherein the compound comprises the structure of Formula (XII): 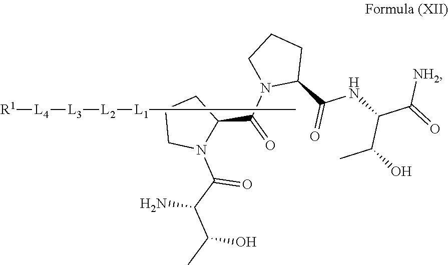
or a salt thereof.

43. The compound, or salt thereof, of claim 42, wherein the compound comprises the structure of Formula (XII-a): 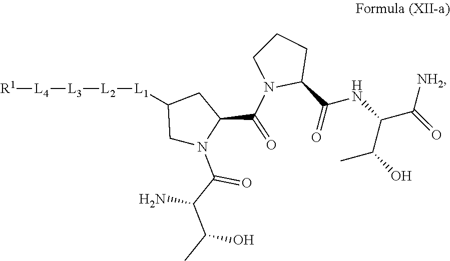
or a salt thereof.

44. The compound, or salt thereof, of claim 42, wherein the compound comprises the structure of Formula (XII-b): 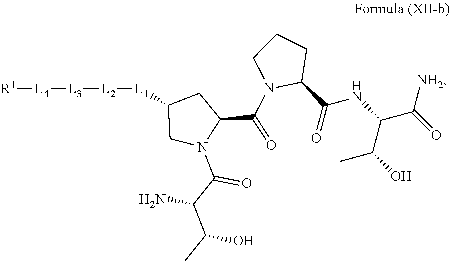
or a salt thereof.

45. The compound, or salt thereof, of claim 1, wherein the compound comprises the structure of Formula (XIII): 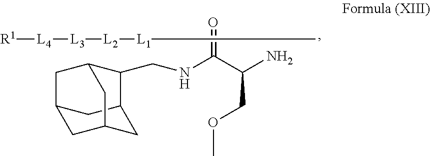
or a salt thereof.

46. The compound, or salt thereof, of claim 45, wherein the compound comprises the structure of Formula (XIII-a): 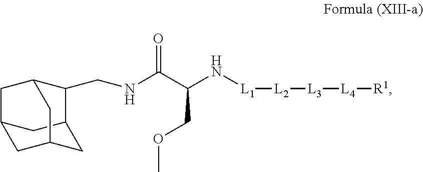
or a salt thereof.

47. The compound, or salt thereof, of claim 1, wherein the compound comprises the structure of Formula (XIV): 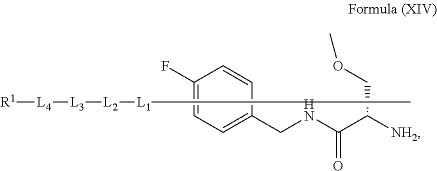
or a salt thereof.

48. The compound, or salt thereof, of claim 47, wherein the compound comprises the structure of Formula (XIV-a): 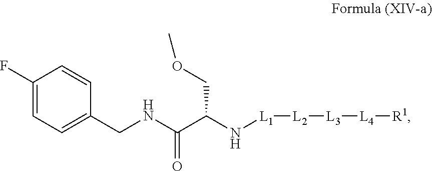
or a salt thereof.

49. The compound, or salt thereof, of claim 1, wherein the compound comprises the structure of Formula (XV): 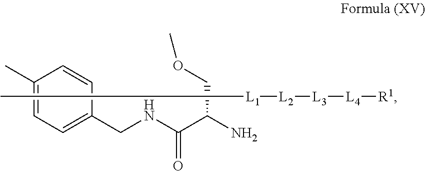
or a salt thereof.

50. The compound, or salt thereof, of claim 49, wherein the compound comprises the structure of Formula (XV-a): 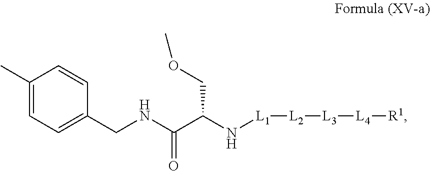
or a salt thereof.

51. The compound, or salt thereof, of claim 1, wherein the compound comprises the structure of Formula (XVI): 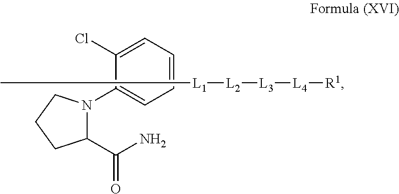
or a salt thereof.

52. The compound, or salt thereof, of claim 51, wherein the compound comprises the structure of Formula (XVI-a): 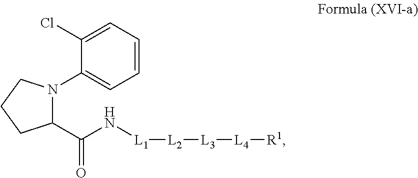
or a salt thereof.

53. The compound, or salt thereof, of claim 1, wherein the compound comprises the structure of Formula (XVII): 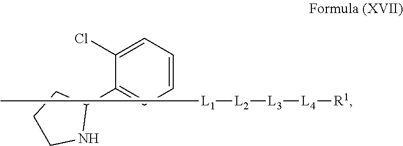
or a salt thereof.

54. The compound, or salt thereof, of claim 53, wherein the compound comprises the structure of Formula (XVII-a): 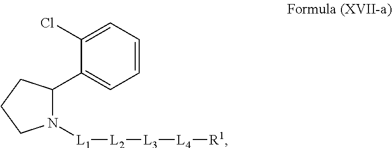
or a salt thereof.

55. The compound, or salt thereof, of claim 1, wherein the compound comprises the structure of Formula (XVIII): 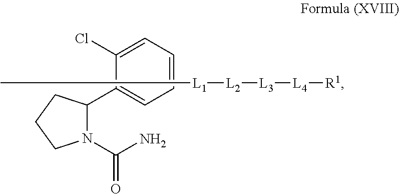
or a salt thereof.

56. The compound, or salt thereof, of claim 55, wherein the compound comprises the structure of Formula (XVIII-a): 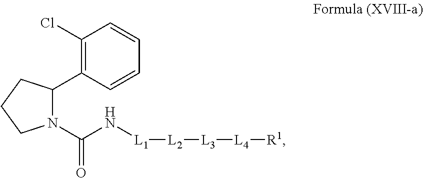
or a salt thereof.

57. The compound, or salt thereof, of any one of claims 1-56, wherein each of L₁, L₂, L₃, and L₄ is independently absent, a bond, an optionally substituted alkyl linker, an optionally substituted polyethylene glycol (PEG) linker, an optionally substituted heteroalkyl linker, an optionally substituted heteroaryl linker, a phosphodiester bond, or a phosphorothioate bond.

58. The compound, or salt thereof, of any one of claims 1-57, wherein L₁ is a bond.

59. The compound, or salt thereof, of any one of claims 1-57, wherein L₁ is an optionally substituted alkyl linker.

60. The compound, or salt thereof, of any one of claims 1-57, wherein L₁ is an optionally substituted C1-C6 alkyl linker.

61. The compound, or salt thereof, of any one of claims 1-57, wherein L₁ is a C1-C6 alkyl linker substituted with ═O.

62. The compound, or salt thereof, of any one of claims 1-57, wherein L₁ comprises the structure

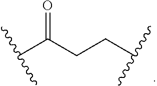 [SMILES: *CCC(*)=O]

63. The compound, or salt thereof, of any one of claims 1-62, wherein L₂ is an optionally substituted alkyl linker.

64. The compound, or salt thereof, of any one of claims 1-62, wherein L₂ is an optionally substituted C1-C15 alkyl linker.

65. The compound, or salt thereof, of any one of claims 1-62, wherein L₂ is an optionally substituted C5-C12 alkyl linker.

66. The compound, or salt thereof, of any one of claims 1-62, wherein L₂ comprises the structure

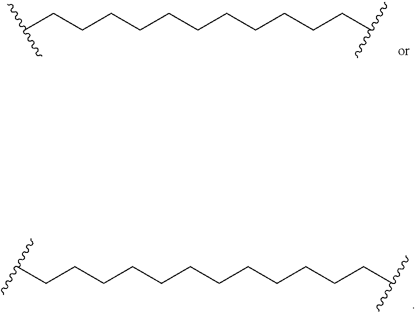 [SMILES: *CCCCCCCCCCC* ; *CCCCCCCCCCCC*]

67. The compound, or salt thereof, of any one of claims 1-62, wherein L₂ is an optionally substituted PEG linker

68. The compound, or salt thereof, of any one of claims 1-62, wherein L₂ is an optionally substituted PEG linker comprising one, two, three, four, five, six, seven, or eight PEG units in length, wherein a PEG unit comprises the structure

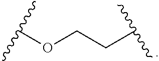 [SMILES: *CCO*]

69. The compound, or salt thereof, of any one of claims 1-62, wherein L₂ is an optionally substituted PEG linker comprising three PEG units in length, wherein a PEG unit comprises the structure

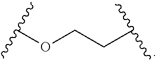 [SMILES: *CCO*]

70. The compound, or salt thereof, of any one of claims 1-62, wherein L₂ is an optionally substituted PEG linker comprising four PEG units in length, wherein a PEG unit comprises the structure

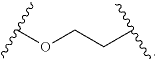 [SMILES: *CCO*]

71. The compound, or salt thereof, of any one of claims 1-62, wherein L₂ comprises the structure

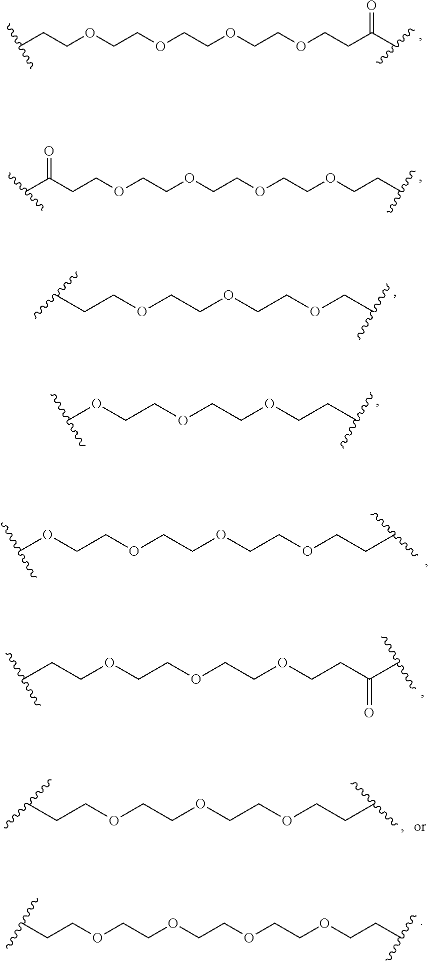

72. The compound, or salt thereof, of any one of claims 1-62, wherein L₂ is an optionally substituted heteroalkyl linker.

73. The compound, or salt thereof, of claim 72, wherein L₂ comprises the structure

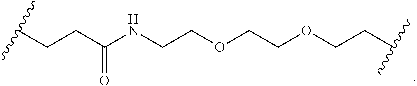 [SMILES: *CCOCCOCCNC(=O)CC*]

74. The compound, or salt thereof, of any one of claims 1-73, wherein L₃ is an optionally substituted heteroaryl linker.

75. The compound, or salt thereof, of any one of claims 1-73, wherein L₃ is an optionally substituted partially unsaturated heterocycloalkyl or optionally unsubstituted heteroaryl linker.

76. The compound, or salt thereof, of claim 74 or 75, wherein L₃ comprises the structure

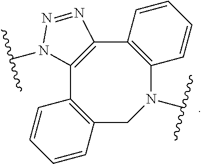 [SMILES: *N1Cc2ccccc2-c2c(nnn2*)-c2ccccc21]

77. The compound, or salt thereof, of any one of claims 1-76, wherein L₄ is an optionally substituted heteroalkyl linker.

78. The compound, or salt thereof, of claim 77, wherein the heteroalkyl linker is substituted with one or more ═O substituents.

79. The compound, or salt thereof, of claim 77 or 78, wherein L₄ comprises the structure 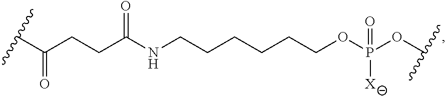
wherein X is O or S.

80. The compound, or salt thereof, of claim 77 or 78, wherein L₄ comprises the structure 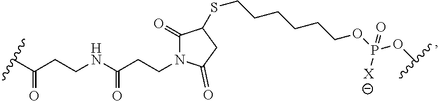
wherein X is S or O

81. The compound, or salt thereof, of any one of claims 1-80, wherein L₁, L₂, L₃, and L₄ together comprise the structure 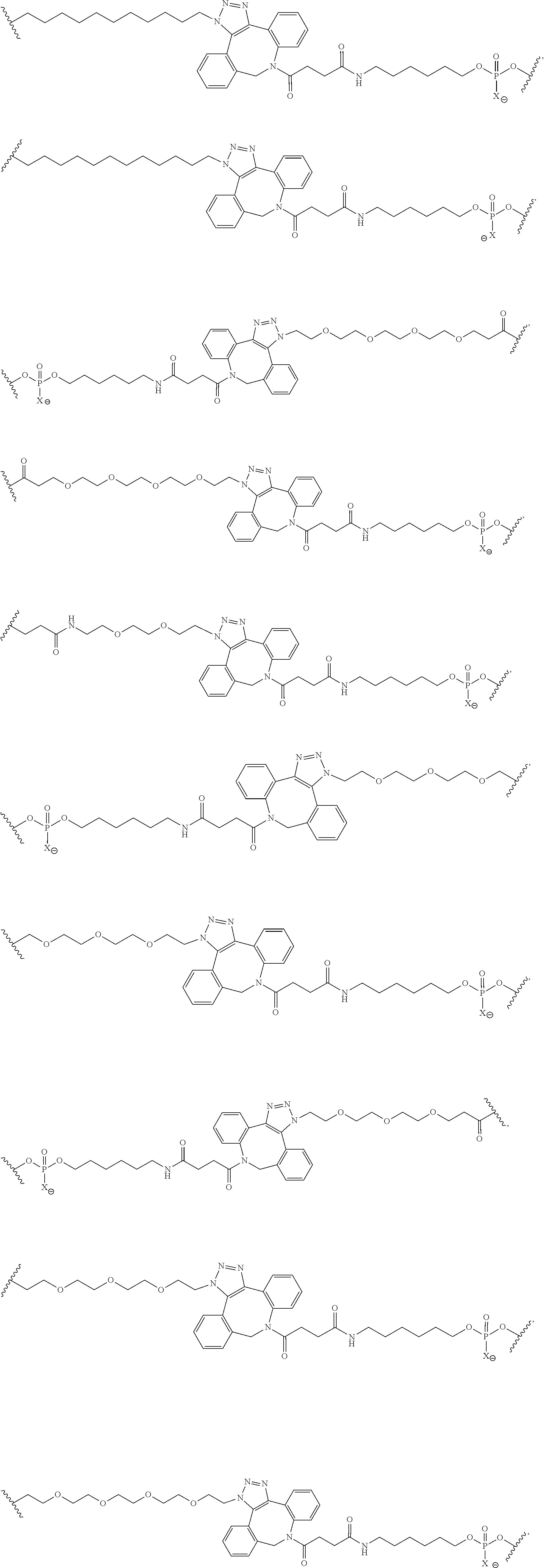 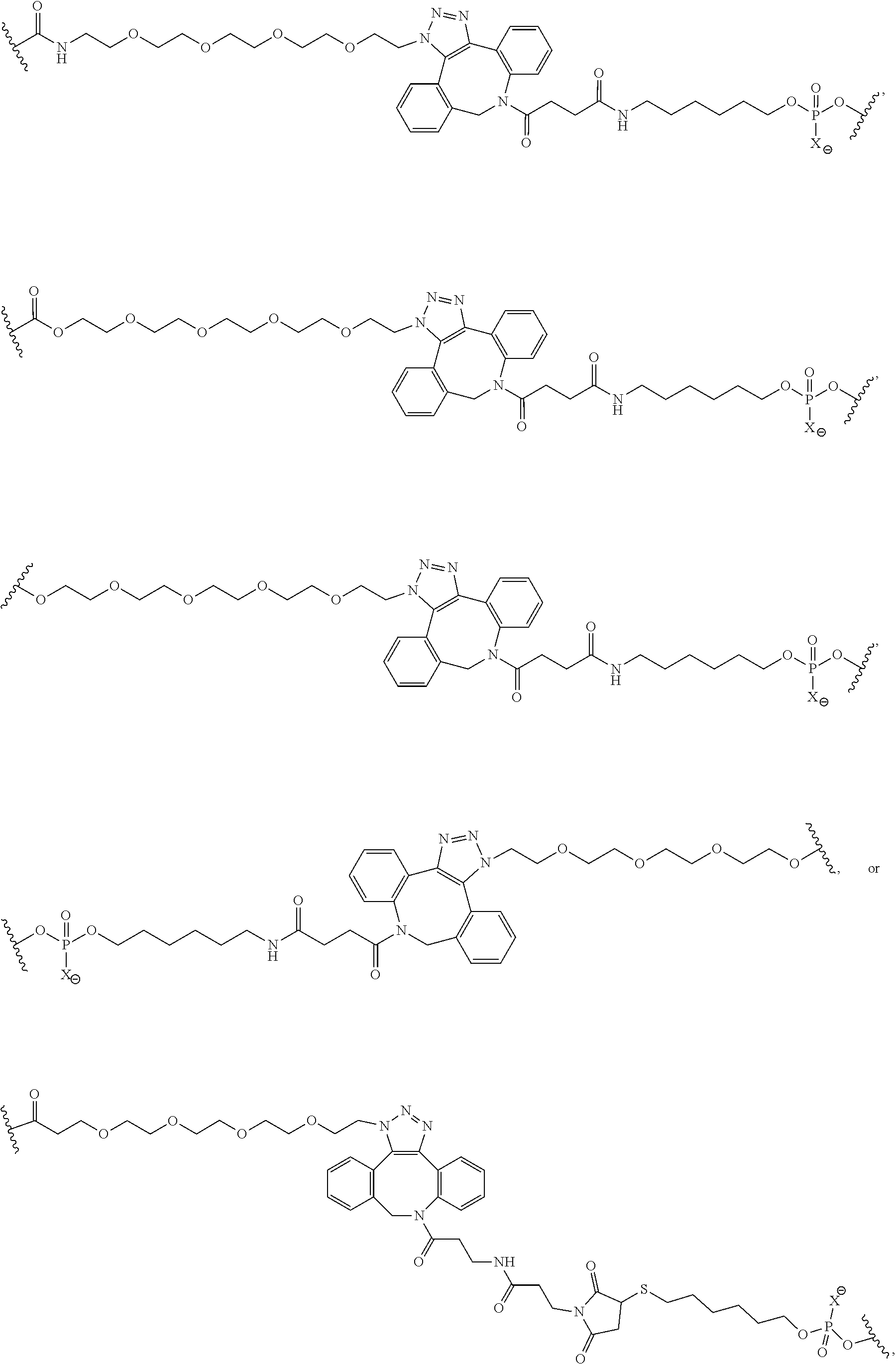
wherein X is O or S.

82. The compound, or salt thereof, of any one of claims 1-81, wherein the compound comprises the structure 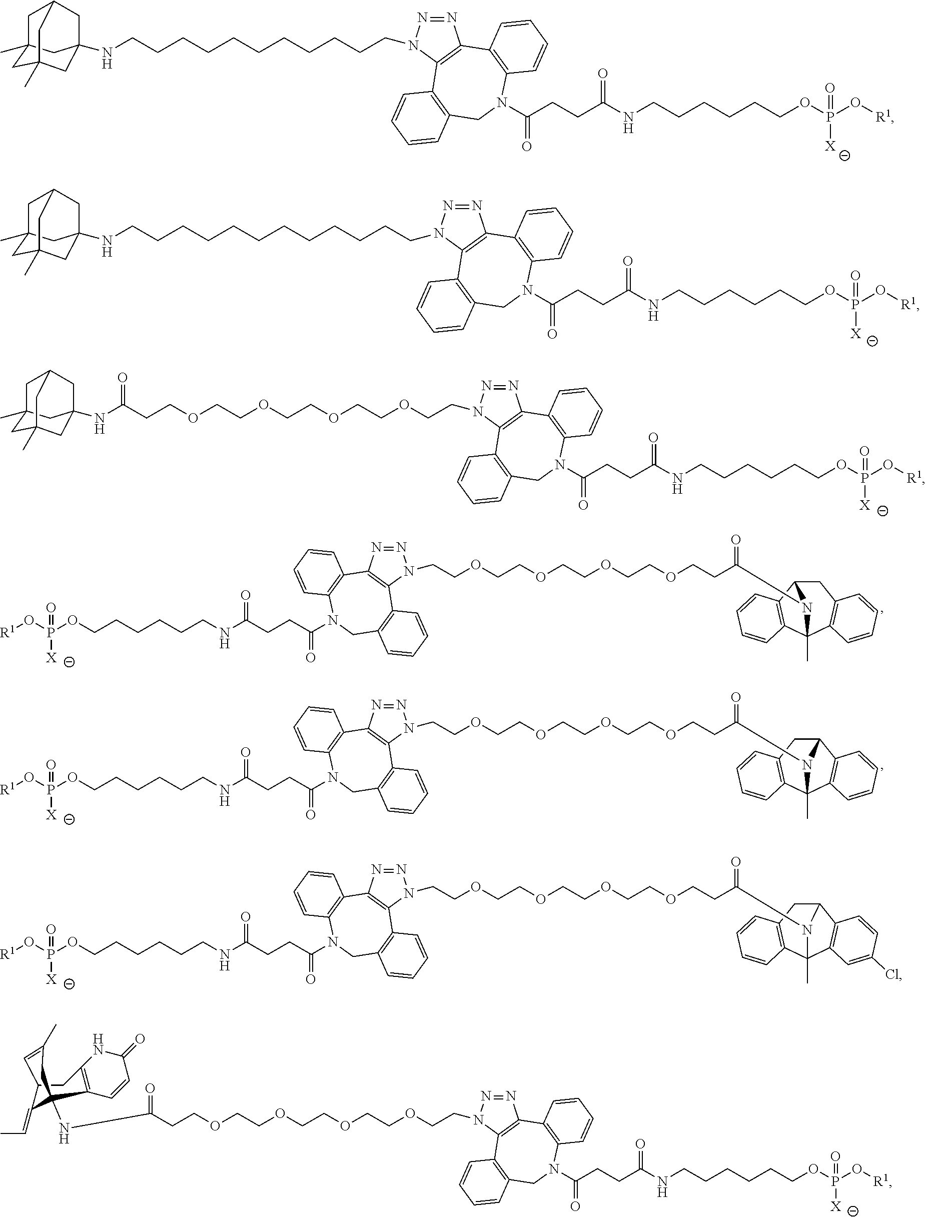 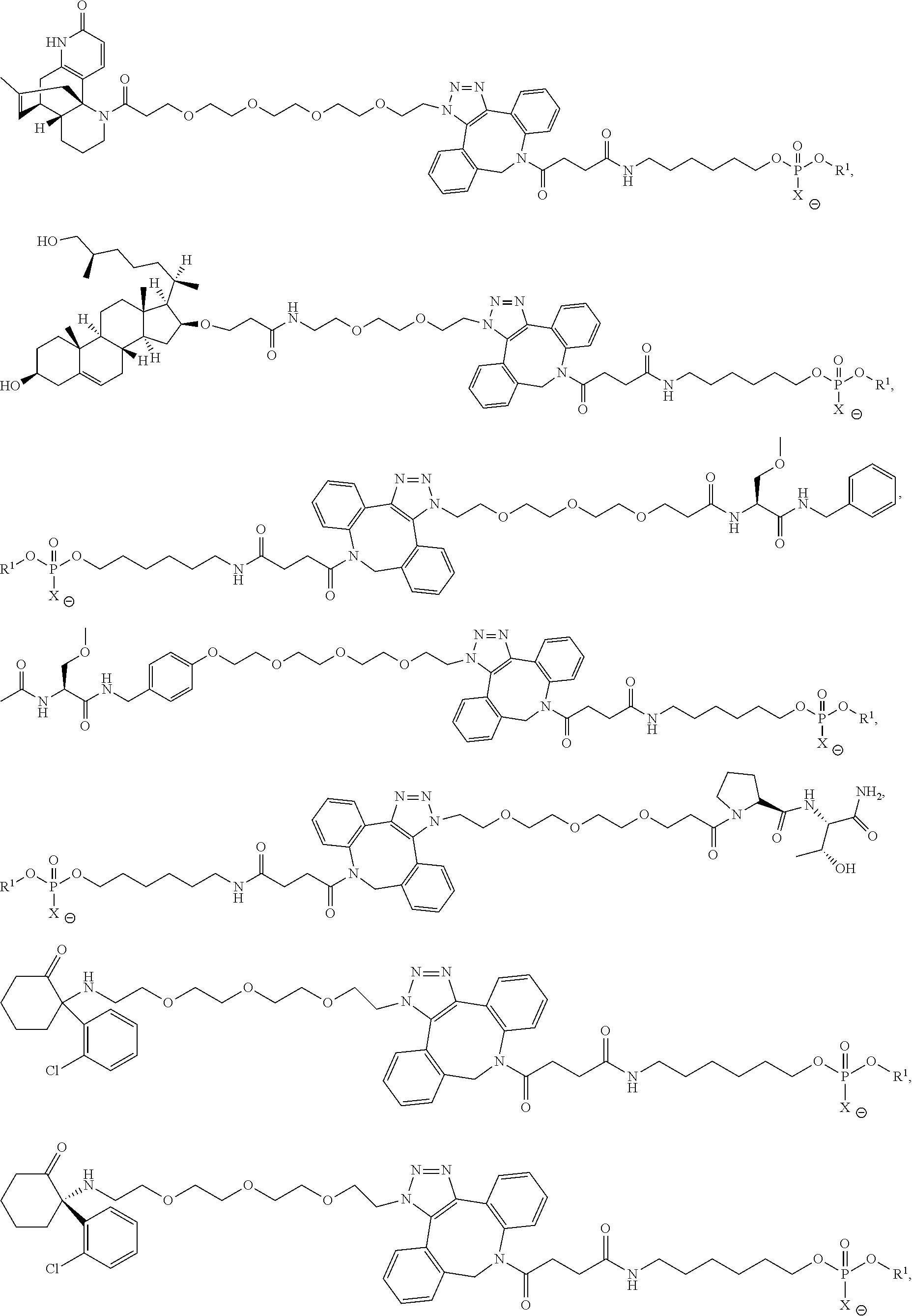 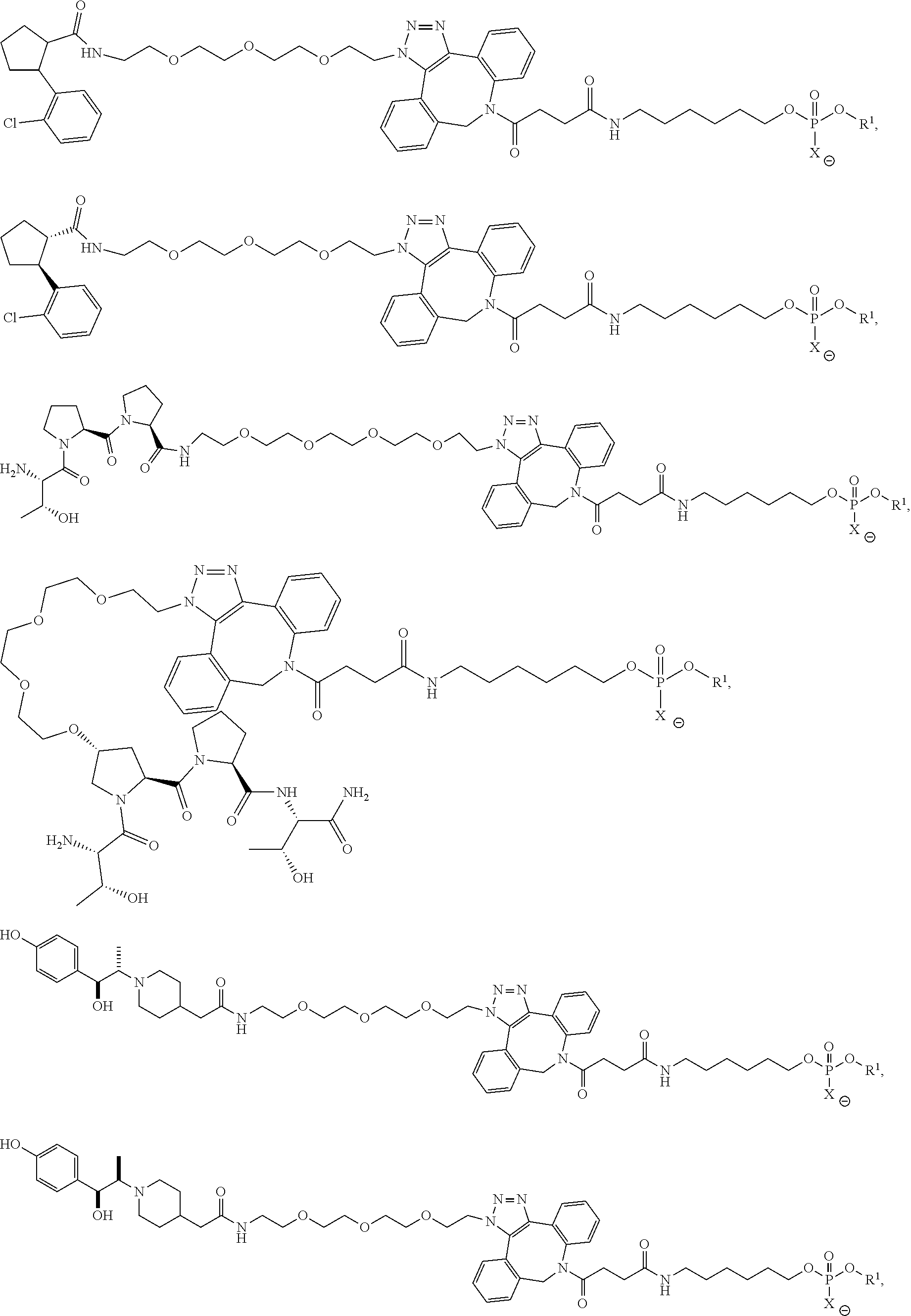 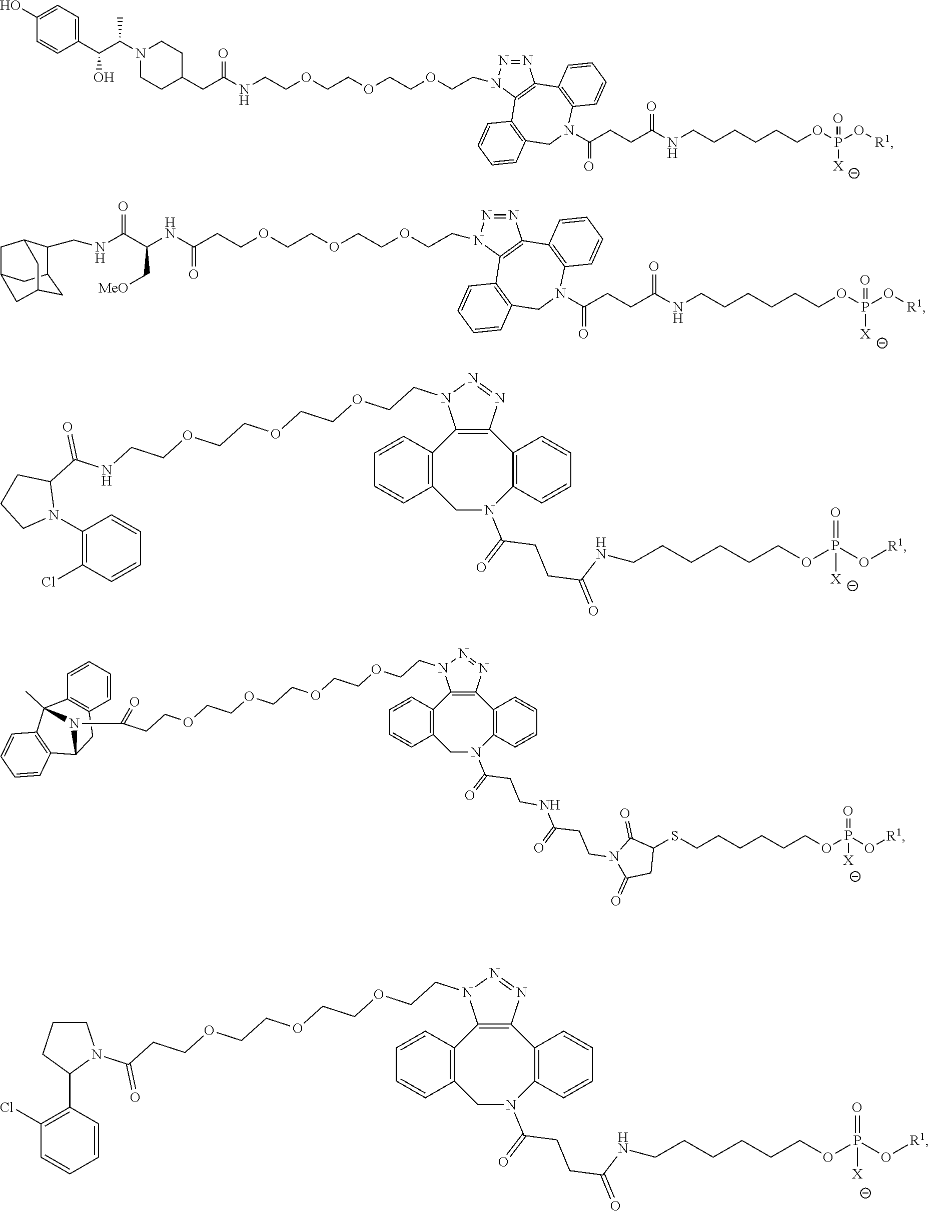 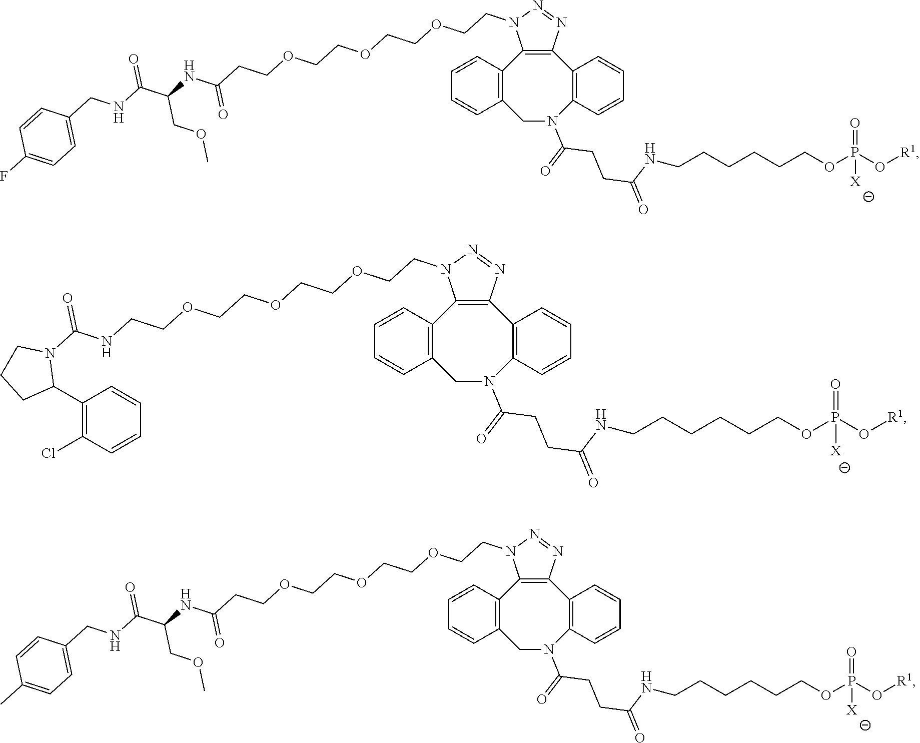
wherein X is O or S.

83. The compound, or salt thereof, of any one of claims 79-82, wherein X is O.

84. The compound, or salt thereof, of any one of claims 79-82, wherein X is S.

85. The compound, or salt thereof, of any one of claims 1-84, wherein R¹ comprises an oligonucleotide.

86. The compound, or salt thereof, of claim 85, wherein the oligonucleotide is attached at its 5′ end.

87. The compound, or salt thereof, of claim 85, wherein the oligonucleotide is attached at its 3′ end.

88. The compound, or salt thereof, of claim 85, wherein the oligonucleotide is attached at an internal position on the oligonucleotide.

89. The compound, or salt thereof, of claim 88, wherein the internal position is an internucleoside linkage.

90. The compound, or salt thereof, of any one of claims 1-89, wherein R¹ comprises an oligonucleotide conjugated to one or more additional NMDA receptor ligands.

91. The compound, or salt thereof, of claim 90, wherein the oligonucleotide is conjugated to two, three, four, five, or more than five additional NMDA receptor ligands.

92. The compound, or salt thereof, of claim 90 or 91, wherein the additional NMDA receptor ligands are conjugated to the oligonucleotide at the 5′ end of the oligonucleotide, the 3′ end of the oligonucleotide, one or more internal positions on the oligonucleotide, or any combination thereof.

93. The compound, or salt thereof, of any one of claims 85-92, wherein the oligonucleotide is a modified oligonucleotide.

94. A composition comprising a compound, or salt thereof, of any one of claims 1-93, and a pharmaceutically acceptable excipient.

95. A method for delivering a therapeutic oligonucleotide to the brain of a subject, comprising administration of a compound, or salt thereof, of any one of claims 1-93, or a composition of claim 94, to the subject.

96. The method of claim 95, wherein the therapeutic oligonucleotide is delivered to one or more brain regions selected from the group consisting of the striatum, the cerebellum, the brain stem, the hippocampus, the frontal cortex, and the spinal cord.

97. A method for treating or ameliorating a disease, disorder, or symptom thereof in a subject, comprising administration of a compound, or salt thereof, of any one of claims 1-93, or a composition of claim 94, to the subject.

98. The method of claim 97, wherein the disease, disorder, or symptom thereof is a central nervous system (CNS) disease, disorder, or symptom thereof.

99. The method of claim 97 or 98, wherein the disease, disorder, or symptom thereof is Alzheimer's disease, or a symptom thereof.

100. The method of any one of claims 97-99, wherein the compound, or salt thereof, is administered to the subject intrathecally.

101. A method for making a compound, or salt thereof, of any one of claims 1-93, comprising one or more compounds and chemical transformations described herein, including Examples 1-77.
